(19) United States
(12) Patent Application Publication
(10) Pub. No.: US 2023/0174660 A1
(43) Pub. Date: Jun. 8, 2023

(54) USE OF A MULTIMERIC ANTI-DR5 BINDING MOLECULE IN COMBINATION WITH A CANCER THERAPY FOR TREATING CANCER

(71) Applicant: IGM Biosciences, Inc., Mountain View, CA (US)

(72) Inventors: Beatrice WANG, Sunnyvale, CA (US); Thomas J. MATTHEW, San Jose, CA (US); Eric William HUMKE, Redwood City, CA (US); Angus SINCLAIR, San Francisco, CA (US); Daniel S. CHEN, Burlingame, CA (US); Bruce Alan KEYT, Hillsborough, CA (US); Ling WANG, San Jose, CA (US)

(21) Appl. No.: 17/998,307

(22) PCT Filed: May 12, 2021
(86) PCT No.: PCT/US2021/032078

§ 371 Date: Nov. 9, 2022

Related U.S. Application Data
(60) Provisional application No. 63/023,635, filed on May 12, 2020.
(60) Provisional application No. 63/078,747, filed on Sep. 15, 2020.
(60) Provisional application No. 63/114,990, filed on Nov. 17, 2020.
(60) Provisional application No. 63/131,698, filed on Dec. 29, 2020.
(60) Provisional application No. 63/136,156, filed on Jan. 11, 2021.

(51) Int. Cl. C07K 16/28; A61P 35/00

(52) U.S. Cl. CPC C07K 16/2878; A61P 35/00; C07K 2317/24; C07K 2317/565

(57) ABSTRACT

This disclosure provides therapeutic methods for treating cancer including combination therapy with a multimeric anti-DR5 antibody and a cancer therapy, e.g., radiation, an anthracycline, a folic acid analog, a platinum-based agent, a taxane, a topoisomerase II inhibitor, a SMAC mimetic, a vinca alkaloid, a Brutons tyrosine kinase (BTK) inhibitor, a phosphoinositide 3-kinase delta (PI3Kδ) inhibitor, a myeloid cell leukemia-1 (Mcl-1) inhibitor, or any combination thereof.

### CROSS-REFERENCE TO RELATED APPLICATIONS

This application claims the benefit of U.S. Provisional Patent Application Serial Nos. 63/023,635, filed May 12, 2020; 63/078,747, filed Sep. 15, 2020; 63/114,990, filed Nov. 17, 2020, 63/131,698, filed Dec. 29, 2020 and 63/136,156, filed Jan. 11, 2021, which are each incorporated herein by reference in their entireties.

### SEQUENCE LISTING

The instant application contains a Sequence Listing which has been submitted electronically in ASCII format and is hereby incorporated by reference in its entirety. The ASCII copy was created on May 11, 2021, is named 030WO1-Sequence-Listing, and is 139,726 bytes in size.

### BACKGROUND

Antibodies and antibody-like molecules that can multimerize, such as IgA and IgM antibodies, have emerged as promising drug candidates in the fields of, e.g., immuno-oncology and infectious diseases allowing for improved specificity. improved avidity, and the ability to bind to multiple binding targets. See, e.g., U.S. Pat. Nos. 9,951,134, 9,938,347, 10,351,631, and 10,400,038, U.S. Patent Application Publication Nos. US 2019-0100597, US 2018-0009897, US 2019-0330374, US 2019-0330360, US 2019-0338040, US 2019-0338041. US 2019-0185570, US 2018-0265596, US 2018-0118816, US 2018-0118814, and US 2019-0002566, and PCT Publication Nos. WO 2018/187702, WO 2019/165340, and WO 2019/169314, the contents of which are incorporated herein by reference in their entireties.

Multimeric IgA or IgM antibodies present a useful tool for application to specific biological systems in which multiple components necessarily must be bound simultaneously to transmit biological signals. For instance, many receptor proteins on the surface of eukaryotic cells require the simultaneous activation of multiple monomers or subunits to achieve activation and transmission of a biological signal across a cell membrane, to the cytoplasm of the cell.

One such receptor is the apoptosis-inducing Tumor Necrosis Factor (TNF) receptor superfamily proteins DR5 (also referred to as TRAILR2). DR5 activation requires that at least three non-interacting receptor monomers be cross-linked, e.g., by a TRAIL ligand or agonist antibody, to form a stabilized receptor trimer, resulting in signal transduction across the cell membrane. Clustering of DR5 protein trimers into “rafts” of trimers can lead to more effective activation the signaling cascade.

Interest-in DR5 is heightened due to the finding that it is expressed in bladder cancer (Li et al., Urology, 79(4):968.e7-15, (2012)), gastric cancer (Lim et al., Carcinogen., 32(5):723-732, (2011)), ovarian cancer (Jiang et al., Mol. Med. Rep., 6(2):316-320, (2012)), pancreatic ductal adenocarcinoma (Rajeshkumar et al., Mol. Cancer Ther., 9(9):2583-92, (2010)), oral squamous cell carcinoma (Chen et al. Oncotarget 4:206-217, (2013)) and non-small cell lung cancer (Reck et al., Lung Canc., 82(3):441-448, (2013)). The current standard of care for certain of these cancers includes radiation or chemotherapeutic agents that disrupt cellular growth and metabolism, e.g., by blocking DNA synthesis, blocking cell division, or promoting apoptosis.

While certain anti-DR5 monoclonal antibodies, such as Tigatuzumab (CS-1008, Daiichi Sankyo Co. Ltd., disclosed in U.S. Pat. No. 7,244,429), have been found to be effective in vitro and in vivo even without additional cross-linkers added, these antibodies have not resulted in significant clinical efficacy. (See, Reck et al., 2013). More recently though, several different anti-DR5 IgM antibodies have been shown to have much higher efficacy both in vitro and in vivo. See, e.g., U.S. Patent Appl. Publication No. 2018-0009897, which is incorporated herein by reference in its entirety.

Better therapies and enhancements to existing therapies for difficult to treat tumors are needed, including combination therapies with anti-DR5 IgM antibodies.

### SUMMARY

Provided herein is a method for inhibiting, delaying, or reducing malignant cell growth in a subject with cancer, comprising administering to a subject in need of treatment a combination therapy comprising: (a) an effective amount of a dimeric IgA or IgA-like antibody or a hexameric or pentameric IgM or IgM-like antibody, or a multimerized antigen-binding fragment, variant, or derivative thereof that specifically and agonistically binds to DR5, wherein three or four of the antigen binding domains of the IgA or IgA-like antibody or multimerized antigen-binding fragment, variant, or derivative thereof or three to twelve of the antigen binding domains of the IgM or IgM-like antibody or multimerized antigen-binding fragment, variant, or derivative thereof are DR5-specific and agonistic; and (b) an effective amount of a cancer therapy, wherein the cancer therapy comprises radiation, a folic acid analog, a platinum-based agent, a taxane, a topoisomerase II inhibitor, second mitochondria-derived activator of caspases (SMAC) mimetic, a vinca alkaloid, a Bruton's tyrosine kinase (BTK) inhibitor, a phosphoinositide 3-kinase delta (PI3Kδ) inhibitor, a myeloid cell leukemia-1 (Mcl-1) inhibitor, an anti-VEGF antibody, or any combination thereof.

Provided herein is a method for inhibiting, delaying, or reducing malignant cell growth in a subject with cancer in need of treatment, comprising administering an effective amount of a pentameric or hexameric IgM or IgM-like antibody or a dimeric IgA or IgA-like antibody, or a multimerized antigen-binding fragment, variant, or derivative thereof that specifically and agonistically binds to DR5, where three to twelve of the antigen binding domains of the IgM or IgM-like antibody or multimerized antigen-binding fragment, variant, or derivative thereof or three or four of the antigen binding domains of the IgA or IgA-like antibody or multimerized antigen-binding fragment, variant, or derivative thereof are DR5-specific and agonistic, where the pentameric or hexameric IgM or IgM-like antibody or the dimeric IgA or IgA-like antibody, or the multimerized antigen-binding fragment, variant, or derivative thereof is administered with an effective amount of a cancer therapy, where the cancer therapy comprises a second mitochondria-derived activator of caspases (SMAC) mimetic, radiation, a folic acid analog, a platinum-based agent, a taxane, a topoisomerase II inhibitor, a vinca alkaloid, a Bruton's tyrosine kinase (BTK) inhibitor, a phosphoinositide 3-kinase delta (PI3Kδ) inhibitor, a myeloid cell leukemia-1 (Mcl-1) inhibitor, an anti-VEGF antibody, or any combination thereof.

Provided herein is a method for inhibiting, delaying, or reducing malignant cell growth in a subject with cancer in need of treatment, comprising administering an effective amount of a cancer therapy, where the cancer therapy comprises a second mitochondria-derived activator of caspases (SMAC) mimetic, radiation, a folic acid analog, a platinum-based agent, a taxane, a topoisomerase II inhibitor, a vinca alkaloid, a Bruton's tyrosine kinase (BTK) inhibitor, a phosphoinositide 3-kinase delta (PI3Kδ) inhibitor, a myeloid cell leukemia-1 (Mcl-1) inhibitor, an anti-VEGF antibody, or any combination thereof, where the cancer therapy is administered with a pentameric or hexameric IgM or IgM-like antibody or a dimeric IgA or IgA-like antibody, or a multimerized antigen-binding fragment, variant, or derivative thereof that specifically and agonistically binds to DR5, where three to twelve of the antigen binding domains of the IgM or IgM-like antibody or multimerized antigen-binding fragment, variant, or derivative thereof or three or four of the antigen binding domains of the IgA or IgA-like antibody or multimerized antigen-binding fragment, variant, or derivative thereof are DR5-specific and agonistic.

Provided herein is a method for inducing apoptosis in a cancer cell in in a subject with cancer in need of treatment, comprising administering to the subject a combination therapy comprising: (a) an effective amount of a pentameric or hexameric IgM or IgM-like antibody or a dimeric IgA or IgA-like antibody, or a multimerized antigen-binding fragment, variant, or derivative thereof that specifically and agonistically binds to DR5, where three to twelve of the antigen binding domains of the IgM or IgM-like antibody or multimerized antigen-binding fragment, variant, or derivative thereof or three or four of the antigen binding domains of the IgA or IgA-like antibody or multimerized antigen-binding fragment, variant, or derivative thereof are DR5-specific and agonistic; and (b) an effective amount of a cancer therapy, where the cancer therapy comprises a second mitochondria-derived activator of caspases (SMAC) mimetic, radiation, a folic acid analog, a platinum-based agent, a taxane, a topoisomerase II inhibitor, a vinca alkaloid, a Bruton's tyrosine kinase (BTK) inhibitor, a phosphoinositide 3-kinase delta (PI3Kδ) inhibitor, a myeloid cell leukemia-1 (Mcl-1) inhibitor, an anti-VEGF antibody, or any combination thereof.

Provided herein is a method for inhibiting, delaying, or reducing malignant cell growth in a subject with cancer in need of treatment, comprising administering an effective amount of a pentameric or hexameric IgM or IgM-like antibody or a dimeric IgA or IgA-like antibody, or a multimerized antigen-binding fragment, variant, or derivative thereof that specifically and agonistically binds to DR5, where three to twelve of the antigen binding domains of the IgM or IgM-like antibody or multimerized antigen-binding fragment, variant, or derivative thereof or three or four of the antigen binding domains of the IgA or IgA-like antibody or multimerized antigen-binding fragment, variant, or derivative thereof are DR5-specific and agonistic, where the pentameric or hexameric IgM or IgM-like antibody or the dimeric IgA or IgA-like antibody, or the multimerized antigen-binding fragment, variant, or derivative thereof is administered with an effective amount of a cancer therapy, where the cancer therapy comprises a second mitochondria-derived activator of caspases (SMAC) mimetic, radiation, a folic acid analog, a platinum-based agent, a taxane, a topoisomerase II inhibitor, a vinca alkaloid, a Bruton's tyrosine kinase (BTK) inhibitor, a phosphoinositide 3-kinase delta (PI3Kδ) inhibitor, a myeloid cell leukemia-1 (Mcl-1) inhibitor, an anti-VEGF antibody, or any combination thereof.

Provided herein is a method for inducing apoptosis in a cancer cell in in a subject with cancer in need of treatment, comprising administering an effective amount of an effective amount of a cancer therapy, where the cancer therapy comprises a second mitochondria-derived activator of caspases (SMAC) mimetic, radiation, a folic acid analog, a platinum-based agent, a taxane, a topoisomerase II inhibitor, a vinca alkaloid, a Bruton's tyrosine kinase (BTK) inhibitor, a phosphoinositide 3-kinase delta (PI3Kδ) inhibitor, a myeloid cell leukemia-1 (Mcl-1) inhibitor, an anti-VEGF antibody, or any combination thereof, where the cancer therapy is administered with a pentameric or hexameric IgM or IgM-like antibody or a dimeric IgA or IgA-like antibody, or a multimerized antigen-binding fragment, variant, or derivative thereof that specifically and agonistically binds to DR5, where three to twelve of the antigen binding domains of the IgM or IgM-like antibody or multimerized antigen-binding fragment, variant, or derivative thereof or three or four of the antigen binding domains of the IgA or IgA-like antibody or multimerized antigen-binding fragment, variant, or derivative thereof are DR5-specific and agonistic.

In some embodiments, the cancer therapy comprises a folic acid analog. In some embodiments, the folic acid analog comprises leucovorin.

In some embodiments, the cancer therapy comprises a platinum-based agent. In some embodiments, the platinum-based agent comprises oxaliplatin, carboplatin, or a combination thereof. In some embodiments, the platinum-based agent comprises oxaliplatin. In some embodiments, the platinum-based agent comprises carboplatin.

In some embodiments, the cancer therapy comprises a taxane. In some embodiments, the taxane comprises paclitaxel. In some embodiments, the paclitaxel comprises solvent-based paclitaxel, nab-paclitaxel, or a combination thereof. In some embodiments, the paclitaxel comprises solvent-based paclitaxel. In some embodiments, the paclitaxel comprises nab-paclitaxel.

In some embodiments, the cancer therapy comprises a topoisomerase II inhibitor. In some embodiments, the topoisomerase II inhibitor comprises an anthracycline. In some embodiments, the anthracycline comprises doxorubicin. In some embodiments, the topoisomerase II inhibitor comprises etoposide.

In some embodiments, the cancer therapy comprises a SMAC mimetic. In some embodiments, the SMAC mimetic comprises birinapant, GDC-0152, HGS-1029/AEG40826, Debio1143, APG-1387, ASTX660, or a combination thereof. In some embodiments, the SMAC mimetic comprises a bivalent SMAC mimetic. In some embodiments, the SMAC mimetic comprises birinapant. In some embodiments, the SMAC mimetic comprises APG-1387. In some embodiments, the SMAC mimetic comprises GDC-0152. In some embodiments, the SMAC mimetic comprises HGS-1029/AEG40826. In some embodiments, the SMAC mimetic comprises Debio1143. In some embodiments, the SMAC mimetic comprises ASTX660. In some embodiments, the SMAC mimetic comprises a monovalent SMAC mimetic.

In some embodiments, the cancer therapy comprises a vinca alkaloid. In some embodiments, the vinca alkaloid comprises vincristine.

In some embodiments, the cancer therapy comprises a BTK inhibitor. In some embodiments, the BTK inhibitor comprises ibrutinib.

In some embodiments, the cancer therapy comprises a PI3Kδ inhibitor. In some embodiments, the PI3Kδ inhibitor comprises idelalisib.

In some embodiments, the cancer therapy comprises a Mcl-1 inhibitor. In some embodiments, the Mcl-1 inhibitor comprises MIK665.

In some embodiments, the cancer therapy comprises an anti-VEGF antibody. In some embodiments, the anti-VEGF antibody is bevacizumab.

In some embodiments, the cancer therapy comprises radiation.

In some embodiments, the method further comprises administering an effective amount of an additional cancer therapy. In some embodiments, the additional cancer therapy comprises a topoisomerase I inhibitor, a nucleoside analog, a platinum-based agent, or any combination thereof. In some embodiments, the additional cancer therapy comprises a topoisomerase I inhibitor. In some embodiments, the topoisomerase I inhibitor comprises irinotecan, topotecan, or a combination thereof. In some embodiments, the topoisomerase I inhibitor comprises irinotecan. In some embodiments, the additional cancer therapy comprises a nucleoside analog. In some embodiments, the nucleoside analog comprises fluorouracil (5-FU), gemcitabine, or any combination thereof. In some embodiments, the nucleoside analog comprises fluorouracil (5-FU). In some embodiments, the nucleoside analog comprises gemcitabine.

In some embodiments, the cancer is a hematologic cancer or a solid tumor. In some embodiments, the cancer is a hematologic cancer. In some embodiments, the hematologic cancer is leukemia, lymphoma, myeloma, any metastases thereof, or any combination thereof. In some embodiments, the hematologic cancer is acute myeloid leukemia (AML), chronic myeloid leukemia (CML), acute lymphocytic leukemia (ALL), small lymphocytic lymphoma (SLL), chronic lymphocytic leukemia, hairy cell leukemia, Hodgkin lymphoma, non-Hodgkin lymphoma, multiple myeloma, any metastases thereof, or any combination thereof. In some embodiments, the hematologic cancer is acute myeloid leukemia (AML). In some embodiments, the cancer therapy comprises doxorubicin.

In some embodiments, the cancer is a solid tumor. In some embodiments, the cancer is bladder cancer, colorectal cancer, sarcoma, gastric cancer, lung cancer, pancreatic cancer, melanoma, ovarian cancer, head and neck cancer, or breast cancer.

In some embodiments, the cancer is sarcoma. In some embodiments, the sarcoma is fibrosarcoma, chondrosarcoma, or osteosarcoma. In some embodiments, the sarcoma is fibrosarcoma. In some embodiments, the cancer therapy comprises doxorubicin.

In some embodiments, the cancer is colorectal cancer. In some embodiments, the cancer therapy comprises oxaliplatin. In some embodiments, the additional therapy comprises 5-FU. In some embodiments, the cancer therapy comprises leucovorin. In some embodiments, the additional therapy comprises oxaliplatin or irinotecan.

In some embodiments, the cancer is gastric cancer. In some embodiments, the cancer therapy comprises carboplatin. In some embodiments, the cancer therapy comprises oxaliplatin. In some embodiments, the cancer therapy comprises paclitaxel.

In some embodiments, the cancer is lung cancer. In some embodiments, the lung cancer is non-small cell lung cancer (NSCLC). In some embodiments, the cancer therapy comprises carboplatin. In some embodiments, the cancer therapy comprises paclitaxel.

In some embodiments, the cancer is pancreatic cancer. In some embodiments, the cancer therapy comprises paclitaxel. In some embodiments, the additional therapy comprises gemcitabine.

In some embodiments, the cancer is head and neck cancer. In some embodiments, the head and neck cancer is head and neck sarcoma. In some embodiments, the cancer is breast cancer. In some embodiments, the breast cancer is triple negative breast cancer (TNBC). In some embodiments, the cancer therapy comprises a SMAC mimetic.

In some embodiments, the three or four antigen-binding domains or the three to twelve antigen-binding domains of the antibody or multimerized antigen-binding fragment, variant, or derivative thereof comprise a heavy chain variable region (VH) and a light chain variable region (VL), wherein the VH and VL comprise six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3 comprise the CDRs of an antibody comprising the VH and VL amino acid sequences SEQ ID NO: 1 and SEQ ID NO: 2; SEQ ID NO: 3 and SEQ ID NO: 4; SEQ ID NO: 5 or SEQ ID NO: 90 and SEQ ID NO: 6; SEQ ID NO: 7 and SEQ ID NO: 8; SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; SEQ ID NO: 23 and SEQ ID NO: 24; SEQ ID NO: 25 and SEQ ID NO: 26; SEQ ID NO: 27 and SEQ ID NO: 28; SEQ ID NO: 29 and SEQ ID NO: 30; SEQ ID NO: 31 and SEQ ID NO: 32; SEQ ID NO: 33 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46. SEQ ID NO: 47 and SEQ ID NO: 48; SEQ ID NO: 49 and SEQ ID NO: 50; SEQ ID NO: 51 and SEQ ID NO: 52; SEQ ID NO: 53 and SEQ ID NO: 54; SEQ ID NO: 55 and SEQ ID NO: 56; SEQ ID NO: 82 and SEQ ID NO: 83; SEQ ID NO: 84 and SEQ ID NO: 85; SEQ ID NO: 86 and SEQ ID NO: 87; or SEQ ID NO: 88 and SEQ ID NO: 89; respectively, or the ScFv sequence SEQ ID NO: 57, SEQ ID NO: 58, SEQ ID NO: 59, SEQ ID NO: 60, SEQ ID NO: 61, SEQ ID NO: 62. SEQ ID NO: 63. SEQ ID NO: 64. SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, or SEQ ID NO: 73 or the six CDRs with one or two amino acid substitutions in one or more of the CDRs.

In some embodiments, the three or four antigen-binding domains or the three to twelve antigen-binding domains of the antibody or multimerized antigen-binding fragment, variant, or derivative thereof comprise a heavy chain variable region (VH) and a light chain variable region (VL), wherein the VH and VL comprise six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3 comprise the CDRs of an antibody comprising the VH and VL amino acid sequences SEQ ID NO: 5 or SEQ ID NO: 90 and SEQ ID NO: 6; or SEQ ID NO: 7 and SEQ ID NO: 8, respectively.

In some embodiments, the three or four antigen-binding domains or the three to twelve antigen-binding domains of the antibody or multimerized antigen-binding fragment, variant, or derivative thereof comprise a heavy chain variable region (VH) and a light chain variable region (VL), wherein the VH and VL comprise six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3 comprise the CDRs of an antibody comprising the VH and VL amino acid sequences SEQ ID NO: 5 or SEQ ID NO: 90 and SEQ ID NO: 6, respectively. In some embodiments, the three or four antigen-binding domains or the three to twelve antigen-binding domains of the antibody or multimerized antigen-binding fragment, variant, or derivative thereof comprise a heavy chain variable region (VH) and a light chain variable region (VL), wherein the VH and VL comprise six immunoglobulin complementarity determining regions HCDR1, HCDR2, HCDR3, LCDR1, LCDR2, and LCDR3, wherein the HCDR1, HCDR2. HCDR3, LCDR1, LCDR2, and LCDR3 comprise the CDRs of an antibody comprising the VH and VL amino acid sequences SEQ ID NO: 7 and SEQ ID NO: 8, respectively.

In some embodiments, the three or four antigen-binding domains or the three to twelve antigen-binding domains of the antibody or multimerized antigen-binding fragment, variant, or derivative thereof comprise an antibody VH and a VL, wherein the VH and VL comprise amino acid sequences at least 90% identical to SEQ ID NO: 1 and SEQ ID NO: 2; SEQ ID NO: 3 and SEQ ID NO: 4; SEQ ID NO: 5 or SEQ ID NO: 90 and SEQ ID NO: 6; SEQ ID NO: 7 and SEQ ID NO: 8; SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; SEQ ID NO: 23 and SEQ ID NO: 24; SEQ ID NO: 25 and SEQ ID NO: 26; SEQ ID NO: 27 and SEQ ID NO: 28; SEQ ID NO: 29 and SEQ ID NO: 30; SEQ ID NO: 31 and SEQ ID NO: 32; SEQ ID NO: 33 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46; SEQ ID NO: 47 and SEQ ID NO: 48; SEQ ID NO: 49 and SEQ ID NO: 50; SEQ ID NO: 51 and SEQ ID NO: 52: SEQ ID NO: 53 and SEQ ID NO: 54; SEQ ID NO: 55 and SEQ ID NO: 56; SEQ ID NO: 82 and SEQ ID NO: 83; SEQ ID NO: 84 and SEQ ID NO: 85; SEQ ID NO: 86 and SEQ ID NO: 87; or SEQ ID NO: 88 and SEQ ID NO: 89; respectively, or wherein the VH and VL are contained in an ScFv with an amino acid sequence at least 90% identical to SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, or SEQ ID NO: 73, respectively.

In some embodiments, the three or four antigen-binding domains or the three to twelve antigen-binding domains of the antibody or multimerized antigen-binding fragment, variant, or derivative thereof comprise an antibody VH and a VL, wherein the VH and VL comprise amino acid sequences at least 90% identical to SEQ ID NO: 5 or SEQ ID NO: 90 and SEQ ID NO: 6; or SEQ ID NO: 7 and SEQ ID NO: 8, respectively. In some embodiments, the three or four antigen-binding domains or the three to twelve antigen-binding domains of the antibody or multimerized antigen-binding fragment, variant, or derivative thereof comprise an antibody VH and a VL, wherein the VH and VL comprise amino acid sequences at least 90% identical to SEQ ID NO: 5 or SEQ ID NO: 90 and SEQ ID NO: 6, respectively. In some embodiments, the three or four antigen-binding domains or the three to twelve antigen-binding domains of the antibody or multimerized antigen-binding fragment, variant, or derivative thereof comprise an antibody VH and a VL, wherein the VH and VL comprise amino acid sequences at least 90% identical to SEQ ID NO: 7 and SEQ ID NO: 8, respectively.

In some embodiments, the antibody or multimerized antigen-binding fragment, variant, or derivative thereof is a dimeric IgA or IgA-like antibody comprising two bivalent IgA binding units or multimerizing fragments thereof and a J-chain or fragment or variant thereof, wherein each binding unit comprises two IgA heavy chain constant regions or multimerizing fragments thereof each associated with an antigen-binding domain. In some embodiments, the IgA or IgA-like antibody or multimerized antigen-binding fragment, variant, or derivative thereof further comprises a secretory component, or fragment or variant thereof. In some embodiments, the IgA heavy chain constant regions or multimerizing fragments thereof each comprise a Cα3-tp domain. In some embodiments, the IgA heavy chain constant regions or multimerizing fragments thereof each comprise a Cα1 domain and/or a Cα2 domain. In some embodiments, the IgA heavy chain constant region is a human IgA constant region. In some embodiments, each binding unit comprises two IgA heavy chains each comprising a VH situated amino terminal to the IgA constant region or multimerizing fragment thereof, and two immunoglobulin light chains each comprising a VL situated amino terminal to an immunoglobulin light chain constant region.

In some embodiments, the antibody or multimerized antigen-binding fragment, variant, or derivative thereof is a pentameric or a hexameric IgM antibody comprising five or six bivalent IgM binding units, respectively, wherein each binding unit comprises two IgM heavy chain constant regions or multimerizing fragments thereof each associated with an antigen-binding domain. In some embodiments, the IgM heavy chain constant regions or multimerizing fragments thereof each comprise a Cμ4-tp domain. In some embodiments, the IgM heavy chain constant regions or multimerizing fragments thereof each comprise a Cμ1 domain, a Cμ2 domain, and/or a Cμ3 domain. In some embodiments, the antibody or multimerized antigen-binding fragment, variant, or derivative thereof is pentameric, and further comprises a J-chain, or functional fragment thereof, or variant thereof. In some embodiments, the IgM heavy chain constant region is a human IgM constant region. In some embodiments, each binding unit comprises two IgM heavy chains each comprising a VH situated amino terminal to the IgM constant region or multimerizing fragment thereof, and two immunoglobulin light chains each comprising a VL situated amino terminal to an immunoglobulin light chain constant region.

In some embodiments, the J-chain or functional fragment or variant thereof is a variant J-chain comprising one or more single amino acid substitutions, deletions, or insertions relative to a wild-type J-chain that can affect serum half-life of the multimeric binding molecule, and wherein the multimeric binding molecule exhibits an increased serum half-life upon administration to an animal relative to a reference multimeric binding molecule that is identical except for the one or more single amino acid substitutions, deletions, or insertions, and is administered in the same way to the same animal species.

In some embodiments, the J-chain or functional fragment thereof comprises an amino acid substitution at the amino acid position corresponding to amino acid Y102 of the wild-type human J-chain (SEQ ID NO: 97). In some embodiments, the amino acid corresponding to Y102 of SEQ ID NO: 97 is substituted with alanine (A), serine (S), or arginine (R). In some embodiments, the amino acid corresponding to Y102 of SEQ ID NO: 97 is substituted with alanine (A). In some embodiments, the J-chain is a variant human J-chain and comprises the amino acid sequence SEQ ID NO: 98.

In some embodiments, the J-chain or functional fragment thereof comprises an amino acid substitution at the amino acid position corresponding to amino acid N49, amino acid S51, or both N49 and S51 of the human J-chain (SEQ ID NO: 97), wherein a single amino acid substitution corresponding to position S51 of SEQ ID NO: 97 is not a threonine (T) substitution. In some embodiments, the position corresponding to N49 of SEQ ID NO: 97 is substituted with alanine (A), glycine (G), threonine (T), serine (S) or aspartic acid (D). In some embodiments, the position corresponding to N49 of SEQ ID NO: 97 is substituted with alanine (A). In some embodiments, the position corresponding to S51 of SEQ ID NO: 97 is substituted with alanine (A) or glycine (G). In some embodiments, the position corresponding to S51 of SEQ ID NO: 97 is substituted with alanine (A).

In some embodiments, the J-chain or functional fragment or variant thereof further comprises a heterologous polypeptide, wherein the heterologous polypeptide is directly or indirectly fused to the J-chain or functional fragment or variant thereof. In some embodiments, the heterologous polypeptide is fused to the 1-chain or functional fragment thereof via a peptide linker. In some embodiments, the peptide linker comprises at least 5 amino acids, but no more than 25 amino acids. In some embodiments, the peptide linker consists of GGGGS (SEQ ID NO: 99), GGGGSGGGGS (SEQ ID NO: 100), GGGSGGGGSGGGGS(SEQ ID NO: 101), GGGGSGGGGSGGGGSGGGGS(SEQ ID NO: 102), or GGGGSGGGGSGGGGSGGGGSGGGGS (SEQ ID NO: 103). In some embodiments, the heterologous polypeptide is fused to the N-terminus of the J-chain or functional fragment or variant thereof, the C-terminus of the J-chain or functional fragment or variant thereof, or to both the N-terminus and C-terminus of the J-chain or functional fragment or variant thereof.

In some embodiments, the heterologous polypeptide can influence the absorption, distribution, metabolism and/or excretion (ADME) of the multimeric binding molecule. In some embodiments, the heterologous polypeptide comprises an antigen binding domain. In some embodiments, the antigen binding domain of the heterologous polypeptide is an antibody or antigen-binding fragment thereof. In some embodiments, the antigen-binding fragment comprises an Fab fragment, an Fab′ fragment, an F(ab′)₂ fragment, an Fd fragment, an Fv fragment, a single-chain Fv (scFv) fragment, a disulfide-linked Fv (sdFv) fragment, or any combination thereof. In some embodiments, the antigen-binding fragment is a scFv fragment.

In some embodiments, administration of the combination therapy results in enhanced therapeutic efficacy relative to administration of the antibody or multimerized antigen-binding fragment, variant, or derivative thereof or the cancer therapy alone. In some embodiments, the enhanced therapeutic efficacy comprises a reduced tumor growth rate, tumor regression, or increased survival. In some embodiments, the subject is human.

### BRIEF DESCRIPTION OF THE DRAWINGS/FIGURES

FIGS. 1A-1D show 3D surface plots of synergy scores from a continuum of dose combinations of Mab A and doxorubicin applied to MOLM13 (FIG. 1A), MV411 (FIG. 1B), HT1080 (FIG. 1C), or primary human hepatocytes (FIG. 1D) cells in vitro, with valleys reflecting antagonism and hills representing synergy.

FIGS. 2A-2I show 3D surface plots of synergy scores from a continuum of dose combinations of Mab A and paclitaxel applied to NCIH460 (FIG. 2A), NCIH2228 (FIG. 2B), NCIN87 (FIG. 2C), PANC1 (FIG. 2D), primary human hepatocytes (FIG. 2E), SNU5 (FIG. 2F), NUCG4 (FIG. 2G), ASPC1 (FIG. 2H), or BXPC3 (FIG. 2I) cells in vitro, with valleys reflecting antagonism and hills representing synergy.

FIGS. 3A-3E show 3D surface plots of synergy scores from a continuum of dose combinations of Mab A and carboplatin applied to NCIH460 (FIG. 3A), NCIH2228 (FIG. 3B), NCIN87 (FIG. 3C), NUGC4 (FIG. 3D), or SNU5 (FIG. 3E) cells in vitro, with valleys reflecting antagonism and hills representing synergy.

FIGS. 4A-4H show 3D surface plots of synergy scores from a continuum of dose combinations of Mab A and doxorubicin applied to NUGC4 (FIG. 4A), NCIN87 (FIG. 4B), SNU5 (FIG. 4C), NCIH508 (FIG. 4D), HCT15 (FIG. 4E), HT55 (FIG. 4F), NCI-H2228 (FIG. 4G), or primary human hepatocytes (FIG. 4H) cells in vitro, with valleys reflecting antagonism and hills representing synergy.

FIGS. 5A, 5C, 5E, 5G, and 5I show tumor volume over time for mice treated with Mab A and/or radiation (FIG. 5A), oxaliplatin (FIG. 5C), paclitaxel (FIG. 5E), irinotecan (FIG. 5G), or ABT-199 (FIG. 5I). FIGS. 5B, 5D, 5F, 5H, and 5J show survival over time for mice treated with Mab A and/or radiation (FIG. 5B), oxaliplatin (FIG. 5D), paclitaxel (FIG. 5F), irinotecan (FIG. 5H), or ABT-199 (FIG. 5J).

FIGS. 6A-6B show cell viability curves for single agent Mab A (FIG. 6A) or SMAC mimetics (FIG. 6B) on MDA-MB-231 tumor cells.

FIGS. 7A-7B show cell viability curves for combinations of Mab A and birinapant on MDA-MB-231 tumor cells (FIG. 7A) or primary human hepatocytes (FIG. 7B).

FIGS. 8A-8B show cell viability curves for combinations of Mab A and GDC-0152 on MDA-MB-231 tumor cells (FIG. 8A) or primary human hepatocytes (FIG. 8B).

FIGS. 9A-9B show 3D surface plots of synergy scores from a continuum of dose combinations of Mab A and birinapant (FIG. 9A) or GDC-0152 (FIG. 9B) applied to MDA-MB-231 tumor cells.

FIGS. 10A-10B show cell viability curves for single agent birinapant (FIG. 10A) or GDC-0152 (FIG. 6B) on DR5 agonist-resistant tumor cells.

FIGS. 11A-11B show cell viability curves for combinations of Mab A and birinapant (FIG. 11A) or GDC-0152 (FIG. 11B) on DR5 agonist-resistant tumor cells.

FIGS. 12A-12C shows cell viability curves for U-937 cells treated with Mab A alone (FIG. 12A), ibrutinib alone (FIG. 12B), or Mab A and ibrutinib (FIG. 12C) at various concentrations. FIG. 12D shows 3D surface plots of synergy scores from a continuum of dose combinations of Mab A and ibrutinib on U-937 cells.

FIGS. 13A-13C shows cell viability curves for OCI-LY7 cells treated with Mab A alone (FIG. 13A), ibrutinib alone (FIG. 13B), or Mab A and ibrutinib (FIG. 13C) at various concentrations. FIG. 13D shows 3D surface plots of synergy scores from a continuum of dose combinations of Mab A and ibrutinib on OCI-LY7 cells.

FIGS. 14A-14C shows cell viability curves for DOHH-2 cells treated with Mab A alone (FIG. 14A), idelalisib alone (FIG. 14B), or Mab A and idelalisib (FIG. 14C) at various concentrations. FIG. 14D shows 3D surface plots of synergy scores from a continuum of dose combinations of Mab A and idelalisib on DOHH-2 cells.

FIGS. 15A-15C shows cell viability curves for WSU-DLCL2 cells treated with Mab A alone (FIG. 15A), MIK665 alone (FIG. 15B), or Mab A and MIK665 (FIG. 15C) at various concentrations. FIG. 15D shows 3D surface plots of synergy scores from a continuum of dose combinations of Mab A and MIK665 on WSU-DLCL2 cells.

FIGS. 16A-16C shows cell viability curves for U-937 cells treated with Mab A alone (FIG. 16A), MIK665 alone (FIG. 16B), or Mab A and MIK665 (FIG. 16C) at various concentrations. FIG. 16D shows 3D surface plots of synergy scores from a continuum of dose combinations of Mab A and MIK665 on U-937 cells.

FIGS. 17A-17C show cell viability curves for U-937 cells treated with Mab A alone (FIG. 17A), vincristine alone (FIG. 17B), or Mab A and vincristine (FIG. 17C) at various concentrations. FIG. 17D shows 3D surface plots of synergy scores from a continuum of dose combinations of Mab A and vincristine on U-937 cells.

FIGS. 18A-18D show cell viability curves for human hepatocytes treated with Mab A and ibrutinib (FIG. 18A), Mab A and idelalisib (FIG. 18B), Mab A and MIK665 (FIG. 18C), or Mab A and vincristine (FIG. 18D) at various concentrations.

FIGS. 19A, 19C, 19E, 19G, 19I, 19K, 19M, 19O, 19Q, 19S, 19U, 19W, 19Y, 19AA, 19AC, and 19AE show cell viability curves for A2058 (FIG. 19A), BT-20 (FIG. 19C), DV-90 (FIG. 19E), ES-2 (FIG. 19G), HCC15 (FIG. 19I), HCT 116 (FIG. 19K), HT 1080 (FIG. 19M), KYSE 410 (FIG. 19O), MEWO (FIG. 19Q), OVCAR-5 (FIG. 19S), SK-LU-1 (FIG. 19U), SK-MEL-5 (FIG. 19W), SNU-1 (FIG. 19Y), SW780 (FIG. 19AA), SW1353 (FIG. 19AC), and T24 (FIG. 19AE) cells treated with Mab A and birinapant at various concentrations. FIGS. 19B, 19D, 19F, 19H, 19J, 19L, 19N, 19P, 19R, 19T, 19V, 19X, 19Z, 19AB, 19AD, and 19AF show 3D surface plots of synergy scores from a continuum of dose combinations of Mab A and birinapant on A2058 (FIG. 19B), BT-20 (FIG. 19D), DV-90 (FIG. 19F), ES-2 (FIG. 19H), HCC15 (FIG. 19J), HCT 116 (FIG. 19L), HT 1080 (FIG. 19N), KYSE 410 (FIG. 19P), MEWO (FIG. 19R), OVCAR-5 (FIG. 19T), SK-LU-1 (FIG. 19V), SK-MEL-5 (FIG. 19X), SNU-1 (FIG. 19Z), SW780 (FIG. 19AB), SW1353 (FIG. 19AD), and T24 (FIG. 19AF) cells.

FIG. 20A shows MDA-MB-231 TNBC tumor volumes over time through day 26 for mice treated with vehicle, Mab A IgM, birinapant, Mab B IgG, Mab A IgM+birinapant, or Mab B IgG+birinapant. FIG. 20B shows tumor volumes over time through day 54 for mice treated with vehicle, Mab A IgM, birinapant, Mab B IgG, Mab A IgM+birinapant, or Mab B IgG+birinapant. FIG. 20C shows survival over time for mice treated with vehicle, Mab A IgM, birinapant, Mab B IgG, Mab A IgM+birinapant, or Mab B IgG+birinapant.

FIGS. 21A-21D show tumor volumes over time for mice treated with vehicle, Mab A IgM, birinapant, or Mab A IgM+birinapant in an EBC-1 NSCLC model (FIG. 21A), HT-1080 fibrosarcoma model (FIG. 21B), HCT 116 colorectal cancer model (FIG. 21C), or SA3840 osteosarcoma PDX model (FIG. 21D).

FIGS. 22A and 22C show cell viability curves for Detroit 562 (FIG. 22A) and KYSE270 (FIG. 22C) cells treated with Mab A and birinapant at various concentrations. FIGS. 22B and 22D show 3D surface plots of synergy scores from a continuum of dose combinations of Mab A and birinapant on Detroit 562 (FIG. 22B) and KYSE270 (FIG. 22D) cells.

FIGS. 23A-23D show cell viability curves for EBC-1 cells treated with Mab A and APG-1387 (FIG. 23A), birinapant (FIG. 23B), ASTX660 (FIG. 23C), or Debio1143 (FIG. 23D) at various concentrations.

FIG. 24A shows Colo205 tumor volumes over time for mice treated with vehicle, Mab A IgM, bevacizumab, or Mab A IgM+bevacizumab. FIG. 24B shows survival over time for mice treated with vehicle, Mab A IgM, bevacizumab, or Mab A IgM+bevacizumab.

### DETAILED DESCRIPTION

### Definitions

As used herein, the term “a” or “an” entity refers to one or more of that entity; for example, “a binding molecule,” is understood to represent one or more binding molecules. As such, the terms “a” (or “an”), “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range. Unless otherwise indicated, amino acid sequences are written left to right in amino to carboxy orientation. The headings provided herein are not limitations of the various embodiments or embodiments of the disclosure, which can be had by reference to the specification as a whole. Accordingly, the terms defined immediately below are more fully defined by reference to the specification in its entirety.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of any of these terms. The term “polypeptide” is also intended to refer to the products of post-expression modifications of the polypeptide, including without limitation glycosylation, acetylation, phosphorylation, amidation, and derivatization by known protecting/blocking groups, proteolytic cleavage, or modification by non-naturally occurring amino acids. A polypeptide can be derived from a biological source or produced by recombinant technology but is not necessarily translated from a designated nucleic acid sequence. It can be generated in any manner, including by chemical synthesis.

A polypeptide as disclosed herein can be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides can have a defined three-dimensional structure, although they do not necessarily have such structure. Polypeptides with a defined three-dimensional structure are referred to as folded, and polypeptides which do not possess a defined three-dimensional structure, but rather can adopt many different conformations and are referred to as unfolded. As used herein, the term glycoprotein refers to a protein coupled to at least one carbohydrate moiety that is attached to the protein via an oxygen-containing or a nitrogen-containing side chain of an amino acid, e.g., a serine or an asparagine.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are native or recombinant polypeptides which have been separated, fractionated, or partially or substantially purified by any suitable technique. Synthetically produced polypeptides are considered isolated, which have been separated, fractionated, or partially or substantially purified by any suitable technique.

As used herein, the term “a non-naturally occurring polypeptide” or any grammatical variants thereof, is a conditional definition that explicitly excludes, but only excludes, those forms of the polypeptide that are, or might be, determined or interpreted by a judge or an administrative or judicial body, to be “naturally-occurring.”

Other polypeptides disclosed herein are fragments, derivatives, analogs, or variants of the foregoing polypeptides, and any combination thereof. The terms “fragment.” “variant.” “derivative” and “analog” as disclosed herein include any polypeptides which retain at least some of the properties of the corresponding native antibody or polypeptide, for example, specifically binding to an antigen. Fragments of polypeptides include, for example, proteolytic fragments, as well as deletion fragments, in addition to specific antibody fragments discussed elsewhere herein. Variants of, e.g., a polypeptide include fragments as described above, and also polypeptides with altered amino acid sequences due to amino acid substitutions, deletions, or insertions. In certain embodiments, variants can be non-naturally occurring. Non-naturally occurring variants can be produced using art-known mutagenesis techniques. Variant polypeptides can comprise conservative or non-conservative amino acid substitutions, deletions, or additions. Derivatives are polypeptides that have been altered so as to exhibit additional features not found on the original polypeptide. Examples include fusion proteins. As used herein a “derivative” of a polypeptide can also refer to a subject polypeptide having one or more amino acids chemically derivatized by reaction of a functional side group. Also included as “derivatives” are those polypeptides that contain one or more derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and omithine can be substituted for lysine.

A “conservative amino acid substitution” is one in which one amino acid is replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the sequences of the polypeptides, binding molecules, and antibodies of the present disclosure do not abrogate the binding of the polypeptide, binding molecule or antibody containing the amino acid sequence, to the antigen to which the polypeptide, binding molecule, or antibody binds. Methods of identifying nucleotide and amino acid conservative substitutions which do not eliminate antigen-binding are well-known in the art (see, e.g., Brummell et al., Biochem. 32: 1180-1 187 (1993); Kobayashi et al., Protein Eng. 12(10):879-884 (1999); and Burks et al., Proc. Natl. Acad. Sci. USA 94:412-417 (1997)).

The term “polynucleotide” is intended to encompass a singular nucleic acid as well as plural nucleic acids and refers to an isolated nucleic acid molecule or construct, e.g., messenger RNA (mRNA), cDNA, or plasmid DNA (pDNA). A polynucleotide can comprise a conventional phosphodiester bond or a non-conventional bond (e.g., an amide bond, such as found in peptide nucleic acids (PNA)). The terms “nucleic acid” or “nucleic acid sequence” refer to any one or more nucleic acid segments, e.g., DNA or RNA fragments, present in a polynucleotide.

By an “isolated” nucleic acid or polynucleotide is intended any form of the nucleic acid or polynucleotide that is separated from its native environment. For example, gel-purified polynucleotide, or a recombinant polynucleotide encoding a polypeptide contained in a vector would be considered to be “isolated.” Also, a polynucleotide segment, e.g., a PCR product, which has been engineered to have restriction sites for cloning is considered to be “isolated.” Further examples of an isolated polynucleotide include recombinant polynucleotides maintained in heterologous host cells or purified (partially or substantially) polynucleotides in a non-native solution such as a buffer or saline. Isolated RNA molecules include in vivo or in vitro RNA transcripts of polynucleotides, where the transcript is not one that would be found in nature. Isolated polynucleotides or nucleic acids further include such molecules produced synthetically. In addition, polynucleotide or a nucleic acid can be or can include a regulatory element such as a promoter, ribosome binding site, or a transcription terminator. Synthetically produced nucleic acids or polynucleotides are considered isolated, which have been separated, fractionated, or partially or substantially purified by any suitable technique.

As used herein, the term “a non-naturally occurring polynucleotide” or any grammatical variants thereof, is a conditional definition that explicitly excludes, but only excludes, those forms of the nucleic acid or polynucleotide that are, or might be, determined or interpreted by a judge, or an administrative or judicial body, to be “naturally-occurring.”

As used herein, a “coding region” is a portion of nucleic acid which consists of codons translated into amino acids. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. Furthermore, any vector can contain a single coding region, or can comprise two or more coding regions, e.g., a single vector can separately encode an immunoglobulin heavy chain variable region and an immunoglobulin light chain variable region. In addition, a vector, polynucleotide, or nucleic acid can include heterologous coding regions, either fused or unfused to another coding region. Heterologous coding regions include without limitation, those encoding specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally can include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter were capable of effecting transcription of that nucleic acid. The promoter can be a cell-specific promoter that directs substantial transcription of the DNA in predetermined cells. Other transcription control elements, besides a promoter, for example enhancers, operators, repressors, and transcription termination signals, can be operably associated with the polynucleotide to direct cell-specific transcription.

A variety of transcription control regions are known to those skilled in the art. These include, without limitation, transcription control regions that function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide can be RNA, for example, in the form of messenger RNA (mRNA), transfer RNA, or ribosomal RNA.

Polynucleotide and nucleic acid coding regions can be associated with additional coding regions which encode secretory or signal peptides, which direct the secretion of a polypeptide encoded by a polynucleotide as disclosed herein. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells can have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, can be used. For example, the wild-type leader sequence can be substituted with the leader sequence of human tissue plasminogen activator (TPA) or mouse β-glucuronidase.

As used herein, the terms “DR5” or “TRAILR2” refer to a member of the family of Tumor Necrosis Factor transmembrane receptor proteins expressed on the surface of various cells and tissues, which, upon activation, can induce apoptosis of the cell.

Disclosed herein are certain binding molecules, or antigen-binding fragments, variants, or derivatives thereof that bind to DR5, thereby eliciting cellular apoptosis. Unless specifically referring to full-sized antibodies, the term “binding molecule” encompasses full-sized antibodies as well as antigen-binding subunits, fragments, variants, analogs, or derivatives of such antibodies, e.g., engineered antibody molecules or fragments that bind antigen in a manner similar to antibody molecules, but which use a different scaffold. Where a binding molecule is a polymeric binding molecule, e.g., a pentameric or hexameric IgM antibody or a dimeric IgA antibody, it is understood when referring to multimeric fragments, variants, or derivatives, that the fragment, variant, or derivative continues to be multimeric.

As used herein, the term “binding molecule” refers in its broadest sense to a molecule that specifically binds to a receptor or target, e.g., an epitope or an antigenic determinant. As described further herein, a binding molecule can comprise one of more “binding domains,” e.g., “antigen-binding domains” described herein. A non-limiting example of a binding molecule is an antibody or antibody-like molecule as described in detail herein that retains antigen-specific binding. In certain embodiments a “binding molecule” comprises an antibody or antibody-like or antibody-derived molecule as described in detail herein.

As used herein, the terms “binding domain” or “antigen-binding domain” (can be used interchangeably) refer to a region of a binding molecule, e.g., an antibody or antibody-like, or antibody-derived molecule, that is necessary and sufficient to specifically bind to a target. e.g., an epitope, a polypeptide, a cell, or an organ. For example, an “Fv.” e.g., a heavy chain variable region and a light chain variable region of an antibody, either as two separate polypeptide subunits or as a single chain, is considered to be a “binding domain.” Other antigen-binding domains include, without limitation, a single domain heavy chain variable region (VHH) of an antibody derived from a camelid species, or six immunoglobulin complementarity determining regions (CDRs) expressed in a fibronectin scaffold. A “binding molecule.” e.g., an “antibody” as described herein can include one, two, three, four, five, six, seven, eight, nine, ten, eleven, twelve, or more “antigen-binding domains.”

The terms “antibody” and “immunoglobulin” can be used interchangeably herein. An antibody (or a fragment, variant, or derivative thereof as disclosed herein, e.g., an IgM-like antibody) includes at least the variable domain of a heavy chain (e.g., from a camelid species) or at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988). Unless otherwise stated, the term “antibody” encompasses anything ranging from a small antigen-binding fragment of an antibody to a full sized antibody, e.g., an IgG antibody that includes two complete heavy chains and two complete light chains, an IgA antibody that includes four complete heavy chains and four complete light chains and includes a J-chain and/or a secretory component, or an IgM-derived binding molecule, e.g., an IgM antibody or IgM-like antibody, that includes ten or twelve complete heavy chains and ten or twelve complete light chains and optionally includes a J-chain or functional fragment or variant thereof.

The term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4 or α1-α2)). It is the nature of this chain that determines the “isotype” of the antibody as IgG, IgM, IgA IgD, or IgE, respectively. The immunoglobulin subclasses (subtypes) e.g., IgG₁, IgG₂, IgG₃, IgG₄, IgA₁, IgA₂, etc. are well characterized and arc known to confer functional specialization. Modified versions of each of these immunoglobulins are readily discernible to the skilled artisan in view of the instant disclosure and, accordingly, are within the scope of this disclosure.

Light chains are classified as either kappa or lambda (κ, λ). Each heavy chain class can be bound with either a kappa or lambda light chain. In general, the light and heavy chains arc covalently bonded to each other, and the “tail” portions of the two heavy chains are bonded to each other by covalent disulfide linkages or non-covalent linkages when the immunoglobulins are expressed, e.g., by hybridomas, B cells or genetically engineered host cells. In the heavy chain, the amino acid sequences run from an N-terminus at the forked ends of the Y configuration to the C-terminus at the bottom of each chain. The basic structure of certain antibodies, e.g., IgG antibodies, includes two heavy chain subunits and two light chain subunits covalently connected via disulfide bonds to form a “Y” structure, also referred to herein as an “H2L2” structure, or a “binding unit.”

The term “binding unit” is used herein to refer to the portion of a binding molecule, e.g., an antibody, antibody-like molecule, or antibody-derived molecule, antigen-binding fragment thereof, or multimerizing fragment thereof, which corresponds to a standard “H2L2” immunoglobulin structure, i.e., two heavy chains or fragments thereof and two light chains or fragments thereof. In certain embodiments, e.g., where the binding molecule is a bivalent IgG antibody or antigen-binding fragment thereof, the terms “binding molecule” and “binding unit” are equivalent. In other embodiments, e.g., where the binding molecule is a “multimeric binding molecule,” e.g., a dimeric IgA antibody, a dimeric IgA-like antibody, a dimeric IgA-derived binding molecule, a pentameric or hexameric IgM antibody, a pentameric or hexameric IgM-like antibody, or a pentameric or hexameric IgM-derived binding molecule or any derivative thereof, the binding molecule comprises two or more “binding units.” Two in the case of an IgA dimer, or five or six in the case of an IgM pentamer or hexamer, respectively. A binding unit need not include full-length antibody heavy and light chains, but will typically be bivalent, i.e., will include two “antigen-binding domains,” as defined above. As used herein, certain binding molecules provided in this disclosure are “dimeric,” and include two bivalent binding units that include IgA constant regions or multimerizing fragments thereof. Certain binding molecules provided in this disclosure are “pentameric” or “hexameric,” and include five or six bivalent binding units that include IgM constant regions or multimerizing fragments or variants thereof. A binding molecule, e.g., an antibody or antibody-like molecule or antibody-derived binding molecule, comprising two or more, e.g., two, five, or six binding units, is referred to herein as “multimeric.”

The term “J-chain” as used herein refers to the J-chain of IgM or IgA antibodies of any animal species, any functional fragment thereof, derivative thereof, and/or variant thereof, including a mature human J-chain, the amino acid sequence of which is presented as SEQ ID NO: 97. Various J-chain variants and modified J-chain derivatives are disclosed herein. As persons of ordinary skill in the art will recognize, “a functional fragment” or “a functional variant” includes those fragments and variants that can associate with IgM heavy chain constant regions to form a pentameric IgM antibody.

The term “modified J-chain” is used herein to refer to a derivative of a J-chain polypeptide comprising a heterologous moiety, e.g., a heterologous polypeptide, e.g., an extraneous binding domain or functional domain introduced into or attached to the J-chain sequence. The introduction can be achieved by any means, including direct or indirect fusion of the heterologous polypeptide or other moiety or by attachment through a peptide or chemical linker. The term “modified human J-chain” encompasses, without limitation, a native sequence human J-chain comprising the amino acid sequence of SEQ ID NO: 97 or functional fragment thereof, or functional variant thereof, modified by the introduction of a heterologous moiety, e.g., a heterologous polypeptide, e.g., an extraneous binding domain. In certain embodiments the heterologous moiety does not interfere with efficient polymerization of IgM into a pentamer or IgA into a dimer and binding of such polymers to a target. Exemplary modified J-chains can be found, e.g., in U.S. Pat. Nos. 9,951,134 and 10,400,038, and in U.S. Patent Application Publication Nos. US-2019-0185570 and US-2018-0265596, each of which is incorporated herein by reference in its entirety.

As used herein the term “IgM-derived binding molecule” refers collectively to native IgM antibodies, IgM-like antibodies, as well as other IgM-derived binding molecules comprising non-antibody binding and/or functional domains instead of an antibody antigen binding domain or subunit thereof, and any fragments, e.g., multimerizing fragments, variants, or derivatives thereof.

As used herein, the term “IgM-like antibody” refers generally to a variant antibody or antibody-derived binding molecule that still retains the ability to form hexamers or pentamers, e.g., in association with a J-chain. An IgM-like antibody or other IgM-derived binding molecule typically includes at least the Cμ4-tp domains of the IgM constant region but can include heavy chain constant region domains from other antibody isotypes, e.g., IgG, from the same species or from a different species. An IgM-like antibody or other IgM-derived binding molecule can likewise be an antibody fragment in which one or more constant regions are deleted, as long as the IgM-like antibody is capable of forming hexamers and/or pentamers. Thus, an IgM-like antibody or other IgM-derived binding molecule can be, e.g., a hybrid IgM/IgG antibody or can be a “multimerizing fragment” of an IgM antibody.

As used herein the term “IgA-derived binding molecule” refers collectively to native IgA antibodies, IgA-like antibodies, as well as other IgA-derived binding molecules comprising non-antibody binding and/or functional domains instead of an antibody antigen binding domain or subunit thereof, and any fragments, e.g., multimerizing fragments, variants, or derivatives thereof.

As used herein, the term “IgA-like antibody” refers generally to a variant antibody or antibody-derived binding molecule that still retains the ability to form dimers, e.g., in association with a J-chain. An IgA-like antibody or other IgA-derived binding molecule typically includes at least the Cα3-tp domains of the IgA constant region but can include heavy chain constant region domains from other antibody isotypes, e.g., IgG, from the same species or from a different species. An IgA-like antibody or other IgA-derived binding molecule can likewise be an antibody fragment in which one or more constant regions are deleted, as long as the IgA-like antibody is capable of forming dimers. Thus, an IgA-like antibody or other IgA-derived binding molecule can be, e.g., a hybrid IgA/IgG antibody or can be a “multimerizing fragment” of an IgA antibody.

The terms “valency,” “bivalent,” “multivalent” and grammatical equivalents, refer to the number of binding domains, e.g., antigen-binding domains in given binding molecule, e.g., antibody, antibody-derived, or antibody-like molecule, or in a given binding unit. As such, the terms “bivalent”, “tetravalent”, and “hexavalent” in reference to a given binding molecule, e.g., an IgM antibody. IgM-like antibody, other IgM-derived binding molecule, or multimerizing fragment thereof, denote the presence of two antigen-binding domains, four antigen-binding domains, and six antigen-binding domains, respectively. A typical IgM antibody. IgM-like antibody, or other IgM-derived binding molecule, where each binding unit is bivalent, can have 10 or 12 valencies. A bivalent or multivalent binding molecule, e.g., antibody or antibody-derived molecule, can be monospecific, i.e., all of the antigen-binding domains are the same, or can be bispecific or multispecific, e.g., where two or more antigen-binding domains are different, e.g., bind to different epitopes on the same antigen, or bind to entirely different antigens.

The term “epitope” includes any molecular determinant capable of specific binding to an antigen-binding domain of an antibody, antibody-like, or antibody-derived molecule. In certain embodiments, an epitope can include chemically active surface groupings of molecules such as amino acids, sugar side chains, phosphoryl groups, or sulfonyl groups, and, in certain embodiments, can have three-dimensional structural characteristics, and or specific charge characteristics. An epitope is a region of a target that is bound by an antigen-binding domain of an antibody.

The term “target” is used in the broadest sense to include substances that can be bound by a binding molecule, e.g., antibody, antibody-like, or antibody-derived molecule. A target can be, e.g., a polypeptide, a nucleic acid, a carbohydrate, a lipid, or other molecule, or a minimal epitope on such molecule. Moreover, a “target” can, for example, be a cell, an organ, or an organism, e.g., an animal, plant, microbe, or virus, that comprises an epitope that can be bound by a binding molecule, e.g., antibody, antibody-like, or antibody-derived molecule.

Both the light and heavy chains of antibodies, antibody-like, or antibody-derived molecules are divided into regions of structural and functional homology. The terms “constant” and “variable” are used functionally. In this regard, it will be appreciated that the variable domains of both the variable light (VL) and variable heavy (VH) chain portions determine antigen recognition and specificity. Conversely, the constant region domains of the light chain (CL) and the heavy chain (e.g., CH1, CH2, CH3, or CH4) confer biological properties such as secretion, transplacental mobility, Fc receptor binding, complement binding, and the like. By convention, the numbering of the constant region domains increases as they become more distal from the antigen-binding site or amino-terminus of the antibody. The N-terminal portion is a variable region and at the C-terminal portion is a constant region; the CH3 (or CH4, e.g., in the case of IgM) and CL domains actually comprise the carboxy-terminus of the heavy and light chain, respectively.

A “full length IgM antibody heavy chain” is a polypeptide that includes, in N-terminal to C-terminal direction, an antibody heavy chain variable domain (VH), an antibody heavy chain constant domain 1 (CM1 or Cμ1), an antibody heavy chain constant domain 2 (CM2 or Cμ2), an antibody heavy chain constant domain 3 (CM3 or Cμ3), and an antibody heavy chain constant domain 4 (CM4 or Cμ4) that can include a tailpiece.

A “full length IgA antibody heavy chain” is a polypeptide that includes, in N-terminal to C-terminal direction, an antibody heavy chain variable domain (VH), an antibody constant heavy chain constant domain 1 (CA1 or Cα1), an antibody heavy chain constant domain 2 (CA2 or Cα2), and an antibody heavy chain constant domain 3 (CA3 or Cα3) that can include a tailpiece.

As indicated above, variable region(s) allow a binding molecule, e.g., antibody, antibody-like, or antibody-derived molecule, to selectively recognize and specifically bind epitopes on antigens. That is, the VL domain and VH domain, or subset of the complementarity determining regions (CDRs), of a binding molecule, e.g., an antibody, antibody-like, or antibody-derived molecule, combine to form the antigen-binding domain. More specifically, an antigen-binding domain can be defined by three CDRs on each of the VH and VL chains. Certain antibodies form larger structures. For example, IgA can form a molecule that includes two H2L2 binding units and a J-chain covalently connected via disulfide bonds, which can be further associated with a secretory component, and IgM can form a pentameric or hexameric molecule that includes five or six H2L2 binding units and optionally a J-chain covalently connected via disulfide bonds.

The six “complementarity determining regions” or “CDRs” present in an antibody antigen-binding domain are short, non-contiguous sequences of amino acids that are specifically positioned to form the antigen-binding domain as the antibody assumes its three-dimensional configuration in an aqueous environment. The remainder of the amino acids in the antigen-binding domain, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids that make up the CDRs and the framework regions, respectively, can be readily identified for any given heavy or light chain variable region by one of ordinary skill in the art, since they have been defined in various different ways (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. These particular regions have been described, for example, by Kabat et al., U.S. Dept. of Health and Human Services, “Sequences of Proteins of Immunological Interest” (1983) and by Chothia et al., J Mol. Biol. 196:901-917 (1987), which are incorporated herein by reference. The Kabat and Chothia definitions include overlapping or subsets of amino acids when compared against each other. Nevertheless, application of either definition (or other definitions known to those of ordinary skill in the art) to refer to a CDR of an antibody or variant thereof is intended to be within the scope of the term as defined and used herein, unless otherwise indicated. The appropriate amino acids which encompass the CDRs as defined by each of the above cited references are set forth below in Table 1 as a comparison. The exact amino acid numbers which encompass a particular CDR will vary depending on the sequence and size of the CDR. Those skilled in the art can routinely determine which amino acids comprise a particular CDR given the variable region amino acid sequence of the antibody.

TABLE 1
CDR Definitions*

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

*Numbering of all CDR definitions in Table 1 is according to the numbering conventions set forth by Kabat et al. (see below).

Antibody variable domains can also be analyzed, e.g., using the IMGT information system (imgt dot cines dot fr/) (IMGTVN-Quest) to identify variable region segments, including CDRs. (See, e.g., Brochet et al., Nucl. Acids Res, 36:W503-508, 2008).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless use of the Kabat numbering system is explicitly noted, however, consecutive numbering is used for all amino acid sequences in this disclosure.

The Kabat numbering system for the human IgM constant domain can be found in Kabat, et. al. “Tabulation and Analysis of Amino acid and nucleic acid Sequences of Precursors, V-Regions, C-Regions, J-Chain, T-Cell Receptors for Antigen, T-Cell Surface Antigens, β-2 Microglobulins, Major Histocompatibility Antigens, Thy-1, Complement, C-Reactive Protein, Thymopoietin, Integrins, Post-gamma Globulin, α-2 Macroglobulins, and Other Related Proteins,” U.S. Dept. of Health and Human Services (1991). IgM constant regions can be numbered sequentially (i.e., amino acid #1 starting with the first amino acid of the constant region, or by using the Kabat numbering scheme. A comparison of the numbering of two alleles of the human IgM constant region sequentially (presented herein as SEQ ID NO: 91 (allele IGHM*03) and SEQ ID NO: 92 (allele IGHM*04)) and by the Kabat system is set out below. The underlined amino acid residues are not accounted for in the Kabat system (“X,” double underlined below, can be serine (S) (SEQ ID NO: 91) or glycine (G) (SEQ ID NO: 92)):

| Sequential (SEQ ID NO: 91 or SEQ ID NO: 92)/KABAT numbering | |
|---|---|
| key for IgM heavy chain | |
| 1/127 | GSASAPTLFP LVSCENSPSD TSSVAVGCLA QDFLPDSITF SWKYKNNSDI |
| 51/176 | SSTRGFPSVL RGGKYAATSQ VLLPSKDVMQ GTDEHVVCKV QHPNGNKEKN |
| 101/226 | VPLPVIAELP PKVSVFVPPR DGFFGNPRKS KLICQATGFS PRQIQVSWLR |
| 151/274 | EGKQVGSGVT TDQVQAEAKE SGPTTYKVTS TLTIKESDWL XQSMFTCRVD |
| 201/324 | HRGLTFQQNA SSMCVPDQDT AIRVFAIPPS FASIFLTKST KLTCLVTDLT |
| 251/374 | TYDSVTISWT RQNGEAVKTH TNISESHPNA TFSAVGEASI CEDDWNSGER |
| 301/424 | FTCTVTHTDL PSPLKQTISR PKGVALHRPD VYLLPPAREQ LNLRESATIT |
| 351/474 | CLVTGFSPAD VFVQWMQRGQ PLSPEKYVTS APMPEPQAPG RYFAHSILTV |
| 401/524 | SEEEWNTGET YTCVVAHEAL PNRVTERTVD KSTGKPTLYN VSLVMSDTAG |
| 451/574 | TCY |

Binding molecules. e.g., antibodies, antibody-like, or antibody-derived molecules, antigen-binding fragments, variants, or derivatives thereof, and/or multimerizing fragments thereof include, but are not limited to, polyclonal, monoclonal, human, humanized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019.

By “specifically binds,” it is generally meant that a binding molecule, e.g., an antibody or fragment, variant, or derivative thereof binds to an epitope via its antigen-binding domain, and that the binding entails some complementarity between the antigen-binding domain and the epitope. According to this definition, a binding molecule, e.g., antibody, antibody-like, or antibody-derived molecule, is said to “specifically bind” to an epitope when it binds to that epitope, via its antigen-binding domain more readily than it would bind to a random, unrelated epitope. The term “specificity” is used herein to qualify the relative affinity by which a certain binding molecule binds to a certain epitope. For example, binding molecule “A” can be deemed to have a higher specificity for a given epitope than binding molecule “B,” or binding molecule “A” can be said to bind to epitope “C” with a higher specificity than it has for related epitope “D.”

A binding molecule, e.g., an antibody or fragment, variant, or derivative thereof disclosed herein can be said to bind a target antigen with an off rate (k(off)) of less than or equal to 5×10⁻² sec⁻¹, 10⁻² sec⁻¹, 5×10⁻³ sec⁻¹, 10⁻³ sec⁻¹, 5×10⁻⁴ sec⁻¹, 10⁻⁴ sec⁻¹, 5×10⁻⁵ sec⁻¹, or 10⁻⁵ sec⁻¹, 5×10⁻⁶ sec⁻¹, 10⁻⁶ sec⁻¹, 5×10⁻⁷ sec⁻¹ or 10⁻⁷ sec⁻¹.

A binding molecule, e.g., an antibody or antigen-binding fragment, variant, or derivative disclosed herein can be said to bind a target antigen with an on rate (k(on)) of greater than or equal to 10³ M⁻¹ sec⁻¹, 5×10³ M⁻¹ sec⁻¹, 10⁴ M⁻¹ sec⁻¹, 5×10⁴ M⁻¹ sec⁻¹, 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹.

A binding molecule, e.g., an antibody or fragment, variant, or derivative thereof is said to competitively inhibit binding of a reference antibody or antigen-binding fragment to a given epitope if it preferentially binds to that epitope to the extent that it blocks, to some degree, binding of the reference antibody or antigen-binding fragment to the epitope. Competitive inhibition can be determined by any method known in the art, for example, competition ELISA assays. A binding molecule can be said to competitively inhibit binding of the reference antibody or antigen-binding fragment to a given epitope by at least 90%, at least 80%, at least 70%, at least 60%, or at least 50%.

As used herein, the term “affinity” refers to a measure of the strength of the binding of an individual epitope with one or more antigen-binding domains, e.g., of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of antigen-binding domains and an antigen. See, e.g., Harlow at pages 29-34. Avidity is related to both the affinity of individual antigen-binding domains in the population with specific epitopes, and also the valencies of the immunoglobulins and the antigen. For example, the interaction between a bivalent monoclonal antibody and an antigen with a highly repeating epitope structure, such as a polymer, would be one of high avidity. An interaction between a bivalent monoclonal antibody with a receptor present at a high density on a cell surface would also be of high avidity.

Binding molecules, e.g., antibodies or fragments, variants, or derivatives thereof as disclosed herein can also be described or specified in terms of their cross-reactivity. As used herein, the term “cross-reactivity” refers to the ability of a binding molecule, e.g., an antibody or fragment, variant, or derivative thereof, specific for one antigen, to react with a second antigen; a measure of relatedness between two different antigenic substances. Thus, a binding molecule is cross reactive if it binds to an epitope other than the one that induced its formation. The cross-reactive epitope generally contains many of the same complementary structural features as the inducing epitope, and in some cases, can actually fit better than the original.

A binding molecule, e.g., an antibody or fragment, variant, or derivative thereof can also be described or specified in terms of their binding affinity to an antigen. For example, a binding molecule can bind to an antigen with a dissociation constant or KL) no greater than 5×10⁻² M, 10⁻² M, 5×10⁻³ M, 10⁻⁴ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴M, 10⁻¹⁴ M, 5×10⁻¹⁵ M, or 10⁻¹⁵ M.

“Antigen-binding antibody fragments” including single-chain antibodies or other antigen-binding domains can exist alone or in combination with one or more of the following: hinge region, CH1, CH2, CH3, or CH4 domains, J-chain, or secretory component. Also included are antigen-binding fragments that can include any combination of variable region(s) with one or more of a hinge region, CH1, CH2, CH3, or CH4 domains, a J-chain, or a secretory component. Binding molecules, e.g., antibodies, or antigen-binding fragments thereof can be from any animal origin including birds and mammals. The antibodies can be human, murine, donkey, rabbit, goat, guinea pig, camel, llama, horse, or chicken antibodies. In another embodiment, the variable region can be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and can in some instances express endogenous immunoglobulins and some not, as described infra and, for example in. U.S. Pat. No. 5,939,598 by Kucherlapati et al. According to embodiments of the present disclosure, an IgM antibody, IgM-like antibody, or other IgM-derived binding molecule as provided herein can include an antigen-binding fragment of an antibody. e.g., a scFv fragment, so long as the IgM antibody, IgM-like antibody, or other IgM-derived binding molecule is able to form a multimer, e.g., a hexamer or a pentamer, and an IgA antibody, IgA-like antibody, or other IgA-derived binding molecule as provided herein can include an antigen-binding fragment of an antibody, e.g., a scFv fragment, so long as the IgA antibody, IgA-like antibody, or other IgA-derived binding molecule is able to form a multimer, e.g., a dimer. As used herein such a fragment comprises a “multimerizing fragment.”

As used herein, the term “heavy chain subunit” includes amino acid sequences derived from an immunoglobulin heavy chain, a binding molecule, e.g., an antibody, antibody-like, or antibody-derived molecule comprising a heavy chain subunit can include at least one of: a VH domain, a CH1 domain, a hinge (e.g., upper, middle, and/or lower hinge region) domain, a CH2 domain, a CH3 domain, a CH4 domain, or a variant or fragment thereof. For example, a binding molecule, e.g., an antibody, antibody-like, or antibody-derived molecule, or fragment, e.g., multimerizing fragment, variant, or derivative thereof can include without limitation, in addition to a VH domain; a CH1 domain; a CH1 domain, a hinge, and a CH2 domain; a CH1 domain and a CH3 domain; a CH1 domain, a hinge, and a CH3 domain; or a CH1 domain, a hinge domain, a CH2 domain, and a CH3 domain. In certain embodiments a binding molecule, e.g., an antibody, antibody-like, or antibody-derived molecule, or fragment, e.g., multimerizing fragment, variant, or derivative thereof can include, in addition to a VH domain, a CH3 domain and a CH4 domain; or a CH3 domain, a CH4 domain, and a J-chain. Further, a binding molecule, e.g., an antibody, antibody-like, or antibody-derived molecule, for use in the disclosure can lack certain constant region portions, e.g., all or part of a CH2 domain. It will be understood by one of ordinary skill in the art that these domains (e.g., the heavy chain subunit) can be modified such that they vary in amino acid sequence from the original immunoglobulin molecule. According to embodiments of the present disclosure, an IgM antibody, IgM-like antibody, or other IgM-derived binding molecule as provided herein comprises sufficient portions of an IgM heavy chain constant region to allow the IgM antibody, IgM-like antibody, or other IgM-derived binding molecule to form a multimer, e.g., a hexamer or a pentamer. As used herein such a fragment comprises a “multimerizing fragment.”

As used herein, the term “light chain subunit” includes amino acid sequences derived from an immunoglobulin light chain. The light chain subunit includes at least a VL, and can further include a CL (e.g., Cc or C) domain.

Binding molecules, e.g., antibodies, antibody-like molecules, antibody-derived molecules, antigen-binding fragments, variants, or derivatives thereof, or multimerizing fragments thereof can be described or specified in terms of the epitope(s) or portion(s) of a target, e.g., a target antigen that they recognize or specifically bind. The portion of a target antigen that specifically interacts with the antigen-binding domain of an antibody is an “epitope,” or an “antigenic determinant.” A target antigen can comprise a single epitope or at least two epitopes, and can include any number of epitopes, depending on the size, conformation, and type of antigen.

As used herein the term “disulfide bond” includes the covalent bond formed between two sulfur atoms, e.g., in cysteine residues of a polypeptide. The amino acid cysteine comprises a thiol group that can form a disulfide bond or bridge with a second thiol group. Disulfide bonds can be “intra-chain,” i.e., linking to cysteine residues in a single polypeptide or polypeptide subunit, or can be “inter-chain,” i.e., linking two separate polypeptide subunits, e.g., an antibody heavy chain and an antibody light chain, to antibody heavy chains, or an IgM or IgA antibody heavy chain constant region and a J-chain.

As used herein, the term “chimeric antibody” refers to an antibody in which the immunoreactive region or site is obtained or derived from a first species and the constant region (which can be intact, partial, or modified) is obtained from a second species. In some embodiments the target binding region or site will be from a non-human source (e.g., mouse or primate) and the constant region is human.

The terms “multispecific antibody” or “bispecific antibody” refer to an antibody, antibody-like, or antibody-derived molecule that has antigen-binding domains for two or more different epitopes within a single antibody molecule. Other binding molecules in addition to the canonical antibody structure can be constructed with two binding specificities. Epitope binding by bispecific or multispecific antibodies can be simultaneous or sequential. Triomas and hybrid hybridomas arc two examples of cell lines that can secrete bispecific antibodies. Bispecific antibodies can also be constructed by recombinant means. (Ströhlein and Heiss, Future Oncol. 6:1387-94 (2010); Mabry and Snavely, IDrugs. 13:543-9 (2010)). A bispecific antibody can also be a diabody.

As used herein, the term “engineered antibody” refers to an antibody in which a variable domain, constant region, and/or J-chain is altered by at least partial replacement of one or more amino acids. In certain embodiments entire CDRs from an antibody of known specificity can be grafted into the framework regions of a heterologous antibody. Although alternate CDRs can be derived from an antibody of the same class or even subclass as the antibody from which the framework regions are derived, CDRs can also be derived from an antibody of different class, e.g., from an antibody from a different species. An engineered antibody in which one or more “donor” CDRs from a non-human antibody of known specificity are grafted into a human heavy or light chain framework region is referred to herein as a “humanized antibody.” In certain embodiments not all of the CDRs are replaced with the complete CDRs from the donor variable region and yet the antigen-binding capacity of the donor can still be transferred to the recipient variable domains. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing, to obtain a functional engineered or humanized antibody.

As used herein the term “engineered” includes manipulation of nucleic acid or polypeptide molecules by synthetic means (e.g., by recombinant techniques, in vitro peptide synthesis, by enzymatic or chemical coupling of peptides, nucleic acids, or glycans, or some combination of these techniques).

As used herein, the terms “linked,” “fused” or “fusion” or other grammatical equivalents can be used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments can be physically or spatially separated by, for example, in-frame linker sequence. For example, polynucleotides encoding the CDRs of an immunoglobulin variable region can be fused, in-frame, but be separated by a polynucleotide encoding at least one immunoglobulin framework region or additional CDR regions, as long as the “fused” CDRs are co-translated as part of a continuous polypeptide.

As used herein, the term “cross-linked” refers to joining together of two or more molecules by a third molecule. For example, a bivalent antibody with two binding domains that specifically bind to the same antigen can “cross-link” two copies of that antigen, e.g., as they are expressed on a cell. Many TNF superfamily receptor proteins, including DR5, require cross-linking of three or more receptors on the surface of a cell for activation. Cross-linking of DR5 proteins means, for instance, contacting a binding molecule, as disclosed herein, with DR5 expressed on the surface of a cell such that at least three DR5 monomers are simultaneously bound together by one or more binding molecules, thereby activating the receptors.

In the context of polypeptides, a “linear sequence” or a “sequence” is an order of amino acids in a polypeptide in an amino to carboxyl terminal direction in which amino acids that neighbor each other in the sequence are contiguous in the primary structure of the polypeptide. A portion of a polypeptide that is “amino-terminal” or “N-terminal” to another portion of a polypeptide is that portion that comes earlier in the sequential polypeptide chain. Similarly, a portion of a polypeptide that is “carboxy-terminal” or “C-terminal” to another portion of a polypeptide is that portion that comes later in the sequential polypeptide chain. For example, in a typical antibody, the variable domain is “N-terminal” to the constant region, and the constant region is “C-terminal” to the variable domain.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into RNA, e.g., messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide that is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

As used herein, the terms “cancer” and “cancerous” refer to or describe the physiological condition in mammals in which a population of cells are characterized by unregulated cell growth. Cancers can be categorized, e.g., as solid tumors or malignancies, or hematological cancers or malignancies. Both types can migrate to remote sites as metastases. A solid tumor can be categorized, e.g., as a sarcoma, a carcinoma, a melanoma, or a metastasis thereof.

The terms “proliferative disorder” and “proliferative disease” refer to disorders associated with abnormal cell proliferation such as cancer. “Tumor” and “neoplasm” as used herein refer to any mass of tissue that result from excessive cell growth or proliferation, either benign (noncancerous) or malignant (cancerous) including pre-cancerous lesions.

The terms “metastasis.” “metastases,” “metastatic,” and other grammatical equivalents as used herein refer to cancer cells which spread or transfer from the site of origin (e.g., a primary tumor) to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures. The terms also refer to the process of metastasis, which includes, but is not limited to detachment of cancer cells from a primary tumor, intravasation of the tumor cells to circulation, their survival and migration to a distant site, attachment and extravasation into a new site from the circulation, and microcolonization at the distant site, and tumor growth and development at the distant site.

Examples of such solid tumors can include, e.g., squamous cell carcinoma, adenocarcinoma, basal cell carcinoma, renal cell carcinoma, ductal carcinoma of the breast, soft tissue sarcoma, osteosarcoma, melanoma, small-cell lung cancer, non-small cell lung cancer (NSCLC), adenocarcinoma of the lung, cancer of the peritoneum, hepatocellular carcinoma, gastrointestinal cancer, gastric cancer, pancreatic cancer, neuroendocrine cancer, glioblastoma, cervical cancer, ovarian cancer, liver cancer, bladder cancer, brain cancer, hepatoma, breast cancer, colon cancer, colorectal cancer, endometrial or uterine carcinoma, esophageal cancer, salivary gland carcinoma, kidney cancer, prostate cancer, vulval cancer, thyroid cancer, head and neck cancer, any metastases thereof, or any combination thereof.

Examples of hematologic cancers or malignancies include without limitation leukemia, lymphoma, myeloma, acute myeloid leukemia, chronic myeloid leukemia, acute lymphocytic leukemia, chronic lymphocytic leukemia, hairy cell leukemia, Hodgkin lymphoma, non-Hodgkin lymphoma, multiple myeloma, any metastases thereof, or any combination thereof.

In certain embodiments, cancers that are amenable to treatment via the methods provided herein include, but are not limited to sarcomas, breast carcinomas, ovarian cancer, cervical cancer, head and neck cancer, NSCLC, esophageal cancer, gastric cancer, kidney cancer, liver cancer, bladder cancer, colorectal cancer, and pancreatic cancer.

The term “therapeutically effective amount” refers to an amount of an antibody, polypeptide, polynucleotide, small organic molecule, or other drug effective to “treat” or in some instances, “prevent” a disease or disorder in a subject, e.g., a human. In the case of cancer, the therapeutically effective amount of the drug can reduce the number of cancer cells; retard or stop cancer cell division, reduce or retard an increase in tumor size; inhibit, e.g., suppress, retard, prevent, stop, delay, or reverse cancer cell infiltration into peripheral organs including, for example, the spread of cancer into soft tissue and bone; inhibit, e.g., suppress, retard, prevent, shrink, stop, delay, or reverse tumor metastasis; inhibit, e.g., suppress, retard, prevent, stop, delay, or reverse tumor growth; relieve to some extent one or more of the symptoms associated with the cancer, reduce morbidity and mortality: improve quality of life; or a combination of such effects. To the extent the drug prevents growth and/or kills existing cancer cells, it can be referred to as cytostatic and/or cytotoxic.

Terms such as “treating” or “treatment” or “to treat” or “alleviating” or “to alleviate” refer to therapeutic measures that cure, slow down, lessen symptoms of, and/or halt or slow the progression of a diagnosed pathologic condition or disorder. Terms such as “prevent,” “prevention,” “avoid,” “deterrence” and the like refer to prophylactic or preventative measures that prevent the development of an undiagnosed targeted pathologic condition or disorder. Thus, “those in need of treatment” can include those already with the disorder and/or those prone to have the disorder.

A subject is successfully “treated” according to the methods of the present disclosure if the patient shows one or more of the following: a reduction in the number of or complete absence of cancer cells; a reduction in the tumor size; or retardation or reversal of tumor growth, inhibition, e.g., suppression, prevention, retardation, shrinkage, delay, or reversal of metastases, e.g., of cancer cell infiltration into peripheral organs including, for example, the spread of cancer into soft tissue and bone; inhibition of, e.g., suppression of, retardation of, prevention of, shrinkage of, reversal of, delay of, or an absence of tumor metastases; inhibition of, e.g., suppression of, retardation of, prevention of, shrinkage of, reversal of, delay of, or an absence of tumor growth; relief of one or more symptoms associated with the specific cancer; reduced morbidity and mortality; improvement in quality of life; or some combination of effects. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject. In certain embodiments, the subject is a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, swine, cows, bears, and so on.

As used herein, as the term “a subject that would benefit from therapy” refers to a subset of subjects, from amongst all prospective subjects, which would benefit from administration of a given therapeutic agent, e.g., a binding molecule such as an antibody, comprising one or more antigen-binding domains. Such binding molecules, e.g., antibodies, can be used, e.g., for a diagnostic procedure and/or for treatment or prevention of a disease.

As used herein the terms “serum half-life” or “plasma half-life” refer to the time it takes (e.g., in minutes, hours, or days) following administration for the serum or plasma concentration of a drug, e.g., a binding molecule such as an antibody, antibody-like, or antibody-derived molecule or fragment, e.g., multimerizing fragment thereof as described herein, to be reduced by 50%. Two half-lives can be described: the alpha half-life, α half-life, or tin, which is the rate of decline in plasma concentrations due to the process of drug redistribution from the central compartment, e.g., the blood in the case of intravenous delivery, to a peripheral compartment (e.g., a tissue or organ), and the beta half-life, β half-life, or t_{1/2}β which is the rate of decline due to the processes of excretion or metabolism.

As used herein the term “area under the plasma drug concentration-time curve” or “AUC” reflects the actual body exposure to drug after administration of a dose of the drug and is expressed in mg*h/L. This area under the curve can be measured, e.g., from time 0 (to) to infinity (or) and is dependent on the rate of elimination of the drug from the body and the dose administered.

As used herein, the term “mean residence time” or “MRT” refers to the average length of time the drug remains in the body.

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug Administration.

“Pharmaceutically acceptable salts” are those salts which retain at least some of the biological activity of the free (non-salt) compound and which can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases which can be used to prepared salts include aluminum hydroxide, calcium hydroxide, potassium hydroxide, sodium carbonate, sodium hydroxide, and the like. Pharmaceutically acceptable salts can be prepared in situ in the manufacturing process, or by separately reacting a purified compound of the invention in its free acid or base form with a suitable organic or inorganic base or acid, respectively, and isolating the salt thus formed during subsequent purification.

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey de, lactose (anhydrate or monohydrate; optionally in combination with aspartame, cellulose, or microcrystalline cellulose), starch de, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

IgM Antibodies, IgM-Like Antibodies, and Other IgM-Derived Binding Molecules

IgM is the first immunoglobulin produced by B cells in response to stimulation by antigen. Naturally-occurring IgM is naturally present at around 1.5 mg/ml in serum with a half-life of about 5 days. IgM is a pentameric or hexameric molecule and thus includes five or six binding units. An IgM binding unit typically includes two light and two heavy chains. While an IgG heavy chain constant region contains three heavy chain constant domains (CH1, CH2 and CH3), the heavy (μ) constant region of IgM additionally contains a fourth constant domain (CH4) and includes a C-terminal “tailpiece.” The human IgM constant region typically comprises the amino acid sequence SEQ ID NO: 91 (identical to, e.g., GenBank Accession Nos. pir∥S37768, CAA47708.1, and CAA47714.1, allele IGHM*03) or SEQ ID NO: 92 (identical to, e.g., GenBank Accession No. sp|P01871.4, allele IGHM*04). The human Cμ1 region ranges from about amino acid 5 to about amino acid 102 of SEQ ID NO: 91 or SEQ ID NO: 92; the human Cμ2 region ranges from about amino acid 114 to about amino acid 205 of SEQ ID NO: 91 or SEQ ID NO: 92, the human Cμ3 region ranges from about amino acid 224 to about amino acid 319 of SEQ ID NO: 91 or SEQ ID NO: 92, the Cμ 4 region ranges from about amino acid 329 to about amino acid 430 of SEQ ID NO: 91 or SEQ ID NO: 92, and the tailpiece ranges from about amino acid 431 to about amino acid 453 of SEQ ID NO: 91 or SEQ ID NO: 92.

Other forms and alleles of the human IgM constant region with minor sequence variations exist, including, without limitation, GenBank Accession Nos. CAB37838.1, and pir∥MHHU. The amino acid substitutions, insertions, and/or deletions at positions corresponding to SEQ ID NO: 91 or SEQ ID NO: 92 described and claimed elsewhere in this disclosure can likewise be incorporated into alternate human IgM sequences, as well as into IgM constant region amino acid sequences of other species.

Each IgM heavy chain constant region can be associated with a binding domain, e.g., an antigen-binding domain, e.g., a scFv or VHH, or a subunit of an antigen-binding domain, e.g., a VH region. Exemplary antigen-binding domains, e.g., binding domains that specifically and agonistically bind DR5 are described elsewhere herein. In certain embodiments the binding domain can be a non-antibody binding domain, e.g., a receptor ectodomain, a ligand or receptor-binding fragment thereof, a cytokine or receptor-binding fragment thereof, a growth factor or receptor binding fragment thereof, a neurotransmitter or receptor binding fragment thereof, a peptide or protein hormone or receptor binding fragment thereof, an immune checkpoint modulator ligand or receptor-binding fragment thereof, or a receptor-binding fragment of an extracellular matrix protein. See, e.g., PCT Application No. PCT US2019/057702, which is incorporated herein by reference in its entirety.

Five IgM binding units can form a complex with an additional small polypeptide chain (the J-chain), or a functional fragment, variant, or derivative thereof, to form a pentameric IgM antibody or IgM-like antibody. as discussed elsewhere herein. The precursor form of the human J-chain is presented as SEQ ID NO: 96. The signal peptide extends from amino acid 1 to about amino acid 22 of SEQ ID NO: 96, and the mature human J-chain extends from about amino acid 23 to amino acid 159 of SEQ ID NO: 96. The mature human J-chain includes the amino acid sequence SEQ ID NO: 97.

Exemplary variant and modified J-chains are provided elsewhere herein. Without the J-chain, an IgM antibody or IgM-like antibody typically assembles into a hexamer, comprising up to twelve antigen-binding domains. With a J-chain, an IgM antibody or IgM-like antibody typically assembles into a pentamer, comprising up to ten antigen-binding domains, or more, if the J-chain is a modified J-chain comprising one or more heterologous polypeptides comprising additional antigen-binding domain(s). The assembly of five or six IgM binding units into a pentameric or hexameric IgM antibody or IgM-like antibody is thought to involve the Cμ4 and tailpiece domains. See. e.g., Braathcn, R., et al., J. Biol. Chem. 277:42755-42762 (2002). Accordingly, a pentameric or hexameric IgM antibody provided in this disclosure typically includes at least the Cμ4 and tailpiece domains (also referred to herein collectively as Cμ4-tp). A “multimerizing fragment” of an IgM heavy chain constant region thus includes at least the Cμ4-tp domains. An IgM heavy chain constant region can additionally include a Cμ3 domain or a fragment thereof, a Cμ2 domain or a fragment thereof, a Cμ1 domain or a fragment thereof, and/or other IgM heavy chain domains. In certain embodiments, an IgM-derived binding molecule, e.g., an IgM antibody, IgM-like antibody, or other IgM-derived binding molecule as provided herein can include a complete IgM heavy (μ) chain constant domain, e.g., SEQ ID NO: 91 or SEQ ID NO: 92, or a variant, derivative, or analog thereof, e.g., as provided herein.

In certain embodiments, the disclosure provides a pentameric or hexameric binding molecule, where the binding molecule includes ten or twelve IgM-derived heavy chains, and where the IgM-derived heavy chains comprise IgM heavy chain constant regions each associated with a binding domain that specifically binds to a target, such as DR5. In certain embodiments, the disclosure provides an IgM antibody, IgM-like antibody, or IgM-derived binding molecule as provided herein can possess improved binding characteristics or biological activity as compared to a binding molecule composed of a single binding unit, e.g., a bivalent IgG antibody. For example, a pentameric or hexameric binding molecule can more efficiently cross-link three or more DR5 molecules on the surface of a cell, e.g., a tumor cell, thereby facilitating apoptosis of the cell. A binding molecule as provided herein can likewise possess distinctive characteristics compared to multivalent binding molecule composed of synthetic or chimeric structures. For example, use of human IgM constant regions can afford reduced immunogenicity and thus increased safety relative to a binding molecule containing chimeric constant regions or synthetic structures. Moreover, an IgM-based binding molecule can consistently form hexameric or pentameric oligomers resulting in a more homogeneous expression product. Superior complement fixation can also be an advantageous effector function of IgM-based binding molecules.

In certain embodiments, the disclosure provides an IgM antibody, IgM-like antibody, or IgM-derived binding molecule that includes five or six bivalent binding units, where each binding unit includes two IgM or IgM-like heavy chain constant regions or multimerizing fragments or variants thereof, each associated with an antigen-binding domain or subunit thereof. In certain embodiments, the two IgM heavy chain constant regions included in each binding unit arc human heavy chain constant regions. In some embodiments, the heavy chains are glycosylated. In some embodiments, the heavy chains can be mutated to affect glycosylation.

Where the IgM antibody, IgM-like antibody, or other IgM-derived binding molecule provided in this disclosure is pentameric, the IgM antibody, IgM-like antibody, or other IgM-derived binding molecule typically further include a J-chain, or functional fragment or variant thereof. In certain embodiments, the J-chain is a modified J-chain or variant thereof that further comprises one or more heterologous moieties attached to the J-chain, as described elsewhere herein. In certain embodiments, the J-chain can be mutated to affect, e.g., enhance, the serum half-life of the IgM antibody, IgM-like antibody, or other IgM-derived binding molecule provided herein, as discussed elsewhere in this disclosure. In certain embodiments the J-chain can be mutated to affect glycosylation, as discussed elsewhere in this disclosure.

An IgM heavy chain constant region can include one or more of a Cμ1 domain or fragment or variant thereof, a Cμ2 domain or fragment or variant thereof, a Cμ3 domain or fragment or variant thereof, and/or a Cμ4 domain or fragment or variant thereof, provided that the constant region can serve a desired function in the IgM antibody, IgM-like antibody, or other IgM-derived binding molecule, e.g., associate with second IgM constant region to form a binding unit with one, two, or more antigen-binding domain(s), and/or associate with other binding units (and in the case of a pentamer, a J-chain) to form a hexamer or a pentamer. In certain embodiments the two IgM heavy chain constant regions or fragments or variants thereof within an individual binding unit each comprise a Cμ4 domain or fragment or variant thereof, a tailpiece (tp) or fragment or variant thereof, or a combination of a Cμ4 domain and a TP or fragment or variant thereof. In certain embodiments the two IgM heavy chain constant regions or fragments or variants thereof within an individual binding unit each further comprise a Cμ3 domain or fragment or variant thereof, a Cμ2 domain or fragment or variant thereof, a Cμ1 domain or fragment or variant thereof, or any combination thereof.

In certain embodiments each of the two IgM heavy chain constant regions in a given binding unit is associated with an antigen-binding domain, for example an Fv portion of an antibody, e.g., a VH and a VL of a human or murine antibody, where the VL can be associated with a light chain constant region. In a binding molecule as provided herein at least three antigen-binding domains of the binding molecule are DR5 binding domains, i.e., binding domains that can specifically bind to DR5, e.g., human DR5.

In some embodiments, the binding units of the IgM antibody, IgM-like antibody, or other IgM-derived binding molecule comprise two light chains. In some embodiments, the binding units of the IgM antibody, IgM-like antibody, or other IgM-derived binding molecule comprise two fragments light chains. In some embodiments, the light chains are kappa light chains. In some embodiments, the light chains are lambda light chains. In some embodiments, each binding unit comprises two immunoglobulin light chains each comprising a VL situated amino terminal to an immunoglobulin light chain constant region.

IgA Antibodies, IgA-Like Antibodies, Other IgA-Derived Binding Molecules

IgA plays a critical role in mucosal immunity and comprises about 15% of total immunoglobulin produced. IgA is a monomeric or dimeric molecule. An IgA binding unit includes two light and two heavy chains. IgA contains three heavy chain constant domains (Cα1, Cα2 and Cα3), and includes a C-terminal “tailpiece.” Human IgA has two subtypes, IgA1 and IgA2. The human IgA1 constant region typically includes the amino acid sequence SEQ ID NO: 93. The human Cα1 domain extends from about amino acid 6 to about amino acid 98 of SEQ ID NO: 93; the human IgA1 hinge region extends from about amino acid 102 to about amino acid 124 of SEQ ID NO: 93, the human Cα3 domain extends from about amino acid 228 to about amino acid 330 of SEQ ID NO: 93, and the tailpiece extends from about amino acid 331 to about amino acid 352 of SEQ ID NO: 93. The human IgA2 constant region typically includes the amino acid sequence SEQ ID NO: 94. The human Cα1 domain extends from about amino acid 6 to about amino acid 98 of SEQ ID NO: 94; the human IgA2 hinge region extends from about amino acid 102 to about amino acid 111 of SEQ ID NO: 94, the human Cα2 domain extends from about amino acid 113 to about amino acid 206 of SEQ ID NO: 94, the human Cα3 domain extends from about amino acid 215 to about amino acid 317 of SEQ ID NO: 94, and the tailpiece extends from about amino acid 318 to about amino acid 340 of SEQ ID NO: 94.

Two IgA binding units can form a complex with two additional polypeptide chains, the J-chain (e.g., SEQ ID NO: 97 or SEQ ID NO: 98) and the secretory component (precursor, SEQ ID NO: 95, mature: amino acids 19 to 603 of SEQ ID NO: 95) to form a secretory IgA (sIgA) antibody. The assembly of IgA binding units into a dimeric sIgA antibody is thought to involve the Cα3 and tailpiece domains (also referred to herein collectively as the Cα3-tp domain). Accordingly, a dimeric sIgA antibody provided in this disclosure typically includes IgA constant regions that include at least the Cα3 and tailpiece domains.

An IgA heavy chain constant region can additionally include a Cα2 domain or a fragment thereof, an IgA hinge region, a Cα1 domain or a fragment thereof, and/or other IgA heavy chain domains. In certain embodiments, an IgA antibody or IgA-like binding molecule as provided herein can include a complete IgA heavy (a) chain constant domain (e.g., SEQ ID NO: 93 or SEQ ID NO: 94), or a variant, derivative, or analog thereof. In some embodiments, the IgA heavy chain constant regions or multimerizing fragments thereof are human IgA constant regions.

In some embodiments, the binding units of the IgA antibody, IgA-like antibody, or other IgA-derived binding molecule comprise two light chains. In some embodiments, the binding units of the IgA antibody, IgA-like antibody, or other IgA-derived binding molecule comprise two light chains. In some embodiments, the light chains are kappa light chains. In some embodiments, the light chains are lambda light chains. In some embodiments, each binding unit comprises two immunoglobulin light chains each comprising a VL situated amino terminal to an immunoglobulin light chain constant region.

In some embodiments, this disclosure provides a dimeric binding molecule, e.g., a binding molecule with two IgA “binding units” or fragments, variants, or derivatives thereof as defined herein, that can specifically bind to DR5. A binding molecule as provided herein can possess improved binding characteristics or biological activity as compared to a binding molecule composed of a single binding unit, e.g., a bivalent IgG antibody. For example, an IgA binding molecule can more efficiently cross-link three or more DR5 monomers on the surface of a cell, e.g., a tumor cell, thereby facilitating apoptosis of the cell. Moreover, an IgA binding molecule can reach mucosal sites providing greater tissue distribution for the binding molecules provided herein. Use of an IgA-based binding molecule can allow, for example, greater tissue distribution for a binding molecule provided herein. Mucosal distribution could be beneficial for certain cancers, e.g., lung cancer, gastric cancer, ovarian cancer, colorectal cancer, or squamous cell carcinoma. Likewise, a dimeric binding molecule as provided herein can possess binding characteristics or biological activity that can be distinguished from a binding molecule comprising five or six binding units, e.g., a hexameric or pentameric IgM antibody. For example, a dimeric binding molecule would be smaller, and could, for example, achieve better tissue penetration in solid tumors.

In certain embodiments, the disclosure provides a dimeric binding molecule comprising two bivalent binding units, where each binding unit includes two IgA heavy chain constant regions or fragments thereof. In certain embodiments, the two IgA heavy chain constant regions are human heavy chain constant regions.

A dimeric IgA binding molecule as provided herein can further comprise a J chain, or fragment thereof, or variant thereof. A dimeric IgA binding molecule as provided herein can further comprise a secretory component, or fragment thereof, or variant thereof.

An IgA heavy chain constant region can include one or more of a Cα1 domain, a Cα2 domain, and/or a Cα3 domain, provided that the constant region can serve a desired function in the binding molecule, e.g., associate with a light chain constant region to facilitate formation of an antigen binding domain or associate with another IgA binding unit to form a dimeric binding molecule. In certain embodiments the two IgA heavy chain constant regions or fragments thereof within an individual binding unit each comprise a Cα3 domain or fragment thereof, a tailpiece (TP) or fragment thereof, or any combination of a Cα3 domain, a TP, or fragment thereof. In certain embodiments the two IgA heavy chain constant regions or fragments thereof within an individual binding unit each further comprise a Cα2 domain or fragment thereof, a Cα1 domain or fragment thereof, or a Cα1 domain or fragment thereof and a Cα2 domain or fragment thereof.

In certain embodiments each of the two IgA heavy chain constant regions in a given binding unit is associated with an antigen binding domain, for example an Fv portion of an antibody. e.g., a VH and a VL of a human or murine antibody, where the VL can be associated with a light chain constant region. In a binding molecule as provided herein at least three antigen-binding domains of the binding molecule are DR5 binding domains, i.e., binding domains that can specifically bind to DR5, e.g., human DR5.

J-Chains and Functional Fragments or Variants Thereof

In certain embodiments, the dimeric or pentameric binding molecules provided herein comprises a J-chain or functional fragment or variant thereof. In certain embodiments, the multimeric binding molecule provided herein is pentameric and comprises a J-chain or functional fragment or variant thereof. In certain embodiments, the binding molecule provided herein is dimeric and comprises a J-chain or functional fragment or variant thereof. In some embodiments, the dimeric or pentameric binding molecule can comprise a naturally occurring J-chain sequence, such as a mature human J-chain sequence (e.g., SEQ ID NO: 97). Alternatively, in some embodiments, the dimeric or pentameric binding molecule can comprise a variant J-chain sequence, such as a variant sequence described herein with reduced glycosylation or reduced binding to polymeric Ig receptor (e.g., pIgR). In some embodiments, the dimeric or pentameric binding molecule can comprise a functional fragment of a naturally occurring or variant J-chain. As persons of ordinary skill in the art will recognize, “a functional fragment” or a “functional variant” in this context includes those fragments and variants that can associate with binding units, e.g., IgM or IgA heavy chain constant regions, to form a pentameric IgM antibody, IgM-like antibody, or IgM-derived binding molecule or a dimeric IgA antibody, IgA-like antibody, or IgA-derived binding molecule, and/or can associate with certain immunoglobulin receptors, e.g., pIgR.

In certain embodiments, the J-chain can be modified, e.g., by introduction of a heterologous moiety, or two or more heterologous moieties, e.g., polypeptides, without interfering with the ability of binding molecule to assemble and bind to its binding target(s). See U.S. Pat. Nos. 9,951,134 and 10,400,038, and U.S. Patent Application Publication Nos. US-2019-0185570 and US-2018-0265596, each of which is incorporated herein by reference in its entirety.

Accordingly, a binding molecule provided by this disclosure, including multispecific IgA, IgA-like, IgM, or IgM-like antibodies as described elsewhere herein, can comprise a modified J-chain or functional fragment or variant thereof comprising a heterologous moiety, e.g., a heterologous polypeptide, introduced, e.g., fused or chemically conjugated, into the J-chain or fragment or variant thereof. In certain embodiments, the heterologous polypeptide can be fused to the N-terminus of the J-chain or functional fragment or variant thereof, the C-terminus of the J-chain or functional fragment or variant thereof, or to both the N-terminus and C-terminus of the J-chain or functional fragment or variant thereof. In certain embodiments the heterologous polypeptide can be fused internally within the J-chain or functional fragment or variant thereof. In some embodiments, the heterologous polypeptide can be introduced into the J-chain at or near a glycosylation site. In some embodiments, the heterologous polypeptide can be introduced into the J-chain within about 10 amino acid residues from the C-terminus, or within about 10 amino acids from the N-terminus. In certain embodiments, the heterologous polypeptide can be introduced into the mature human J-chain of SEQ ID NO: 97 between cysteine residues 92 and 101 of SEQ ID NO: 97, or an equivalent location in a J-chain sequence, e.g., a J-chain variant or functional fragment of a J-chain. In a further embodiment, the heterologous polypeptide can be introduced into the mature human J-chain of SEQ ID NO: 97 at or near a glycosylation site. In a further embodiment, the heterologous polypeptide can be introduced into the mature human J-chain of SEQ ID NO: 97 within about 10 amino acid residues from the C-terminus, or within about 10 amino acids from the N-terminus.

In certain embodiments the heterologous moiety can be a peptide or polypeptide sequence fused in frame to the J-chain or chemically conjugated to the J-chain or fragment or variant thereof. In certain embodiments, the heterologous polypeptide is fused to the J-chain or functional fragment thereof via a peptide linker. Any suitable linker can be used, for example the peptide linker can include at least 5 amino acids, at least ten amino acids, and least 20 amino acids, at least 30 amino acids or more, and so on. In certain embodiments, the peptide linker includes least 5 amino acids, but no more than 25 amino acids. In certain embodiments the peptide linker can consist of 5 amino acids, 10 amino acids, 15 amino acids, 20 amino acids, or 25 amino acids. In certain embodiments, the peptide linker consists of GGGGS(SEQ ID NO: 99), GGGGSGGGGS (SEQ ID NO: 100), GGGGSGGGGSGGGGS (SEQ ID NO: 101), GGGGSGGGGSGGGGSGGGGS (SEQ ID NO: 102), or GGGGSGGGGSGGGGSGGGGSGGGGS (SEQ ID NO: 103).

In certain embodiments the heterologous moiety can be a chemical moiety conjugated to the J-chain. Heterologous moieties to be attached to a J-chain can include, without limitation, a binding moiety, e.g., an antibody or antigen-binding fragment thereof, e.g., a single chain Fv (scFv) molecule, a cytokine, e.g., IL-2 or IL-15 (see, e.g., PCT Application No. PCT US2019/057702, which is incorporated herein by reference in its entirety), a stabilizing peptide that can increase the half-life of the binding molecule, e.g., human serum albumin (HSA) or an HSA binding molecule, or a heterologous chemical moiety such as a polymer or a cytotoxin.

In some embodiments, a modified J-chain can comprise an antigen-binding domain that can include without limitation a polypeptide capable of specifically binding to a target antigen. In certain embodiments, an antigen-binding domain associated with a modified J-chain can be an antibody or an antigen-binding fragment thereof. In certain embodiments the antigen-binding domain can be a scFv antigen-binding domain or a single-chain antigen-binding domain derived, e.g., from a camelid or condricthoid antibody. In certain embodiments, the target is a target epitope, a target antigen, a target cell, or a target organ.

The antigen-binding domain can be introduced into the J-chain at any location that allows the binding of the antigen-binding domain to its binding target without interfering with J-chain function or the function of an associated multimeric binding molecule, e.g., a pentameric IgM or dimeric IgA antibody. Insertion locations include but are not limited to at or near the C-terminus, at or near the N-terminus or at an internal location that, based on the three-dimensional structure of the J-chain, is accessible.

Variant J-Chains that Confer Increased Serum Half-Life

In certain embodiments, the J-chain is a functional variant J-chain that includes one or more single amino acid substitutions, deletions, or insertions relative to a reference J-chain identical to the variant J-chain except for the one or more single amino acid substitutions, deletions, or insertions. For example, certain amino acid substitutions, deletions, or insertions can result in the IgM-derived binding molecule exhibiting an increased serum half-life upon administration to a subject animal relative to a reference IgM-derived binding molecule that is identical except for the one or more single amino acid substitutions, deletions, or insertions in the variant J-chain, and is administered using the same method to the same animal species. In certain embodiments the variant J-chain can include one, two, three, or four single amino acid substitutions, deletions, or insertions relative to the reference J-chain.

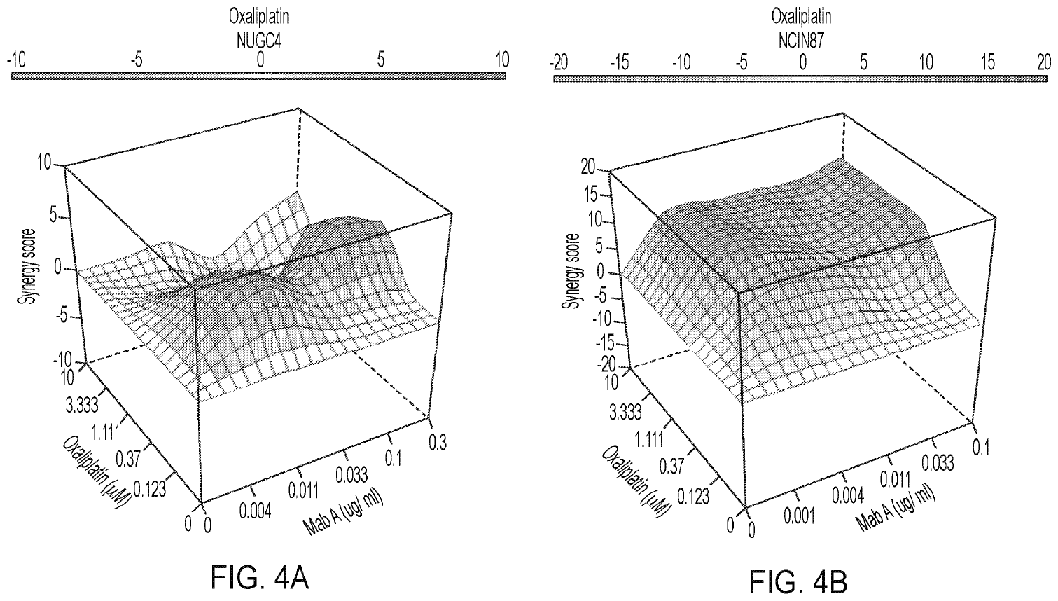

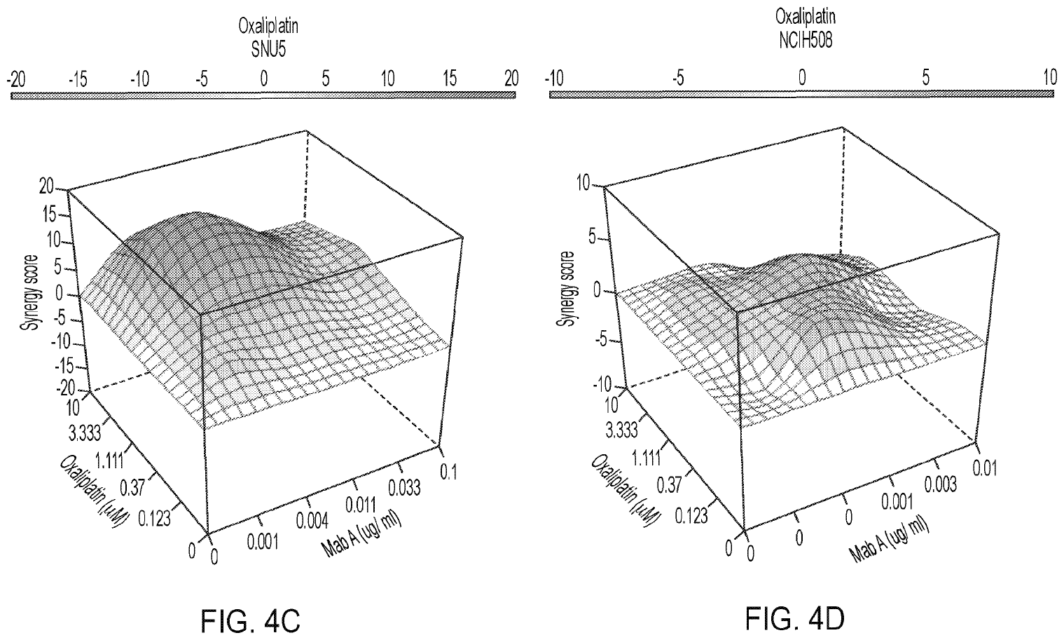

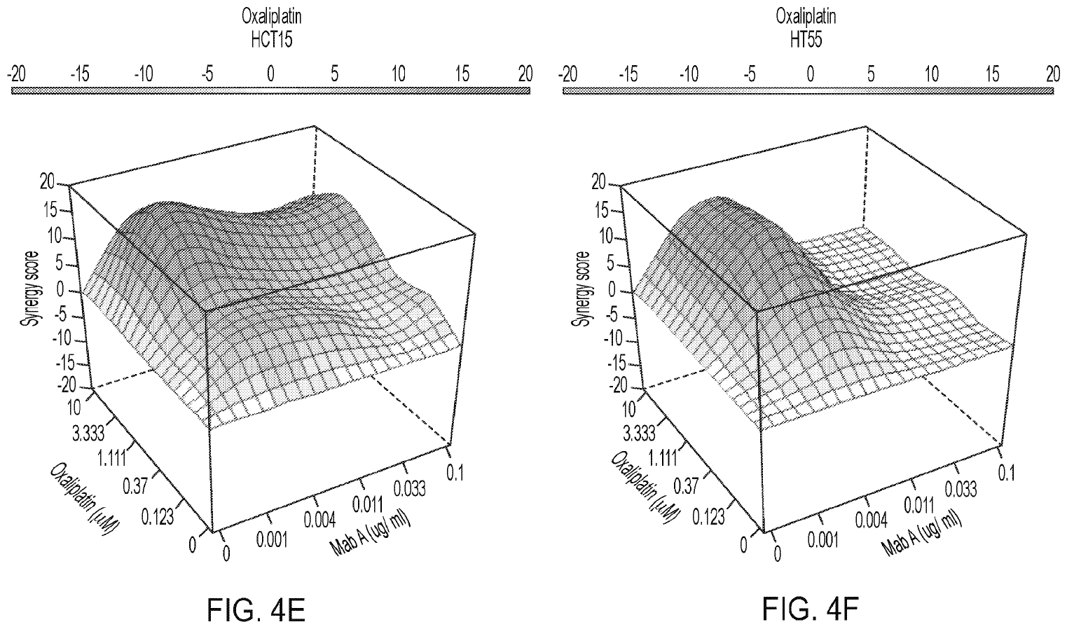

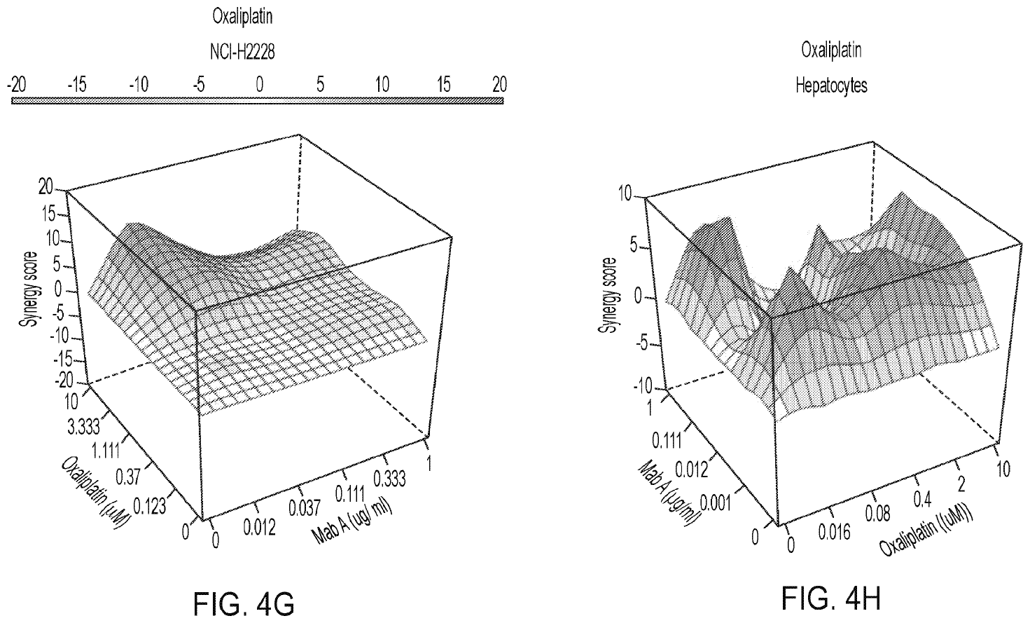

In certain embodiments, the J-chain, such as a modified J-chain, comprises an amino acid substitution at the amino acid position corresponding to amino acid Y102 of the mature wild-type human J-chain (SEQ ID NO: 97). By “an amino acid corresponding to amino acid Y102 of the mature wild-type human J-chain” is meant the amino acid in the sequence of the J-chain, which is homologous to Y102 in the human J-chain. For example, see PCT Publication No. WO 2019/169314, which is incorporated herein by reference in its entirety. The position corresponding to Y102 in SEQ ID NO: 97 is conserved in the J-chain amino acid sequences of at least 43 other species. See FIG. 4 of U.S. Pat. No. 9,951,134, which is incorporated by reference herein. Certain mutations at the position corresponding to Y102 of SEQ ID NO: 97 can inhibit the binding of certain immunoglobulin receptors, e.g., the human or murine Fcαμ receptor, the murine Fcμ receptor, and/or the human or murine polymeric Ig receptor (pIgR) to an IgM pentamer comprising the variant J-chain.

A multimeric binding molecule comprising a mutation at the amino acid corresponding to Y102 of SEQ ID NO: 97 has an improved serum half-life when administered to an animal than a corresponding multimeric binding molecule that is identical except for the substitution, and which is administered to the same species in the same manner. In certain embodiments, the amino acid corresponding to Y102 of SEQ ID NO: 97 can be substituted with any amino acid. In certain embodiments, the amino acid corresponding to Y102 of SEQ ID NO: 97 can be substituted with alanine (A), serine (S) or arginine (R). In a particular embodiment, the amino acid corresponding to Y102 of SEQ ID NO: 97 can be substituted with alanine. In a particular embodiment the J-chain or functional fragment or variant thereof is a variant human J-chain referred to herein as “*,” and comprises the amino acid sequence SEQ ID NO: 98.

Wild-type J-chains typically include one N-linked glycosylation site. In certain embodiments, a variant J-chain or functional fragment thereof of a multimeric binding molecule as provided herein includes a mutation within the asparagine (N)-linked glycosylation motif N—X₁—S/T, e.g., starting at the amino acid position corresponding to amino acid 49 (motif N6) of the mature human J-chain (SEQ ID NO: 97) or J* (SEQ ID NO: 98), where N is asparagine, X₁ is any amino acid except proline, and S/T is serine or threonine, and where the mutation prevents glycosylation at that motif. As demonstrated in PCT Publication No. WO 2019/169314, mutations preventing glycosylation at this site can result in the multimeric binding molecule as provided herein, exhibiting an increased serum half-life upon administration to a subject animal relative to a reference multimeric binding molecule that is identical except for the mutation or mutations preventing glycosylation in the variant J-chain, and is administered in the same way to the same animal species.

For example, in certain embodiments the variant J-chain or functional fragment thereof of a pentameric IgM-derived or dimeric IgA-derived binding molecule as provided herein can include an amino acid substitution at the amino acid position corresponding to amino acid N49 or amino acid S51 of SEQ ID NO: 97 or SEQ ID NO: 98, provided that the amino acid corresponding to S51 is not substituted with threonine (T), or where the variant J-chain comprises amino acid substitutions at the amino acid positions corresponding to both amino acids N49 and S51 of SEQ ID NO: 97 or SEQ ID NO: 98. In certain embodiments, the position corresponding to N49 of SEQ ID NO: 97 or SEQ ID NO: 98 is substituted with any amino acid, e.g., alanine (A), glycine (G), threonine (T), serine (S) or aspartic acid (D). In a particular embodiment, the position corresponding to N49 of SEQ ID NO: 97 or SEQ ID NO: 98 can be substituted with alanine (A). In another particular embodiment, the position corresponding to N49 of SEQ ID NO: 97 or SEQ ID NO: 98 can be substituted with aspartic acid (D).

Variant IgM Constant Regions

IgM heavy chain constant regions of a multimeric binding molecule as provided herein can be engineered to confer certain desirable properties to the multimeric binding molecules provided herein. For example, in certain embodiments, IgM heavy chain constant regions can be engineered to confer enhanced serum half-life to multimeric binding molecules as provided herein. Exemplary IgM heavy chain constant region mutations that can enhance serum half-life of an IgM-derived binding molecule are disclosed in PCT Publication No. WO 2019/169314, which is incorporated by reference herein in its entirety. For example, a variant IgM heavy chain constant region of the IgM antibody, IgM-like antibody, or IgM-derived binding molecule as provided herein can include an amino acid substitution at a position corresponding to amino acid S401, E402, E403, R344, and/or E345 of a wild-type human IgM constant region (e.g., SEQ ID NO: 91 or SEQ ID NO: 92). By “an amino acid corresponding to amino acid S401, E402, E403. R344, and/or E345 of a wild-type human IgM constant region” is meant the amino acid in the sequence of the IgM constant region of any species which is homologous to S401, E402, E403, R344, and/or E345 in the human IgM constant region. In certain embodiments, the amino acid corresponding to S401, E402, E403, R344, and/or E345 of SEQ ID NO: 91 or SEQ ID NO: 92 can be substituted with any amino acid, e.g., alanine.

In certain embodiments, an IgM antibody, IgM-like antibody, or other IgM-derived binding molecule as provided herein, can be engineered to exhibit reduced complement-dependent cytotoxicity (CDC) activity to cells in the presence of complement, relative to a reference IgM antibody, IgM-like antibody, or other IgM-derived binding molecule with corresponding reference human IgM constant regions identical, except for the mutations conferring reduced CDC activity. These CDC mutations can be combined with any of the mutations to confer increased serum half-life as provided herein. By “corresponding reference human IgM constant region” is meant a human IgM constant region that is identical to the variant IgM constant region except for the modification or modifications in the constant region affecting CDC activity. In certain embodiments, the variant human IgM constant region includes one or more amino acid substitutions, e.g., in the Cμ3 domain, relative to a wild-type human IgM constant region as described, e.g., in PCT Publication No. WO/2018/187702, which is incorporated herein by reference in its entirety. Assays for measuring CDC are well known to those of ordinary skill in the art, and exemplary assays are described e.g., in PCT Publication No. WO/2018/187702.

In certain embodiments, a variant human IgM constant region conferring reduced CDC activity includes an amino acid substitution corresponding to the wild-type human IgM constant region at position L310, P311, P313, and/or K315 of SEQ ID NO: 91 (human IgM constant region allele IGHM*03) or SEQ ID NO: 92 (human IgM constant region allele IGHM*04). In certain embodiments, a variant human IgM constant region conferring reduced CDC activity includes an amino acid substitution corresponding to the wild-type human IgM constant region at position P311 of SEQ ID NO: 91 or SEQ ID NO: 92. In other embodiments the variant IgM constant region as provided herein contains an amino acid substitution corresponding to the wild-type human IgM constant region at position P313 of SEQ ID NO: 91 or SEQ ID NO: 92. In other embodiments the variant IgM constant region as provided herein contains a combination of substitutions corresponding to the wild-type human IgM constant region at positions P311 of SEQ ID NO: 91 or SEQ ID NO: 92 and P313 of SEQ ID NO: 91 or SEQ ID NO: 92. These proline residues can be independently substituted with any amino acid, e.g., with alanine, serine, or glycine.

Human and certain non-human primate IgM constant regions typically include five (5) naturally-occurring asparagine (N)-linked glycosylation motifs or sites. As used herein “an N-linked glycosylation motif” comprises or consists of the amino acid sequence N—X₁—S/T, where N is asparagine, X₁ is any amino acid except proline (P), and S/T is serine (S) or threonine (T). The glycan is attached to the nitrogen atom of the asparagine residue. See, e.g., Drickamer K. Taylor M E (2006), Introduction to Glycobiology (2nd ed.). Oxford University Press, USA. N-linked glycosylation motifs occur in the human IgM heavy chain constant regions of SEQ ID NO: 91 or SEQ ID NO: 92 starting at positions 46 (“N I”), 209 (“N2”), 272 (“N3”), 279 (“N4”), and 440 (“N5”). These five motifs are conserved in non-human primate IgM heavy chain constant regions, and four of the five are conserved in the mouse IgM heavy chain constant region. Accordingly, in some embodiments, IgM heavy chain constant regions of a multimeric binding molecule as provided herein comprise 5 N-linked glycosylation motifs: N1, N2, N3, N4. and N5. In some embodiments, at least three of the N-linked glycosylation motifs (e.g., N1, N2, and N3) on each IgM heavy chain constant region are occupied by a complex glycan.

In certain embodiments, at least one, at least two, at least three, or at least four of the N—X₁—S/T motifs can include an amino acid insertion, deletion, or substitution that prevents glycosylation at that motif. In certain embodiments, the IgM-derived multimeric binding molecule can include an amino acid insertion, deletion, or substitution at motif N1, motif N2, motif N3, motif N5, or any combination of two or more, three or more, or all four of motifs N1, N2, N3, or N5, where the amino acid insertion, deletion, or substitution prevents glycosylation at that motif. In some embodiment, the IgM constant region comprises two or more substitutions relative to a wild-type human IgM constant region at positions 46, 209, 272, or 440 of SEQ ID NO: 91 (human IgM constant region allele IGHM*03) or SEQ ID NO: 92 (human IgM constant region allele IGHM*04). See, e.g., U.S. Provisional Application No. 62/891,263, which is incorporated herein by reference in its entirety.

DR5 Binding Domains

A DR5 binding molecule, e.g., an anti-DR5 antibody or fragment, variant, or derivative thereof as provided herein can be dimeric, pentameric, or hexameric, comprising two, five, or six bivalent binding units, respectively. The binding units can be full length or variants or fragments thereof that retain binding function.

Each binding unit comprises two IgA or IgM heavy chain constant regions or fragments thereof, each associated with an antigen-binding domain. As noted above, an antigen binding domain is a region of a binding molecule that is necessary and sufficient to specifically bind to an epitope. A “binding molecule” as described herein can include one, two, three, four, five, six, seven, eight, nine, ten, eleven, twelve or more “antigen binding domains.”

A dimeric, pentameric, or hexameric binding molecule as provided herein can include at least three antigen-binding domains which specifically and agonistically bind to DR5. As noted above DR5, upon activation, can induce apoptosis of the cell expressing the DR5 proteins which were bound. Apoptosis will occur, as presently understood, when multiple receptor proteins are bound together, causing cross-linking of the receptor molecules such that a signal is transmitted across the cell membrane into the cytosol of the cell expressing DR5.

A dimeric, pentameric, or hexameric binding molecule as provided herein can cross-link at least three DR5 monomers expressed on the surface of a cell. Due to the dimeric, pentameric, or hexameric nature of a DR5 binding molecule as provided herein, the molecule can cross-link as many as three, four, five, six, seven, eight, nine, ten, eleven, or twelve DR5 monomers on a cell. The receptor proteins are then spatially brought into proximity of each other, thereby contributing to their cross-linking and activation. When all five or all six of the bivalent binding units a DR5 binding molecule as provided herein bind to a receptor, binding up to ten or twelve DR5 monomers on a single cell, respectively, cross-linking and activation of the receptors can occur.

Because each of the binding units is bivalent, each binding molecule can bind to as many as 4 (for dimeric binding molecules), 10 (for pentameric binding molecules), or 12 (for hexameric binding molecules) DR5 monomers.

Upon activation of the receptors by the binding of a dimeric, pentameric, or hexameric binding molecule as provided herein, the cell can either undergo apoptosis as described above.

In certain embodiments, a dimeric, pentameric, or hexameric binding molecule as presently disclosed can induce DR5-mediated apoptosis in a DR5-expressing cell at a higher potency than an equivalent amount of a bivalent IgG antibody or fragment thereof, which also specifically binds to and agonizes DR5. Not wishing to be bound by theory, because a provided binding molecule is dimeric, pentameric, or hexameric, and because each binding unit is bivalent, such a binding molecule can induce receptor-mediated functions previously characterized for DR5 at a higher potency than any single binding unit alone, such as an equivalent IgG binding unit. IgG binding units are bivalent, containing two binding sites, but as previous clinical studies have shown, binding of two DR5 receptors with a single IgG molecule can be ineffective without addition of other components, such as cross-linkers, etc.

By “potency” or “improved binding characteristics” is meant the least amount of a given binding molecule necessary to achieve a given biological result, e.g., activation of 20%, 50%, or 90% of DR5 monomers in a given assay, e.g., an ELISA or Western blot-based caspase assays, annexin-v staining as seen by FACS analysis, or other assay. Or a reduced tumor growth rate or increased survival in an in vivo tumor assay.

Because a binding molecule as provided herein is dimeric, pentameric, or hexameric, it can contain as many as 4, 10, or 12, respectively, antigen-binding domains. Each of the antigen-binding domains can specifically bind to and agonize DR5. Further, each antigen-binding domain can be specific for one particular epitope of DR5.

Thus, a single dimeric. pentameric, or hexameric binding molecule can: a) simultaneously bind a single epitope on DR5, or b) bind many different epitopes on DR5.

The binding units of a dimeric, pentameric, or hexameric binding molecule as provided herein can be human, humanized, or chimeric immunoglobulin binding units. Methods of humanizing immunoglobulin sequences are well known in the art. Thus, the nucleotide sequences encoding a dimeric, pentameric, or hexameric binding molecule polypeptide can be directly from human sequences, or can be humanized or chimeric, i.e., encoded by sequences from multiple different species.

The cells which express DR5 can be any animal cell. For instance, in one embodiment, the cell is a human cell. For example, the cell can be any one or more of primate, rodent, canine, equine, etc., cells. Further, the cell expressing DR5 can be a cancer cell. That is, the cell can be a cell in a tumor which is malignant or benign.

A dimeric, pentameric, or hexameric binding molecule as provided herein can be genetically engineered such that its antigen-binding domains are encoded by sequences known to specifically bind DR5. Many groups have published sequences of variable regions of monoclonal antibodies, most of the IgG isotype that are characterized and are known to specifically bind to DR5. Non-limiting immunoglobulin variable domain sequences that are known to specifically bind to DR5 are provided in Tables 2 and 3. One of skill in the art is capable of engineering these published sequences into immunoglobulin structures, such as an IgG, IgA, IgM structure, or biologically active or functional multimeric fragments variants, or derivatives thereof. Methods for genetically engineering cloned variable regions into immunoglobulin domains, and expressing and purifying such constructs are published and within the capability of one skilled in the art.

Thus, in certain embodiments, a DR5 binding domain as provided herein comprises six immunoglobulin complementarity determining regions HCDR1, HCDR2, HCDR3, LCDR1, LCDR2, and LCDR3, or the six immunoglobulin complementarity determining regions with one, two, three, four, or five single amino acid substitutions in one or more CDR, of an anti-DR5 mAb comprising the VH and VL amino acid sequences SEQ ID NO: 1 and SEQ ID NO: 2; SEQ ID NO: 3 and SEQ ID NO: 4; SEQ ID NO: 5 or SEQ ID NO: 90 and SEQ ID NO: 6; SEQ ID NO: 7 and SEQ ID NO: 8; SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; SEQ ID NO: 23 and SEQ ID NO: 24; SEQ ID NO: 25 and SEQ ID NO: 26; SEQ ID NO: 27 and SEQ ID NO: 28; SEQ ID NO: 29 and SEQ ID NO: 30; SEQ ID NO: 31 and SEQ ID NO: 32; SEQ ID NO: 33 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46; SEQ ID NO: 47 and SEQ ID NO: 48; SEQ ID NO: 49 and SEQ ID NO: 50; SEQ ID NO: 51 and SEQ ID NO: 52; SEQ ID NO: 53 and SEQ ID NO: 54; SEQ ID NO: 55 and SEQ ID NO: 56; SEQ ID NO: 82 and SEQ ID NO: 83; SEQ ID NO: 84 and SEQ ID NO: 85; SEQ ID NO: 86 and SEQ ID NO: 87; or SEQ ID NO: 88 and SEQ ID NO: 89; respectively, or the ScFv sequence SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67. SEQ ID NO: 68, SEQ ID NO: 69. SEQ ID NO: 70, SEQ ID NO: 71. SEQ ID NO: 72, or SEQ ID NO: 73.

In some embodiments, a DR5 binding domain as provided herein comprises six immunoglobulin complementarity determining regions HCDR1, HCDR2, HCDR3, LCDR1, LCDR2, and LCDR3, or the six immunoglobulin complementarity determining regions with one, two, three, four, or five single amino acid substitutions in one or more CDR, of an anti-DR5 mAb comprising the VH and VL amino acid sequences SEQ ID NO: 5 or SEQ ID NO: 90 and SEQ ID NO: 6; or SEQ ID NO: 7 and SEQ ID NO: 8, respectively. In some embodiments. a DR5 binding domain as provided herein comprises six immunoglobulin complementarity determining regions HCDR1, HCDR2, HCDR3, LCDR1, LCDR2, and LCDR3, or the six immunoglobulin complementarity determining regions with one, two, three, four, or five single amino acid substitutions in one or more CDR, of an anti-DR5 mAb comprising the VH and VL amino acid sequences SEQ ID NO: 5 and SEQ ID NO: 6, respectively. In some embodiments, a DR5 binding domain as provided herein comprises six immunoglobulin complementarity determining regions HCDR1. HCDR2, HCDR3, LCDR1, LCDR2, and LCDR3, or the six immunoglobulin complementarity determining regions with one, two, three, four, or five single amino acid substitutions in one or more CDR, of an anti-DR5 mAb comprising the VH and VL amino acid sequences SEQ ID NO: 90 and SEQ ID NO: 6; or SEQ ID NO: 7 and SEQ ID NO: 8, respectively. In some embodiments, a DR5 binding domain as provided herein comprises six immunoglobulin complementarity determining regions HCDR1, HCDR2, HCDR3, LCDR1, LCDR2, and LCDR3, or the six immunoglobulin complementarity determining regions with one, two, three, four, or five single amino acid substitutions in one or more CDR, of an anti-DR5 mAb comprising the VH and VL amino acid sequences SEQ ID NO: 7 and SEQ ID NO: 8, respectively.

TABLE 2
Anti-DR5 Antibody VH (or Heavy Chain) and VL (or Light Chain) Sequences

| SEQ | | SEQ | | Reference |
|---|---|---|---|---|
| ID | VH or Heavy Chain | ID | VL or Light Chain | |
| 1 | EVQLVQSGGGVERPGGSLRLSCAASGFTFDD | 2 | SSELTQDPAVSVALGQTVRITCQGDSLRSYY | U.S. Patent |
| | YGMSWVRQAPGKGLEWVSGINWNGGSTGYAD | | ASWYQQKPGQAPVLVIYGKNNRPSGIPDRFS | App. Pub. |
| | SVKGRVTISRDNAKNSLYLQMNSLRAEDTAV | | GSSSGNTASLTITGAQAEDEADYYCNSRDSS | No. |
| | YYCAKILGAGRGWYFDLWGKGTTVTVSS | | GNHVVFGGGTKLTVL | 20060269555A1 |
| 3 | EVQLVQSGGGVERPGGSLRLSCAASGFTFDD | 4 | SELTQDPAVSVALGQTVRITCSGDSLRSYYA | U.S. Pat. No. |
| | YAMSWVRQAPGKGLEWVSGINWQGGSTGYAD | | SWYQQKPGQAPVLVIYGANNRPSGIPDRFSG | 8,029,783 |
| | SVKGRVTISRDNAKNSLYLQMNSLRAEDTAV | | SSSGNTASLTITGAQAEDEADYYCNSADSSG | |
| | YYCAKILGAGRGWYFDYWGKGTTVTVSS | | NHVVFGGGTKLTVL | |
| 5 | QVQLQESGPGLVKPSQTLSLTCTVSGGSISS | 6 | EIVLTQSPGTLSLSPGERATLSCRASQGISR | U.S. Pat. No. |
| | GDYFWSWIRQLPGKGLECIGHIHNSGTTYYN | | SYLAWYQQKPGQAPSLLIYGASSRATGIPDR | 7,521,048 |
| | PSLKSRVTISVDTSKKQFSLRLSSVTAADTA | | FSGSGSGTDFTLTISRLEPEDFAVYYCQQFG | |
| | VYYCARDRGGDYYYGMDVWGQGTTVTVSS | | SSPWTFGQGTKVEIK | |
| 7 | EVQLVESGGGLVQPGGSLRLSCAASGFTFSS | 8 | DIQMTQSPSSLSASVGDRVTITCKASQDVGT | U.S. Pat. No. |
| | YVMSWVRQAPGKGLEWVATISSGGSYTYYPD | | AVAWYQQKPGKAPKLLIYWASTRHTGVPSRF | 7,790,165 |
| | SVKGRFTISRDNAKNTLYLQMNSLRAEDTAV | | SGSGSGTDFTLTISSLQPEDFATYYCQQYSS | |
| | YYCARRGDSMITTDYWGQGTLVTVSS | | YRTFGQGTKVEIK | |
| 9 | QIQLVQSGPELKKPGETVKISCKASGYTFTD | 10 | DVVMTQTPLSLPVSLGDQASISCRSSQSLVH | U.S. Pat. No. |
| | FSMNWVKQAPGKGLKWMGWINTETGEPTYAD | | SNGNTYLHWYLQKPGQSPKLLIYKVSNRFSG | 7,893,216 |
| | DFKGRFALSMETSASTAYLQINNLKNEDTAT | | VPDRFSGSGSGTDFTLKISRVEAEDLGVYFC | |
| | YFCVRIDYWGQGTTLTVSS | | FQSTHVPHTFGGGTKLEIKR | |
| 11 | MDWTWRILFLVAAATSAHSQVQLVQSGAEMK | 12 | MEAPAQLLFLLLLWLPDTTGEIVLTQSPATL | U.S. Pat. No. |
| | KPGASVKVSCKTSGYTFTNYKINWVRQAPGQ | | SLSPGERATLSCRASQSVSSYLAWYQQKPGQ | 7,115,717 |
| | GLEWMGWMNPDTDSTGYPQKFQGRVTMTRNT | | APRLLIYDASNRATGIPARFSGSGSGTDFTL | |
| | SISTAYMELSSLRSEDTAVYYCARSYGSGSY | | TISSLEPEDFAVYYCQQRSNWPLTFGGGTKV | |
| | YRDYYYGMDVWGQGTTVTVSS | | EIKR | |
| 13 | EVQLQQSGPELVKPGASVKISCKASGYSFIG | 14 | DVVMTQTPLSLPVSLGDQASISCRSSQSLVH | EP Patent |
| | YFMNWMKQSHGKSLEWIGRFNPYNGDTFYNQ | | SNGNTYLHWYLQKPGQSPKLLIYKVSNRFSG | Publication |
| | KFKGKATLTVDKSSTTAHMELLSLTSEDSAV | | VPDRFSGSGSGTDFTLKISRVEAEDLGIYFC | No. |
| | YFCGRSAYYFDSGGYFDYWGQGTTLTVSS | | SQSTHVPWTFGGGTKLEIK | EP2636736A1 |
| 15 | QVQLVQSGSELKKPGASVKVSCKASGYTFTD | 16 | DIVMTQSPLSLPVTPGEPASISCRSSQSLVH | PCT Publi- |
| | FSMNWVRQAPGQGLEWMGWINTETGEPTYAD | | SNGNTYLHWYLQKPGQSPQLLIYKVSNRFSG | cation No. |
| | DFKGRFVFSLDTSVSTAYLQISSLKAEDTAV | | VPDRFSGSGSGTDFTLKISRVEAEDVGVYYC | WO 2014/ |
| | YYCARIDYWGQGTTVTVSS | | FQSTHVPHTFGQGTKLEIKR | 063368 A1 |
| 17 | GVQCEVHLVESGGGLVRPGGSLKLSCAASGF | 18 | DIQMTQSSSSFSVSLGDRVTITCKASEDIYN | U.S. Pat. No. |
| | AFSSYDMSWVRQTPEKRLEWVAYISDGGGIT | | RLAWYQQKPGNAPRLLISGATSLETGVPSRF | 7,897,730 |
| | YYPDTMKGRFTISRDNAKNTLSLQMSSLKSE | | SGSGSGKDYTLSITSLQTEDVATYYCQQYWS | |
| | DTAMYYCARHITMVVGPFAYWGQGTLVTVSA | | TPLTFGAGTKLELKR | |
| 19 | EVQLQQSGPELVKPGASVRMSCKASGYTFTS | 20 | DIVMTQSHKFMSTSVGDRVSITCKASQDVST | U.S. Pat. No. |
| | YFIHWVKQRPGQGLEWIGWIYPGNVNTKYSE | | AVAWYQQKPGQSPRLLIYWASTRHTGVPDRF | 7,897,730 |
| | KFKGKATLTADKSSSTAYMQFSSLTSEDSAV | | TGSGSGTDYTLTISSVQAEDQALYYCQQHYR | |
| | YFCARGEAGYFDYWGQGTTLTVSS | | TPWTFGGGTKLEIK | |
| 21 | QVQLVQSGAEVKKPGASVKVSCKASGYTFTS | 22 | DIQMTQSPSSLSASVGDRVTITCRASQSISI | U.S. Pat. No. |
| | YDINWVRQATGQGLEWMGWMNPNSDNTGYAQ | | YLNWYQQKPGKAPKLLIYAASSLQSGVPLRF | 7,521,048 |
| | KFQGRVTMTRNTSISTAYMELSSLRSEDTAV | | SGSGSGTDFTLTISSLQPEDIATYYCQQSYK | |
| | YYCARWNHYGSGSHFDYWGQGTLVTVSS | | TPLTFGGGTKVEIK | |
| 23 | QVQLQESGPGLVKPSQTLSLTCTVSGGSISS | 24 | DIQMTQSPSSLSASVGDRVTITCRASQGLRN | U.S. Pat. No. |
| | GGHYWSWIRQHPGKGLEWIGYIYYSGSTYYN | | DLGWFQQKPGKVTKRLIYAASSLQRGVPSRF | 7,521,048 |
| | PSLKSRVTISVDTSKNQFSLKLSSVTAADTA | | SGSGSGTEFTLTISSLQPEDFATYYCLQHYS | |
| | VYYCARDDSSGWGFDYWGQGILVTVSS | | FPWTFGQGTKVEIK | |
| 25 | QVQLQESGPGLVKPSQTLSLTCTVSGGSISS | 26 | DIQMTQSPSSLSASVGDRVTITCRASQGLRN | U.S. Pat. No. |
| | GGHYWSWIRQHPGKGLEWIGYIYYSGSAYYN | | DLGWFQQKPGKAPKRLIYAASSLQRGVPSRF | 7,521,048 |
| | PSLKSRVTISVDTSKNQFSLKLSSVTAADTA | | SGSGSGTEFTLTISSLQPEDFTTYFCLQHNS | |
| | VYYCARDDSSGWGFDYWGQGILVTVSS | | FPWTFGQGTKVEIK | |
| 27 | QVQLQESGPGLVKPSQTLSLTCTVSGGSISS | 28 | DIQMTQSPSSLSASVGDRVTITCRASQGLRN | U.S. Pat. No. |
| | GGHYWSWIRQHPGKGLEWIGYIYYSGSAYYN | | DLGWFQQKPGKAPKRLIYAASSLQRGVPSRF | 7,521,048 |
| | PSLKSRVTISVDTSKNQFSLKLSSVTAADTA | | SGSGSGTEFTLTISSLQPEDFTTYFCLQHNS | |
| | VYYCARDDSSGWGFDYWGQGILVTVSS | | FPWTFGQGTKVEIK | |
| 29 | QVQLVESGGGLVKPGGSLRLSCAASGFTFSD | 30 | DIQMTQSPSSLSASVGDRVTITCRSSQSISN | U.S. Pat. No. |
| | YYMNWIRQAPGKGLEWVSHISSSGSILDYAD | | YINWYQQRPGKAPNLLIHDVSSFQSAVPSRF | 7,521,048 |
| | SVKGRFTISRDNAKNSLYLQMNSLRVEDTAV | | SRSGSGTVFTLTISSLQPEDFATYFCQQTYI | |
| | YYCARDGAAAGTDAFDLWGQGTMVTVSS | | TPFTFGPGTKVDIK | |
| 31 | QVQLVESGGGVVQPGRSLRLSCAASGFTFSY | 32 | DIQMTQSPSSLSASVGDRVTITCRASQGISN | U.S. Pat. No. |
| | YGIHWVRQAPGKGLEWVAVIWYDGSNKYYAD | | YLAWYQQKPGKVPKLLIYAASTLQSGVPSRF | 7,521,048 |
| | SVKGRFTISRDNSKNTLYLQMNSLRAEDTAV | | SGSGSGTDFTLTISSLQPEDVATYYCQKYNS | |
| | YYCARGRYSSSSWWYFDLWGRGTLVTVSS | | APLTFGGGTKVEIK | |
| 33 | QVQAEQSGPGLVKPSETLSLTCTVSGGSISN | 34 | DIVMTQSPDSLAVSLGERATINCKSSQSVLY | U.S. Pat. No. |
| | YYWSWIRQPPGKGLEWIGYIYYSGSTKYNPS | | RSNNKIYLAWYQQKPGQPPKLLIYWASTRES | 7,521,048 |
| | LKSRVTISVDTSKNQFSLKLTSVTTADTAVY | | GVPDRFSGSGSGTDFTLTISSLLAEDVAVYY | |
| | YCARDSPRGFSGYEAFDSWGQGTLVTVSS | | CQQYYSTPFTFGPGTKVDIK | |
| 35 | QVQLQESGPGLVKPSQTLSLTCTVSGGSISS | 36 | DIVMTQSPLSLPVTPGEPASISCRSSQSLLR | U.S, Pat. No. |
| | DNYYWSWIRQHPGKGLEWIGYIYYSGSTYYN | | RNGYNYLDWYLQKPGQSPQLLIYLGSNRASG | 7,521,048 |
| | PSLKSRVTISVDTSKNQFSLKLSSVTAADTA | | VPDRFSGSGSGTDFTLKISRVEAEDVGVYYC | |
| | VYYCARQVNWNFLFDIWGQGTMVTVSS | | MQALQTPLTFGGGTEVEIK | |
| 37 | QVQLVESGGGLVKPGGSLRLSCAASGFTFSD | 38 | DIVMTQFPDSLAVSLGERATINCKSSQSVLH | U.S. Pat. No. |
| | YYMSWIRQAPGKGLEWVSYISRSGSTIYYAD | | SSNNKNYLTWYQLKPGQPPKLLIYWASTRES | 7,521,048 |
| | SVKGRFTISRDNAKNSLYLQMNSLRAEDTAV | | GVPDRFSGSGSGTDFTLTISSLQAEDVAVYY | |
| | YYCARSLGGMDVWGQGTTVTVSS | | CHQYYSTPSSFGQGTKLEIK | |
| 39 | QVQLVESGGGVVQPGRSLRLSCAASGFTFNN | 40 | DIQMTQSPSSLSASVGDRVTITCRTSQSIST | U.S. Pat. No. |
| | YGMHWVRQAPGKGLEWVAVIWYDGSNKYYAD | | YLNWYQQKPGKAPKLLISATSSLQSGVPSRF | 7,521,048 |
| | SVKGRFTISRDNSKNTLYLQMNSLRAEDTAV | | SGSGSGTDFTLTISSLQPEDFATYYCQQSYS | |
| | YYCARDRTVYSNSSPFYYYYYGMDVWGQGTT | | TPLTFGGGTKVEIK | |
| | VTVSS | | | |
| 41 | QVQLVESGGGVVQPGRSLRLSCAASGFTFST | 42 | DIQMTQSPSSLSASVGDRVTITCRASQSISS | U.S. Pat. No. |
| | YGMHWVRQAPGKGLEWVAVIWYDGSNKYYAD | | YLNWYQQKPGKAPKLLISATSSFQSGVPSRF | 7,521,048 |
| | SVKGRFTISRDNSKNTLYLQMNSLRAEDTAV | | SGSGSGTDFTLTISSLQPEDFAAYYCQQSYS | |
| | YYCARDRTVYSSSSPFYYYYYGMDVWGQGTT | | TPLTFGGGTKVEIK | |
| | VTVSS | | | |
| 43 | QVQLQQWGARLLKPSETLSLTCAVYGGSFSG | 44 | DIVMTQSPDSLAVSLGERATINCKSSQSVLH | U.S. Pat. No. |
| | YYWSWIRQPPGKGLEWIGEINHSGSTNYNPS | | SSNNKNYLVWYQQKPGQPPKLLIYWASTRES | 7,521,048 |
| | LKSRVTISVDTSKNQFSLKLRSVTAADTAVY | | GVPDRFSGSGSGTDFTLTISSLQAEDVAVYY | |
| | YCARGGSSGYWYFDLWGRGTLVTVSS | | CQQYYSTPLTFGGGTKVEIK | |
| 45 | EVQVVESGGGLVKPGGSLRLSCAASGFTFSS | 46 | DIQMTQSPSSVSASVGDRVTITCRASQGISS | U.S. Pat. No. |
| | YSMNWVRQAPGKGLEWVSSISSSSSYIYYAD | | WLVWYQQKPGKAPKLLIYAASSLQSGVPSRF | 7,521,048 |
| | SVKGRFTISRDNAKNSLYLQMNSLRAEDTAV | | SGSGSGTDFTLTISSLQPEDFATYYCQQANS | |
| | YYCARGGSSWYGDWFDPWGQGTLVTVSS | | FPFTFGGGTKVEIK | |
| 47 | QLVESGGGVVQPGRSLRLSCAASGFTFSSYG | 48 | DIQMTQSPSSLSASVGDRVTITCRASQGISN | U.S. Pat. No. |
| | MHWVRQAPGKGLEWVAVIWYDGRNKYYADSV | | YLAWFQQKPGKAPKSLIYAASSLQSGVPSKF | 7,521,048 |
| | KGRFTISRDNSKNTLYLQMNSLRAEDTAVYY | | SGSGSGTDFTLTISSLQPEDFATYYCQQYNS | |
| | CAREVGYCTNGVCSYYYYGMDVWGQGTTVTV | | YPLTFGGGTKVEIK | |
| | SS | | | |
| 49 | QVQLQESGPGLVKPSQTLSLTCSVSGGSISS | 50 | DIQMTQSPSSVSASVGDRVTITCRASQGISS | U.S. Pat. No. |
| | GGYYWSWIRQHPGKGLEWIGYIYYSGSTYCN | | WLAWYQQKPGKAPKFLIFVASSFQSGVPSRF | 7,521,048 |
| | PSLKSRVTISVDTSKNQFSLKLSSVTAADTA | | SGSGSGTDFTLTISSLQPEDFATYYCQQANS | |
| | VYYCARDNGSGSYDWFDPWGQGILVTVSS | | FPRTFGQGTKVEIK | |
| 51 | QVQMQESGPGLVKPSQTLSLTCTVSGGSISS | 52 | DIQMTQSPSSVSASVGDRVTITCRASQGISS | U.S. Pat. No. |
| | GDYYWSWIRQHPGKNLEWIGYIYYSGSTYYN | | WLAWYQQKPGKAPKFLIFVASSLQSGVPSRF | 7,521,048 |
| | PSLKSRVTISVDTSKNQFSLKLSSVTAADTA | | SGSGSGTDFTLTISSLQPEDFATYYCQQANS | |
| | VYYCARDNGSGSYDWFDPWGQGTLVTVSS | | FPRTFGQGTKVEIK | |
| 53 | KVQLQQSGAELVKPGASVKLSCKASGYTFTD | 54 | DIAMTQSHKFMSTLVGDRVSITCKASQDVNT | U.S. Pat. No. |
| | YTIHWVKQRSGQGLEWIGWFYPGGGYIKYNE | | AIAWYQQKPGQSPKLLIYWASTRHTGVPDRF | 7,229,617 |
| | KFKDRATLTADKSSNTVYMELSRLTSEGSAV | | TGSGSGTDYTLTISSMEAEDAATYYCQQWSS | |
| | YFCARHEEGIYFDYWGQGTTLTVSS | | NPLTFGAGTKLELKRA | |
| 55 | KVQLQQSGAELVKPGASVKLSCKASGYTFTD | 56 | DIVMTQSHKFMSTSVGDRVSITCKASQDVNT | U.S. Pat.t No. |
| | YTIHWVKQRSGQGLEWIGWFYPGGGYIKYNE | | AIAWYQQKPGQSPKLLIYWASTRHTGVPDRF | 7,229,618 |
| | KFKDRATLTADKSSNTVYMELSRLTSEDSAV | | TGSGSGTDYTLTISSVQAEDLALYYCQQHYT | |
| | YFCARHEEGIYFDYWGQGTTLTVSS | | PHTEGSGTKL | |
| 82 | MDLMCKKMKHLWFFLLLVAAPRWVLSQLQLQ | 83 | MEAPAQLLFLLLLWLPDTTGEIVLTQSPATL | U.S. Pat. No. |
| | ESGPGLVKPSETLSLTCTVSGGSIISKSSYW | | SLSPGERATLSCRASQSVSSFLAWYQQKPGQ | 7,115,717 |
| | GWIRQPPGKGLEWIGSIYYSGSTFYNPSLKS | | APRLLIYDASNRATGIPARFSGSGSGTDFTL | |
| | RVTISVDTSKNQFSLKLSSVTAADTAVYYCA | | TISSLEPEDFAVYYCQQRSNWPLTFGPGTKV | |
| | RLTVAEFDYWGQGTLVTVSSAS | | DIKRT | |
| 84 | MDLMCKKMKHLWFFLLLVAAPRWVLSQLQLQ | 85 | MEAPAQLLFLLLLWLPDTTGEIVLTQSPATL | U.S. Pat. No. |
| | ESGPGLVKPSETLSLTCTVSGGSISSRSNYW | | SLSPGERATLSCRASQSVSSFLAWYQQKPGQ | 7,115,717 |
| | GWIRQPPGKGLEWIGNVYYRGSTYYNSSLKS | | APRLLIYDASNRATGSPARFSGSGSGTDFTL | |
| | RVTISVDTSKNQFSLKLSSVTVADTAVYYCA | | TISSLEPEDFAVYYCQQRSDWPLTFGPGTKV | |
| | RLSVAEFDYWGQGILVTVSSAS | | DIKRT | |
| 86 | MDLMCKKMKHLWFFLLLVAAPRWVLSQLQLQ | 87 | METPAQLLFLLLLWLPDTTGEIVLTQSPGTL | U.S. Pat. No. |
| | ESGPGLVKPSETLSLTCTVSGGSISSSSYYW | | SLSPGERATLSCRASQSVSSSYLAWYQQKPG | 7,115,717 |
| | GWVRQPPGKGLEWIGSIHYSGSTFYNPSLKS | | QAPRLLIYGASSRATGIPDRFSGSGSGTDFT | |
| | RVTISVDTSKNQFSLKLSSVTAADTTVYYCA | | LTISRLEPEDFAVYYCQQYGSSPLYTFGQGT | |
| | RQGSTVVRGVYYYGMDVWGQGTTVTVSSAS | | KLEIKRT | |
| 88 | EVQLLESGGGLVQPGRSLRLSCAASGFTFSS | 89 | EIVLTQSPDFQSVTPKEKVTITCRASQSIGS | U.S. Pat. No. |
| | YAMSWVRQAPGKGLEWVSAISGSGGSRYYAD | | SLHWYQQKPDQSPKLLIKYASQSFSGVPSRF | 7,115,717 |
| | SVKGRFTISRDNSKNTLYLQMNSLRAEDTAV | | SGSGSGTDFTLTINSLEAEDAAAYYCHQSSS | |
| | YYCAKESSGWFGAFDYWGQGTLVTVSS | | LPITFGQGTRLEIKR | |
| 90 | QVQLQESGPGLVKPSQTLSLTCTVSGGSISS | 6 | EIVLTQSPGTLSLSPGERATLSCRASQGISR | U.S. Pat. No. |
| | GDYFWSWIRQLPGKGLEWGHIHNSGTTYYNP | | SYLAWYQQKPGQAPSLLIYGASSRATGIPDR | 7,521,048 |
| | SLKSRVTISVDTSKKQFSLRLSSVTAADTAV | | FSGSGSGTDFTLTISRLEPEDPAVYYCQQFG | |
| | YYCARDRGGDYYYGMDVWGQGTTVTVSS | | SSPWTFGQGTKVEIK | |

TABLE 3
Anti DR5 ScFv Sequences

| SEQ | | |
|---|---|---|
| ID | SEQUENCE | Reference |
| 57 | EVQLVQSGGGVERPGGSLRLSCAASGFTFDDYGMSWVRQAPGKGLEWV | U.S. Patent |
| | SGINWNGGSTGYADSVKGRVTISRDNAKNSLYLQMNSLRAEDTAVYYCA | Application |
| | KILGAGRGWYFDLWGKGTTVTVSSGGGGSGGGGSGGGGSSELTQDPAVS | Publication |
| | VALGQTVRITCQGDSLRSYYASWYQQKPGQAPVLVIYGKNNRPSGIPDRF | No. |
| | SGSSSGNTASLTITGAQAEDEADYYCNSRDSSGNHVVFGGGTKLTVLG | 2006/0269555 |
| 58 | EVQLVETGGGLVQPGGSLRLSCAASGFTFSSYAMSWVRQAPGKGLEWVS | U.S. Patent |
| | AISGSGGSTYYADSVKGRFTISRDNSKNTLYLQMNSLRAEDTAVYHCARG | Application |
| | GYSSSRSAAYDIWGQGTLVTVSSGGGGSGGGGSGGGGSSELTQDPAVSV | Publication |
| | ALGQTVRITCQGDSLRSYYASWYQQKPGQAPVLVIYGKNNRPSGIPDRFS | No. |
| | GSSSGNTASLTITGAQAEDEADYYCNSRDSSGNHVVFGGGTKLTVLG | 2006/0269556 |
| 59 | QVQIVQSGAEVKKPGASVKISCEGSGYTFNSYTLHWLRQAPGQRLEWM | U.S. Patent |
| | GRINAGNGNTKYSQNFQGRLSITRDTSATTAYMELSSERSEDTGVYYCAR | Application |
| | VFTYSFGMDVWGRGTLVTVSSGGGGSGGGGSGGGGSAQSVLTQPPSASG | Publication |
| | TPGQRVTISCSGGGSNIGRNSVSWYQQLPGTAPKLILYSNNQRPSGVPDRF | No. |
| | SGSKSGTSASLAISGLRSEDEALYYCAAWDDSLSGGVFGGGTKLTVLG | 2006/0269557 |
| 60 | QVQLVESGGGLVQPGGSLRLSCAASGFTFSSYAMSWVRQAPGKGLEWVS | U.S. Patent |
| | AISGSGGSTYYADSVKGRFTISRDNSKNTLYLQMNSLRAEDTAVYYCAK | Application |
| | VHRPGRSGYFDYWGRGTLVTVSSGGGGSGGGGSGGGGSSELTQDPAVSV | Publication |
| | ALGQTVRITCQGDSLRSYYASWYQQKPGQAPVLVIYGKNNRPSGIFDRFS | No. |
| | GSSSGNTASLTITGAQAEDEADYYCNSRDSSGNHVVFGGGTKLTVLG | 2006/0269558 |
| 61 | QVQLQQSGAEVKKPGASVRVSCQASGYSLSEYYIHWVRQAPGQGLEWM | U.S. Patent |
| | GWLNPNSGVTDYAQKFQGRVSMTRDTSISTAYMELSSLTFNDTAVYFCA | Application |
| | RGNGDYWGKGTLVTVSPGGGGSGGGGSGGGGSSELTQDPAVSVALGQT | Publication |
| | VRITCQGDSLRSYYTNWFQQKPGQAPLLVVYAKNKRPSGIPDRFSGSSSG | No. |
| | NTASLTITGAQAEDEADYYCHSRDSSGWVFGGGTKLTVLG | 2006/0269559 |
| 62 | QVQLVQSGGGVVQPGRSLRLSCAASGFTFSPDAMHWVRQAPGKGLEWM | U.S. Patent |
| | GVISFDGSQTFYADSVKGRFTISRDNSQNTLYLQMNSLRSDDTAVYYCAR | Application |
| | APARFFPLHFDIWGRGTMVTVSSGGGGSGGGGSGGGGSALSSELTQDPA | Publication |
| | VSVALGQTVRITCQGDSLRTHYASWYHQRPGRAPVLVNYPKDSRPSGIPD | No. |
| | RFSGSSSGNTASLTIIGAQAADEGDYYCQSRDSSGVLFGGGTKVTVLG | 2006/0269560 |
| 63 | EVQLVESGGGLVQPGGSLRLSCAASGFTFSSYWMSWVRQAPGKGLEWV | U.S. Patent |
| | ANIKQDGSEKYYVDSVKGRFTISRDNAKNSLYLQMNSLRAEDTAVYYCA | Application |
| | RDFSGYGDYLDYWGKGTLVTVSSGGGGSGGGGSGGGGSAQSALTQPPS | Publication |
| | ASGSPGQSVTISCTGTSSDIGNYNYVSWYQQHPGKAPKLMIYEVNERPSG | No. |
| | VPDRFSGSKSGNTASLTVSGLRPEDEADYYCSSYAGNNAVIFGGGTQLTV | 2006/0269561 |
| | LG | |
| 64 | QVQLVQSGAEVKKPGASVKVSCKASGYTFTTHAMHWVRQAPGQSLEW | U.S. Patent |
| | MGWINTGNGNTKYSQSEQGRVSITRDTSANTAYMELSSLKSEDTAMYYC | Application |
| | ARASRDSSGYYYVPPGDFFDIWGQGTLVTVSSGGGGSGGGGSGGGGSAQ | Publication |
| | SALTQPASVSGSPGQSITISCTGSRSDIGGYNFVSWYQQHPGKAPKLLIYD | No. |
| | VYNRPSGISDHFSGSKSDNTASLTISGLQSEDDADYYCSSYAGYHTWIFGG | 2006/0269562 |
| | GTKVTVLG | |
| 65 | EVQLVQSGAEVKKPGASVKLSCKASGYTLVNYFMHWVRQAPGQGPEW | U.S. Patent |
| | MGMINPSGGTTKNRQKFQDRVTMTRDTSTRTVYMELSGLTSEDTAVYYC | Application |
| | ATDFKGTDILFRDWGRGTLVTVSSGGGGSGGGGSGGGGSAQSVLTQPPS | Publication |
| | ASGTPGQRVSISCSGSSSNIGSNTVIWYQQLPGTAPKLLMYSNDRRPSGVP | No. |
| | DRFSGSKSGTSASLAISGLQSEDEADYYCATWDDSLNGHYVFGTGTKLTV | 2006/0269563 |
| | LG | |
| 66 | QMQLVQSGGGLVKPGGSLRLSCAASGFTFSDYYMSWIRQAPGKGLEWVS | U.S. Patent |
| | AISGSGGSTYYADSVKGRFTISRDNSKNTLYLQMNSLRAEDTAVYYCARG | Application |
| | GSTFDIWGRGTMVTVSSGGGGSGGGGSGGGGSAQPVLTQPPSASGTPGQ | Publication |
| | RVTISCSGSNSNIGSRPVNWYQQLPGTAPKLLIQGNNQRPSGVPDRFSGSK | No. |
| | SGTSASLAISGLQSEDEADYYCAAWDDSLTGYVFGPGTKLTVLG | 2006/0269564 |
| 67 | QMQLVQSGGAVVQPGRSLRLSCAASGFTFSSYGMHWVRQAPGKGLEWV | U.S. Patent |
| | AVISYDGSIKYYADSVKGRFTISRDNSKNTLYLQMNSLRAEDTAVYYCAR | Application |
| | ERLRGLDPWGQGTMVTVSSGGGGSGGGGSGGGGSSELTQDPAVSVALG | Publication |
| | QTVRITCQGDSLRSYYASWYQQKPGQAPVLVIYGKNNRPSGIPDRFSGSSS | No. |
| | GNTASLTITGAQAEDEADYYCNSRDSSGNHVVFGGGTKLTVLG | 2006/0269565 |
| 68 | EVQLVETGGGLVQPGGSLRLSCAASGFTFSPYYMSWVRQAPGKGLEWVS | U.S. Patent |
| | AISGSGGSIYYADSVKGRFTISRDNSKNTLYLQMNSLRAEDTALYYCARG | Application |
| | ASGPDYWGRGTMVTVSSGGGGSGGGGSGGGGSAQSVLTQPPSVSAAPG | Publication |
| | QKVTISCSGSTSNIGNNYVSWYQQVPGTAPKLLIYDNNKRPSGIPDRFSGS | No. |
| | KSGTSATLGITGLQTGDEADYYCGTWDSSLSALVFGGGTKVTVLG | 2006/0269566 |
| 69 | QVQLQQSGAEVKTPGSSVKVSCKASGGTFRNNAISWVRQAPGQGLEWM | U.S. Patent |
| | GGFIPKFGTTNHAQKFQGRVTMTADDSTNTVYMELSSLRSEDTAVYYCA | Application |
| | RGGAYCGGGRCYLYGMDVWGQGTLVTVSSGGGGSGGGGSGGGGSAQA | Publication |
| | VVIQEPSLTVSPGGTVTLTCGSSTGAVTSGHYPYWFQQKPGQAPRTLIYDT | No. |
| | SNKRSWTPARFSGSLLGGKAALTLSGAQPEDEAEYYCLVSYSGSLVVFGG | 2006/0269567 |
| | GTKLTVLG | |
| 70 | EVQLLESGGGLVQPGGSLRLSCAASGFTFSSYAMSWVRQAPGKGLEWVS | ELS. Patent |
| | AISGSGGSTYYADSVKGRFTISRDNSKNTLYLQMNSLRAEDTAVYYCVK | Application |
| | GAWLDYWGRGTMVTVSSGGGGSGGGGSGGGGSALNFMLTQPHSVSESP | Publication |
| | GKTVTISCTGSSGSVARNYVQWYQQRPGSAPTIVIYEDNRRPSGVPGRFSG | No. |
| | SIDRSSNSASLTISGLQTEDEADYYCQSYNYNTWVFGGGTKLTVLG | 2006/0269568 |
| 71 | EVQLVQSGAEVKKPGASVKVSCRASGYTFTSYGITWVRQAPGQGLEWM | U.S. Patent |
| | GWISAYNGKTNYVQELQGRVTMTTDTSTSTVYMELTSLRSDDTAVYYCA | Application |
| | RRGNNYRFGYFDFWGQGTLVTVSSGGGGSGGGGSGGGGSALETTLTQSP | Publication |
| | GTLSLSPGERATLSCRASQSISSSNLAWYQQKPGRAPRLLIYGASSRAIGIP | No. |
| | DRFSGSGSGTDFTLTISRLEAEDFAVYYCQQYGSSPITFGQGTRLEIKR | 2006/0269569 |
| 72 | QVQLQQSGPGLVKPSQTLSLTCAISGDSVSSTTVAWDWIRQSPSRGLEWL | U.S. Pat. |
| | GRTYYRSKWYNEYAVSVKSRITINVDTSKNQISLQLNSVTPEDTAVYYCA | No. |
| | REPDAGRGAFD1WGQGTTVTSPLRWGRFGWRGLGRGWLRSPVTQSPGTL | 8,097,704 |
| | SLSPGERATLSCRASQSVSSSHLAWYQQKPGQAPRLLIYGASSRATGIPDR | |
| | FSGSGSGTDFTLTISSLEPEDFAVYYCQQRSNWPPRAVFGQGTRLEIK | |
| 73 | QVQLQQSGPGRVQPSQTLSLTCAISGDSVSNNNAAWYWIRQSPSRGLEW | U.S. Pat. |
| | LGRTYYRSKWYNDYAVSVKSRITISPDTSKNQFSLQLNSVTPEDTAVYYC | No. |
| | ARRGDGNSYFDYWGQGTLVTVSSGILRWGRFGWRGLGRGWLEIVLTQSP | 8,097,705 |
| | GTLSLSPGERATLSCRASQSVSSGYVSWYRQKPGQAPRLLIYGASTRATGI | |
| | PDRFSGSGSGTDFTLTISRLEPEDFAVYYCHQYGSSPNTYGQGTKVGIK | |

In certain embodiments, the DR5 binding domain comprises a VH and a VL, where the VH and VL comprise amino acid sequences at least 60%, at least 65%, at least 70%, at least 75%, at least 80%, at least 85%, at least 90%, at least 95% or 100% identical to SEQ ID NO: 1 and SEQ ID NO: 2; SEQ ID NO: 3 and SEQ ID NO: 4; SEQ ID NO: 5 or SEQ ID NO: 90 and SEQ ID NO: 6; SEQ ID NO: 7 and SEQ ID NO: 8; SEQ ID NO: 9 and SEQ ID NO: 10: SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; SEQ ID NO: 23 and SEQ ID NO: 24; SEQ ID NO: 25 and SEQ ID NO: 26; SEQ ID NO: 27 and SEQ ID NO: 28; SEQ ID NO: 29 and SEQ ID NO: 30; SEQ ID NO: 31 and SEQ ID NO: 32; SEQ ID NO: 33 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44. SEQ ID NO: 45 and SEQ ID NO: 46; SEQ ID NO: 47 and SEQ ID NO: 48; SEQ ID NO: 49 and SEQ ID NO: 50; SEQ ID NO: 51 and SEQ ID NO: 52; SEQ ID NO: 53 and SEQ ID NO: 54; SEQ ID NO: 55 and SEQ ID NO: 56; SEQ ID NO: 82 and SEQ ID NO: 83; SEQ ID NO: 84 and SEQ ID NO: 85; SEQ ID NO: 86 and SEQ ID NO: 87, or SEQ ID NO: 88 and SEQ ID NO: 89; respectively, or where the VH and VL are contained in an ScFv with an amino acid sequence at least 90% identical to SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, or SEQ ID NO: 73.

In certain embodiments, the DR5 binding domain comprises a VH and a VL, where the VH and VL comprise amino acid sequences at least 60%, at least 65%, at least 70%, at least 75%, at least 80%, at least 85%, at least 90%, at least 95% or 100% identical to SEQ ID NO: 5 or SEQ ID NO: 90 and SEQ ID NO: 6; or SEQ ID NO: 7 and SEQ ID NO: 8, respectively. In certain embodiments, the DR5 binding domain comprises a VH and a VL, where the VH and VL comprise amino acid sequences at least 60%, at least 65%, at least 70%, at least 75%, at least 80%, at least 85%, at least 90%, at least 95% or 100% identical to SEQ ID NO: 5 and SEQ ID NO: 6, respectively. In certain embodiments, the DR5 binding domain comprises a VH and a VL, where the VH and VL comprise amino acid sequences at least 60%, at least 65%, at least 70%, at least 75%, at least 80%, at least 85%, at least 90%, at least 95% or 100% identical to SEQ ID NO: 90 and SEQ ID NO: 6. In certain embodiments, the DR5 binding domain comprises a VH and a VL, where the VH and VL comprise amino acid sequences at least 60%, at least 65%, at least 70/%, at least 75%, at least 80%, at least 85%, at least 90%, at least 95% or 100% identical to SEQ ID NO: 7 and SEQ ID NO: 8, respectively.

While a variety of different dimeric, pentameric, and hexameric binding molecules can be contemplated by a person of ordinary skill in the art based on this disclosure, and as such are included in this disclosure, in certain embodiments, a binding molecule as described above is provided in which each binding unit comprises two IgA or IgM heavy chains each comprising a VH situated amino terminal to the IgA or IgM constant region or fragment thereof, and two immunoglobulin light chains each comprising a VL situated amino terminal to an immunoglobulin light chain constant region.

Moreover, in certain embodiments, at least one binding unit of the binding molecule, or at least two, at least three, at least four, at least five, or at least six binding units of the binding molecule, comprises or comprise two of the DR5 binding domains as described above. In certain embodiments the two DR5 binding domains in the at least one binding unit of the binding molecule, or at least two, at least three, at least four, at least five, or at least six binding units of the binding molecule, can be different from each other, or they can be identical.

In certain embodiments, the two IgA or IgM heavy chains within the at least one binding unit of the binding molecule, or at least two, at least three, at least four, at least five, or at least six binding units of the binding molecule, are identical. In certain embodiments, two identical IgA or IgM heavy chains within at least one binding unit, or within at least two, at least three, at least four, at least five, or at least six binding units of the binding molecule comprise the heavy chain variable domain amino acid sequences as disclosed in Tables 2 and 3.

In certain embodiments, the two light chains within the at least one binding unit of the binding molecule, or at least two, at least three, at least four, at least five, or at least six binding units of the binding molecule, are identical. In certain embodiments, two identical light chains within at least one binding unit, or within at least two, at least three, at least four, at least five, or at least six binding units of the binding molecule are kappa light chains, e.g., human kappa light chains, or lambda light chains, e.g., human lambda light chains. In certain embodiments, two identical light chains within at least one binding unit, or within at least two, at least three, at least four, at least five, or at least six binding units of the binding molecule each comprise the light chain variable domain amino acid sequences as disclosed in Tables 2 and 3.

In certain embodiments at least one, at least two, at least three, at least four, at least five, or at least six binding units of a dimeric, pentameric, or hexameric binding molecule provided by this disclosure comprises or each comprise two identical IgA or IgM heavy chain constant regions each comprising identical heavy chain variable domain amino acid sequences as disclosed in Tables 2 and 3, and two identical light chains each comprising identical heavy chain variable domain amino acid sequences as disclosed in Tables 2 and 3. According to this embodiment, the DR5 binding domains in the at least one binding unit of the binding molecule, or at least two, at least three, at least four, at least five, or at least six binding units of the binding molecule, can be identical. Further according to this embodiment, a dimeric, pentameric, or hexameric binding molecule as provided herein can comprise at least one, at least two, at least three, at least four, at least five, at least six, at least seven, at least eight, at least nine, at least ten, at least eleven, or at least twelve copies of an DR5 binding domain as described above. In certain embodiments at least two, at least three, at least four, at least five, or at least six of the binding units can be identical and, in certain embodiments the binding units can comprise identical binding domains, e.g., at least two, at least three, at least four, at least five, at least six, at least seven, at least eight, at least nine, at least ten, at least eleven, or at least twelve DR5 binding domains can be identical.

In certain embodiments, a dimeric, pentameric, or hexameric DR5 binding molecule as provided herein can possess advantageous structural or functional properties compared to other binding molecules. For example, the dimeric, pentameric, or hexameric DR5 binding relative to a corresponding bivalent binding molecule having the same antigen binding domains. Biological assays include, but are not limited to ELISA and Western blot caspase assays, and FACS analyses using stains indicative of apoptotic cell death such as annexin-v. In certain embodiments a dimeric, pentameric, or hexameric binding molecule as provided herein can trigger apoptosis of a DR5-expressing cell at higher potency than an equivalent amount of a monospecific, bivalent IgG1 antibody or fragment thereof that specifically binds to the same DR5 epitope as the DR5 binding domain. In certain embodiments a dimeric, pentameric, or hexameric binding molecule as provided herein can trigger apoptosis of a DR5-expressing cell at higher potency than an equivalent amount of monospecific, bivalent anti-DR5 monoclonal antibody or fragment thereof, where the antibody is, or comprises the same VH and VL regions as, the antibodies provided in Tables 2 and 3.

Methods of Use

This disclosure provides a method for inhibiting, delaying, or reducing malignant cell growth in a subject with cancer by administering to the subject a combination therapy comprising an effective amount of a dimeric IgA or IgA-like antibody or a hexameric or pentameric IgM or IgM antibody, or a multimerized antigen-binding fragment thereof that specifically and agonistically binds to DR5, where three to twelve antigen binding domains of the IgA or IgA-like antibody or IgM or IgM-like antibody or fragment thereof are DR5-specific and agonistic, in combination with an effective amount of a cancer therapy, e.g., radiation, an anthracycline, a folic acid analog, a platinum-based agent, a taxane, a topoisomerase II inhibitor, or any combination thereof. Exemplary anti-DR5 IgA or IgA-like antibodies and IgM or IgM-like antibodies and exemplary cancer therapies are described in detail elsewhere herein. Additional combination therapies are provided, e.g., in PCT Publication No. WO 2019/165340, which is incorporated herein by reference in its entirety. In certain embodiments, administration of the combination therapy provided herein can inhibit tumor or malignant cell growth partially or completely, can delay the progression of tumor and malignant cell growth in the subject, can prevent metastatic spread in the subject, can reduce the subject's tumor size, e.g., to allow more successful surgical removal, and/or can result in any combination of positive therapeutic responses in the subject. Exemplary therapeutic responses that can be achieved are described herein.

In certain embodiments, administration of the combination therapy can result in enhanced therapeutic efficacy relative to administration of the anti-DR5 IgA or IgA-like antibody or IgM or IgM-like antibody or the cancer therapy, e.g., radiation, an anthracycline, a folic acid analog, a platinum-based agent, a taxane, a topoisomerase II inhibitor, or any combination thereof, alone. In certain embodiments the improved treatment efficacy can be greater than the additive efficacy of each individual therapy. In certain embodiments the improved treatment efficacy over either therapy administered alone, measured, e.g., in increased tumor growth delay (TGD), increased frequency of tumor regression, e.g., complete tumor regression, or increased survival is at least 5%, at least 10%, at least 20%, at least 5%, at least 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, or at least 1000%. In certain embodiments the improved treatment efficacy over the additive efficacy of both therapies administered individually, measured, e.g., in increased tumor growth delay (TGD), increased frequency of tumor regression, e.g., complete tumor regression, or increased survival is at least 5%, at least 10%, at least 20%, at least 5%, at least 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, at least 450%, at least 500%, at least 550%, at least 600%, at least 650%, at least 700%, at least 750%, at least 800%, at least 850%, at least 900%, at least 950%, or at least 1000%. In certain embodiments the improvement can be complete tumor regression and/or full survival. The improved activity can, for example, allow a reduced dose to be used, or can result in more effective killing of cells that are resistant to killing by standard treatments. By “resistant” is meant any degree of reduced activity of “standard of care” for a given tumor or cancer type.

In certain embodiments the combination treatment methods provided herein can facilitate cancer treatment, e.g., by slowing tumor growth, stalling tumor growth, or reducing the size of existing tumors, when administrated as an effective dose to a subject in need of cancer treatment.

In certain embodiments the DR5-expressing cell is an immortalized cell line, e.g., a cancer cell. The terms “cancer”, “tumor”, “cancerous”, and “malignant” refer to or describe the physiological condition in mammals that is typically characterized by unregulated cell growth. Examples of cancers include but are not limited to, carcinoma including adenocarcinomas, lymphomas, blastomas, melanomas, sarcomas, and leukemias. More particular examples of such cancers include osteosarcoma, chondrosarcoma, fibrosarcoma, squamous cell cancer, small-cell lung cancer, non-small cell lung cancer, gastrointestinal cancer, Hodgkin's and non-Hodgkin's lymphoma, pancreatic cancer, glioblastoma, glioma, cervical cancer, ovarian cancer, liver cancer such as hepatic carcinoma and hepatoma, bladder cancer, breast cancer (including hormonally mediated breast cancer, see, e.g., Innes et al. (2006) Br. J. Cancer 94:1057-1065) and triple negative breast cancer (TNBC), colon cancer, colorectal cancer, endometrial carcinoma, myeloma (such as multiple myeloma), salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, various types of head and neck cancer including, but not limited to, squamous cell cancers, and cancers of mucinous origins, such as, mucinous ovarian cancer, cholangiocarcinoma (liver) and renal papillary carcinoma. Mucosal distribution, for example as provided by an IgA-based binding molecule as provided herein, could be beneficial for certain cancers, e.g., lung cancer, ovarian cancer, colorectal cancer, or squamous cell carcinoma.

Effective doses of compositions for treatment of cancer vary depending upon many different factors, including means of administration, target site, physiological state of the patient, whether the patient is human or an animal, other medications administered, and whether treatment is prophylactic or therapeutic. In certain embodiments the treatment methods provided herein can provide increased safety, in that the composition exhibits greater cytotoxicity (e.g., induces apoptosis to a greater extent) on cancer cells than on non-cancer cells, e.g., normal human hepatocytes. Usually, the patient is a human, but non-human mammals including transgenic mammals can also be treated. Treatment dosages can be titrated using routine methods known to those of skill in the art to optimize safety and efficacy.

The compositions of the disclosure can be administered by any suitable method, e.g., parenterally, intraventricularly, orally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

The subject to be treated can be any animal, e.g., mammal, in need of treatment, in certain embodiments, subject is a human subject.

In its simplest form, a preparation to be administered to a subject is a dimeric, pentameric, or hexameric anti-DR5 antibody as provided herein, or an antigen-binding, multimerizing fragment, variant, or derivative thereof, administered in conventional dosage form in combination with a cancer therapy. In some embodiments, the cancer therapy is a chemotherapeutic agent. Accordingly, in some embodiments, the anti-DR5 antibody and the chemotherapeutic agent can be combined with a pharmaceutical excipient, carrier or diluent as described elsewhere herein. In some embodiments, the anti-DR5 antibody and the chemotherapeutic agent can be administered in separate pharmaceutical compositions.

A DR5 binding molecule as provided herein or an antigen-binding, multimerizing fragment, variant, or derivative thereof can be administered by any suitable method as described elsewhere herein, e.g., via IV infusion. In certain embodiments, a DR5 binding molecule as provided herein or an antigen-binding, multimerizing fragment, variant, or derivative thereof can be introduced into a tumor, or in the vicinity of a tumor cell.

All types of tumors are potentially amenable to treatment by this approach including, without limitation, carcinoma of the breast, lung, pancreas, ovary, kidney, colon, and bladder, as well as melanomas, sarcomas, and lymphomas. Mucosal distribution could be beneficial for certain cancers, e.g., lung cancer, ovarian cancer, colorectal cancer, or squamous cell carcinoma.

Accordingly, in some embodiments, the method provided herein is a method for inhibiting, delaying, or reducing malignant cell growth in a subject with cancer, where the cancer is a hematologic cancer or a solid tumor. In some embodiments, the cancer is a hematologic cancer, such as acute myeloid leukemia (AML), chronic myeloid leukemia (CML), acute lymphocytic leukemia (ALL), chronic lymphocytic leukemia, hairy cell leukemia, Hodgkin lymphoma, non-Hodgkin lymphoma, multiple myeloma, any metastases thereof, or any combination thereof, a solid tumor. In some embodiments, the cancer is a solid tumor, such as bladder cancer, colorectal cancer, sarcoma (e.g., fibrosarcoma), gastric cancer, lung cancer (e.g., non-small cell lung cancer (NSCLC)), or pancreatic cancer.

Radiation

Radiation therapy is the localized application of ionizing radiation to a cancerous tumor. The goal of radiation therapy is to damage the DNA of the cancerous cells leading to cell death. Radiation therapy is widely used in the treatment of a variety of cancers, including bladder cancer, colorectal cancer, sarcoma, gastric cancer, lung cancer, and pancreatic cancer. In some embodiments, the cancer therapy comprises radiation therapy, the cancer is non-metastatic cancer, such as non-metastatic bladder cancer, colorectal cancer, sarcoma, gastric cancer, lung cancer, and pancreatic cancer. In some embodiments, the cancer therapy comprises radiation therapy, the method further comprises administering an effective amount of one or more chemotherapeutic agents, such as a chemotherapeutic agent disclosed herein. In some embodiments, the chemotherapeutic agent is a topoisomerase I inhibitor, a topoisomerase II inhibitor, a nucleoside analog, a folic acid analog, a platinum-based agent, a taxane, a b-cell lymphoma-2 (BCL-2) inhibitor, or any combination thereof. Exemplary chemotherapeutic agents and combinations of chemotherapeutic agents are discussed in greater detail elsewhere herein and in PCT Publication No. WO 2019/165340.

Topoisomerase II Inhibitors

Topoisomerase II has become an important target for chemotherapeutics as inhibition of this enzyme leads to DNA breaks and cancer cell apoptosis. Examples of a topoisomerase II inhibitors etoposide and anthracyclines such as daunorubicin and doxorubicin. Doxorubicin is used to treat a variety of cancers including breast cancer, sarcoma, ovarian cancer, bladder cancer, lung cancer, and multiple myeloma.

Daunorubicin (CAS Registry No. 20830-81-3) has the following formula:

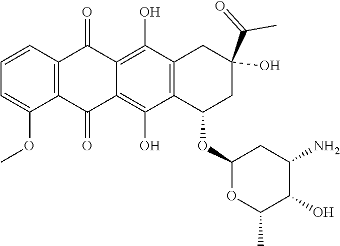 [SMILES: COc1cccc2c1C(=O)c1c(O)c3c(c(O)c1C2=O)C[C@@](O)(C(C)=O)C[C@@H]3O[C@H]1C[C@H](N)[C@H](O)[C@H](C)O1]

Doxorubicin (CAS Registry No. 23214-92-8) has the following formula:

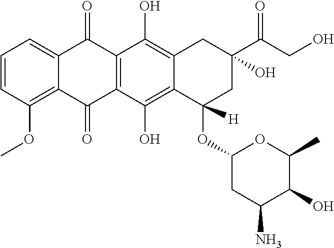 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1]

Etoposide (CAS Registry No. 33419-42-0) has the following formula:

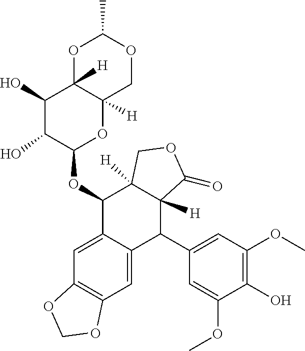

In some embodiments, the cancer therapy comprises a topoisomerase II inhibitor. In some embodiments, the topoisomerase II inhibitor is administered intravenously. In some embodiments, the cancer therapy comprises a topoisomerase II inhibitor, and the cancer is a cancer disclosed herein. In some embodiments, the cancer therapy comprises a topoisomerase II inhibitor and the cancer is a lung cancer, a sarcoma or hematologic cancer, such as a hematologic cancer disclosed herein, e.g., acute myeloid leukemia (AML). In some embodiments, the cancer therapy topoisomerase II inhibitor, and the method further comprises administering an effective amount of one or more additional cancer therapies disclosed herein. In some embodiments, the cancer therapy comprises topoisomerase inhibitor, and the method further comprises administering an effective amount of radiation therapy.

In some embodiments, the cancer therapy comprises etoposide, and the cancer is a lung cancer. In some embodiments, the cancer therapy comprises etoposide, and the cancer is a hematologic cancer. In some embodiments, the cancer therapy comprises etoposide, and the cancer is acute myeloid leukemia (AML). In some embodiments, the cancer therapy comprises etoposide, and the method further comprises administering an effective amount of one or more additional cancer therapies disclosed herein. In some embodiments, the cancer therapy comprises etoposide, and the method further comprises administering an effective amount of radiation therapy. In some embodiments, the cancer therapy comprises etoposide, the method further comprises administering an effective amount of radiation therapy, and the cancer is a sarcoma or hematologic cancer, such as a hematologic cancer disclosed herein, e.g., acute myeloid leukemia (AML).

In some embodiments, the cancer therapy comprises an anthracycline, such as doxorubicin, and the cancer is a cancer disclosed herein. In some embodiments, the cancer therapy comprises an anthracycline, such as doxorubicin, and the cancer is a sarcoma or hematologic cancer, such as a hematologic cancer disclosed herein, e.g., acute myeloid leukemia (AML). In some embodiments, the cancer therapy comprises doxorubicin, and the cancer is a sarcoma. In some embodiments, the cancer therapy comprises doxorubicin, and the cancer is a hematologic cancer. In some embodiments, the cancer therapy comprises doxorubicin, and the cancer is acute myeloid leukemia (AML). In some embodiments, the cancer therapy comprises an anthracycline, such as doxorubicin, and the method further comprises administering an effective amount of one or more additional cancer therapies disclosed herein. In some embodiments, the cancer therapy comprises an anthracycline, such as doxorubicin, and the method further comprises administering an effective amount of radiation therapy. In some embodiments, the cancer therapy comprises an anthracycline, such as doxorubicin, the method further comprises administering an effective amount of radiation therapy, and the cancer is a sarcoma or hematologic cancer, such as a hematologic cancer disclosed herein, e.g., acute myeloid leukemia (AML).

Folic Acid Analogs

Folic acid analogs, such a leucovorin, have been used to reduce the toxic effects of certain chemotherapies. Leucovorin, e.g., leucovorin calcium (calcium folinate) is a component of the “FOLFOX” and “FOLFIRI” chemotherapeutic regimens. “FOLFOX” comprises leucovorin calcium (calcium folinate), 5-fluorouracil, and oxaliplatin. “FOLFIRI” regimen comprises leucovorin calcium (calcium folinate), 5-fluorouracil, and Irinotecan. FOLFIRI and FOLFOX are widely used in the treatment of advanced-stage and metastatic colorectal cancer.

Leucovorin (CAS Registry No. 1492-18-8) has the following formula:

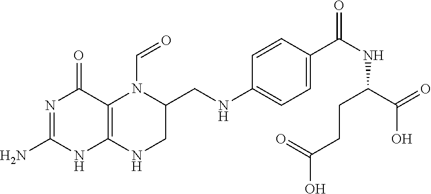 [SMILES: Nc1nc(=O)c2c([nH]1)NCC(CNc1ccc(C(=O)N[C@@H](CCC(=O)O)C(=O)O)cc1)N2C=O]

In some embodiments, the cancer therapy comprises a folic acid analog, such as leucovorin, and the cancer is a cancer disclosed herein. In some embodiments, the folic acid analog is administered intravenously. In some embodiments, the cancer therapy comprises a folic acid analog, such as leucovorin, the cancer is colorectal cancer. In some embodiments, the cancer therapy comprises a folic acid analog, such as leucovorin, the method further comprising administering an effective amount of one or more additional cancer therapies disclosed herein. In some embodiments, the cancer therapy comprises a folic acid analog, such as leucovorin, the method further comprises administering an effective amount of 5-fluorouracil. In some embodiments, the cancer therapy comprises a folic acid analog, such as leucovorin, the method further comprises administering an effective amount of irinotecan. In some embodiments, the cancer therapy comprises a folic acid analog, such as leucovorin, the method further comprises administering an effective amount of oxaliplatin. In some embodiments, the cancer therapy comprises a folic acid analog, such as leucovorin, the method further comprises administering an effective amount of 5-fluorouracil and irinotecan. In some embodiments, the cancer therapy comprises a folic acid analog, such as leucovorin, the method further comprises administering an effective amount of 5-fluorouracil and oxaliplatin. In some embodiments, the cancer therapy comprises a folic acid analog, such as leucovorin, and the method further comprises administering an effective amount of radiation therapy, optionally in combination with one or more other components of FOLFOX or FOLFIRI. In some embodiments, the cancer therapy comprises a folic acid analog, such as leucovorin, and the method further comprises administering an effective amount of bevacizumab, optionally in combination with one or more other components of FOLFOX or FOLFIRI.

In some embodiments, the cancer therapy comprises a folic acid analog, such as leucovorin, the method further comprises administering an effective amount of 5-fluorouracil and the cancer is colorectal cancer. In some embodiments, the cancer therapy comprises a folic acid analog, such as leucovorin, the method further comprises administering an effective amount of irinotecan and the cancer is colorectal cancer. In some embodiments, the cancer therapy comprises a folic acid analog, such as leucovorin, the method further comprises administering an effective amount of oxaliplatin and the cancer is colorectal cancer. In some embodiments, the cancer therapy comprises a folic acid analog, such as leucovorin, the method further comprises administering an effective amount of 5-fluorouracil and irinotecan and the cancer is colorectal cancer. In some embodiments, the cancer therapy comprises a folic acid analog, such as leucovorin, the method further comprises administering an effective amount of 5-fluorouracil and oxaliplatin and the cancer is colorectal cancer. In some embodiments, the cancer therapy comprises a folic acid analog, such as leucovorin, and the method further comprises administering an effective amount of radiation therapy, optionally in combination with one or more other components of FOLFOX or FOLFIRI and the cancer is colorectal cancer. In some embodiments, the cancer therapy comprises a folic acid analog, such as leucovorin, and the method further comprises administering an effective amount of bevacizumab, optionally in combination with one or more other components of FOLFOX or FOLFIRI, and the cancer is colorectal cancer.

Platinum-Based Agents

Platinum-based agents are commonly used treat various cancer. Platinum-based agents are believed to cause crosslinking of DNA leading to cancer cell death. Examples of platinum-based agents include cisplatin, carboplatin, and oxaliplatin.

Cisplatin (CAS Registry No. 15663-27-1) has the following formula:

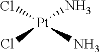

Carboplatin (CAS Registry No. 41575-94-4) has the following formula:

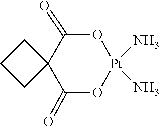

Oxaliplatin (CAS Registry No. 63121-00-6) has the following formula:

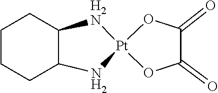

In some embodiments, the cancer therapy comprises a platinum-based agent, such as cisplatin, carboplatin, or oxaliplatin, and the cancer is a cancer disclosed herein. In some embodiments, the cancer therapy comprises a platinum-based agent, such as cisplatin, carboplatin, or oxaliplatin, and the cancer therapy is administered intravenously. In some embodiments, the cancer therapy comprises a platinum-based agent, such as cisplatin, carboplatin, or oxaliplatin, the cancer is a gastric cancer, a lung cancer, such as non-small cell lung cancer (NSCLC), or colorectal cancer. In some embodiments, the cancer therapy comprises a platinum-based agent, such as cisplatin, carboplatin, or oxaliplatin, the method further comprises administering an effective amount of radiation therapy.

In some embodiments, the cancer therapy comprises oxaliplatin, the cancer is gastric cancer or colorectal cancer. In some embodiments, the cancer therapy comprises oxaliplatin, the cancer is gastric cancer. In some embodiments, the cancer therapy comprises oxaliplatin, the method further comprises administering an effect amount of radiation therapy. In some embodiments, the cancer therapy comprises oxaliplatin, the method further comprises administering an effect amount of radiation therapy and the cancer is gastric cancer.

In some embodiments, the cancer therapy comprises oxaliplatin, the method further comprises administering an effect amount of leucovorin and/or 5-fluorouracil. In some embodiments, the cancer therapy comprises oxaliplatin, the method further comprises administering an effect amount of leucovorin and/or 5-fluorouracil and the cancer is colorectal cancer. In some embodiments, the cancer therapy comprises oxaliplatin, the method further comprises administering an effect amount of 1) leucovorin and/or 5-fluorouracil and 2) radiation therapy. In some embodiments, the cancer therapy comprises oxaliplatin, the method further comprises administering an effect amount of 1) leucovorin and/or 5-fluorouracil and 2) radiation therapy, and the cancer is colorectal cancer.

In some embodiments, the cancer therapy comprises carboplatin, the cancer is gastric cancer or lung cancer, such as NSCLC. In some embodiments, the cancer therapy comprises carboplatin, the cancer is gastric cancer. In some embodiments, the cancer therapy comprises carboplatin, the method further comprises administering an effect amount of radiation therapy. In some embodiments, the cancer therapy comprises carboplatin, the method further comprises administering an effect amount of radiation therapy and the cancer is gastric cancer. In some embodiments, the cancer therapy comprises carboplatin, the cancer is lung cancer. In some embodiments, the cancer therapy comprises carboplatin, the cancer is NSCLC. In some embodiments, the cancer therapy comprises carboplatin, the method further comprises administering an effect amount of radiation therapy and the cancer is lung cancer. In some embodiments, the cancer therapy comprises carboplatin, the method further comprises administering an effect amount of radiation therapy and the cancer is NSCLC.

Taxanes

Taxanes are widely used chemotherapeutic agents. Taxanes are believed to act by disrupting microtubule function preventing cancer cell division. Examples of taxanes include paclitaxel and docetaxel. Taxanes are poorly soluble in water. Typically, taxanes are formulated with solvents such as CREMOPHOR EL® (Polyoxyl 35 Hydrogenated Castor Oil). Alternative formulations have also been developed, such as albumin nanoparticles (nab), e.g., ABRAXANE® (nab-paclitaxel).

Paclitaxel (CAS Registry No. 33069-62-4) has the following formula:

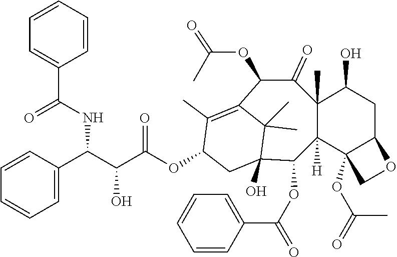 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

Docetaxel (CAS Registry No. 114977-28-5) has the following formula:

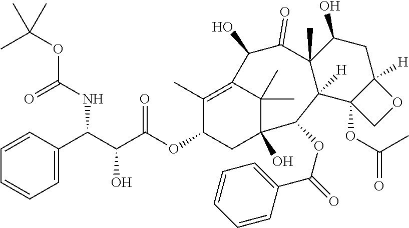

In some embodiments, the cancer therapy comprises a taxane, such as paclitaxel or docetaxel. In some embodiments, the taxane is administered intravenously. In some embodiments, the cancer therapy comprises paclitaxel, such as solvent-based paclitaxel or albumin nanoparticle (nab)-paclitaxel. In some embodiments, the cancer therapy comprises a taxane, such as paclitaxel or docetaxel, and the cancer is a cancer disclosed herein. In some embodiments, the cancer therapy comprises a taxane, such as paclitaxel or docetaxel, and the cancer is lung cancer, such as non-small cell lung cancer (NSCLC) or pancreatic cancer. In some embodiments, the cancer therapy comprises paclitaxel, and the cancer is lung cancer. In some embodiments, the cancer therapy comprises paclitaxel, and the cancer is NSCLC. In some embodiments, the cancer therapy comprises paclitaxel, and the cancer is pancreatic cancer. In some embodiments, the cancer therapy comprises paclitaxel, and the method further comprises administering radiation therapy. In some embodiments, the cancer therapy comprises paclitaxel, the method further comprises administering radiation therapy, and the cancer is NSCLC. In some embodiments, the cancer therapy comprises paclitaxel, the method further comprises administering radiation therapy, and the cancer is pancreatic cancer. In some embodiments, the cancer therapy comprises paclitaxel, the method further comprises administering gemcitabine. In some embodiments, the cancer therapy comprises paclitaxel, the method further comprises administering gemcitabine and radiation therapy. In some embodiments, the cancer therapy comprises paclitaxel, the method further comprises administering gemcitabine, and the cancer is pancreatic cancer. In some embodiments, the cancer therapy comprises paclitaxel, the method further comprises administering gemcitabine and radiation therapy, and the cancer is pancreatic cancer.

Topoisomerase I Inhibitors

Topoisomerases are popular targets for cancer chemotherapy, and a variety of inhibitors have been or are currently being developed. Compounds that inhibit type I topoisomerase are currently in use or are being developed as cancer chemotherapeutic agents. In particular, two derivatives of the natural type I topoisomerase inhibitor camptothecin, irinotecan (7-ethyl-10-[4-(1-piperidino)-1-piperidino]carbonyloxy-camptothecin, also called CPT-11), and topotecan (9-[(dimethylamino)Methyl]-10-hydroxy-(4S)-camptothecin, are currently marketed for the treatment of various cancers. Irinotecan is part of the “FOLFIRI” regimen of leucovorin calcium (calcium folinate), 5-fluorouracil, and Irinotecan widely used in the treatment of advanced-stage and metastatic colorectal cancer. In some embodiments, the topoisomerase I inhibitor is administered intravenously.

Irinotecan (CAS Registry No. 100286-90-6) has the following formula:

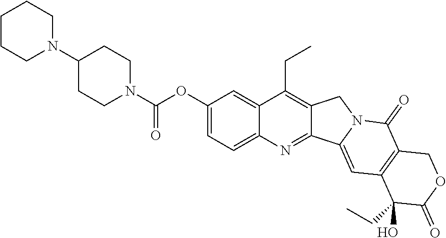 [SMILES: CCc1c2c(nc3ccc(OC(=O)N4CCC(N5CCCCC5)CC4)cc13)-c1cc3c(c(=O)n1C2)COC(=O)[C@]3(O)CC]

Topotecan (CAS Registry No. 123948-87-8) has the following formula:

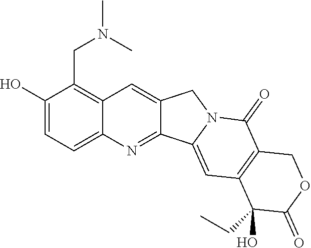 [SMILES: CC[C@@]1(O)C(=O)OCc2c1cc1n(c2=O)Cc2cc3c(CN(C)C)c(O)ccc3nc2-1]

Chemotherapeutic Nucleoside Analogs

Gemcitabine (2′,2′-difluoro 2′deoxycytidine, or dFdC) (CAS Registry No. 95058-814) is a nucleoside analog used as chemotherapy. It is FDA approved for treatment of, e.g., breast, pancreatic, lung, and ovarian cancers. It has the following formula:

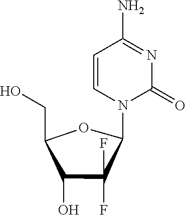

As a pyrimidine analog, the drug replaces one of the building blocks of nucleic acids in rapidly growing tumor cells, in this case cytidine, during DNA replication. The process arrests tumor growth, as new nucleosides cannot be attached to the “faulty” nucleoside, resulting in apoptosis (cellular “suicide”). Gemcitabine is used in various carcinomas: non-small cell lung cancer, pancreatic cancer, bladder cancer and breast cancer. Gemcitabine is the standard of care for many pancreatic cancers.

Other FDA-approved nucleoside analogs for cancer treatments include cytosine arabinoside (ara-C or Cytarabine) for treatment of acute myeloid leukemia (AML), acute lymphocytic leukemia (ALL), chronic myelogenous leukemia (CML) and non-Hodgkin's lymphoma (www_dot_drugs_dot_com/monograph/cytarabine.html (visited Nov. 14, 2018)), and fluorouracil (5-FU) for the treatment of colon cancer, esophageal cancer, stomach cancer, pancreatic cancer, breast cancer, basal cell carcinoma, and cervical cancer (www_dot_drugs_dot_com/monograph/fluorouracil.html (visited Nov. 14, 2018)). Ara-C (CAS Registry No. 147-94-4) has the following formula:

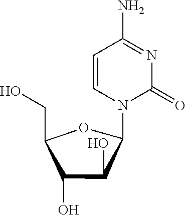

5-FU (CAS Registry No. 51-21-8) has the following formula:

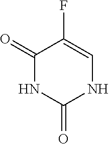

In some embodiments, the chemotherapeutic nucleoside analog is administered intravenously, intrathecally, or subcutaneously. In some embodiments, the chemotherapeutic nucleoside analog is administered intravenously.

SMAC Mimetics

Second mitochondria-derived activator of caspases (SMAC) is a mitochondrial protein that binds inhibitor of apoptosis proteins (IAPs) inhibiting IAPs ability to bind caspases, a class of pro-apoptotic proteins. IAPs antagonistically bind caspases, therefore, SMAC binding of TAPs is pro-apoptotic. Various SMAC mimetics have been developed to mimic the activity of SMAC, e.g., birinapant. APG-1387, Debio 1143, ASTX660. GDC-0152, and HGS-1029/AEG40826. Endogenous SMAC is bivalent, and similarly, some SMAC mimetics are also bivalent, e.g., birinapant, APG-1387, and HGS-1029/AEG40826. Alternatively, some SMAC mimetics are monovalent, e.g., Debio 1143, ASTX660, and GDC-0152.

Birinapant (U.S. Pat. No. 8,283,372, CAS Registry No. 1260251-31-7) has the following formula:

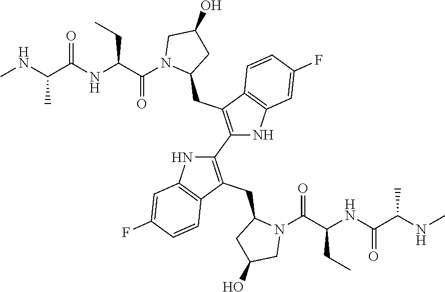

APG-1387 (Li, N. et al., Cancer Letters, 2016, 381:14-22) has the following formula:

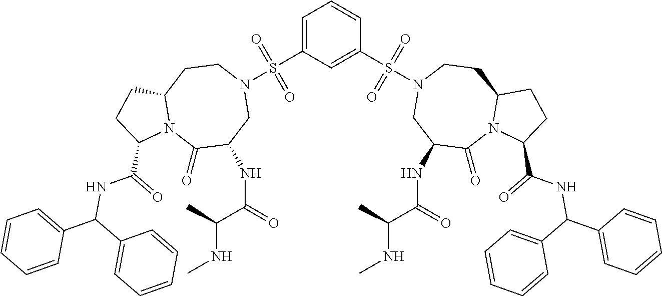 [SMILES: CN[C@@H](C)C(=O)N[C@H]1CN(S(=O)(=O)c2cccc(S(=O)(=O)N3CC[C@H]4CC[C@@H](C(=O)NC(c5ccccc5)c5ccccc5)N4C(=O)[C@@H](NC(=O)[C@H](C)NC)C3)c2)CC[C@H]2CC[C@@H](C(=O)NC(c3ccccc3)c3ccccc3)N2C1=O]

Debio 1143 (AT-406/SM-406; Cai et al., J Med Chem. 2011; 54(8): 2714-2726) has the following formula:

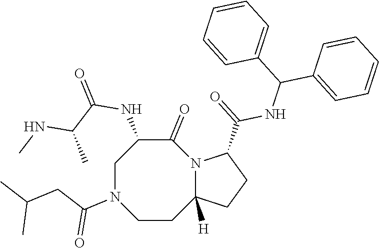 [SMILES: CN[C@@H](C)C(=O)N[C@H]1CN(C(=O)CC(C)C)CC[C@H]2CC[C@@H](C(=O)NC(c3ccccc3)c3ccccc3)N2C1=O]

ASTX660 (Ward. G A, et al., Mol Cancer Ther. 2018 July; 17(7)1381-1391) has the following formula:

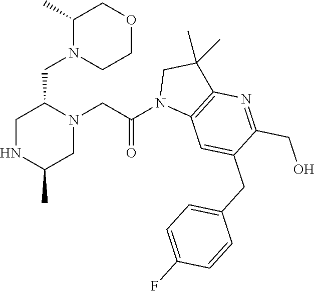 [SMILES: C[C@@H]1CN(CC(=O)N2CC(C)(C)c3nc(CO)c(Cc4ccc(F)cc4)cc32)[C@@H](CN2CCOC[C@H]2C)CN1]

GDC-0152/RG-7419 (Flygare, J A, et al., J. Med. Chem. 55:4101-41 13 (2012), CAS Registry No. 873652-48-3) has the following formula:

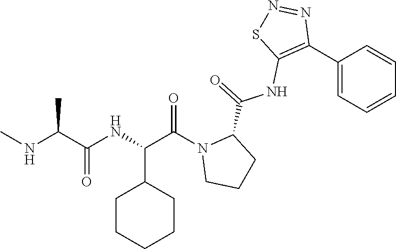 [SMILES: CN[C@@H](C)C(=O)N[C@H](C(=O)N1CCC[C@H]1C(=O)Nc1snnc1-c1ccccc1)C1CCCCC1]

HGS-1029/AEG40826 (CAS Registry No. 1107664-44-7) is described in U.S. Pat. No. 7,579,320.

In some embodiments, the cancer therapy comprises a SMAC mimetic. In some embodiment, the SMAC mimetic is a bivalent SMAC mimetic, such as birinapant, APG-1387, or HGS-1029/AEG40826. In some embodiments, the SMAC mimetic is a monovalent SMAC mimetic, such as Debio 1143, ASTX660, and GDC-0152. In some embodiments, the SMAC mimetic is administered orally or intravenously. In some embodiments, the SMAC mimetic is a bivalent SMAC mimetic, and the SMAC mimetic is administered intravenously. In some embodiments, the SMAC mimetic is a monovalent SMAC mimetic, and the SMAC mimetic is administered orally. In some embodiments, the cancer therapy comprises a SMAC mimetic, such as birinapant, APG-1387, Debio 1143, ASTX660, GDC-0152, or HGS-1029/AEG40826, and the cancer is a cancer disclosed herein.

In some embodiments, the cancer therapy comprises a SMAC mimetic, such as a monovalent or bivalent SMAC mimetic, such as birinapant, APG-1387, or HGS-1029/AEG40826, and the cancer is head and neck cancer, such as head and neck sarcoma. In some embodiments, the cancer therapy comprises a bivalent SMAC mimetic, such as birinapant, and the cancer is head and neck cancer. In some embodiments, the cancer therapy comprises a bivalent SMAC mimetic, such as birinapant, and the cancer is head and neck sarcoma.

In some embodiments, the cancer therapy comprises a SMAC mimetic, such as a monovalent or bivalent SMAC mimetic, such as birinapant, APG-1387, or HGS-1029/AEG40826, and the cancer is colorectal cancer. In some embodiments, the cancer therapy comprises a bivalent SMAC mimetic, such as birinapant, and the cancer is colorectal cancer.

In some embodiments, the cancer therapy comprises a SMAC mimetic, such as a monovalent or bivalent SMAC mimetic, such as birinapant, APG-1387, or HGS-1029/AEG40826, and the cancer is breast cancer, such as triple negative breast cancer. In some embodiments, the cancer therapy comprises a bivalent SMAC mimetic, such as birinapant, and the cancer is breast cancer, such as triple negative breast cancer.

In some embodiments, the cancer therapy comprises a SMAC mimetic, such as a monovalent or bivalent SMAC mimetic, such as birinapant, APG-1387, or HGS-1029/AEG40826, and the method further comprises administering radiation therapy. In some embodiments, the cancer therapy comprises a SMAC mimetic, such as a monovalent or bivalent SMAC mimetic, such as birinapant, APG-1387, or HGS-1029/AEG40826, the method further comprises administering radiation therapy, and the cancer is head and neck cancer, such as head and neck sarcoma. In some embodiments, the cancer therapy comprises a SMAC mimetic, such as a monovalent or bivalent SMAC mimetic, such as birinapant, APG-1387, or HGS-1029/AEG40826, the method further comprises administering radiation therapy, and the cancer is colorectal cancer. In some embodiments, the cancer therapy comprises a SMAC mimetic, such as a monovalent or bivalent SMAC mimetic, such as birinapant, APG-1387, or HGS-1029/AEG40826, the method further comprises administering radiation, and the cancer is breast cancer, such as triple negative breast cancer.

Vinca Alkaloids

Vinca alkaloids are a class of anti-microtubule and anti-mitotic agents that block microtubule polymerization and therefore cellular division. For this reason, vinca alkaloids are used as cancer chemotherapy. Various vinca alkaloids have been developed e.g., vincristine. Vincristine (CAS Registry No. 57-22-7) has the following formula:

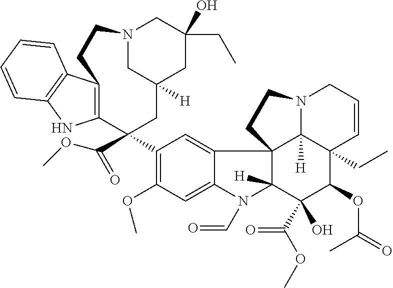

In some embodiments, the cancer therapy is a vinca alkaloid, such as vincristine, and the cancer is a cancer disclosed herein. In some embodiments, the vinca alkaloid is administered intravenously.

BTK Inhibitors

Bruton's tyrosine kinase (BTK) is a protein that is important for B cell development. Accordingly, various BTK inhibitors, e.g., ibrutinib, have been developed to treat B cell related cancers. Ibrutinib (CAS Registry No. 936563-%-1) has the following formula:

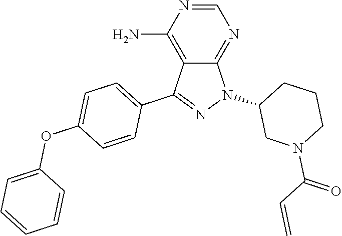 [SMILES: C=CC(=O)N1CCC[C@@H](n2nc(-c3ccc(Oc4ccccc4)cc3)c3c(N)ncnc32)C1]

In some embodiments, the cancer therapy is a BTK inhibitor, such as ibrutinib, and the cancer is a cancer disclosed herein. In some embodiments, the BTK inhibitor is administered orally.

PI3Kδ Inhibitors

Inhibition of phosphoinositide 3-kinase delta (PI3Kδ) prevents proliferation and induces apoptosis in B cells. Accordingly, various PI3Kδ inhibitors, e.g., idelalisib have been developed to treat B cell related cancers. Idelalisib (CAS Registry No. 870281-82-6) has the following formula:

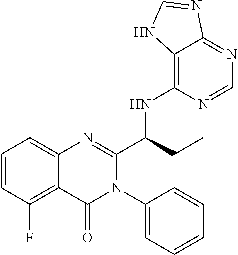 [SMILES: CC[C@H](Nc1ncnc2nc[nH]c12)c1nc2cccc(F)c2c(=O)n1-c1ccccc1]

In some embodiments, the cancer therapy is a PI3Kδ inhibitor, such as idelalisib, and the cancer is a cancer disclosed herein. In some embodiments, the PI3Kδ inhibitor is administered orally.

Mcl-1 Inhibitors

Myeloid cell leukemia-1 (Mcl-1) is an anti-apoptotic anti-proliferative protein. Accordingly, various Mcl-1 inhibitors, e.g., MIK665/S-64315 have been developed to treat cancer MIK665/S-64315 (CAS Registry No. 1799631-75-6) has the following formula:

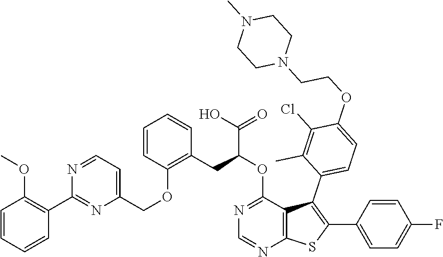

In some embodiments, the cancer therapy is a Mcl-1 inhibitor, such as MIK665/S-64315, and the cancer is a cancer disclosed herein. In some embodiments, the Mcl-1 inhibitor is administered intravenously.

Anti-VEGF Antibodies

Vascular endothelial growth factor (VEGF) is a protein that is known to promote angiogenesis. Bevacizumab (CAS Registry No: 216974-75-3), an antibody that inhibit VEGF, has been approved to treat colorectal cancer, non-small cell lung cancer, glioblastoma, renal cell carcinoma, cervical cancer, epithelial ovarian cancer, fallopian tube cancer, primary peritoneal cancer, or hepatocellular carcinoma. As used herein, the term “bevacizumab” includes bevacizumab and bevacizumab biosimilars, e.g., bevacizumab-awwb and bevacizumab-bvzr.

In some embodiments, the cancer therapy is an anti-VEGF antibody, such as bevacizumab, and the cancer is a cancer disclosed herein. In some embodiments, the anti-VEGF antibody is administered intravenously.

Pharmaceutical Compositions and Administration Methods

Methods of preparing and administering a dimeric, pentameric, or hexameric DR5 binding molecule as provided herein to a subject in need thereof are known or are readily determined in view of this disclosure. The route of administration of a DR5 binding molecule can be, for example, oral, parenteral, by inhalation or topical. The term parenteral as used herein includes, e.g., intravenous, intraarterial, intraperitoneal, intramuscular, subcutaneous, rectal, or vaginal administration. While these forms of administration are contemplated as suitable forms, another example of a form for administration would be a solution for injection, in particular for intravenous or intraarterial injection or drip. A suitable pharmaceutical composition can comprise a buffer (e.g., acetate, phosphate, or citrate buffer), a surfactant (e.g., polysorbate), optionally a stabilizer agent (e.g., human albumin), etc.

As discussed herein, a dimeric, pentameric, or hexameric DR5 binding molecule as provided herein can be administered in a pharmaceutically effective amount for the in vivo treatment of cancers expressing DR5. In this regard, it will be appreciated that the disclosed binding molecules and compounds can be formulated so as to facilitate administration and promote stability of the active agent. Pharmaceutical compositions accordingly can comprise a pharmaceutically acceptable, non-toxic, sterile carrier such as physiological saline, non-toxic buffers, preservatives, and the like. A pharmaceutically effective amount of a dimeric, pentameric, or hexameric DR5 binding molecule as provided herein means an amount sufficient to achieve effective binding to a target and to achieve a therapeutic benefit. A pharmaceutically effective amount of a cancer therapy as provided herein means an amount sufficient to achieve a therapeutic benefit. Suitable formulations are described in Remington's Pharmaceutical Sciences (Mack Publishing Co.) 16th ed. (1980).

Certain pharmaceutical compositions provided herein can be orally administered in an acceptable dosage form including. e.g., capsules, tablets, aqueous suspensions, or solutions. Certain pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents.

The amount of a dimeric, pentameric, or hexameric DR5 binding molecule or cancer therapy that can be combined with carrier materials to produce a single dosage form will vary depending, e.g., upon the subject treated and the particular mode of administration. The composition can be administered as a single dose, multiple doses or over an established period of time in an infusion. Dosage regimens also can be adjusted to provide the optimum desired response (e.g., a therapeutic or prophylactic response).

The compounds described herein can be administered in any pharmaceutically acceptable form, such as in the form of a pharmaceutically acceptable salt, or in free base or free acid form if said form is pharmaceutically acceptable. The compounds described herein, or pharmaceutically acceptable salts thereof, can be administered in pharmaceutically acceptable carriers or excipients.

In keeping with the scope of the present disclosure, a dimeric, pentameric, or hexameric DR5 binding molecule as provided herein can be administered to a subject in need of therapy in an amount sufficient to produce a therapeutic effect. A dimeric, pentameric, or hexameric DR5 binding molecule as provided herein can be administered to the subject in a conventional dosage form prepared by combining the antibody or antigen-binding fragment, variant, or derivative thereof of the disclosure with a conventional pharmaceutically acceptable carrier or diluent according to known techniques. The form and character of the pharmaceutically acceptable carrier or diluent can be dictated by the amount of active ingredient with which it is to be combined, the route of administration and other well-known variables.

By “therapeutically effective dose or amount” or “effective amount” is intended an amount of a dimeric, pentameric, or hexameric DR5 binding molecule, that when administered brings about a positive therapeutic response with respect to treatment of a patient with cancer expressing DR5.

Therapeutically effective doses of the compositions disclosed herein for treatment of cancer can vary depending upon many different factors, including means of administration, target site, physiological state of the patient, whether the patient is human or an animal, other medications administered, and whether treatment is prophylactic or therapeutic. In certain embodiments, the subject or patient is a human, but non-human mammals including transgenic mammals can also be treated. Treatment dosages can be titrated using routine methods known to those of skill in the art to optimize safety and efficacy. In certain embodiments, the effective amount of the DR5 binding molecule or cancer therapy is a lower amount than the effective amount of the DR5 binding molecule or cancer therapy as a single agent.

The amount of a dimeric, pentameric, or hexameric DR5 binding molecule to be administered is readily determined by one of ordinary skill in the art without undue experimentation given this disclosure. Factors influencing the mode of administration and the respective amount of a dimeric, pentameric, or hexameric DR5 binding molecule include, but are not limited to, the severity of the disease, the history of the disease, and the age, height, weight, health, and physical condition of the individual undergoing therapy. Similarly, the amount of a dimeric, pentameric, or hexameric DR5 binding molecule to be administered will be dependent upon the mode of administration and whether the subject will undergo a single dose or multiple doses of this agent.

In some embodiments, the dimeric, pentameric, or hexameric DR5 binding molecule disclosed herein and the cancer therapy disclosed herein are administered simultaneously. In some embodiments, the dimeric, pentameric, or hexameric DR5 binding molecule disclosed herein and the cancer therapy are administered sequentially. In some embodiments, the method comprises administering the dimeric, pentameric, or hexameric DR5 binding molecule prior to administering the cancer therapy. In some embodiments, the dimeric, pentameric, or hexameric DR5 binding molecule is administered at least 1 minute, 5 minutes, 10 minutes, 15 minutes, 30 minutes, 1 hour, 2 hours, 4 hours, 6 hours, 12 hours, 1 day, 2 days, 3 days, 1 week, or 2 weeks prior to administering the cancer therapy. In some embodiments, the dimeric, pentameric, or hexameric DR5 binding molecule is administered 1 minute to 1 month prior to administering the cancer therapy, such as 1 minute to 2 weeks, 1 minute to 3 days, 1 minute to 1 day, 15 minutes to 2 weeks, 15 minutes to 3 days, 15 minutes to 1 day, 1 hour to 2 weeks, 1 hour to 3 days, 1 hour to 1 day, 6 hours to 2 weeks, 6 hours to 3 days, 6 hours to 1 day, 1 day to 2 weeks, or 1 day to 3 days prior to administering the cancer therapy.

In some embodiments, the method comprises administering the cancer therapy prior to administering the dimeric, pentameric, or hexameric DR5 binding molecule. In some embodiments, the cancer therapy is administered at least 1 minute, 5 minutes, 10 minutes, 15 minutes, 30 minutes, 1 hour, 2 hours, 4 hours, 6 hours, 12 hours, 1 day, 2 days, 3 days, 1 week, or 2 weeks prior to administering the dimeric, pentameric, or hexameric DR5 binding molecule. In some embodiments, the cancer therapy is administered 1 minute to 1 month prior to administering the cancer therapy, such as 1 minute to 2 weeks, 1 minute to 3 days, 1 minute to 1 day, 15 minutes to 2 weeks, 15 minutes to 3 days, 15 minutes to 1 day, 1 hour to 2 weeks, 1 hour to 3 days, 1 hour to 1 day, 6 hours to 2 weeks, 6 hours to 3 days, 6 hours to 1 day, 1 day to 2 weeks, or 1 day to 3 days prior to administering the dimeric, pentameric, or hexameric DR5 binding molecule.

This disclosure also provides for the use of a dimeric, pentameric, or hexameric DR5 binding molecule in the manufacture of a medicament for treating, preventing, or managing cancer where the cancer expresses DR5.

Kits and Articles of Manufacture

Also provided herein is a kit comprising a dimeric, pentameric, or hexameric DR5 binding molecule disclosed herein and instructions for use in accordance with any of the methods described herein.

Also provided herein is a kit comprising a dimeric, pentameric, or hexameric DR5 binding molecule disclosed herein and a cancer therapy for use in any of the methods described herein.

Also provided herein is a kit comprising a dimeric, pentameric, or hexameric DR5 binding molecule disclosed herein and/or a cancer therapy, and instructions for use in accordance with any of the methods described herein, where the cancer therapy is a second mitochondria-derived activator of caspases (SMAC) mimetic, a folic acid analog, a platinum-based agent, a taxane, a topoisomerase II inhibitor, a vinca alkaloid, a Bruton's tyrosine kinase (BTK) inhibitor, a phosphoinositide 3-kinase delta (PI3Kδ) inhibitor, a myeloid cell leukemia-1 (Mcl-1) inhibitor, or any combination thereof.

Instructions supplied in the kits are typically written instructions on a label or package insert (e.g., a paper sheet included in the kit), but machine-readable instructions (e.g., instructions carried on a magnetic or optical storage disk) are also acceptable, as are labels or package inserts that provide references to electronically stored instructions, such as a hyperlink or barcode that directs to a website.

This disclosure employs, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Green and Sambrook, ed. (2012) Molecular Cloning A Laboratory Manual (4th ed.; Cold Spring Harbor Laboratory Press); Sambrook et al., ed. (1992) Molecular Cloning: A Laboratory Manual, (Cold Springs Harbor Laboratory, NY); D. N. Glover and B. D. Hames, eds., (1995) DNA Cloning 2d Edition (IRL Press), Volumes 1-4; Gait, ed. (1990) Oligonucleotide Synthesis (IRL Press); Mullis et al. U.S. Pat. No. 4,683,195; Hanies and Higgins, eds. (1985) Nucleic Acid Hybridization (IRL Press); Hames and Higgins, eds. (1984) Transcription And Translation (IRL Press); Freshney (2016) Culture Of Animal Cells, 7th Edition (Wiley-Blackwell); Woodward, J., Immobilized Cells And Enzymes (IRL Press) (1985); Perbal (1988) A Practical Guide To Molecular Cloning; 2d Edition (Wiley-Interscience); Miller and Calos eds. (1987) Gene Transfer Vectors For Mammalian Cells, (Cold Spring Harbor Laboratory); S.C. Makrides (2003) Gene Transfer and Expression in Mammalian Cells (Elsevier Science); Methods in Enzymology, Vols. 151-155 (Academic Press, Inc., N.Y.); Mayer and Walker, eds. (1987) Immunochemical Methods in Cell and Molecular Biology (Academic Press, London); Weir and Blackwell, eds.; and in Ausubel et al. (1995) Current Protocols in Molecular Biology (John Wiley and Sons).

General principles of antibody engineering are set forth, e.g., in Strohl, W. R., and L. M. Strohl (2012), Therapeutic Antibody Engineering (Woodhead Publishing). General principles of protein engineering are set forth, e.g., in Park and Cochran, eds. (2009), Protein Engineering and Design (CDC Press). General principles of immunology are set forth, e.g., in: Abbas and Lichtman (2017) Cellular and Molecular Immunology 9th Edition (Elsevier). Additionally, standard methods in immunology known in the art can be followed, e.g., in Current Protocols in Immunology (Wiley Online Library); Wild, D. (2013), The Immunoassay Handbook 4th Edition (Elsevier Science); Greenfield, ed. (2013), Antibodies, a Laboratory Manual, 2d Edition (Cold Spring Harbor Press); and Ossipow and Fischer, eds., (2014), Monoclonal Antibodies: Methods and Protocols (Humana Press).

### EXEMPLARY EMBODIMENTS

Among the provided embodiments are:

Embodiment 1. A method for inhibiting, delaying, or reducing malignant cell growth in a subject with cancer in need of treatment, comprising administering to the subject a combination therapy comprising:

(a) an effective amount of a pentameric or hexameric IgM or IgM-like antibody or a dimeric IgA or IgA-like antibody, or a multimerized antigen-binding fragment, variant, or derivative thereof that specifically and agonistically binds to DR5, where three to twelve of the antigen binding domains of the IgM or IgM-like antibody or multimerized antigen-binding fragment, variant, or derivative thereof or three or four of the antigen binding domains of the IgA or IgA-like antibody or multimerized antigen-binding fragment, variant, or derivative thereof are DR5-specific and agonistic; and

(b) an effective amount of a cancer therapy, where the cancer therapy comprises a second mitochondria-derived activator of caspases (SMAC) mimetic, radiation, a folic acid analog, a platinum-based agent, a taxane, a topoisomerase II inhibitor, a vinca alkaloid, a Bruton's tyrosine kinase (BTK) inhibitor, a phosphoinositide 3-kinase delta (PI3Kδ) inhibitor, a myeloid cell leukemia-1 (Mcl-1) inhibitor, an anti-VEGF antibody, or any combination thereof.

Embodiment 2. A method for inhibiting, delaying, or reducing malignant cell growth in a subject with cancer in need of treatment, comprising administering an effective amount of a pentameric or hexameric IgM or IgM-like antibody or a dimeric IgA or IgA-like antibody, or a multimerized antigen-binding fragment, variant, or derivative thereof that specifically and agonistically binds to DR5, where three to twelve of the antigen binding domains of the IgM or IgM-like antibody or multimerized antigen-binding fragment, variant, or derivative thereof or three or four of the antigen binding domains of the IgA or IgA-like antibody or multimerized antigen-binding fragment, variant, or derivative thereof are DR5-specific and agonistic.

where the pentameric or hexameric IgM or IgM-like antibody or the dimeric IgA or IgA-like antibody, or the multimerized antigen-binding fragment, variant, or derivative thereof is administered with an effective amount of a cancer therapy, where the cancer therapy comprises a second mitochondria-derived activator of caspases (SMAC) mimetic, radiation, a folic acid analog, a platinum-based agent, a taxane, a topoisomerase II inhibitor, a vinca alkaloid, a Bruton's tyrosine kinase (BTK) inhibitor, a phosphoinositide 3-kinase delta (PI3Kδ) inhibitor, a myeloid cell leukemia-1 (Mcl-1) inhibitor, an anti-VEGF antibody, or any combination thereof.

Embodiment 3. A method for inhibiting, delaying, or reducing malignant cell growth in a subject with cancer in need of treatment, comprising administering an effective amount of a cancer therapy, where the cancer therapy comprises a second mitochondria-derived activator of caspases (SMAC) mimetic, radiation, a folic acid analog, a platinum-based agent, a taxane, a topoisomerase II inhibitor, a vinca alkaloid, a Bruton's tyrosine kinase (BTK) inhibitor, a phosphoinositide 3-kinase delta (PI3Kδ) inhibitor, a myeloid cell leukemia-1 (Mcl-1) inhibitor, an anti-VEGF antibody, or any combination thereof,

where the cancer therapy is administered with a pentameric or hexameric IgM or IgM-like antibody or a dimeric IgA or IgA-like antibody, or a multimerized antigen-binding fragment, variant, or derivative thereof that specifically and agonistically binds to DR5, where three to twelve of the antigen binding domains of the IgM or IgM-like antibody or multimerized antigen-binding fragment, variant, or derivative thereof or three or four of the antigen binding domains of the IgA or IgA-like antibody or multimerized antigen-binding fragment, variant, or derivative thereof are DR5-specific and agonistic.

Embodiment 4. A method for inducing apoptosis in a cancer cell in in a subject with cancer in need of treatment, comprising administering to the subject a combination therapy comprising:

(a) an effective amount of a pentameric or hexameric IgM or IgM-like antibody or a dimeric IgA or IgA-like antibody, or a multimerized antigen-binding fragment, variant, or derivative thereof that specifically and agonistically binds to DR5, where three to twelve of the antigen binding domains of the IgM or IgM-like antibody or multimerized antigen-binding fragment, variant, or derivative thereof or three or four of the antigen binding domains of the IgA or IgA-like antibody or multimerized antigen-binding fragment, variant, or derivative thereof are DR5-specific and agonistic; and

(b) an effective amount of a cancer therapy, where the cancer therapy comprises a second mitochondria-derived activator of caspases (SMAC) mimetic, radiation, a folic acid analog, a platinum-based agent, a taxane, a topoisomerase II inhibitor, a vinca alkaloid, a Bruton's tyrosine kinase (BTK) inhibitor, a phosphoinositide 3-kinase delta (PI3Kδ) inhibitor, a myeloid cell leukemia-1 (Mcl-1) inhibitor, an anti-VEGF antibody, or any combination thereof.

Embodiment 5. A method for inhibiting, delaying, or reducing malignant cell growth in a subject with cancer in need of treatment, comprising administering an effective amount of a pentameric or hexameric IgM or IgM-like antibody or a dimeric IgA or IgA-like antibody, or a multimerized antigen-binding fragment, variant, or derivative thereof that specifically and agonistically binds to DR5, where three to twelve of the antigen binding domains of the IgM or IgM-like antibody or multimerized antigen-binding fragment, variant, or derivative thereof or three or four of the antigen binding domains of the IgA or IgA-like antibody or multimerized antigen-binding fragment, variant, or derivative thereof are DR5-specific and agonistic,

where the pentameric or hexameric IgM or IgM-like antibody or the dimeric IgA or IgA-like antibody, or the multimerized antigen-binding fragment, variant, or derivative thereof is administered with an effective amount of a cancer therapy, where the cancer therapy comprises a second mitochondria-derived activator of caspases (SMAC) mimetic, radiation, a folic acid analog, a platinum-based agent, a taxane, a topoisomerase II inhibitor, a vinca alkaloid, a Bruton's tyrosine kinase (BTK) inhibitor, a phosphoinositide 3-kinase delta (PI3Kδ) inhibitor, a myeloid cell leukemia-1 (Mcl-1) inhibitor, an anti-VEGF antibody, or any combination thereof.

Embodiment 6. A method for inducing apoptosis in a cancer cell in in a subject with cancer in need of treatment, comprising administering an effective amount of an effective amount of a cancer therapy, where the cancer therapy comprises a second mitochondria-derived activator of caspases (SMAC) mimetic, radiation, a folic acid analog, a platinum-based agent, a taxane, a topoisomerase II inhibitor, a vinca alkaloid, a Bruton's tyrosine kinase (BTK) inhibitor, a phosphoinositide 3-kinase delta (PI3Kδ) inhibitor, a myeloid cell leukemia-1 (Mcl-1) inhibitor, an anti-VEGF antibody, or any combination thereof,

where the cancer therapy is administered with a pentameric or hexameric IgM or IgM-like antibody or a dimeric IgA or IgA-like antibody, or a multimerized antigen-binding fragment, variant, or derivative thereof that specifically and agonistically binds to DR5, where three to twelve of the antigen binding domains of the IgM or IgM-like antibody or multimerized antigen-binding fragment, variant, or derivative thereof or three or four of the antigen binding domains of the IgA or IgA-like antibody or multimerized antigen-binding fragment, variant, or derivative thereof are DR5-specific and agonistic.

Embodiment 7. The method of any one of embodiments 1 to 6, where the cancer therapy comprises a folic acid analog.

Embodiment 8. The method of embodiment 7, where the folic acid analog comprises leucovorin.

Embodiment 9. The method of any one of embodiments 1 to 6, where the cancer therapy comprises a platinum-based agent.

Embodiment 10. The method of embodiment 9, where the platinum-based agent comprises oxaliplatin, carboplatin, or a combination thereof.

Embodiment 11. The method of embodiment 9 or embodiment 10, where the platinum-based agent comprises oxaliplatin.

Embodiment 12. The method of any one of embodiments 9 to 11, where the platinum-based agent comprises carboplatin.

Embodiment 13. The method of any one of embodiments 1 to 6, where the cancer therapy comprises a taxane.

Embodiment 14. The method of embodiment 13, where the taxane comprises paclitaxel.

Embodiment 15. The method of embodiment 14, where the paclitaxel comprises solvent-based paclitaxel, nab-paclitaxel, or a combination thereof.

Embodiment 16. The method of embodiment 14 or embodiment 15, where the paclitaxel comprises solvent-based paclitaxel.

Embodiment 17. The method of embodiment 14 or embodiment 15, where the paclitaxel comprises nab-paclitaxel.

Embodiment 18. The method of any one of embodiments 1 to 6, where the cancer therapy comprises a topoisomerase II inhibitor.

Embodiment 19. The method of embodiment 18, where the topoisomerase II inhibitor comprises an anthracycline.

Embodiment 20. The method of embodiment 19, where the anthracycline comprises doxorubicin.

Embodiment 21. The method of embodiment 18, where the topoisomerase II inhibitor comprises etoposide.

Embodiment 22. The method of any one of embodiments 1 to 6, where the cancer therapy comprises radiation.

Embodiment 23. The method of any one of embodiments 1 to 6, where the cancer therapy comprises a SMAC mimetic.

Embodiment 24. The method of embodiment 23, where the SMAC mimetic comprises birinapant, GDC-0152, HGS-1029/AEG40826, Debio1143, APG-1387. ASTX660, or a combination thereof.

Embodiment 25. The method of embodiment 23 or embodiment 24, where the SMAC mimetic comprises a bivalent SMAC mimetic.

Embodiment 26. The method of any one of embodiments 23 to 25, where the SMAC mimetic comprises birinapant.

Embodiment 27. The method of any one of embodiments 23 to 25, where the SMAC mimetic comprises APG-1387.

Embodiment 28. The method of any one of embodiments 23 to 25, where the SMAC mimetic comprises HGS-1029/AEG40826.

Embodiment 29. The method of embodiment 23 or embodiment 24, where the SMAC mimetic comprises a monovalent SMAC mimetic.

Embodiment 30. The method of any one of embodiments 23, 24, or 29, where the SMAC mimetic comprises GDC-0152.

Embodiment 31. The method of any one of embodiments 23, 24, or 29, where the SMAC mimetic comprises Debio1143.

Embodiment 32. The method of any one of embodiments 23, 24, or 29, where the SMAC mimetic comprises ASTX660.

Embodiment 33. The method of any one of embodiments 1 to 6, where the cancer therapy comprises a vinca alkaloid.

Embodiment 34. The method of embodiment 33, where the vinca alkaloid comprises vincristine.

Embodiment 35. The method of any one of embodiments 1 to 6, where the cancer therapy comprises a BTK inhibitor.

Embodiment 36. The method of embodiment 35, where the BTK inhibitor comprises ibrutinib.

Embodiment 37. The method of any one of embodiments 1 to 6, where the cancer therapy comprises a PI3Kδ inhibitor.

Embodiment 38. The method of embodiment 37, where the PI3Kδ inhibitor comprises idelalisib.

Embodiment 39. The method of any one of embodiments 1 to 6, where the cancer therapy comprises a Mcl-1 inhibitor.

Embodiment 40. The method of embodiment 39, where the Mcl-1 inhibitor comprises MIK665.

Embodiment 41. The method of any one of embodiments 1 to 6, where the cancer therapy comprises an anti-VEGF antibody.

Embodiment 42. The method of embodiment 41, where the anti-VEGF antibody is bevacizumab.

Embodiment 43. The method of any one of embodiments 1 to 42, further comprising administering an effective amount of an additional cancer therapy.

Embodiment 44. The method of embodiment 43, where the additional cancer therapy comprises a topoisomerase I inhibitor, a nucleoside analog, a platinum-based agent, or any combination thereof.

Embodiment 45. The method of embodiment 43 or embodiment 44, where the additional cancer therapy comprises a topoisomerase I inhibitor.

Embodiment 46. The method of embodiment 45, where the topoisomerase I inhibitor comprises irinotecan, topotecan, or a combination thereof.

Embodiment 47. The method of embodiment 45 or embodiment 46, where the topoisomerase I inhibitor comprises irinotecan.

Embodiment 48. The method of any one of embodiments 43 to 47, where the additional cancer therapy comprises a nucleoside analog.

Embodiment 49. The method of embodiment 48, where the nucleoside analog comprises fluorouracil (5-FU), gemcitabine, or any combination thereof.

Embodiment 50. The method of embodiment 49, where the nucleoside analog comprises fluorouracil (5-FU).

Embodiment 51. The method of embodiment 49, where the nucleoside analog comprises gemcitabine.

Embodiment 52. The method of any one of embodiments 1 to 51., where the cancer is a hematologic cancer or a solid tumor.

Embodiment 53. The method of embodiment 52, where the cancer is a hematologic cancer.

Embodiment 54. The method of embodiment 52 or 53, where the hematologic cancer is leukemia, lymphoma, myeloma, any metastases thereof, or any combination thereof.

Embodiment 55. The method of any one of embodiments 52 to 54, where the hematologic cancer is acute myeloid leukemia (AML), chronic myeloid leukemia (CML), acute lymphocytic leukemia (ALL), small lymphocytic lymphoma (SLL), chronic lymphocytic leukemia, hairy cell leukemia. Hodgkin lymphoma, non-Hodgkin lymphoma, multiple myeloma, any metastases thereof, or any combination thereof.

Embodiment 56. The method of any one of embodiments 52 to 55, where the hematologic cancer is acute myeloid leukemia (AML).

Embodiment 57. The method of any one of embodiments 53 to 56, where the cancer therapy comprises doxorubicin.

Embodiment 58. The method of embodiment 52, where the cancer is a solid tumor.

Embodiment 59. The method of embodiment 52 or 58, where the cancer is bladder cancer, colorectal cancer, sarcoma, gastric cancer, lung cancer, pancreatic cancer, melanoma, ovarian cancer, head and neck cancer, or breast cancer.

Embodiment 60. The method of any one of embodiments 52, 58, or 59, where the cancer is sarcoma.

Embodiment 61. The method of embodiment 60, where the sarcoma is fibrosarcoma, chondrosarcoma, or osteosarcoma.

Embodiment 62. The method of embodiment 60, where the sarcoma is fibrosarcoma.

Embodiment 63. The method of any one of embodiments 60 to 62, where the cancer therapy comprises doxorubicin.

Embodiment 64. The method of any one of embodiments 52, 58, or 59, where the cancer is colorectal cancer.

Embodiment 65. The method of embodiment 64, where the cancer therapy comprises oxaliplatin.

Embodiment 66. The method of embodiment 65, where the additional therapy comprises 5-FU.

Embodiment 67. The method of embodiment 64, where the cancer therapy comprises leucovorin.

Embodiment 68. The method of embodiment 67, where the additional therapy comprises oxaliplatin or irinotecan.

Embodiment 69. The method of any one of embodiments 52, 58, or 59, where the cancer is gastric cancer.

Embodiment 70. The method of embodiment 69, where the cancer therapy comprises carboplatin.

Embodiment 71. The method of embodiment 69, where the cancer therapy comprises oxaliplatin.

Embodiment 72. The method of embodiment 69, where the cancer therapy comprises paclitaxel.

Embodiment 73. The method of any one of embodiments 52, 58, or 59, where the cancer is lung cancer.

Embodiment 74. The method of embodiment 73, where the lung cancer is non-small cell lung cancer (NSCLC).

Embodiment 75. The method of embodiment 73 or embodiment 74, where the cancer therapy comprises carboplatin.

Embodiment 76. The method of embodiment 73 or embodiment 74, where the cancer therapy comprises paclitaxel.

Embodiment 77. The method of any one of embodiments 52, 58, or 59, where the cancer is pancreatic cancer.

Embodiment 78. The method of embodiment 77, where the cancer therapy comprises paclitaxel.

Embodiment 79. The method of embodiment 78, where the additional therapy comprises gemcitabine.

Embodiment 80. The method of any one of embodiments 52, 58, or 59, where the cancer is head and neck cancer.

Embodiment 81. The method of embodiment 80, where the head and neck cancer is head and neck sarcoma.

Embodiment 82. The method of any one of embodiments 52, 58, or 59, where the cancer is breast cancer.

Embodiment 83. The method of embodiment 82, where the breast cancer is triple negative breast cancer (TNBC).

Embodiment 84. The method of any one of embodiments 1 to 83, where the three or four antigen-binding domains or the three to twelve antigen-binding domains of the antibody or multimerized antigen-binding fragment, variant, or derivative thereof comprise a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL amino acid sequences SEQ ID NO: 1 and SEQ ID NO: 2; SEQ ID NO: 3 and SEQ ID NO: 4; SEQ ID NO: 5 or SEQ ID NO: 90 and SEQ ID NO: 6; SEQ ID NO: 7 and SEQ ID NO: 8; SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; SEQ ID NO: 23 and SEQ ID NO: 24; SEQ ID NO: 25 and SEQ ID NO: 26; SEQ ID NO: 27 and SEQ ID NO: 28; SEQ ID NO: 29 and SEQ ID NO: 30; SEQ ID NO: 31 and SEQ ID NO: 32; SEQ ID NO: 33 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46; SEQ ID NO: 47 and SEQ ID NO: 48; SEQ ID NO: 49 and SEQ ID NO: 50; SEQ ID NO: 51 and SEQ ID NO: 52; SEQ ID NO: 53 and SEQ ID NO: 54; SEQ ID NO: 55 and SEQ ID NO: 56; SEQ ID NO: 82 and SEQ ID NO: 83; SEQ ID NO: 84 and SEQ ID NO: 85; SEQ ID NO: 86 and SEQ ID NO: 87; or SEQ ID NO: 88 and SEQ ID NO: 89; respectively, or the ScFv sequence SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, or SEQ ID NO: 73 or the six CDRs with one or two amino acid substitutions in one or more of the CDRs.

Embodiment 85. The method of embodiment 84, where the three or four antigen-binding domains or the three to twelve antigen-binding domains of the antibody or multimerized antigen-binding fragment, variant, or derivative thereof comprise a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL amino acid sequences SEQ ID NO: 5 or SEQ ID NO: 90 and SEQ ID NO: 6; or SEQ ID NO: 7 and SEQ ID NO: 8, respectively.

Embodiment 86. The method of embodiment 85, where the three or four antigen-binding domains or the three to twelve antigen-binding domains of the antibody or multimerized antigen-binding fragment, variant, or derivative thereof comprise a heavy chain variable region (VH) and a light chain variable region (VL), where the VH and VL comprise six immunoglobulin complementarity determining regions HCDR1. HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL amino acid sequences SEQ ID NO: 5 or SEQ ID NO: 90 and SEQ ID NO: 6, respectively.

Embodiment 87. The method of embodiment 85. where the three or four antigen-binding domains or the three to twelve antigen-binding domains of the antibody or multimerized antigen-binding fragment, variant, or derivative thereof comprise a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL amino acid sequences SEQ ID NO: 7 and SEQ ID NO: 8, respectively.

Embodiment 88. The method of any one of embodiments 1 to 84, where the three or four antigen-binding domains or the three to twelve antigen-binding domains of the antibody or multimerized antigen-binding fragment, variant, or derivative thereof comprise an antibody VH and a VL, where the VH and VL comprise amino acid sequences at least 90% identical to SEQ ID NO: 1 and SEQ ID NO: 2; SEQ ID NO: 3 and SEQ ID NO: 4; SEQ ID NO: 5 or SEQ ID NO: 90 and SEQ ID NO: 6; SEQ ID NO: 7 and SEQ ID NO: 8; SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22. SEQ ID NO: 23 and SEQ ID NO: 24; SEQ ID NO: 25 and SEQ ID NO: 26; SEQ ID NO: 27 and SEQ ID NO: 28; SEQ ID NO: 29 and SEQ ID NO: 30; SEQ ID NO: 31 and SEQ ID NO: 32; SEQ ID NO: 33 and SEQ ID NO: 34; SEQ ID NO: 35 and SEQ ID NO: 36; SEQ ID NO: 37 and SEQ ID NO: 38. SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46; SEQ ID NO: 47 and SEQ ID NO: 48; SEQ ID NO: 49 and SEQ ID NO: 50; SEQ ID NO: 51 and SEQ ID NO: 52; SEQ ID NO: 53 and SEQ ID NO: 54; SEQ ID NO: 55 and SEQ ID NO: 56; SEQ ID NO: 82 and SEQ ID NO: 83; SEQ ID NO: 84 and SEQ ID NO: 85; SEQ ID NO: 86 and SEQ ID NO: 87; or SEQ ID NO: 88 and SEQ ID NO: 89; respectively, or where the VH and VL are contained in an ScFv with an amino acid sequence at least 90% identical to SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71. SEQ ID NO: 72, or SEQ ID NO: 73, respectively.

Embodiment 89. The method of embodiment 88, where the three or four antigen-binding domains or the three to twelve antigen-binding domains of the antibody or multimerized antigen-binding fragment, variant, or derivative thereof comprise an antibody VH and a VL, where the VH and VL comprise amino acid sequences at least 90% identical to SEQ ID NO: 5 or SEQ ID NO: 90 and SEQ ID NO: 6; or SEQ ID NO: 7 and SEQ ID NO: 8, respectively.

Embodiment 90. The method of embodiment 89, where the three or four antigen-binding domains or the three to twelve antigen-binding domains of the antibody or multimerized antigen-binding fragment, variant, or derivative thereof comprise an antibody VH and a VL, where the VH and VL comprise amino acid sequences at least 90% identical to SEQ ID NO: 5 or SEQ ID NO: 90 and SEQ ID NO: 6, respectively.

Embodiment 91. The method of embodiment 89, where the three or four antigen-binding domains or the three to twelve antigen-binding domains of the antibody or multimerized antigen-binding fragment, variant, or derivative thereof comprise an antibody VH and a VL, where the VH and VL comprise amino acid sequences at least 90% identical to SEQ ID NO: 7 and SEQ ID NO: 8, respectively.

Embodiment 92. The method of any one of embodiments 1 to 91, where the antibody or multimerized antigen-binding fragment, variant, or derivative thereof is a dimeric IgA or IgA-like antibody comprising two bivalent IgA binding units or multimerizing fragments thereof and a J-chain or fragment or variant thereof, where each binding unit comprises two IgA heavy chain constant regions or multimerizing fragments thereof each associated with an antigen-binding domain.

Embodiment 93. The method of embodiment 92, where the IgA or IgA-like antibody or multimerized antigen-binding fragment, variant, or derivative thereof further comprises a secretory component, or fragment or variant thereof.

Embodiment 94. The method of embodiment 92 or embodiment 93, where the IgA heavy chain constant regions or multimerizing fragments thereof each comprise a Cα3-tp domain.

Embodiment 95. The method of embodiment 94, where the IgA heavy chain constant regions or multimerizing fragments thereof each comprise a Cα1 domain and/or a Cα2 domain.

Embodiment 96. The method of any one of embodiments 92 to 95, where the IgA heavy chain constant region is a human IgA constant region.

Embodiment 97. The method of any one of embodiments 92 to 96, where each binding unit comprises two IgA heavy chains each comprising a VH situated amino terminal to the IgA constant region or multimerizing fragment thereof, and two immunoglobulin light chains each comprising a VL situated amino terminal to an immunoglobulin light chain constant region.

Embodiment 98. The method of any one of embodiments 1 to 91, where the antibody or multimerized antigen-binding fragment, variant, or derivative thereof is a pentameric or a hexameric IgM antibody comprising five or six bivalent IgM binding units, respectively, where each binding unit comprises two IgM heavy chain constant regions or multimerizing fragments thereof each associated with an antigen-binding domain.

Embodiment 99. The method of embodiment 98, where the IgM heavy chain constant regions or multimerizing fragments thereof each comprise a Cμ4-tp domain.

Embodiment 100. The method of embodiment 99, where the IgM heavy chain constant regions or multimerizing fragments thereof each comprise a Cμ 1 domain, a Cμ2 domain, and/or a Cμ3 domain.

Embodiment 101. The method of any one of embodiments 98 to 100, where the antibody or multimerized antigen-binding fragment, variant, or derivative thereof is pentameric, and further comprises a J-chain, or functional fragment thereof, or variant thereof.

Embodiment 102. The method of any one of embodiments 98 to 101, where the IgM heavy chain constant region is a human IgM constant region.

Embodiment 103. The method of any one of embodiments 98 to 102, where each binding unit comprises two IgM heavy chains each comprising a VH situated amino terminal to the IgM constant region or multimerizing fragment thereof, and two immunoglobulin light chains each comprising a VL situated amino terminal to an immunoglobulin light chain constant region.

Embodiment 104. The method of any one of embodiments 101 to 103, where the J-chain or functional fragment or variant thereof is a variant J-chain comprising one or more single amino acid substitutions, deletions, or insertions relative to a wild-type J-chain that can affect serum half-life of the multimeric binding molecule; and where the multimeric binding molecule exhibits an increased serum half-life upon administration to an animal relative to a reference multimeric binding molecule that is identical except for the one or more single amino acid substitutions, deletions, or insertions, and is administered in the same way to the same animal species.

Embodiment 105. The method of embodiment 104, where the J-chain or functional fragment thereof comprises an amino acid substitution at the amino acid position corresponding to amino acid Y102 of the wild-type human J-chain (SEQ ID NO: 97).

Embodiment 106. The method of embodiment 105, where the amino acid corresponding to Y102 of SEQ ID NO: 97 is substituted with alanine (A), serine (S), or arginine (R).

Embodiment 107. The method of embodiment 106, where the amino acid corresponding to Y102 of SEQ ID NO: 97 is substituted with alanine (A).

Embodiment 108. The method of embodiment 107, where the J-chain is a variant human J-chain and comprises the amino acid sequence SEQ ID NO: 98.

Embodiment 109. The method of embodiment 104, where the J-chain or functional fragment thereof comprises an amino acid substitution at the amino acid position corresponding to amino acid N49, amino acid S51, or both N49 and S51 of the human J-chain (SEQ ID NO: 97), where a single amino acid substitution corresponding to position S51 of SEQ ID NO: 97 is not a threonine (T) substitution.

Embodiment 110. The method of embodiment 109, where the position corresponding to N49 of SEQ ID NO: 97 is substituted with alanine (A). glycine (G), threonine (T), serine (S) or aspartic acid (D).

Embodiment 111. The method of embodiment 110, where the position corresponding to N49 of SEQ ID NO: 97 is substituted with alanine (A).

Embodiment 112. The method of any one of embodiments 109 to 111, where the position corresponding to S51 of SEQ ID NO: 97 is substituted with alanine (A) or glycine (G).

Embodiment 113. The method of embodiment 112, where the position corresponding to S51 of SEQ ID NO: 97 is substituted with alanine (A).

Embodiment 114. The method of any one of embodiments 92 to 97 or 101 to 113, where the J-chain or functional fragment or variant thereof further comprises a heterologous polypeptide, where the heterologous polypeptide is directly or indirectly fused to the J-chain or functional fragment or variant thereof.

Embodiment 115. The method of embodiment 114, where the heterologous polypeptide is fused to the J-chain or functional fragment thereof via a peptide linker.

Embodiment 116. The method of embodiment 115, where the peptide linker comprises at least 5 amino acids, but no more than 25 amino acids.

Embodiment 117. The method of embodiment 115 or 116, where the peptide linker consists of GGGGS (SEQ ID NO: 99), GGGGSGGGGS (SEQ ID NO: 100). GGGGSGGGGSGGGGS (SEQ ID NO: 101), GGGGSGGGGSGGGGSGGGGS (SEQ ID NO: 102), or GGGGSGGGGSGGGGSGGGGSGGGGS (SEQ ID NO: 103).

Embodiment 118. The method of any one of embodiments 114 to 117, where the heterologous polypeptide is fused to the N-terminus of the J-chain or functional fragment or variant thereof, the C-terminus of the J-chain or functional fragment or variant thereof, or to both the N-terminus and C-terminus of the J-chain or functional fragment or variant thereof.

Embodiment 119. The method of any one of embodiments 114 to 118, where the heterologous polypeptide can influence the absorption, distribution, metabolism and/or excretion (ADME) of the multimeric binding molecule.

Embodiment 120. The method of any one of embodiments 114 to 118, where the heterologous polypeptide comprises an antigen binding domain.

Embodiment 121. The method of embodiment 120, where the antigen binding domain of the heterologous polypeptide is an antibody or antigen-binding fragment thereof.

Embodiment 122. The method of embodiment 121, where the antigen-binding fragment comprises an Fab fragment, an Fab′ fragment, an F(ab′)₂ fragment, an Fd fragment, an Fv fragment, a single-chain Fv (scFv) fragment, a disulfide-linked Fv (sdFv) fragment, or any combination thereof.

Embodiment 123. The method of embodiment 121 or embodiment 122, where the antigen-binding fragment is a scFv fragment.

Embodiment 124. The method of any one of embodiments 1 to 123, where administration of the combination therapy results in enhanced therapeutic efficacy relative to administration of the antibody or multimerized antigen-binding fragment, variant, or derivative thereof or the cancer therapy alone.

Embodiment 125. The method of embodiment 124, where the enhanced therapeutic efficacy comprises a reduced tumor growth rate, tumor regression, or increased survival.

Embodiment 126. The method of any one of embodiments 1 to 125, where the subject is human.

All of the references cited above, as well as all references cited herein, arc incorporated herein by reference in their entireties.

The following examples are offered by way of illustration and not by way of limitation.

### EXAMPLES

In the examples that follow, anti-DR5 IgM Mab A and anti-DR5 IgM Mab B were used. Anti-DR5 IgM Mab A and anti-DR5 IgM Mab B were constructed as described in US Patent Application Publication No. 2018-0009897. Anti-DR5 IgM Mab A comprises the VH and VL amino acid SEQ ID NO: 90 and SEQ ID NO: 6 and a J-chain comprising SEQ ID NO: 98 as provided in Table 2, and anti-DR5 IgM Mab B comprises the VH and VL amino acid SEQ ID NO: 7 and SEQ ID NO: 8 as provided in Table 2 and no J-chain.

### Example 1: In Vitro Chemotherapeutic Combinations

The in vitro potency of anti-DR5 IgM Mab A in combination with chemotherapeutic agents was evaluated on tumor cell lines and primary human hepatocytes as follows. Tumor cells (as shown in Table 4) or primary human hepatocytes (BioIVT X008001) were seeded and the next day cells were treated with serial dilutions of anti-DR5 IgM Mab A and a chemotherapeutic agent (as shown in Table 4) in combination. After 72 hours at 37° C., Cell Titer Glo reagent (Promega) was added, and cell viability was read on a luminometer.

Synergy for each tested cell line and each combination of chemotherapeutic agent tested with anti-DR5 IgM Mab A was aggregated into tabular dataframe. This dataframe was used as input to the statistical computing language R, wherein a sigmoidal dose response was fit to each single compound. Synergy, defined as the combinatorial effect of two compounds being greater than their additive effects alone, was also calculated in R. The reference model chosen for synergy scoring was Bliss Independence (BI), expressed in terms effect E on drugs A and B:

K_{A}+E_{B}−E_{A}E_{B}=E_{AB}

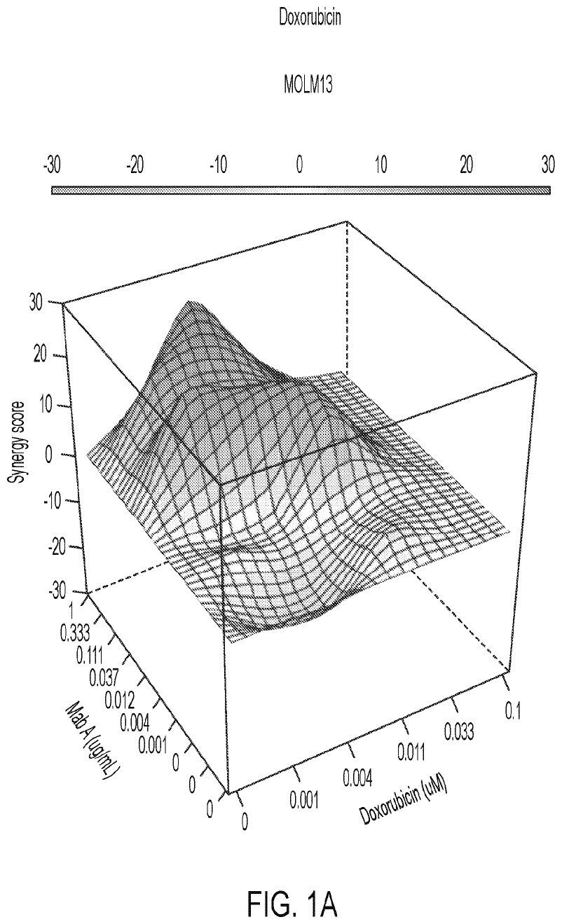

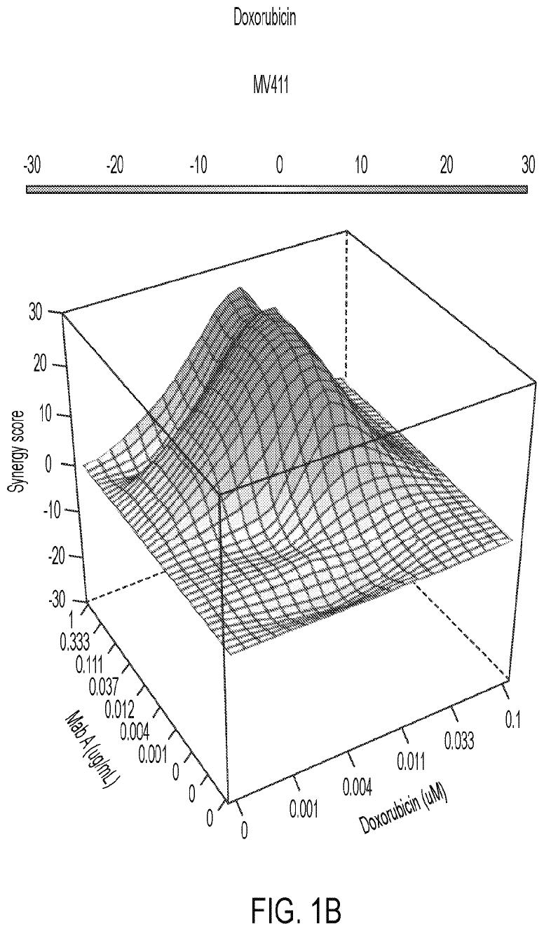

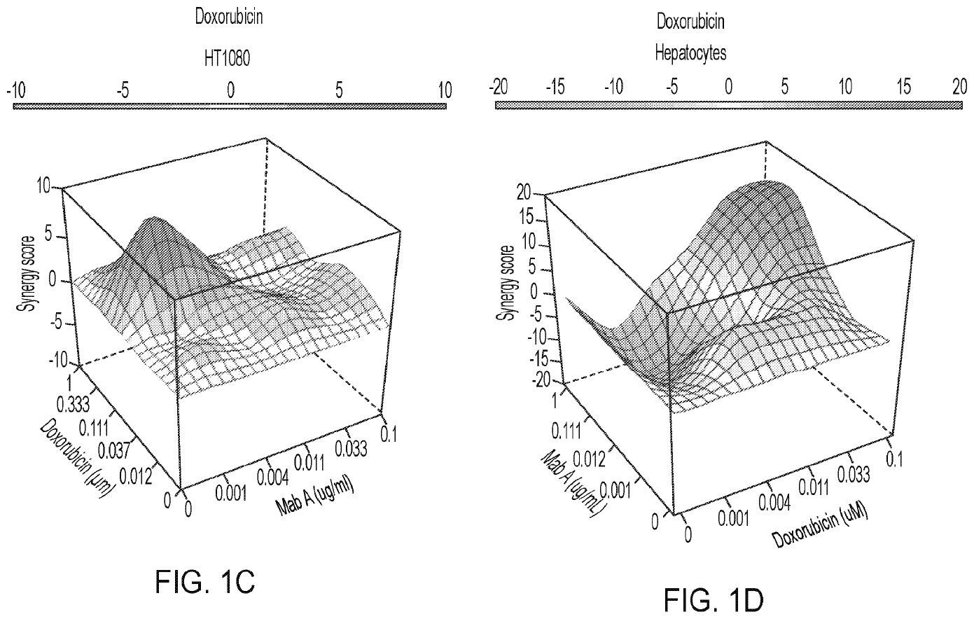

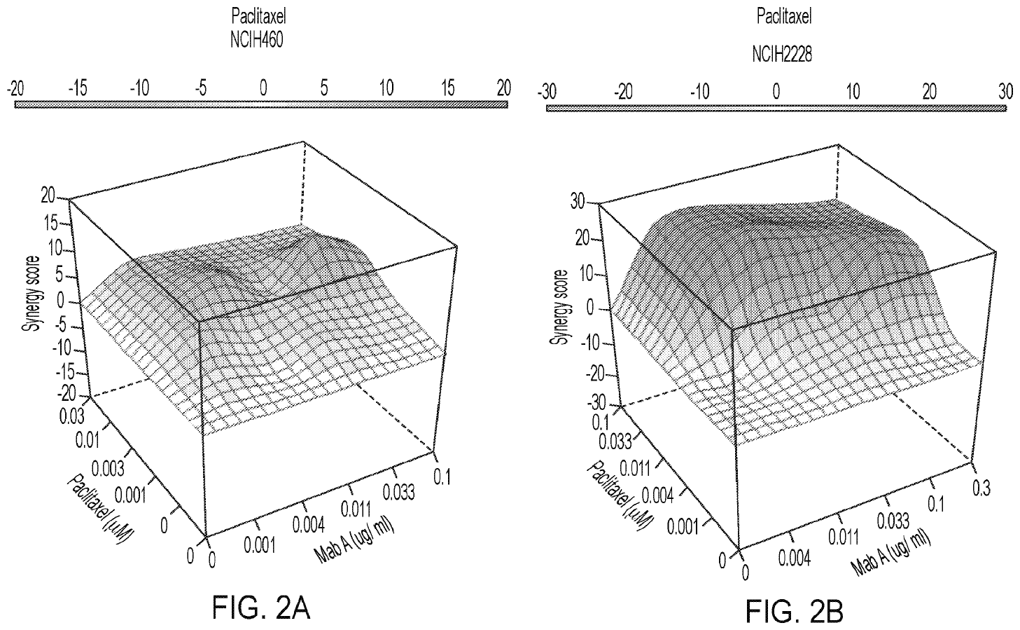

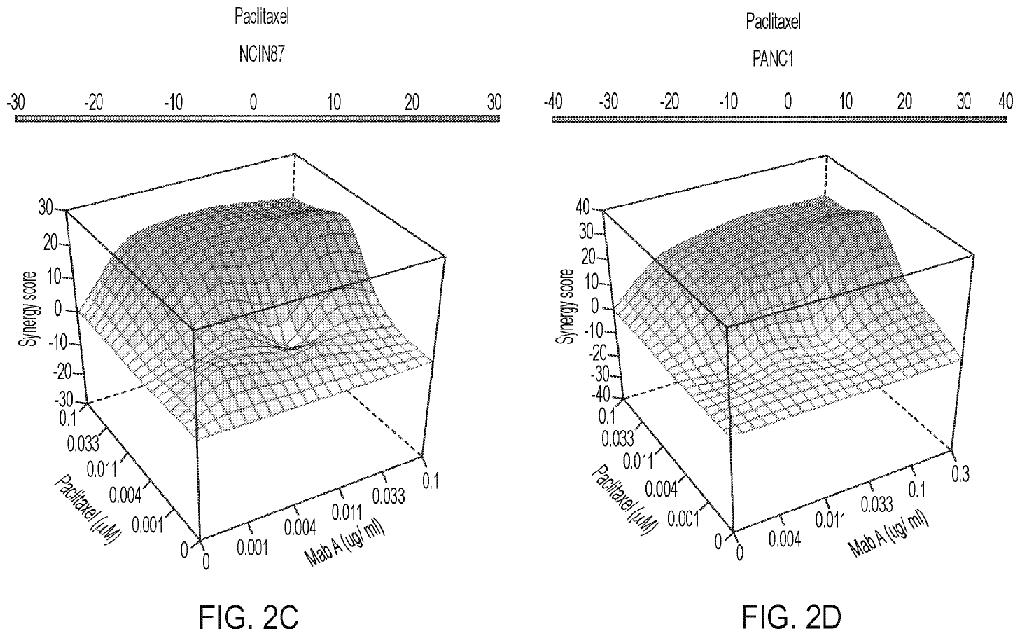

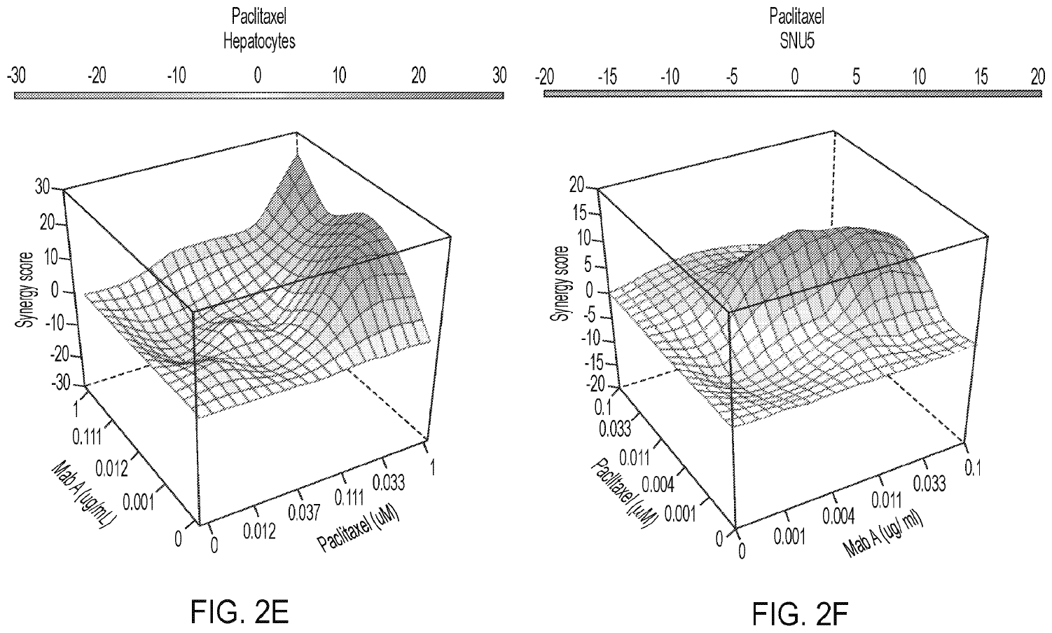

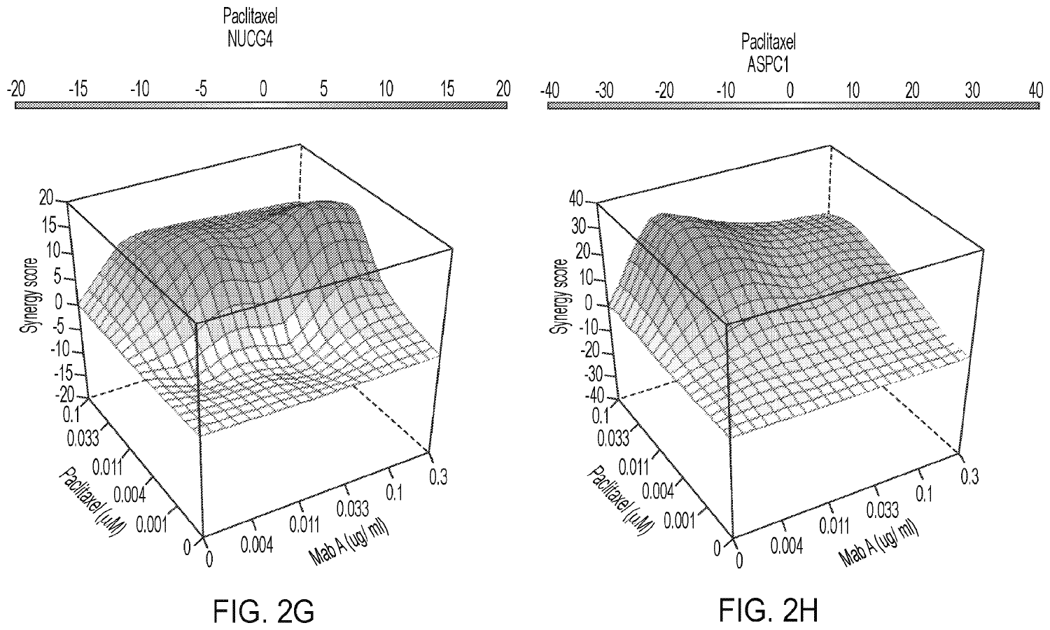

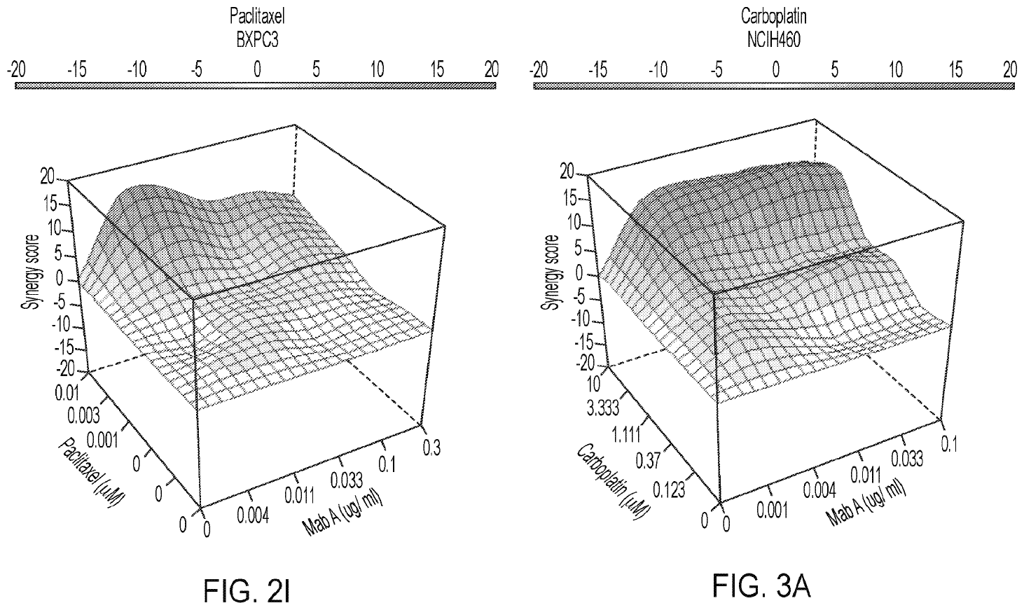

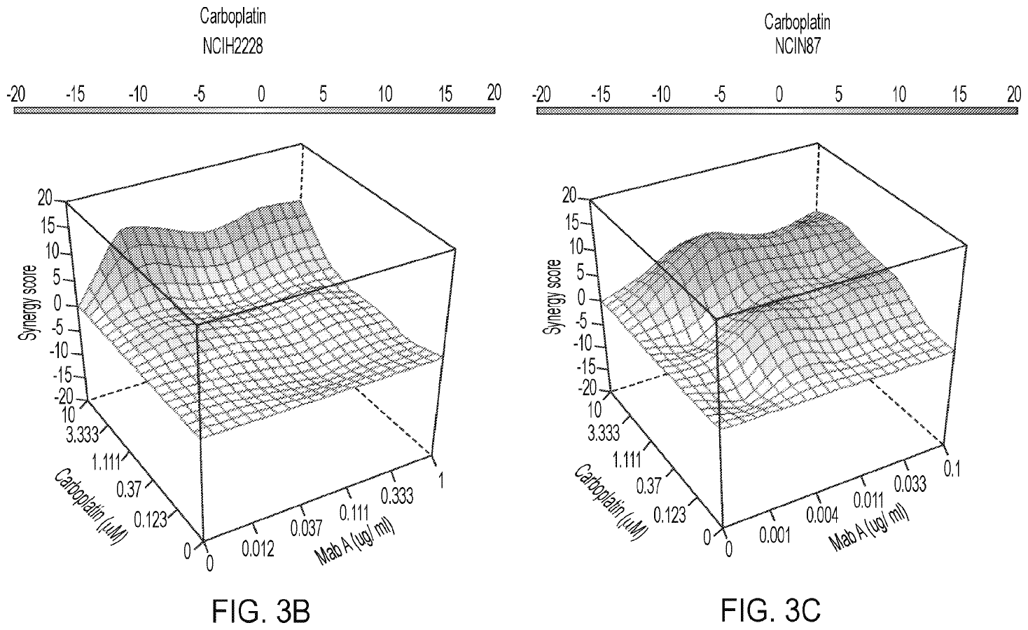

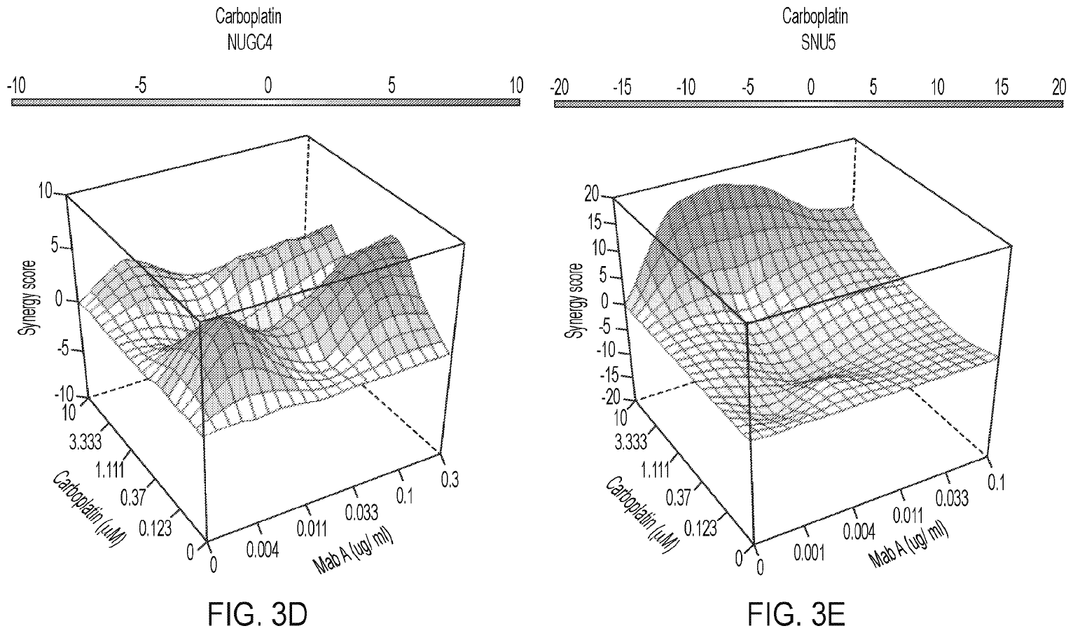

BI assumes the effect of each drug to act independently from one another. The choice to use BI is based on the separate and distinct mechanisms of action of each chemotherapeutic agent when compared to anti-DR5 IgM Mab A. Synergy scores from BI are generated on a continuum of dose combinations, with negative scores reflecting antagonism and positive scores representing synergy. These scores are visualized in 3D surface plots with valleys of antagonism and hills of synergy over the 2D dimension representing the continuum of dose combinations. The average Bliss scores are shown in Table 4. The overall max Bliss score, the Mab A and compound concentration at max Bliss. and the percent cytotoxicity of the compound, Mab A, and the combination at max Bliss for exemplary cancer cell lines or healthy hepatocytes treated with doxorubicin, paclitaxel, carboplatin, and oxaliplatin and with Mab A are shown in Table 5. Exemplary 3D surface plots for doxorubicin, paclitaxel, carboplatin, and oxaliplatin, arc shown in FIGS. 1A-1D, FIGS. 2A-21, FIGS. 3A-3E, and FIGS. 4A-4H, respectively.

TABLE 4
Chemotherapeutic Combinations

| Chemo- | | | | Aver- |
|---|---|---|---|---|
| thera- | | | | age |
| peutic | | | | Bliss |
| agent | Cell line | Tissue | Cell type | score |
| carboplatin | NCIH460 | Lung | Non-small cell | 7.2 |
| carboplatin | HCT15 | Colorectal | adenocarcinoma | 5.6 |
| carboplatin | NCIN87 | Gastric | Adenocarcinoma, | 4.5 |
| | | | tubular | |
| carboplatin | PANC1 | Pancreas | Ductal | 4.5 |
| | | | Adenocarcinoma, | |
| | | | exocrine | |
| carboplatin | UMUC3 | Bladder | Transitional Cell | 2.3 |
| | | | Carcinoma | |
| carboplatin | SNU5 | Gastric | Adenocarcinoma | 2.1 |
| carboplatin | NCIH2228 | Lung | Non-small cell | 1.8 |
| carboplatin | HT1080 | Connective | Fibrosarcoma | 1.7 |
| | | tissue | | |
| carboplatin | NUGC4 | Gastric | Adenocarcinoma | 1.2 |
| carboplatin | BXPC3 | Pancreas | adenocarcinoma | 0.63 |
| carboplatin | HT55 | Colorectal | Carcinoma | −0.39 |
| carboplatin | ASPC1 | Pancreas | Ductal adenocarcinoma | −0.63 |
| carboplatin | LOUNH91 | Lung | Squamous cell | −1 |
| | | | carcinoma | |
| carboplatin | NCIH508 | Colorectal | Caecum | −3.4 |
| | | | Adenocarcinoma | |
| carboplatin | Hepatocytes | Liver | Primary human | −1.9 |
| | | | hepatocytes | |
| doxorbicin | NCIH2228 | Lung | Non-small cell | 16 |
| doxorubicin | LOUNH91 | Lung | Squamous cell | 12 |
| | | | carcinoma | |
| doxorubicin | NCIN87 | Gastric | Adenocarcinoma, | 12 |
| | | | tubular | |
| doxorubicin | SNU5 | Gastric | Adenocarcinoma | 12 |
| doxorubicin | HCT15 | Colorectal | adenocarcinoma | 10 |
| doxorubicin | NUGC4 | Gastric | Adenocarcinoma | 8.9 |
| doxorubicin | UMUC3 | Bladder | Transitional Cell | 8.8 |
| | | | Carcinoma | |
| doxorubicin | PANC1 | Pancreas | Ductal | 7.3 |
| | | | Adenocarcinoma, | |
| | | | exocrine | |
| doxorubicin | MV411 | Blood | Acute myelogenous | 7.1 |
| | | (Leukemia) | leukemia | |
| doxorubicin | MOLM13 | Blood | Acute myelogenous | 5.2 |
| | | (Leukemia) | leukemia | |
| doxorubicin | NCIH460 | Lung | Non-small cell | 4 |
| doxorubicin | HT55 | Colorectal | Carcinoma | 1.6 |
| doxorubicin | BXPC3 | Pancreas | adenocarcinoma | 1.5 |
| doxorubicin | HT1080 | Connective | Fibrosarcoma | 1.5 |
| | | tissue | | |
| doxorubicin | NCIH508 | Colorectal | Caecum | 1.3 |
| | | | Adenocarcinoma | |
| doxorubicin | Hepatocytes | Liver | Primary human | 0.94 |
| | | | hepatocytes | |
| doxorubicin | ASPC1 | Pancreas | Ductal adenocarcinoma | −0.63 |
| etoposide | NCIN87 | Gastric | Adenocarcinoma, | 17 |
| | | | tubular | |
| etoposide | LOUNH91 | Lung | Squamous cell | 16 |
| | | | carcinoma | |
| etoposide | HCT15 | Colorectal | adenocarcinoma | 15 |
| etoposide | NCIH2228 | Lung | Non-small cell | 14 |
| etoposide | PANC1 | Pancreas | Ductal | 10 |
| | | | Adenocarcinoma, | |
| | | | exocrine | |
| etoposide | UMUC3 | Bladder | Transitional Cell | 8.2 |
| | | | Carcinoma | |
| etoposide | ASPC1 | Pancreas | Ductal adenocarcinoma | 5.2 |
| etoposide | HT55 | Colorectal | Carcinoma | 5.1 |
| etoposide | HT1080 | Connective | Fibrosarcoma | 3.6 |
| | | tissue | | |
| etoposide | NCIH460 | Lung | Non-small cell | 3.6 |
| etoposide | SNU5 | Gastric | Adenocarcinoma | 3.6 |
| etoposide | BXPC3 | Pancreas | adenocarcinoma | 2.2 |
| etoposide | NCIH508 | Colorectal | Caecum | −0.99 |
| | | | Adenocarcinoma | |
| etoposide | NUGC4 | Gastric | Adenocarcinoma | −2.3 |
| oxaliplatin | PANC1 | Pancreas | Ductal | 8.9 |
| | | | Adenocarcinoma, | |
| | | | exocrine | |
| oxaliplatin | HCT15 | Colorectal | adenocarcinoma | 8.1 |
| oxaliplatin | NCIN87 | Gastric | Adenocarcinoma, | 8.1 |
| | | | tubular | |
| oxaliplatin | SNU5 | Gastric | Adenocarcinoma | 5.8 |
| oxaliplatin | HT55 | Colorectal | Carcinoma | 4.7 |
| oxaliplatin | UMUC3 | Bladder | Transitional Cell | 4.1 |
| | | | Carcinoma | |
| oxaliplatin | NCIH460 | Lung | Non-small ceil | 3.3 |
| oxaliplatin | NCIH2228 | Lung | Non-small cell | 3.2 |
| oxaliplatin | Hepatocytes | Liver | Primary human | 3.2 |
| | | | hepatocytes | |
| oxaliplatin | LOUNH91 | Lung | Squamous cell | 1.5 |
| | | | carcinoma | |
| oxaliplatin | NUGC4 | Gastric | Adenocarcino ma | 0.88 |
| oxaliplatin | NCIH508 | Colorectal | Caecum | 0.75 |
| | | | Adenocarcinoma | |
| oxaliplatin | BXPC3 | Pancreas | adenocarcinoma | 0.58 |
| oxaliplatin | HT1080 | Connective | Fibrosarcoma | −1 |
| | | tissue | | |
| oxaliplatin | ASPC1 | Pancreas | Ductal adenocarcinoma | −1.5 |
| paclitaxel | NCIH2228 | Lung | Non-small cell | 15 |
| paclitaxel | PANC1 | Pancreas | Ductal | 15 |
| | | | Adenocarcinoma, | |
| | | | exocrine | |
| paclitaxel | ASPC1 | Pancreas | Ductal adenocarcinoma | 14 |
| paclitaxel | NCIN87 | Gastric | Adenocarcinoma, | 13 |
| | | | tubular | |
| paclitaxel | LOUNH91 | Lung | Squamous cell | 9.7 |
| | | | carcinoma | |
| paclitaxel | NCIH508 | Colorectal | Caecum | 8.4 |
| | | | Adenocarcinoma | |
| paclitaxel | HCT15 | Colorectal | adenocarcinoma | 7.6 |
| paclitaxel | NUGC4 | Gastric | Adenocarcinoma | 7 |
| paclitaxel | UMUC3 | Bladder | Transitional Cell | 6.3 |
| | | | Carcinoma | |
| paclitaxel | HT55 | Colorectal | Carcinoma | 6.2 |
| paclitaxel | Hepatocytes | Liver | Primary human | 5.3 |
| | | | hepatocytes | |
| paclitaxel | NCTH460 | Lung | Non-small cell | 4.7 |
| paclitaxel | BXPC3 | Pancreas | adenocarcinoma | 3.8 |
| paclitaxel | SNU5 | Gastric | Adenocarcinoma | 2.8 |
| paclitaxel | HT1080 | Connective | Fibrosarcoma | 2.7 |
| | | tissue | | |

TABLE 5
Max Bliss Comparisons

| | | | | | | % | % | % |
|---|---|---|---|---|---|---|---|---|
| | | | | Compound | Mab A | Cytotox. of | Cytotox. | Cytotox. |
| | | | | Conc. at | Conc. at | Compound | of Mab A | of Combo |
| Combination | Avg. | Avg. % | Max | Max Bliss | Max Bliss | at Max | at Max | at Max |
| Assay | Bliss | Cytotox. | Bliss | (μM) | (μg/mL) | Bliss | Bliss | Bliss |
| Oxaliplatin × | 8.1 | 63.0 | 16.8 | 3.3 | 0.0012 | 44.9 | 23.2 | 74.5 |
| Mab A in | | | | | | | | |
| HCT-15 | | | | | | | | |
| Oxaliplatin × | 3.2 | 15.4 | 9.98 | 0.016 | 0.0014 | 4.80 | −0.514 | 14.29 |
| Mab A in | | | | | | | | |
| Hepatocytes | | | | | | | | |
| Carboplatin × | 1.8 | 38.1 | 12.4 | 10 | 0.012 | 17.7 | 22.9 | 49.0 |
| Mab A in | | | | | | | | |
| NCI-H2228 | | | | | | | | |
| Carboplatin × | −1.9 | 6.43 | 5.00 | 0.4 | 0.012 | −0.176 | 3.38 | 6.69 |
| Mab A in | | | | | | | | |
| Hepatocytes | | | | | | | | |
| Paclitaxel × | 14.7 | 62.0 | 25.6 | 0.011 | 0.011 | 52.0 | 17.3 | 85.9 |
| Mab A in | | | | | | | | |
| NCI-H2228 | | | | | | | | |
| Paclitaxel × | 8.3 | 7.34 | 38.2 | 0.0037 | 1 | −33.2 | 6.73 | 14.0 |
| Mab A in | | | | | | | | |
| Hepatocytes | | | | | | | | |
| Doxorubicin × | 7.1 | 58.4 | 27.4 | 0.011 | 0.037 | 50.3 | 31.8 | 93.6 |
| Mab A in | | | | | | | | |
| MV-411 | | | | | | | | |
| Doxorubicin × | 0.94 | 12.3 | 37.2 | 1 | 0.1111 | 33.1 | 4.87 | 73.65 |
| Mab A in | | | | | | | | |
| Hepatocytes | | | | | | | | |

### Example 2: In Vivo Radiation Combination

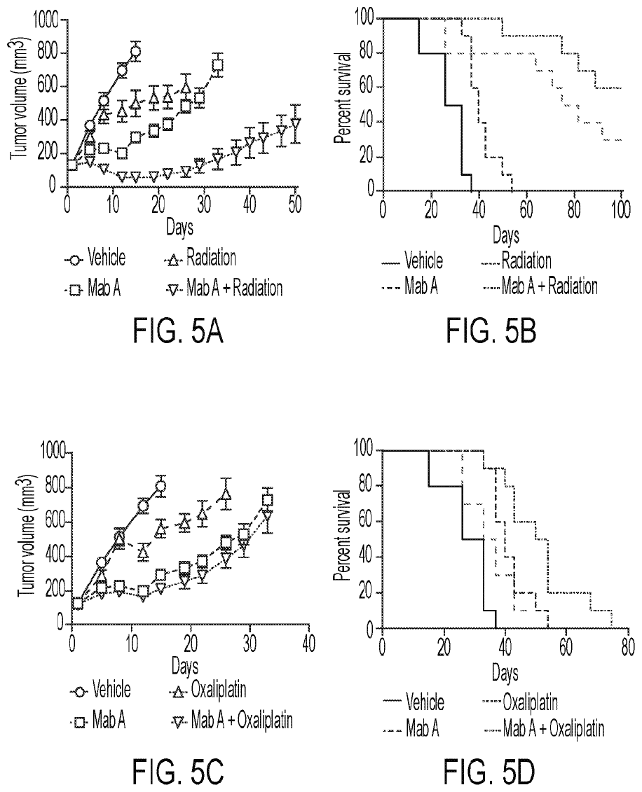

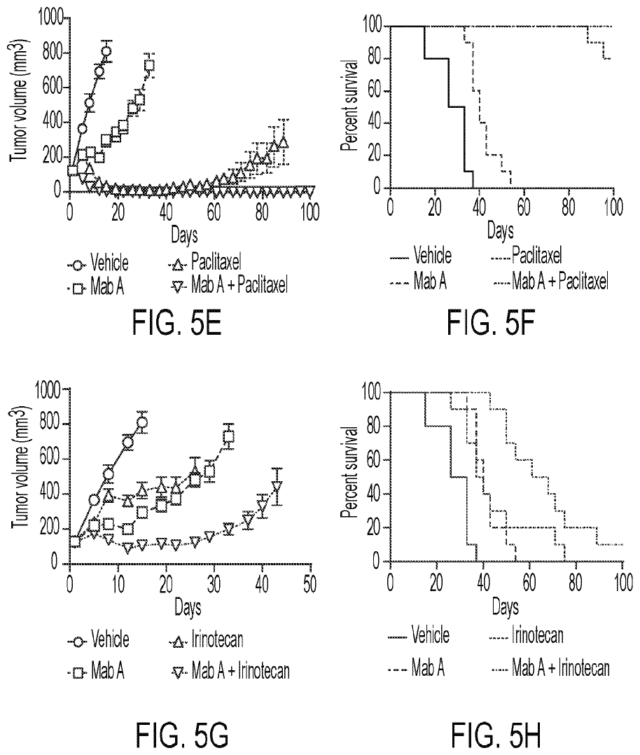

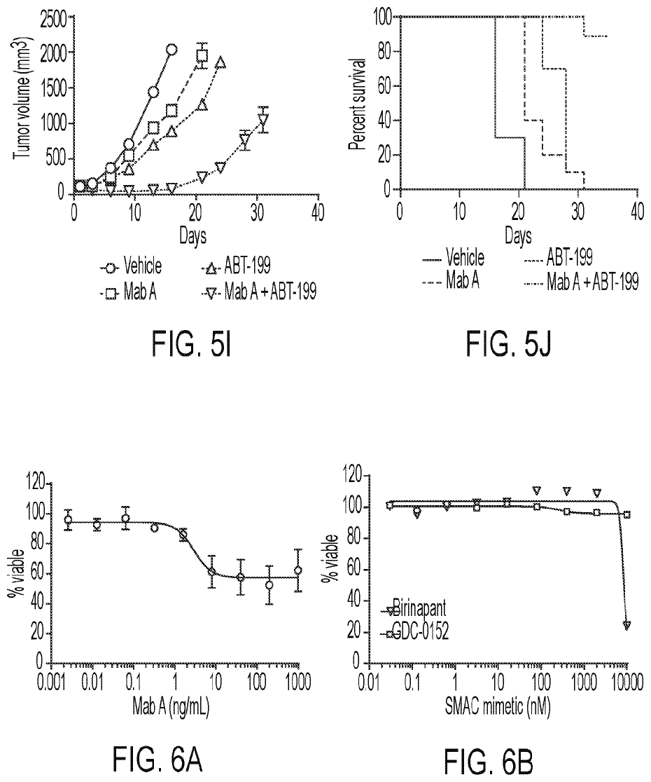

2×10⁶ Colo205 tumor cells (colorectal cancer cells originally isolated from a colon adenocarcinoma tumor) were implanted subcutaneously in the flanks of female NCr nude mice. When mean tumor volume reached 100-150 mm³, mice were dosed with either vehicle i.v. every other day for a total of 7 doses, 5 mg/kg of anti-DR5 IgM Mab A i.v. every other day for a total of 7 doses, 2 Gy/animal of targeted radiation for 5 days on followed by 2 days off followed by 5 days on, or a combination of the anti-DR5 IgM Mab A and radiation treatment regimens. Tumor volume (n=10 animals/group) is shown in FIG. 5A and overall survival is shown in FIG. 5B. On day 15 (the last day that all control animals were on study), the combination therapy with anti-DR5 IgM Mab A and targeted radiation significantly reduced tumor volume compared to targeted radiation alone. The combined treatment did not significantly extend overall survival compared to radiation alone.

### Example 3: In Vivo Oxaliplatin Combination

2×10⁶ Colo205 tumor cells were implanted subcutaneously in the flanks of female NCr nude mice. When mean tumor volume reached 100-150 mm³, mice were dosed with either vehicle i.v. every other day for a total of 7 doses, 5 mg/kg of anti-DR5 IgM Mab A i.v. every other day for a total of 7 doses, 8 mg/kg of oxaliplatin i.p. weekly for 3 weeks, or a combination of the anti-DR5 IgM Mab A and oxaliplatin treatment regimens. Tumor volume (n=10 animals/group) is shown in FIG. 5C and overall survival is shown in FIG. 5D. On day 15 (the last day that all control animals were on study), the combination therapy with anti-DR5 IgM Mab A and oxaliplatin significantly reduced tumor volume compared to oxaliplatin alone. The combined treatment also significantly extended overall survival compared to oxaliplatin alone.

### Example 4: In Vivo Paclitaxel Combination

2×10⁶ Colo205 tumor cells were implanted subcutaneously in the flanks of female NCr nude mice. When mean tumor volume reached 100-150 mm³, mice were dosed with either vehicle i.v. every other day for a total of 7 doses, 5 mg/kg of anti-DR5 IgM Mab A i.v. every other day for a total of 7 doses, 25 mg/kg of paclitaxel i.v. every other day for a total of 5 doses, or a combination of the anti-DR5 IgM Mab A and paclitaxel treatment regimens. Tumor volume (n=10 animals/group) is shown in FIG. 5E and overall survival is shown in FIG. 5F. On day 15 (the last day that all control animals were on study), the combination therapy with anti-DR5 IgM Mab A and paclitaxel did not significantly reduce tumor volume relative to the single agent paclitaxel treated group. The combined treatment also did not significantly extend overall survival compared to paclitaxel alone. However, when the study was terminated on day 100, 2 out of 10 animals in the paclitaxel treated group had no visible tumors, whereas 8 out of 10 animals in the combination arm were tumor-free.

### Example 5: In Vivo Irinotecan Combination

2×10⁶ Colo205 tumor cells were implanted subcutaneously in the flanks of female NCr nude mice. When mean tumor volume reached 100-150 mm³, mice were dosed with either vehicle i.v. every other day for a total of 7 doses, 5 mg/kg of anti-DR5 IgM Mab A i.v. every other day for a total of 7 doses, 100 mg/kg of irinotecan i.p. weekly for 3 weeks, or a combination of the anti-DR5 IgM Mab A and irinotecan treatment regimens. Tumor volume (n=10 animals/group) is shown in FIG. 5G and overall survival is shown in FIG. 5H. On day 15 (the last day that all control animals were on study), the combination therapy with anti-DR5 IgM Mab A and irinotecan significantly reduced tumor volume compared to irinotecan alone. The combined treatment also significantly extended overall survival compared to irinotecan alone.

### Example 6: In Vivo ABT-199 Combination

1×10⁷ DOHH-2 tumor cells were implanted subcutaneously in the flanks of female CB.17 SCID mice. When mean tumor volume reached 100-150 mm³, mice were dosed with either vehicle i.v. every other day for a total of 11 doses, 5 mg/kg of anti-DR5 IgM Mab A i.v. every other day for a total of 11 doses, 100 mg/kg of ABT-199 (Venetoclax) p.o. daily for 21 days, or a combination of the anti-DR5 IgM Mab A and ABT-199 treatment regimens. Tumor volume (n=10 animals/group) is shown in FIG. 5I and overall survival is shown in FIG. 5J. On day 16 (the last day that all control animals were on study), the combination therapy with anti-DR5 IgM Mab A and ABT-199 resulted in reduced tumor volume relative to any of the treatments alone, although the difference between the combined treatment and ABT-199 alone did not reach statistical significance. The combined treatment significantly extended overall survival compared to any of the treatments alone.

### Example 7: In Vitro SMAC Mimetic Combinations

The in vitro potency of anti-DR5 IgM Mab A in combination with SMAC mimetic birinapant or GDC-0152 was evaluated on MDA-MB-231 tumor cells and primary human hepatocytes as follows. Tumor cells or primary human hepatocytes (BioIVT X008001) were seeded and the next day cells were treated with serial dilutions of anti-DR5 IgM Mab A and a pro-apoptotic agent/SMAC mimetic alone or in combination. After 72 hours at 37° C., Cell Titer Glo reagent (Promega) was added, and cell viability was read on a luminometer.

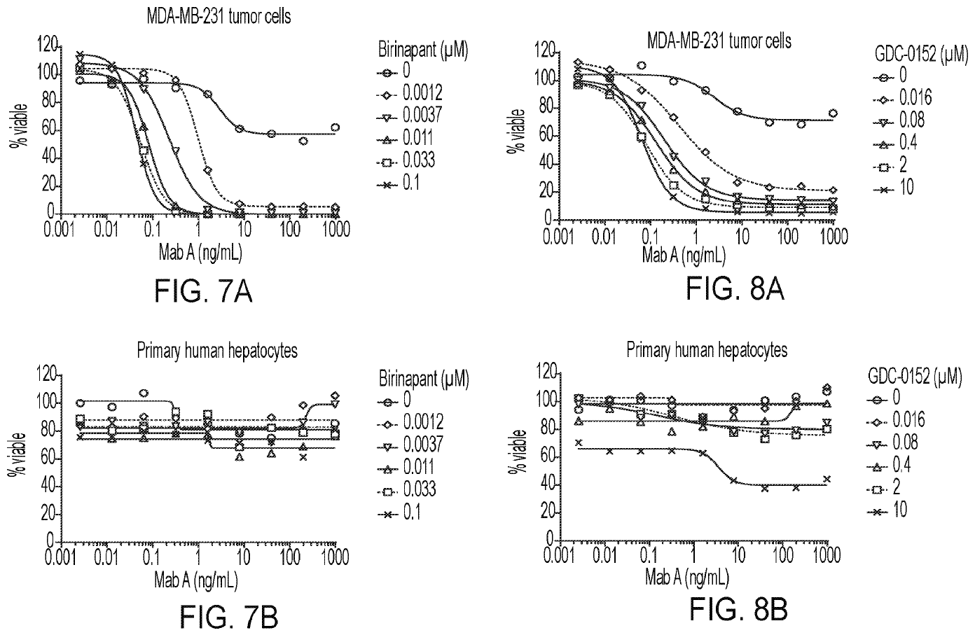

Cell viability curves for single agent Mab A or SMAC mimetics are shown in FIGS. 6A and 6B, respectively. Single agent Mab A shows partial cytotoxicity on MDA-MB-231 cells and single agent birinapant or GDC-0152 show little to no cytotoxicity. Cell viability curves for combinations of Mab A and birinapant on MDA-MB-231 tumor cells or primary human hepatocytes are shown in FIGS. 7A and 7B, respectively. Cell viability curves for combinations of Mab A and GDC-0152 on MDA-MB-231 tumor cells or primary human hepatocytes are shown in FIGS. 8A and 8B, respectively. IC₅₀ values for birinapant and GDC-0152 are shown in Tables 6 and 7, respectively.

TABLE 6
IC₅₀ Values for Birinapant

| Birinapant Concentration (μM) | IC₅₀ |
|---|---|
| 0 | 2.9 |
| 0.0012 | 0.98 |
| 0.0037 | 0.23 |
| 0.011 | 0.082 |
| 0.033 | 0.054 |
| 0.1 | 0.044 |

TABLE 7
IC₅₀ Values for GDC-0152

| GDC-0152 Concentration (μM) | IC₅₀ |
|---|---|
| 0 | 2.5 |
| 0.0016 | 0.42 |
| 0.08 | 0.20 |
| 0.4 | 0.13 |
| 2 | 0.081 |
| 10 | 0.065 |

Synergy for each tested cell line and each combination of SMAC mimetic tested with anti-DR5 IgM Mab A was aggregated into tabular dataframe. This dataframe was used as input to the statistical computing language R, wherein a sigmoidal dose response was fit to each single compound. Synergy, defined as the combinatorial effect of two compounds being greater than their additive effects alone, was also calculated in R. The reference model chosen for synergy scoring was Bliss Independence (BI), expressed in terms effect E on drugs A and B:

E_{A}+E_{B}−E_{A}E_{B}=E_{AB}

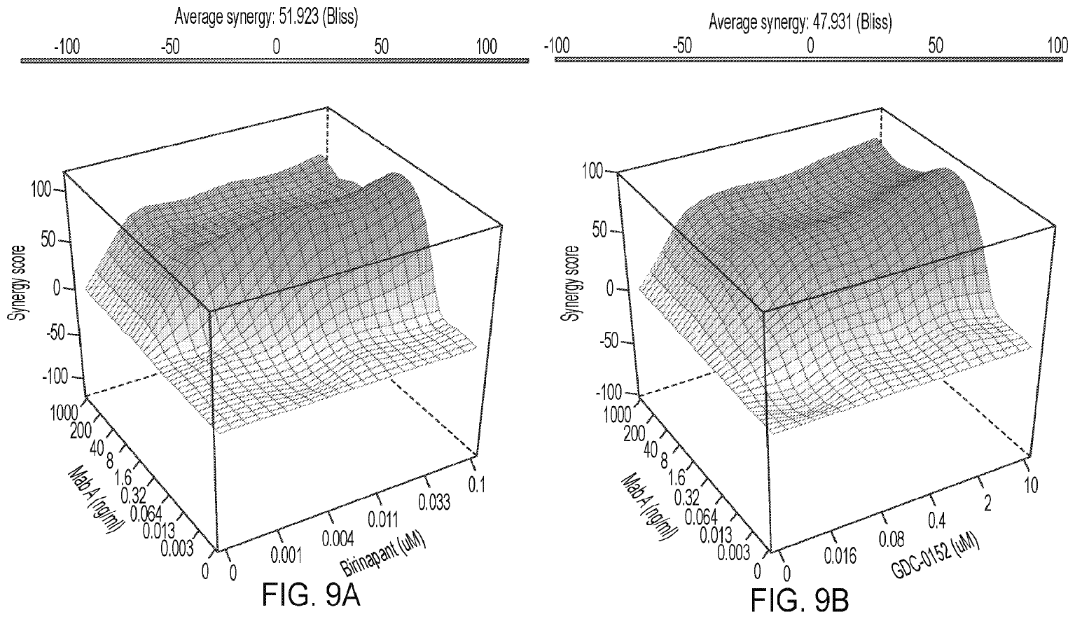

BI assumes the effect of each drug to act independently from one another. The choice to use BI is based on the separate and distinct mechanisms of action of each chemotherapeutic agent when compared to anti-DR5 IgM Mab A. Synergy scores from BI are generated on a continuum of dose combinations, with negative scores reflecting antagonism and positive scores representing synergy. These scores are visualized in 3D surface plots with valleys of antagonism and hills of synergy over the 2D dimension representing the continuum of dose combinations. 3D surface plots for birinapant and GDC-0152 on MDA-MB-231 cells are shown in FIGS. 9A and 9B, respectively. The synergy scoring was also completed using the Loewe model. Similar levels of synergy were found (data not shown).

The Mab A and GDC-0152 or birinapant combinations result in strong synergistic cytotoxicity on MDA-MB-231 cells. These combinations do not result in substantial cytotoxicity in primary human hepatocytes.

### Example 8: In Vitro SMAC Mimetic Combinations on DR5 Agonist-Resistant Tumor Cells

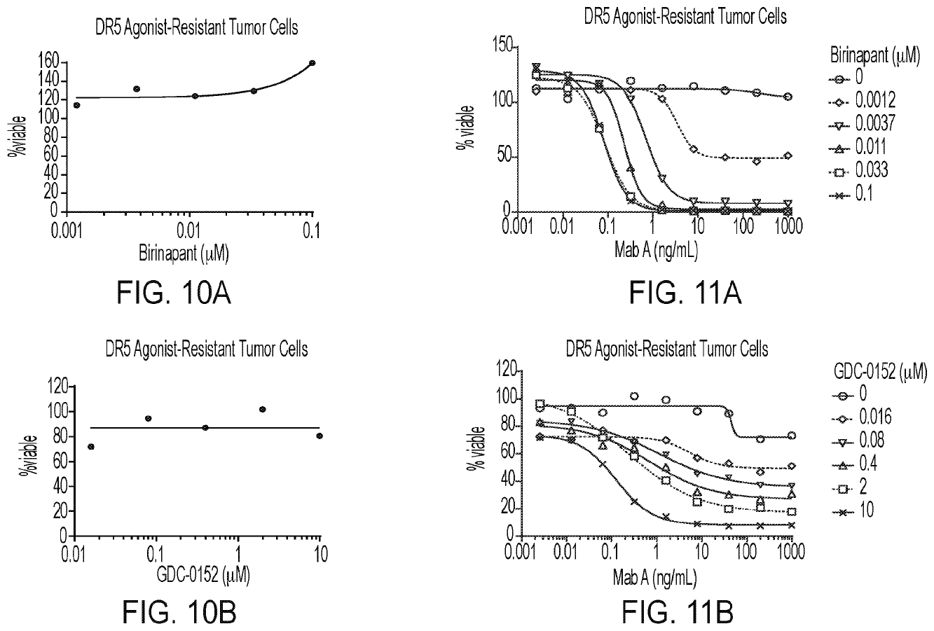

Acquired DR5 agonist-resistant MDA-MB-231 cells were generated by culturing MDA-MB-231 cells in the presence of 0.1 μg/mL of anti-DR5 IgM Mab B to eliminate sensitive cells and enrich the DR5 agonist-resistant cell population. The in vitro potency of anti-DR5 IgM Mab A in combination with SMAC mimetic birinapant or GDC-0152 was evaluated on the DR5 agonist-resistant tumor cells according to the method described in Example 8. Cell viability curves for single agent birinapant or GDC-0152 are shown in FIGS. 10A and 10B, respectively. Single agent birinapant or GDC-0152 show little to no cytotoxicity. Cell viability curves for combinations of Mab A and birinapant or GDC-0152 are shown in FIGS. 11A and 11B, respectively. IC₅₀ values for birinapant and GDC-0152 are shown in Tables 8 and 9. respectively. Mab A and SMAC mimetic combination results in strong synergistic cytotoxicity on MDA-MB-231 cells with acquired resistance to a DR5 agonist.

TABLE 8
IC₅₀ Values for Birinapant

| Birinapant Concentration (μM) | IC₅₀ |
|---|---|
| 0 | |
| 0.0012 | 3.5 |
| 0.0037 | 0.70 |
| 0.011 | 0.23 |
| 0.033 | 0.086 |
| 0.1 | 0.081 |

### Example 9: In Vitro Chemotherapeutic Combinations

TABLE 9
IC₅₀ Values for GDC-0152

| GDC-0152 Concentration (μM) | IC₅₀ |
|---|---|
| 0 | ~45 |
| 0.0016 | 5.3 |
| 0.08 | 1.1 |
| 0.4 | 0.70 |
| 2 | 0.24 |
| 10 | 0.13 |

The in vitro potency of anti-DR5 IgM Mab A in combination with BTK inhibitor ibrutinib, with PI3Kδ inhibitor idelalisib, with Mcl-1 inhibitor MIK665, or with vincristine was evaluated on tumor cell lines and primary human hepatocytes as follows. Tumor cells or primary human hepatocytes (BioIVT X008001) were seeded and the next day cells were treated with serial dilutions of anti-DR5 IgM Mab A and a chemotherapeutic/targeted agent alone or in combination. After 72 hours at 37° C., Cell Titer Glo reagent (Promega) was added, and cell viability was read on a luminometer.

Synergy for each tested cell line and each combination of chemotherapeutic/targeted agent tested with anti-DR5 IgM Mab A was aggregated into tabular dataframe. This dataframe was used as input to the statistical computing language R, wherein a sigmoidal dose response was fit to each single compound. Synergy, defined as the combinatorial effect of two compounds being greater than their additive effects alone, was also calculated in R. The reference model chosen for synergy scoring was Bliss Independence (BI), expressed in terms effect E on drugs A and B:

E_{A}+E_{B}−E_{A}E_{B}=E_{AB}

BI assumes the effect of each drug to act independently from one another. The choice to use BI is based on the separate and distinct mechanisms of action of each chemotherapeutic agent when compared to anti-DR5 IgM Mab A. Synergy scores from BI are generated on a continuum of dose combinations, with negative scores reflecting antagonism, and positive scores representing synergy. These scores are visualized in 3D surface plots with valleys of antagonism and hills of synergy over the 2D dimension representing the continuum of dose combinations. The synergy scoring was also completed using the Loewe model. Similar levels of synergy were found (data not shown).

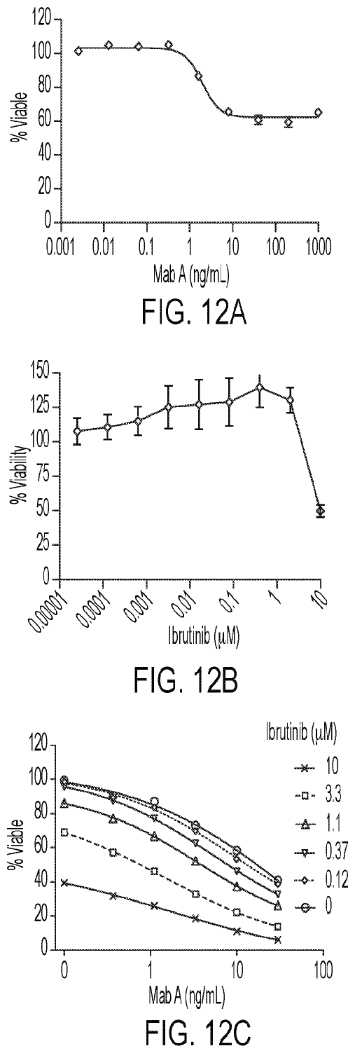

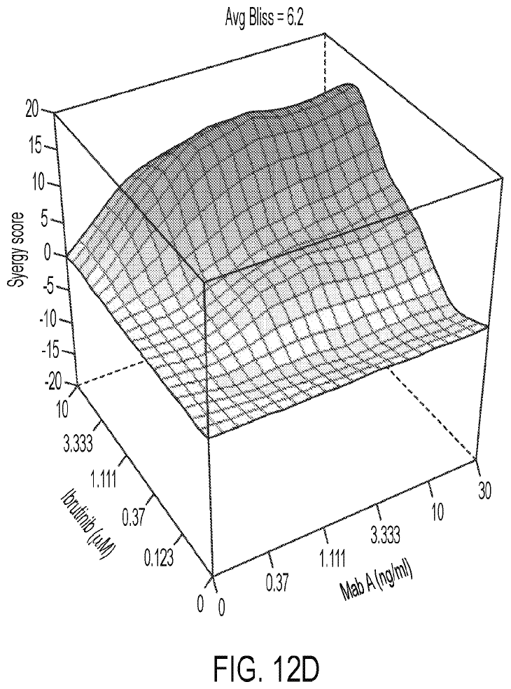

Cell viability curves for single agent Mab A or ibrutinib on U-937 cells are shown in FIGS. 12A and 12B, respectively. Single agent Mab A shows partial cytotoxicity on U-937 cells and single agent ibrutinib shows little to no cytotoxicity. Cell viability curves for combinations of Mab A and ibrutinib on U-937 tumor cells are shown in FIG. 12C. Synergy score 3D surface plots for Mab A and ibrutinib on U-937 cells are shown in FIG. 12D. The Mab A and ibrutinib combination results in weak synergistic cytotoxicity on U-937 cells.

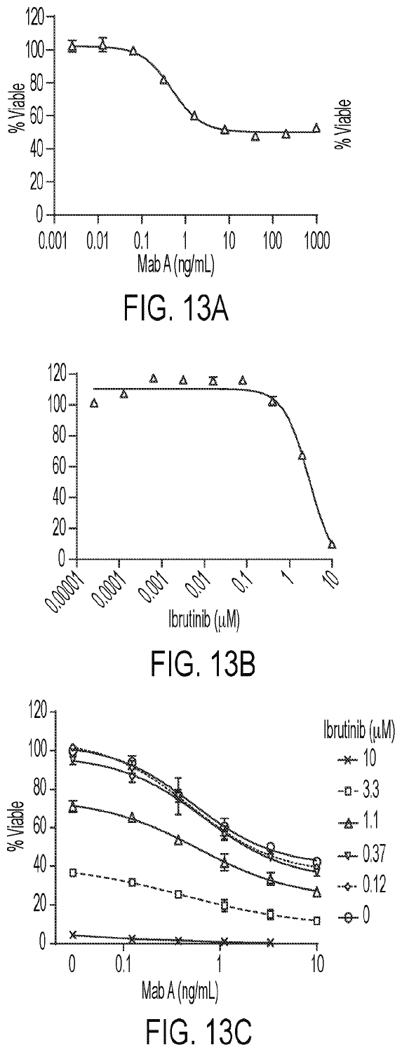

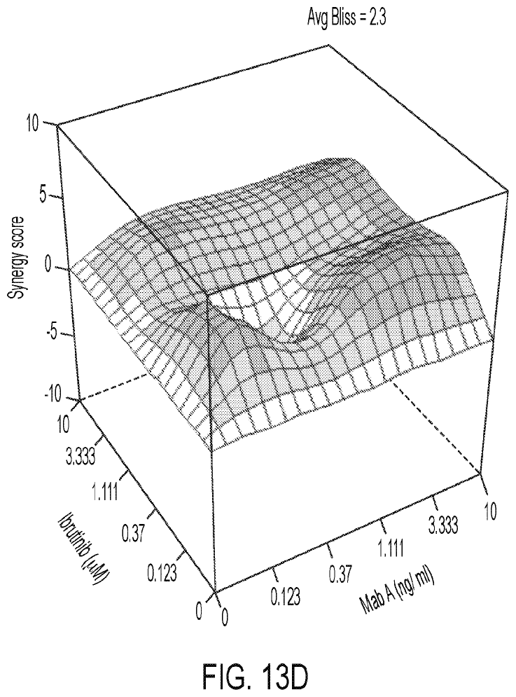

Cell viability curves for single agent Mab A or ibrutinib on OCI-LY7 cells are shown in FIGS. 13A and 13B, respectively. Single agent Mab A shows partial cytotoxicity on OCI-LY7 cells and single agent ibrutinib shows cytotoxicity only at the highest concentrations tested. Cell viability curves for combinations of Mab A and ibrutinib on OCI-LY7 tumor cells are shown in FIG. 13C. Synergy score 3D surface plots for Mab A and ibrutinib on OCI-LY7 cells are shown in FIG. 13D. The Mab A and ibrutinib combination results in neither synergistic nor antagonistic cytotoxicity on OCI-LY7 cells.

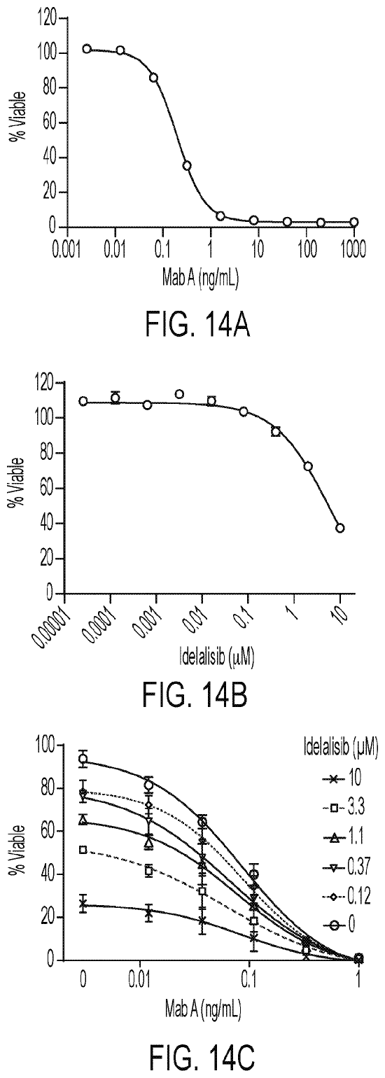

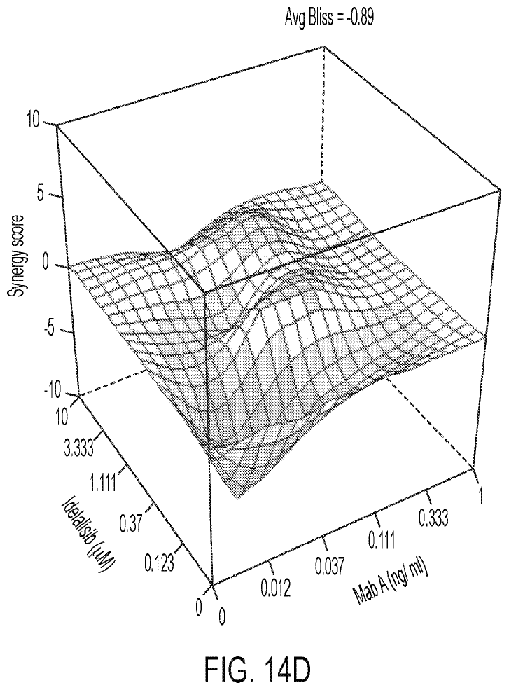

Cell viability curves for single agent Mab A or idelalisib on DOHH-2 cells are shown in FIGS. 14A and 14B, respectively. Single agent Mab A shows complete cytotoxicity on DOHH-2 cells and single agent idelalisib shows cytotoxicity only at the highest concentrations tested. Cell viability curves for combinations of Mab A and idelalisib on DOHH-2 tumor cells are shown in FIG. 14C. Synergy score 3D surface plots for Mab A and idelalisib on DOHH-2 cells are shown in FIG. 14D. The Mab A and idelalisib combination results in neither synergistic nor antagonistic cytotoxicity on DOHH-2 cells.

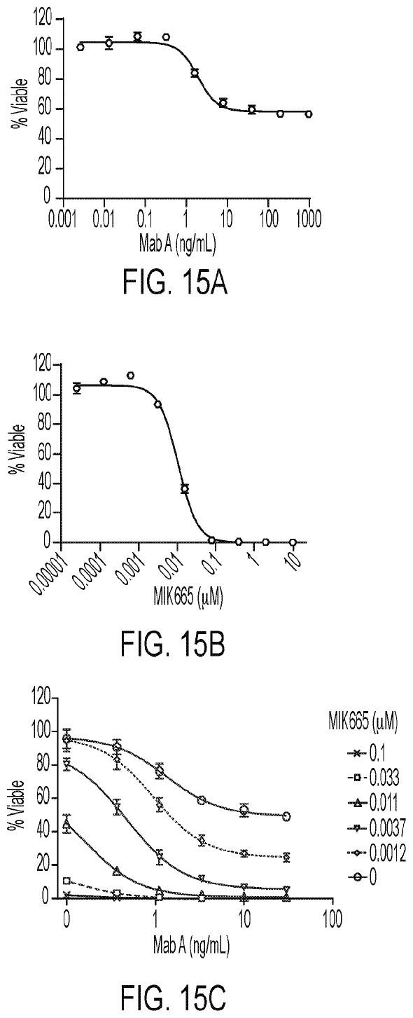

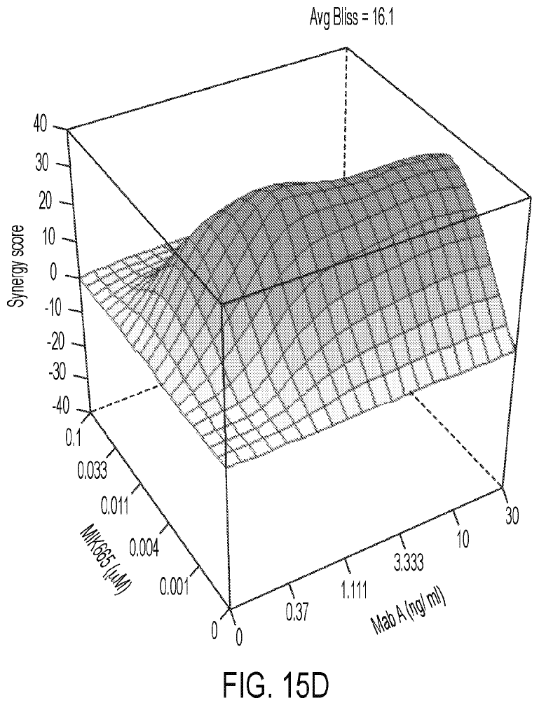

Cell viability curves for single agent Mab A or MIK665 on WSU-DLCL2 cells are shown in FIGS. 15A and 15B, respectively. Single agent Mab A shows partial cytotoxicity on WSU-DLCL2 cells and single agent MIK665 shows complete cytotoxicity. Cell viability curves for combinations of Mab A and MIK665 on WSU-DLCL2 tumor cells are shown in FIG. 15C. Synergy score 3D surface plots for Mab A and MIK665 on WSU-DLCL2 cells are shown in FIG. 15D. The Mab A and MIK665 combination results in synergistic cytotoxicity on WSU-DLCL2 cells.

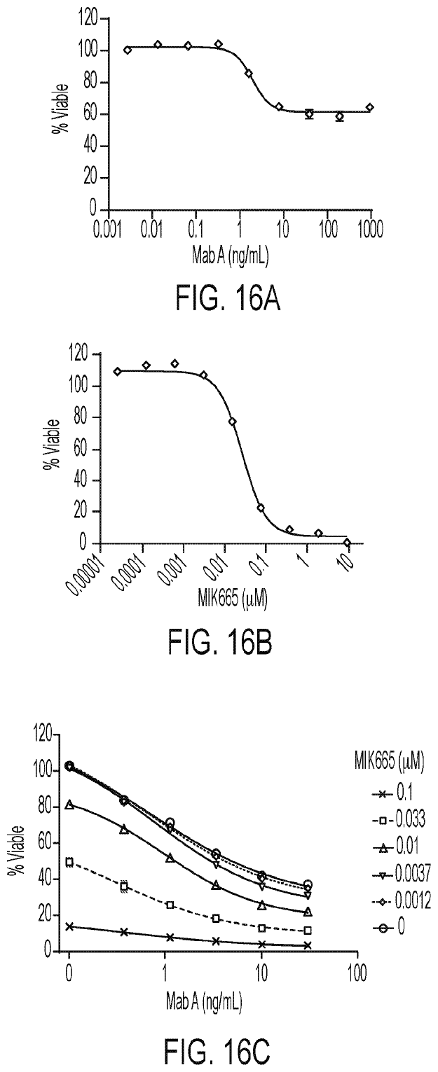

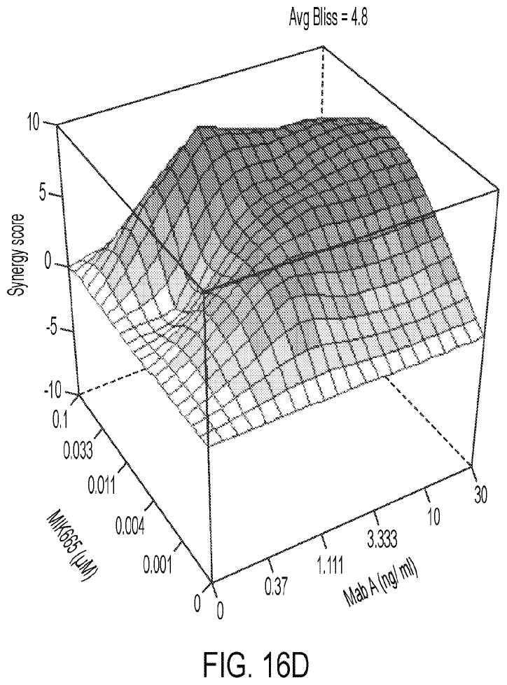

Cell viability curves for single agent Mab A or MIK665 on U-937 cells are shown in FIGS. 16A and 16B, respectively. Single agent Mab A shows partial cytotoxicity on U-937 cells and single agent MIK665 shows complete cytotoxicity. Cell viability curves for combinations of Mab A and MIK665 on U-937 tumor cells are shown in FIG. 16C. Synergy score 3D surface plots for Mab A and MIK665 on U-937 cells are shown in FIG. 16D. The Mab A and MIK665 combination results in weak synergistic cytotoxicity on U-937 cells.

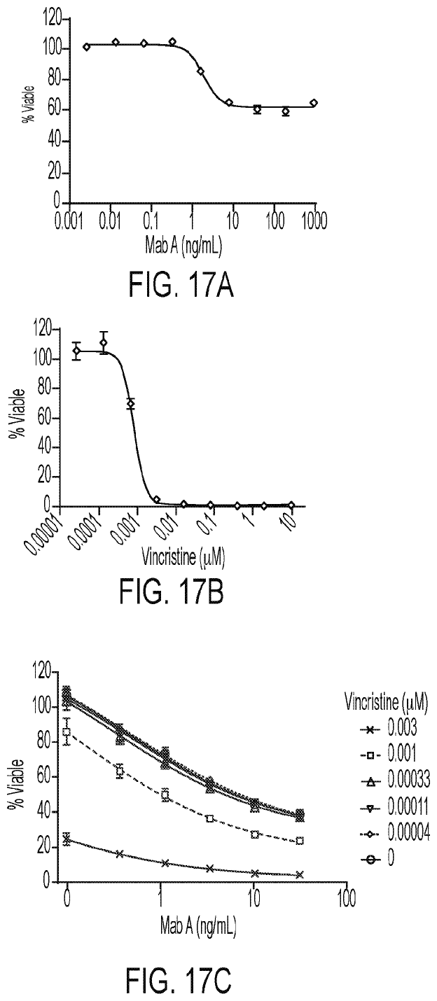

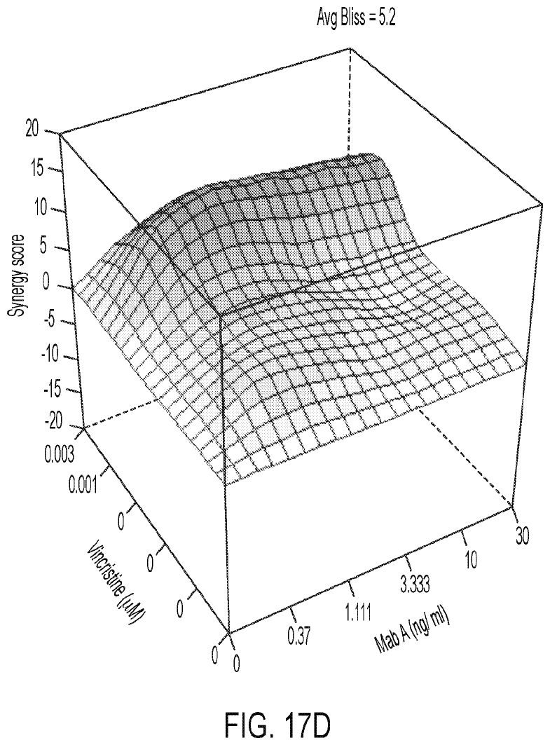

Cell T viability curves for single agent Mab A or vincristine on U-937 cells are shown in FIGS. 17A and 17B, respectively. Single agent Mab A shows partial cytotoxicity on U-937 cells and single agent vincristine shows strong cytotoxicity. Cell viability curves for combinations of Mab A and vincristine on U-937 tumor cells are shown in FIG. 17C. Synergy score 3D surface plots for Mab A and vincristine on U-937 cells are shown in FIG. 17D. The Mab A and vincristine combination results in weak syntergistic cytotoxicity on U-937 cells.

Synergy scores for combinations of Mab A and a chemotherapeutic/targeted agent on non-Hodgkin's lymphoma (NHL) tumor cell lines arc shown in Table 10.

TABLE 10
Combinations with chemotherapeutic and targeted agents in NHL

| Chemotherapeutic/ | | Average |
|---|---|---|
| Targeted agent | Cell line | Bliss score |
| ibrutinib | DOHH-2 | −0.22 |
| ibrutinib | OCI-LY7 | 2.3 |
| ibrutinib | Toledo | −0.60 |
| ibrutinib | U-937 | 6.2 |
| ibrutinib | WSU-DLCL2 | −3.2 |
| idelalisib | DOHH-2 | −0.89 |
| idelalisib | Karpas-422 | −3.2 |
| idelalisib | OC1-LY7 | −1.8 |
| idelalisib | Toledo | −1.6 |
| idelalisib | U-937 | 2.7 |
| idelalisib | WSU-DLCL2 | −7.4 |
| MIK665 | DOHH-2 | 3.1 |
| MIK665 | Karpas-422 | 3.7 |
| MIK665 | OCI-LY7 | 3.5 |
| MIK665 | Toledo | 6.7 |
| MIK665 | U-937 | 4.8 |
| MIK665 | WSU-DLCL2 | 16 |
| vincristine | DOHH-2 | −3.7 |
| vincristine | Karpas-422 | −12 |
| vincristine | OCI-LY7 | −1.0 |
| vincristine | Toledo | 3.0 |
| vincristine | U-937 | 5.2 |
| vincristine | WSU-DLCL2 | −0.73 |

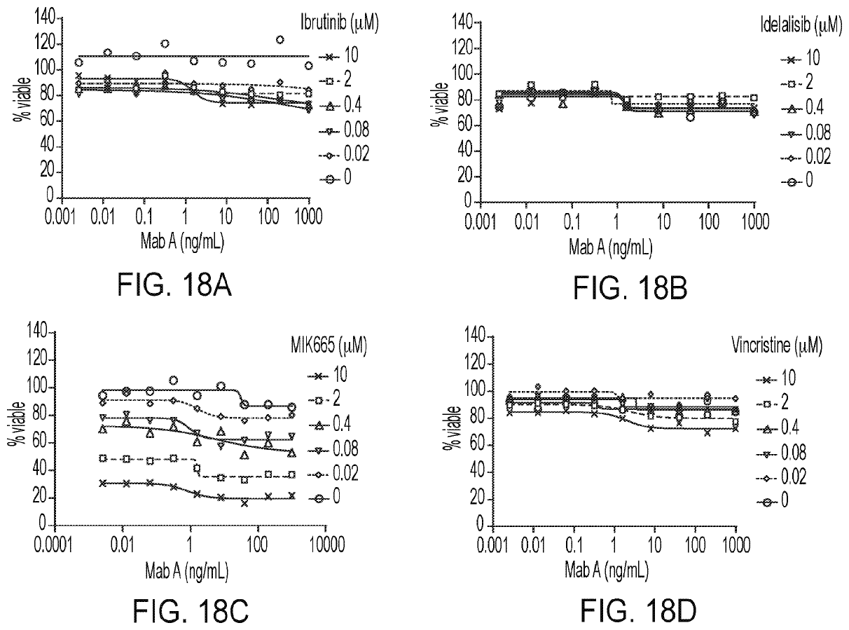

Cell viability curves for combinations of anti-DR-5 IgM Mab A with ibrutinib, idelalisib, MIK665, or vincristine on primary human hepatocytes are shown in FIGS. 18A, 18B, 18C and 18D, respectively. Combinations of Mab A with ibrutinib, idelalisib, and vincristine do not result in substantial cytotoxicity in primary human hepatocytes. Single agent MIK665 causes cytotoxicity in primary human hepatocytes, but this is not substantially enhanced in combination with Mab A.

### Example 10: In Vitro Birinapant Combination

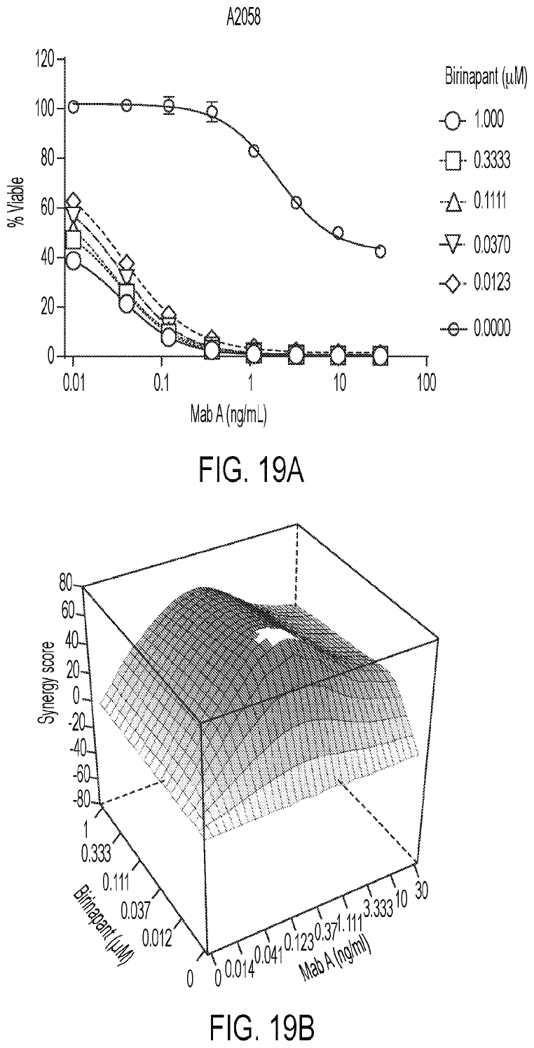

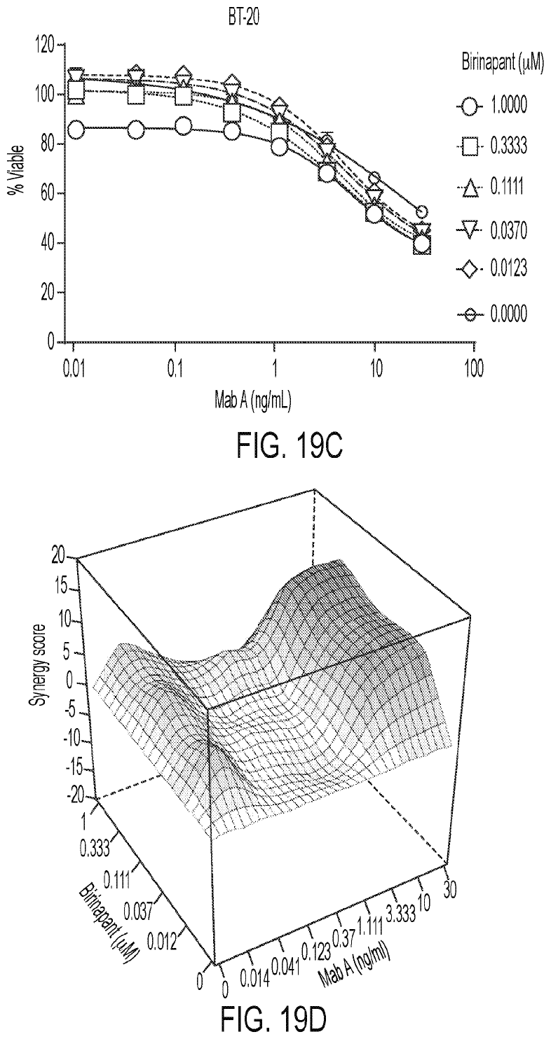

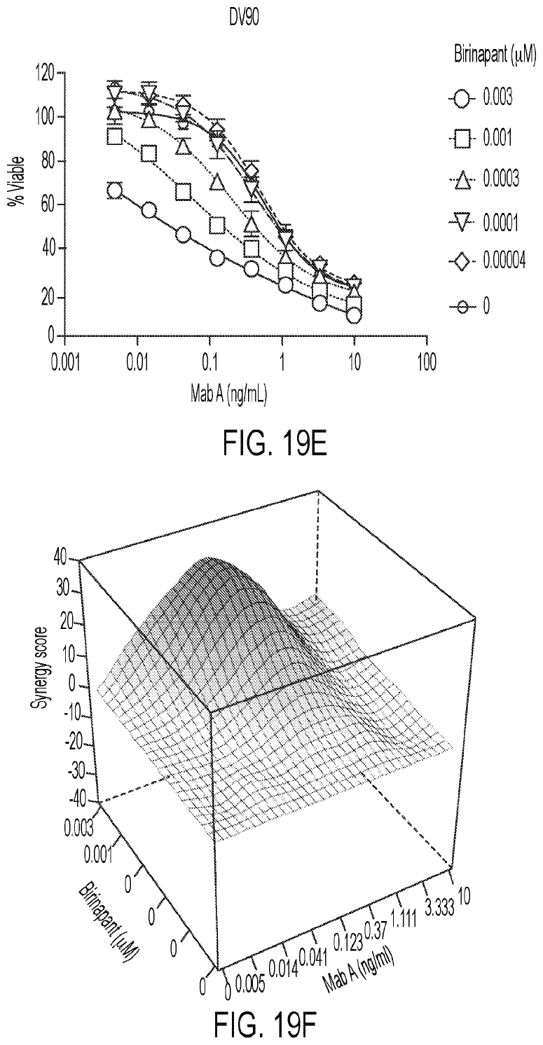

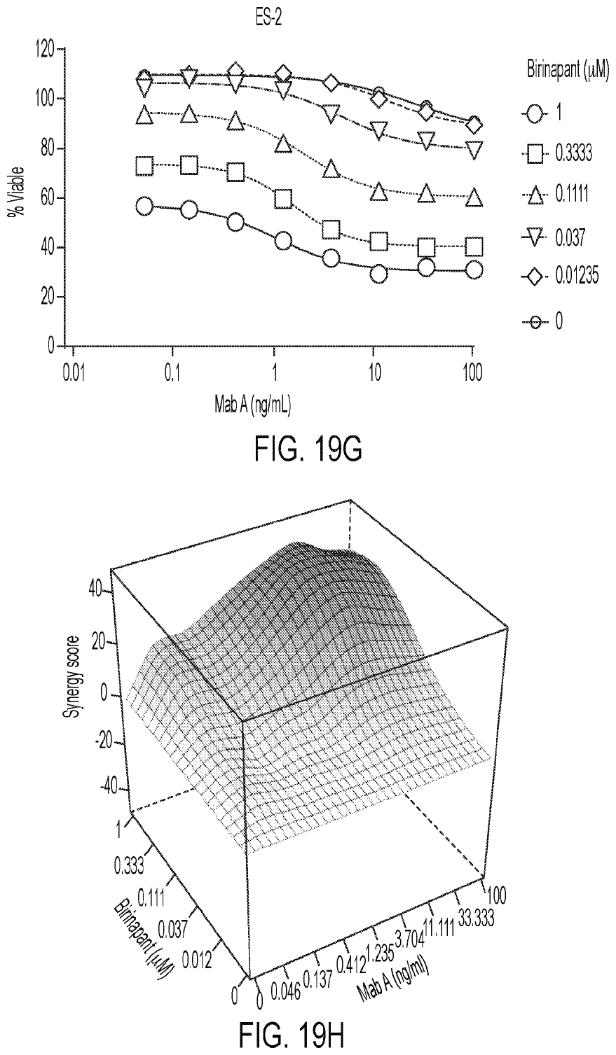

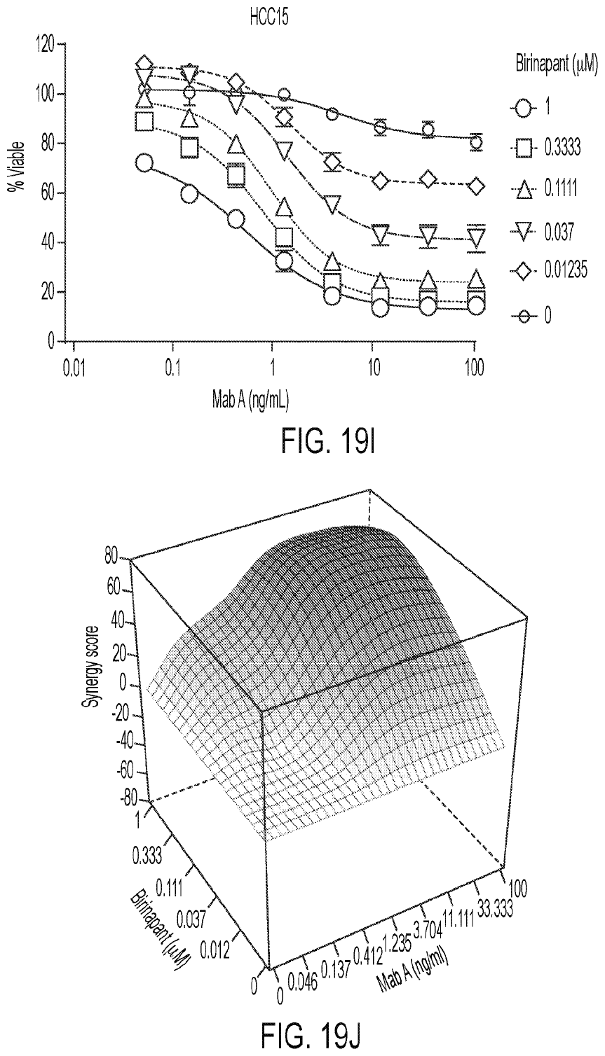

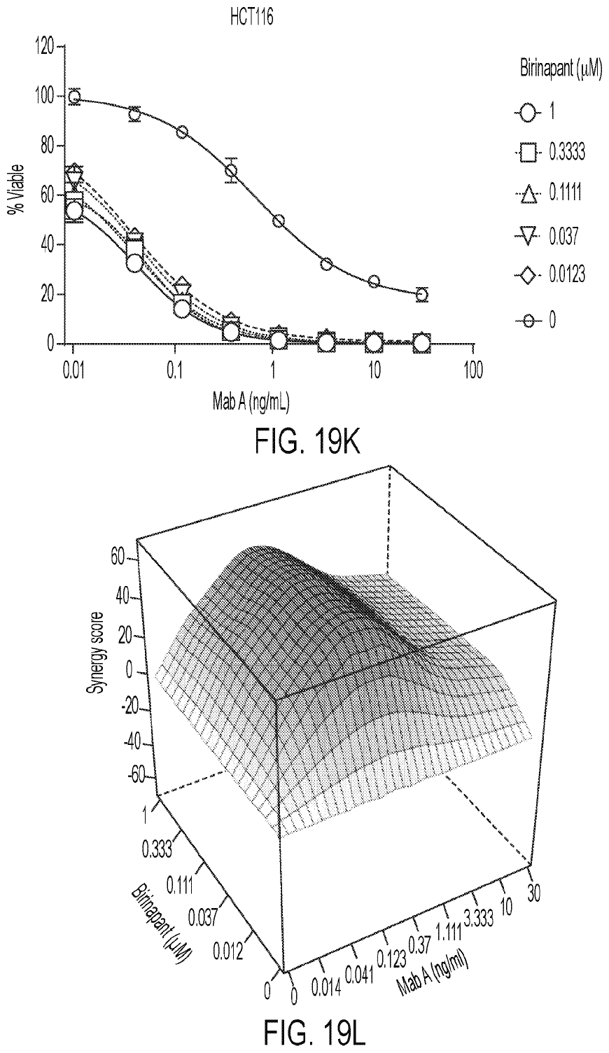

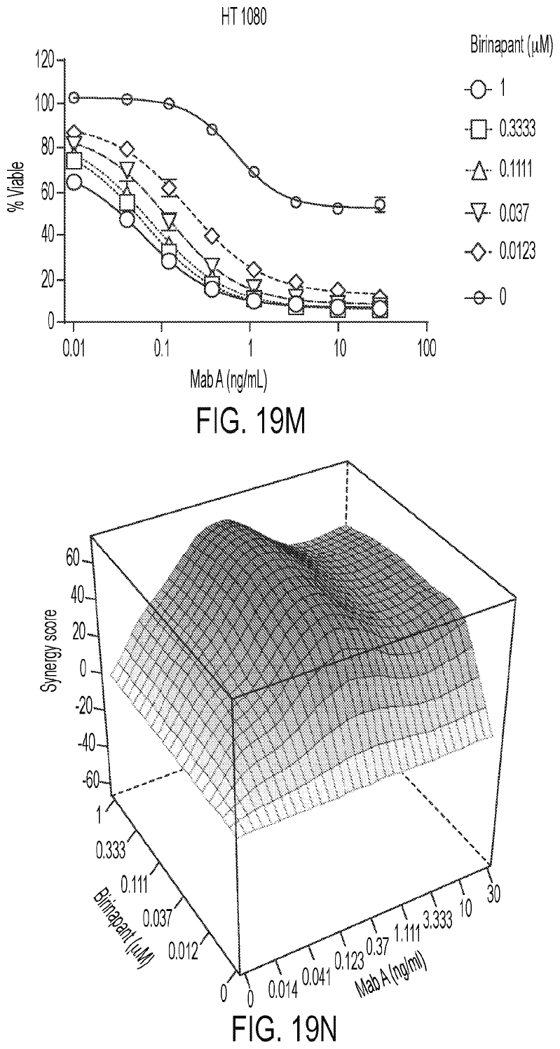

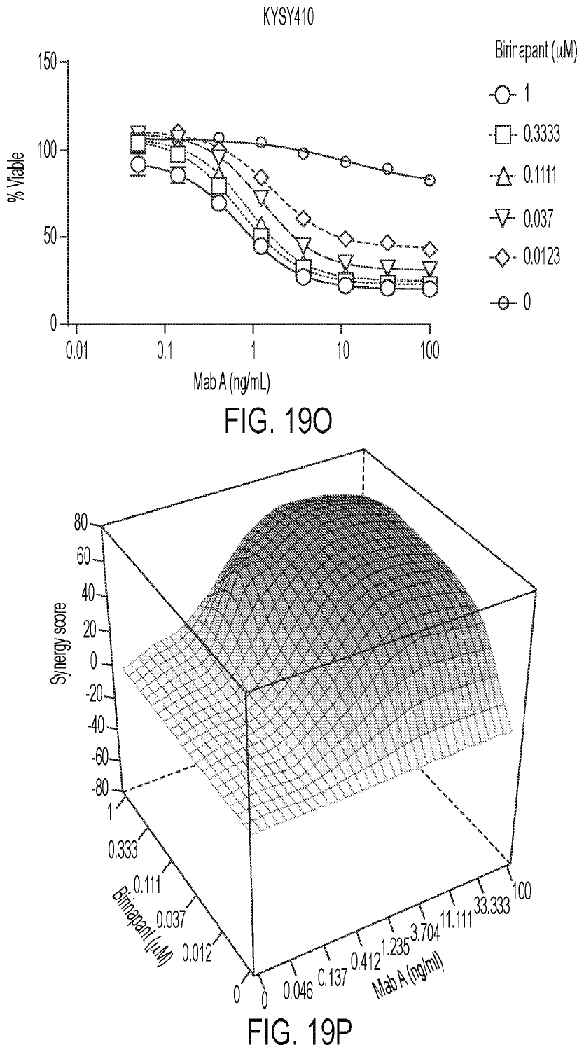

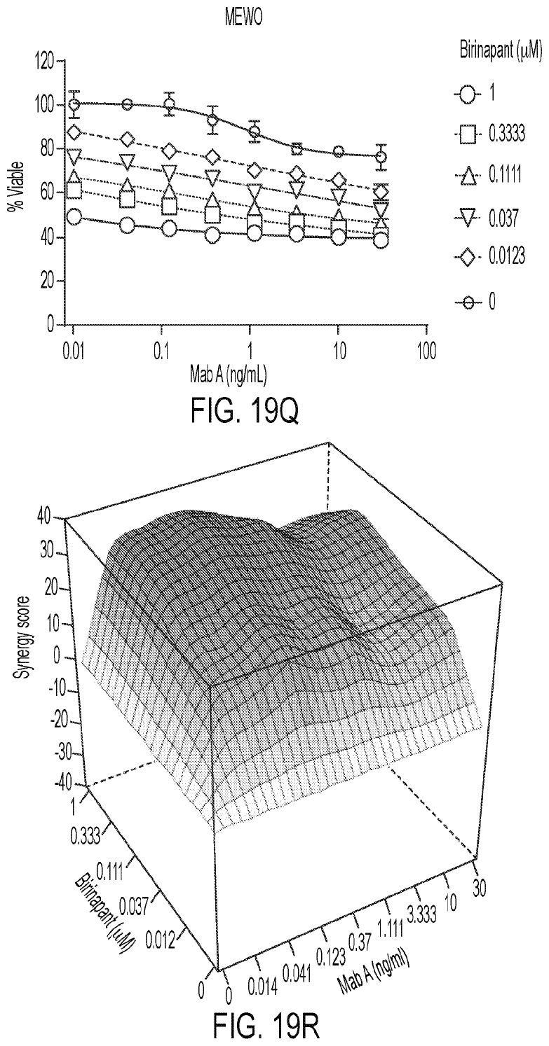

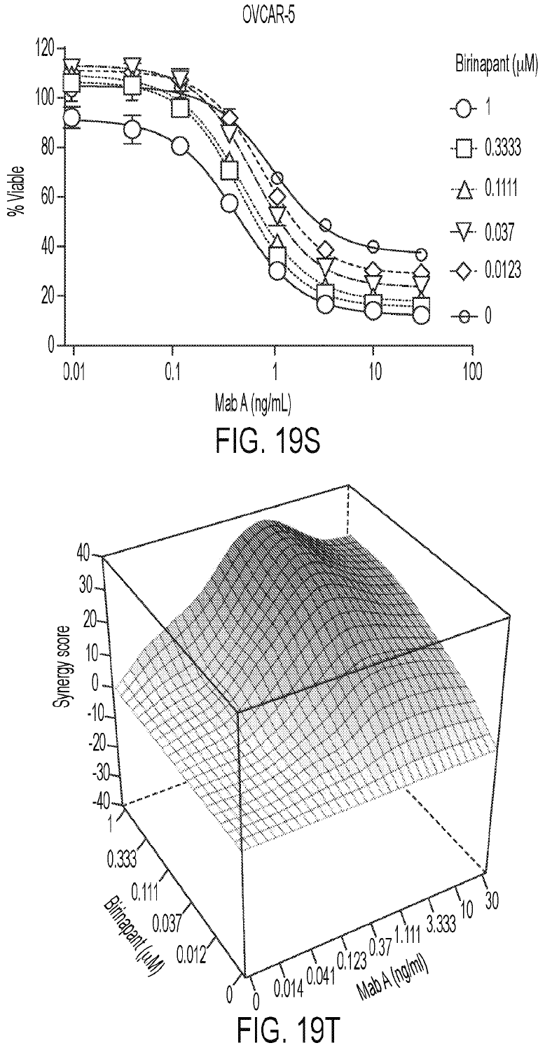

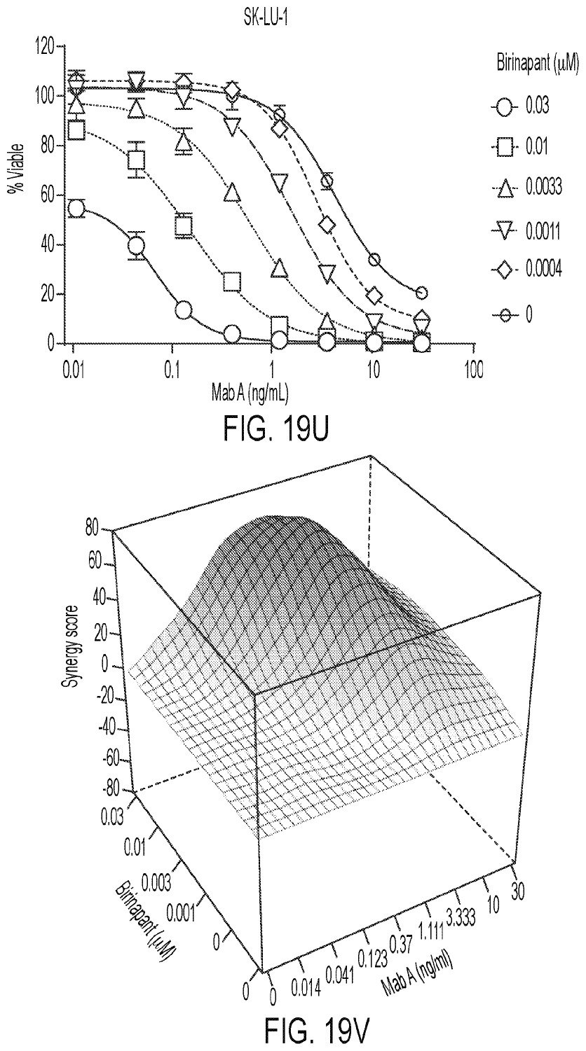

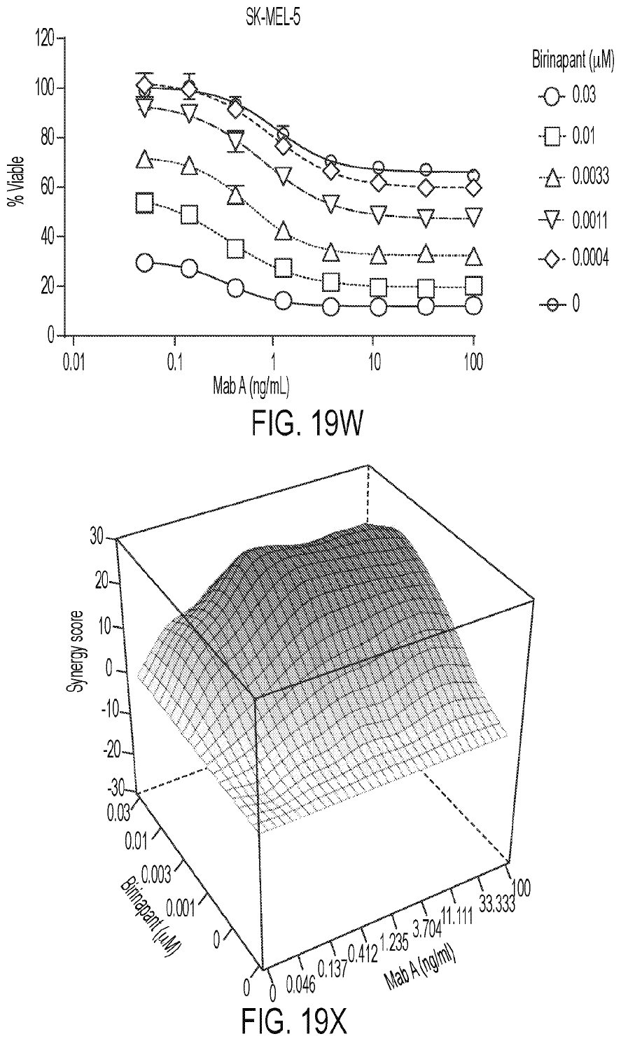

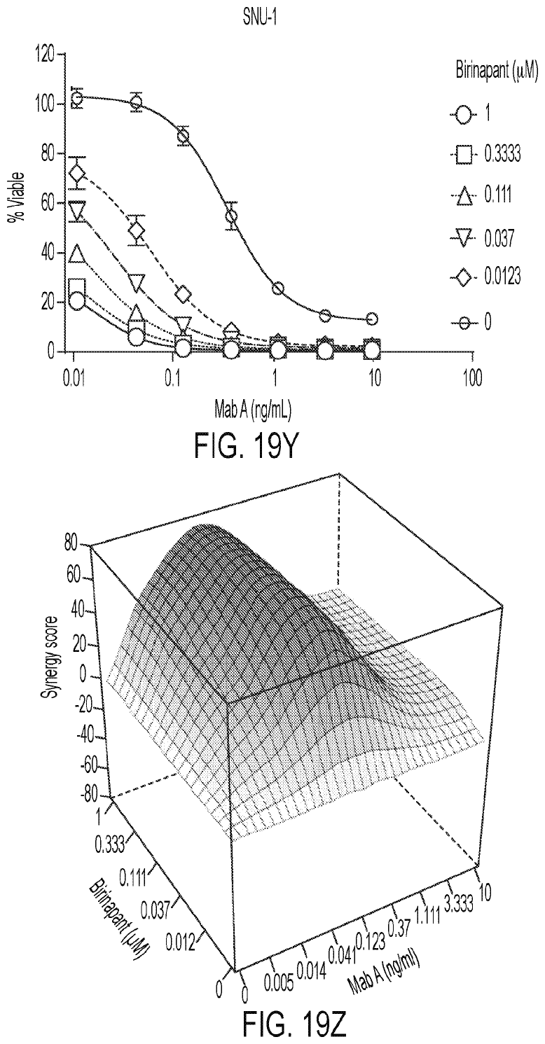

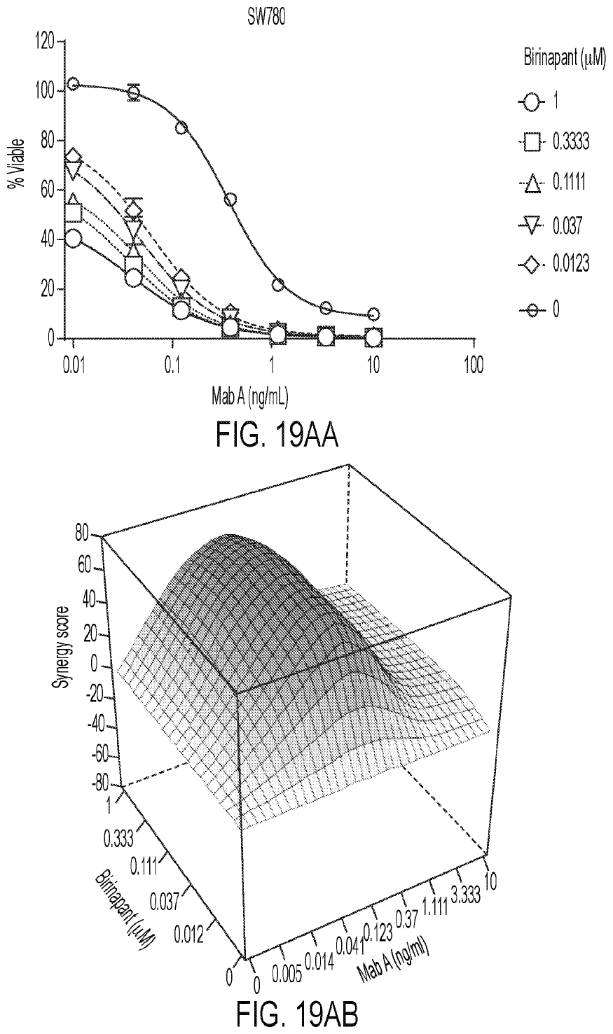

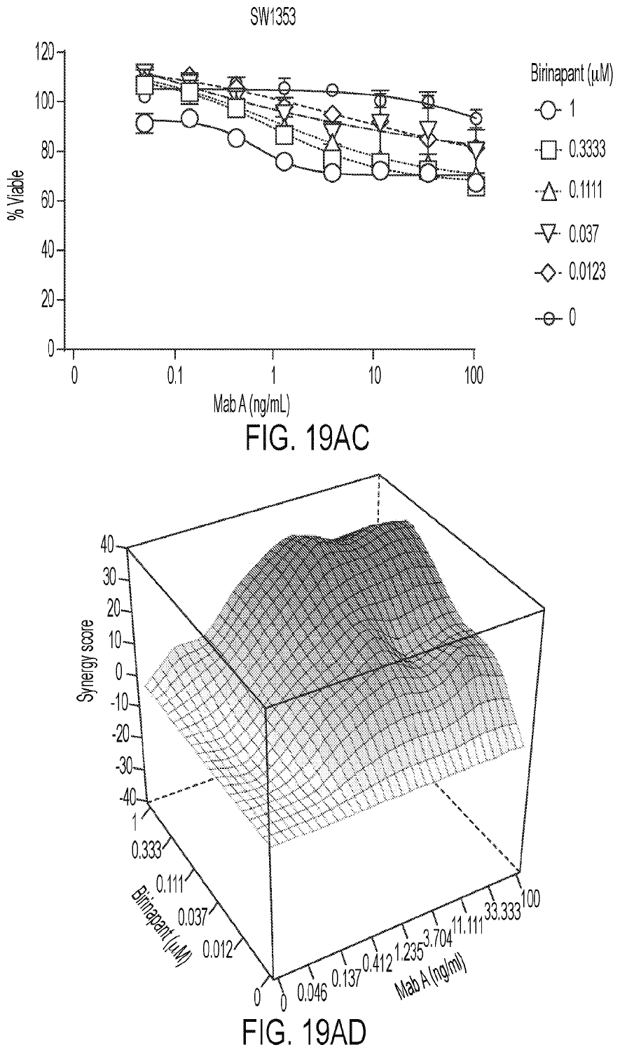

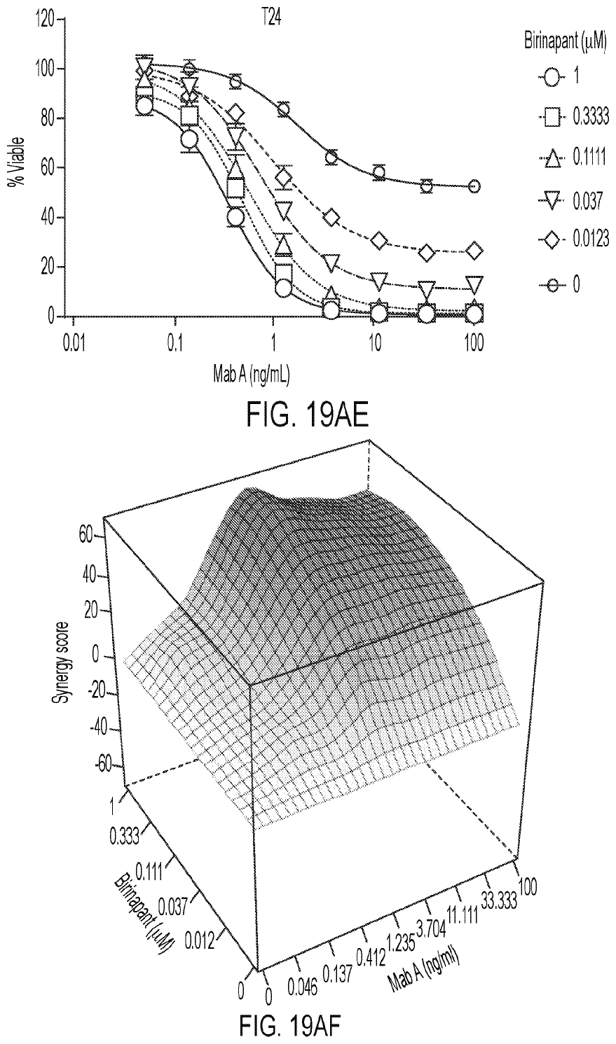

The in vitro potency of anti-DR5 IgM Mab A in combination with birinapant was evaluated on various tumor cell lines as follows. Tumor cells or primary human hepatocytes (BioIVT X008001) were seeded and the next day cells were treated with serial dilutions of anti-DR5 IgM Mab A and birinapant alone or in combination. After 72 hours at 37° C., Cell Titer Glo reagent (Promega) was added, and cell viability was read on a luminometer. Cell viability curves for combinations of anti-DR5 IgM Mab A with birinapant on A2058, BT-20, DV-90, ES-2, HCC15, HCT 116, HT 1080, KYSE 410, MEWO, OVCAR-5, SK-LU-1, SK-MEL-5, SNU-1, SW780, SW1353, and T24 are shown in FIGS. 19A, 19C, 19E, 19G, 19I, 19K, 19M, 19O, 19Q, 19S, 19U, 19W, 19Y, 19AA, 19AC, and 19AE, respectively. Synergy was determined as described in earlier examples. Synergy score 3D surface plots for A2058, BT-20, DV-90, ES-2, HCC15, HCT 116, HT 1080, KYSE 410, MEWO, OVCAR-5, SK-LU-1, SK-MEL-5, SNU-1, SW780, SW1353, and T24 cells are shown in FIGS. 19B, 19D, 19F, 19H, 19J, 19L, 19N, 19P, 19R, 19T, 19V, 19X, 19Z, 19AB, 19AD, and 19AF, respectively. The Average Bliss synergy scores for combinations of Mab A and birinapant on the various tumor cell lines, as well as the IC₅₀ values determined at various concentrations of birinapant are shown in Tables 11-13.

TABLE 11
Average Bliss Score and IC₅₀ Values for Birinapant

| | | Avg Bliss | IC₅₀ at Birinapant Concentration (μM) | | | | | |
|---|---|---|---|---|---|---|---|---|
| Cell Line | Tumor Type | score | 0 | 0.0123 | 0.0370 | 0.1111 | 0.3333 | 1.0000 |
| A2058 | Melanoma | 49 | 2.0 | 0.036 | 0.035 | 0.27 | 0.034 | 0.035 |
| HT 1080 | Fibrosarcoma | 45 | 0.67 | 0.20 | 0.12 | 0.072 | 0.063 | 0.061 |
| KYSE 410 | Esophageal | 42 | 13 | 1.6 | 1.3 | 0.89 | 0.72 | 0.71 |
| HCC15 | Lung | 41 | 4.6 | 1.4 | 1.3 | 0.93 | 0.75 | 0.52 |
| SNU-1 | Gastric | 39 | 0.34 | 0.056 | 0.024 | 0.015 | 0.012 | 0.017 |
| T24 | Bladder | 38 | 1.7 | 0.96 | 0.71 | 0.54 | 0.48 | 0.35 |
| HCT 116 | Colorectal | 36 | 0.65 | 0.038 | 0.043 | 0.039 | 0.052 | 0.041 |
| SW780 | Bladder | 35 | 0.36 | 0.060 | 0.047 | 0.046 | 0.036 | 0.037 |
| MEWO | Melanoma | 26 | 0.89 | 0.41 | NA | 0.18 | NA | NA |
| ES-2 | Ovarian | 19 | 26 | 14 | 4.6 | 2.0 | 1.6 | 1.0 |
| SW1353 | Chondrosarcoma | 18 | 133 | 4.3 | NA | 0.96 | 0.96 | 0.65 |
| OVCAR-5 | Ovarian | 17 | 1.0 | 0.81 | 0.66 | 0.52 | 0.49 | 0.45 |
| BT-20 | TNBC | 4 | 24 | 5.0 | 5.1 | 4.5 | 4.8 | 5.9 |

TABLE 12
Average Bliss Score and IC₅₀ Values for Birinapant

| | | Avg Bliss | IC₅₀ at Birinapant Concentration (μM) | | | | | |
|---|---|---|---|---|---|---|---|---|
| Cell Line | Tumor Type | score | 0 | 0.00004 | 0.0001 | 0.0003 | 0.0001 | 0.003 |
| DV-90 | Lung | 9 | 0.55 | 0.48 | 0.38 | 0.20 | 0.057 | NA |

TABLE 13
Average Bliss Score and IC₅₀ Values for Birinapant

| | | Avg Bliss | IC₅₀ at Birinapant Concentration (μM) | | | | | |
|---|---|---|---|---|---|---|---|---|
| Cell Line | Tumor Type | score | 0 | 0.0004 | 0.0011 | 0.0033 | 0.01 | 0.03 |
| SK-MEL-5 | Melanoma | 15 | 1.1 | 0.91 | 0.77 | 0.57 | 0.34 | 0.34 |
| SK-LU-1 | Lung | 25 | 4.0 | 2.6 | 1.5 | 0.56 | 0.14 | 0.062 |

### Example 11: In Vivo Birinapant Combination

### MDA-MB-231-Triple-Negative Breast Cancer (TNBC) Model

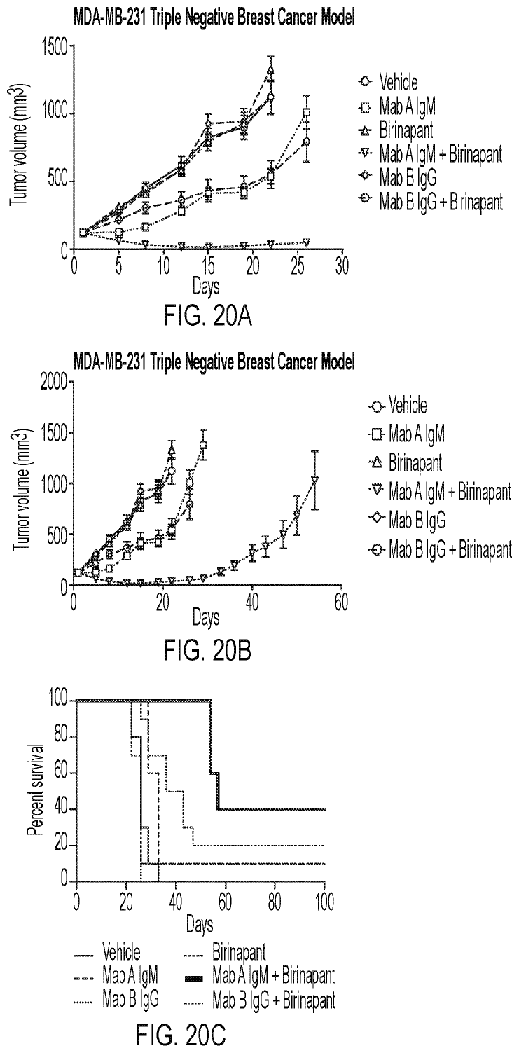

5×10⁶ MDA-MB-231 tumor cells were implanted subcutaneously in the flanks of female NCr nu/nu mice. When mean tumor volume reached 100-150 mm³, mice were dosed with either vehicle i.v. every other day for a total of 11 doses, 5 mg/kg of anti-DR-5 IgM Mab A i.v. every other day for 11 doses, 2.5 mg/kg of birinapant i.p. every 3 days for 7 doses, 5 mg/kg of anti-DR5 IgG Mab B i.v. weekly for 3 doses, a combination of the anti-DR5 IgM Mab A and birinapant treatment regimens, or a combination of the anti-DR5 IgG Mab B and birinapant treatment regimens (n=10 animals/group). Tumor volumes over time through day 26 are shown in FIG. 20A. Tumor volumes through day 54 are shown in FIG. 20B and overall survival is shown in FIG. 20C. On day 22 (the last day that all control animals were on study), the combination therapy with anti-DR-5 IgM Mab A and birinapant significantly reduced tumor volume compared to birinapant alone. The combination of anti-DR5 IgG Mab B with birinapant also significantly reduced tumor volume compared to birinapant alone, although the tumor growth inhibition was much less than with anti-DR5 IgM Mab A. Anti-DR5 IgM Mab A and birinapant combination also significantly extended overall survival compared to birinapant alone. All animals in the anti-DR5 IgM Mab A and birinapant combined treatment group achieved at least a partial response and 4/10 animals were tumor free at 100 days.

### EBC-1—Non-Small Cell Lung Cancer (NSCLC) Model

3×10⁶ EBC-1 tumor cells were implanted subcutaneously in the flanks of female BALB/c nude mice. When mean tumor volume reached 100-200 mm³, mice were dosed with either vehicle i.v. every other day for a total of 11 doses, 5 mg/kg of anti-DR5 IgM Mab A iv. every other day for 11 doses, 30 mg/kg of birinapant i.p. every 3 days for 7 doses, or a combination of the anti-DR5 IgM Mab A and birinapant treatment regimens (n=10 animals/group). Dosing holidays were given if an individual animal body weight loss exceeded 15% and dosing was resumed when body weight loss recovered to less than 10%.

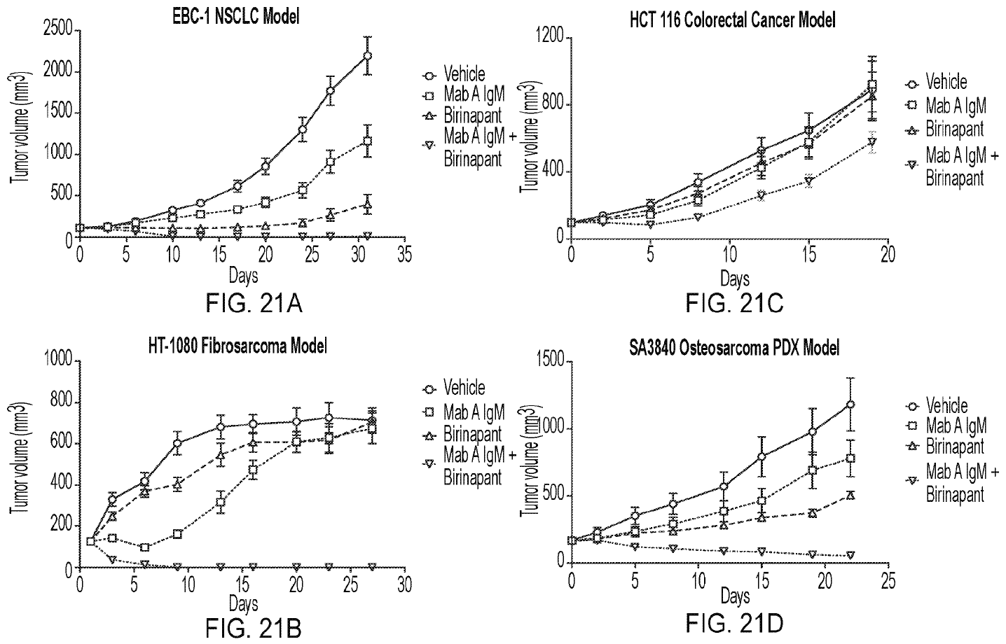

Tumor volumes over time are shown in FIG. 21A. Although 3 mice in the birinapant group and 6 mice in the combined treatment group missed doses due to body weight loss, the combination therapy with anti-DR5 IgM Mab A and birinapant significantly reduced tumor volume compared to anti-DR5 IgM Mab A alone and 9/10 mice were tumor-free as of study day 31.

### HT-1080-Fibrosarcoma Model

1×10⁷ HT-1080 tumor cells were implanted subcutaneously in the flanks of female NCr nu/nu mice. When mean tumor volume reached 100-150 mm³, mice were dosed with either 20 vehicle i.v. every other day for a total of 11 doses, 5 mg/kg of anti-DR5 IgM Mab A i.v. every other day for 11 doses, 30 mg/kg of birinapant i.p. every 3 days for 2 doses followed by 15 mg/kg of birinapant i.p. every 3 days for 5 doses, or a combination of the anti-DR5 IgM Mab A and birinapant treatment regimens (n=10 animals/group).

Tumor volumes over time are shown in FIG. 21B. The combination therapy with anti-DR5 IgM Mab A and birinapant significantly reduced tumor volume compared to either single agent alone, and all mice in the combination treatment group were tumor-free as of study day 27.

HCT 116-Colorectal Cancer Model

5×10⁶ HCT 116 tumor cells were implanted subcutaneously in the flanks of female nu/nu mice. When mean tumor volume reached 75-150 mm³, mice were dosed with either vehicle i.v. every other day for a total of 11 doses, 5 mg/kg of anti-DR5 IgM Mab A i.v. every other day for 11 doses, 15 mg/kg of birinapant i.p. every 3 days for 7 doses, or a combination of the anti-DR5 IgM Mab A and birinapant treatment regimens (n=10 animals/group).

Tumor volumes over time are shown in FIG. 21C. The combination therapy with anti-DR5 IgM Mab A and birinapant partially reduced tumor volume, though this did not reach statistical significance on study day 19.

### S43840—Osteosarcoma PDX Model

SA3840 tumor fragments 2-3 mm in diameter were implanted subcutaneously in the flanks of female NOD/SCID mice. When mean tumor volume reached 100-200 mm³, mice were dosed with either vehicle i.v. every other day for a total of 11 doses, 5 mg/kg of anti-DR5 IgM Mab A i.v. every other day for 11 doses, 30 mg/kg of birinapant i.p. every 3 days for 7 doses, or a combination of the anti-DR5 IgM Mab A and birinapant treatment regimens (n=5 animals/group). Dosing holidays were given if an individual animal body weight loss exceeded 15% and dosing was resumed when body weight loss recovered to less than 10%.

Tumor volumes over time are shown in FIG. 21D. Although 1 animal in each the birinapant group and the combined treatment group missed doses due to body weight loss, the combination therapy with anti-DR5 IgM Mab A and birinapant significantly reduced tumor volume compared to the vehicle control group.

### OV15631 and OV15841 Ovarian PDX Models

OV15631 and OV15841 tumor fragments 2-3 mm in diameter were implanted subcutaneously in the flanks of female NOD/SCID mice. When mean tumor volume reached 100-200 mm³, mice were dosed with either vehicle i.v. every other day for a total of 11 doses, 5 mg/kg of anti-DR5 IgM Mab A i.v. every other day for 11 doses, 30 mg/kg of birinapant i.p. every 3 days for 7 doses, or a combination of the anti-DR5 IgM Mab A and birinapant treatment regimens (n=5 animals/group). No synergy was seen for the combination in these models.

### Example 12: In Vitro Birinapant Combination for Head and Neck Cancers

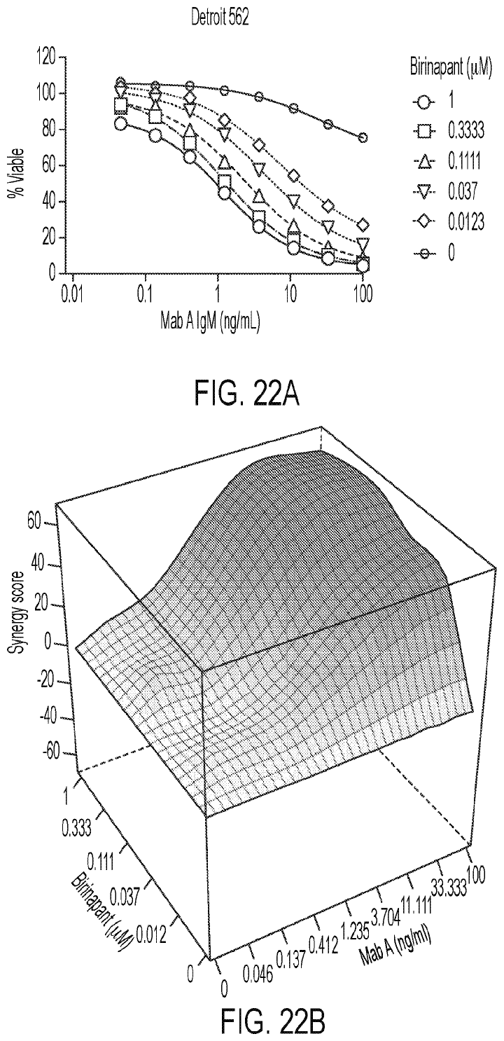

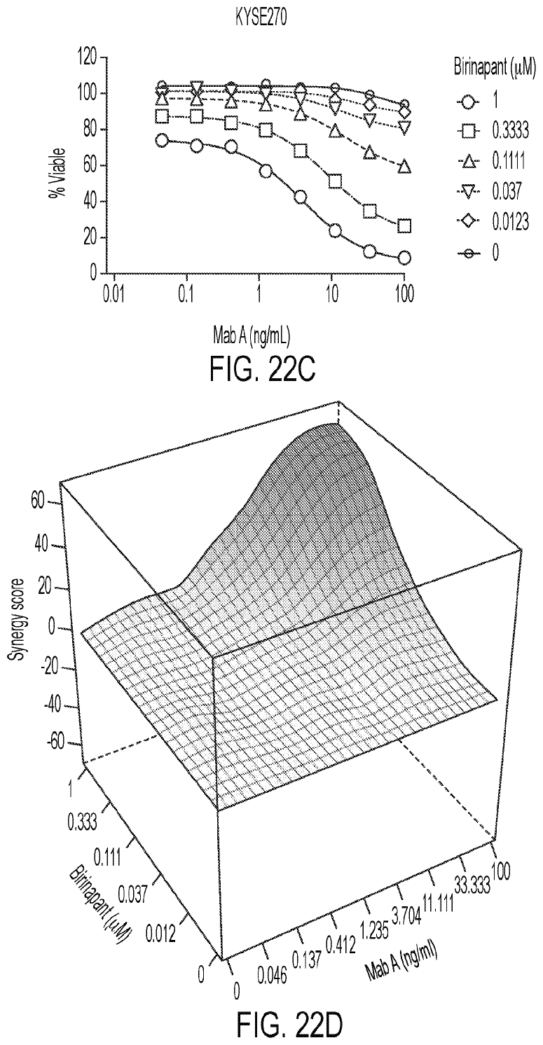

The in vitro potency of anti-DR5 IgM Mab A in combination with birinapant was evaluated on various head and neck tumor cell lines as follows. Tumor cells were seeded and the next day cells were treated with serial dilutions of anti-DR5 IgM Mab A and birinapant alone or in combination. After 72 hours at 37° C., Cell Titer Glo reagent (Promega) was added, and cell viability was read on a luminometer. Exemplary cell viability curves for combinations of anti-DR5 IgM Mab A with birinapant on Detroit 562 and KYSE270 are shown in FIGS. 22A and 22C, respectively. Synergy was determined as described in earlier examples. Synergy score 3D surface plots for Detroit 562 and KYSE270 cells are shown in FIGS. 22B and 22D, respectively. The Average Bliss synergy scores for combinations of Mab A and birinapant on the various head and neck tumor cell lines are shown in Table 14.

TABLE 14
Combinations with birinapant in head and neck cancer cell lines

| Cell line | Average Bliss score |
|---|---|
| Detroit 562 | 36 |
| KYSE410 | 35 |
| TE-1 | 29 |
| 8305C | 27 |
| A253 | 26 |
| KYSE-70 | 25 |
| OE19 | 17 |
| FaDu | 16 |
| KYSE270 | 15 |
| 8505C | 10 |
| KYSE150 | 0.2 |

### Example 13: In Vitro SMAC Mimetic Combination

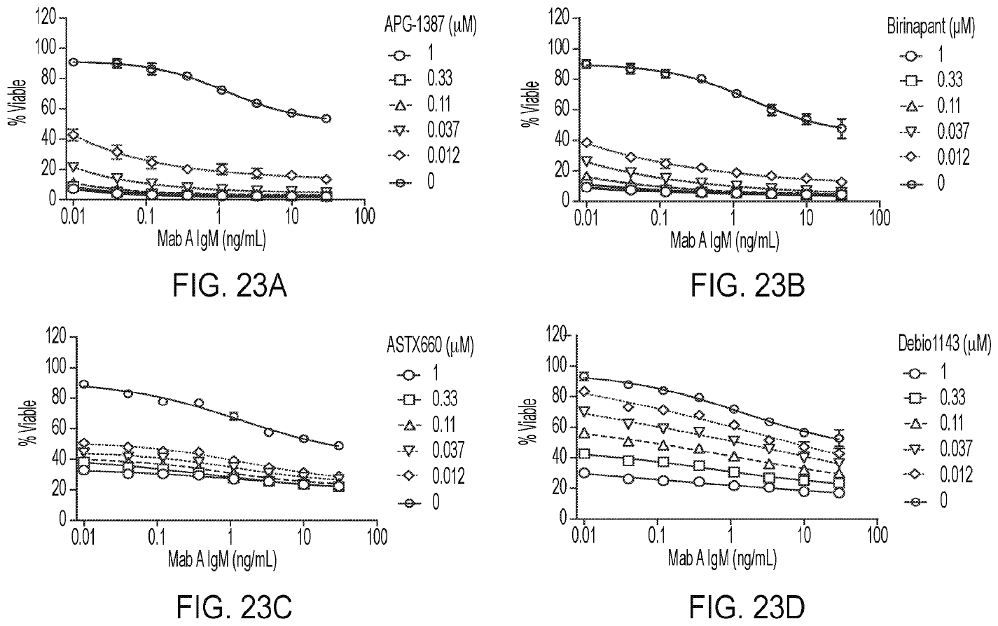

The in vitro potency of anti-DR5 IgM Mab A in combination with various SMAC mimetics was evaluated on various tumor cell lines as follows. Tumor cells or primary human hepatocytes (BioIVT X008001) were seeded and the next day cells were treated with serial dilutions of anti-DR5 IgM Mab A and SMAC mimetic alone or in combination. After 72 hours at 37° C., Cell Titer Glo reagent (Promega) was added, and cell viability was read on a luminometer. Exemplary cell viability curves for combinations of anti-DR5 IgM Mab A with APG-1387, birinapant, ASTX660, and Debio1143 on EBC-1 cells are shown in FIGS. 23A-23D, respectively. Synergy was determined as described in earlier examples. The average Bliss synergy scores for combinations of Mab A and SMAC mimetic on the various tumor cell lines are shown in Table 15. On average, bivalent SMAC mimetics birinapant and APG-1387 have higher average Bliss scores than monovalent SMAC mimetics Debio 1143 and ASTX660.

TABLE 15
Average Bliss scores for combinations with SMAC
mimetics in solid tumor cell lines

| | | APG- | Debio | |
|---|---|---|---|---|
| Cell line | Birinapant | 1387 | 1143 | ASTX660 |
| EBC-1 | 39 | 47 | 23 | 17 |
| HCT 116 | 31 | 34 | 19 | 29 |
| HT 1080 | 30 | 38 | 12 | 20 |
| OVCAR-4 | 14 | 10 | 3 | 9 |
| SK-MEL-5 | 14 | 7 | 4 | 11 |
| SW1353 | −0.6 | −3 | −1 | −2 |

### Example 14: In Vivo Bevacizumab Combination

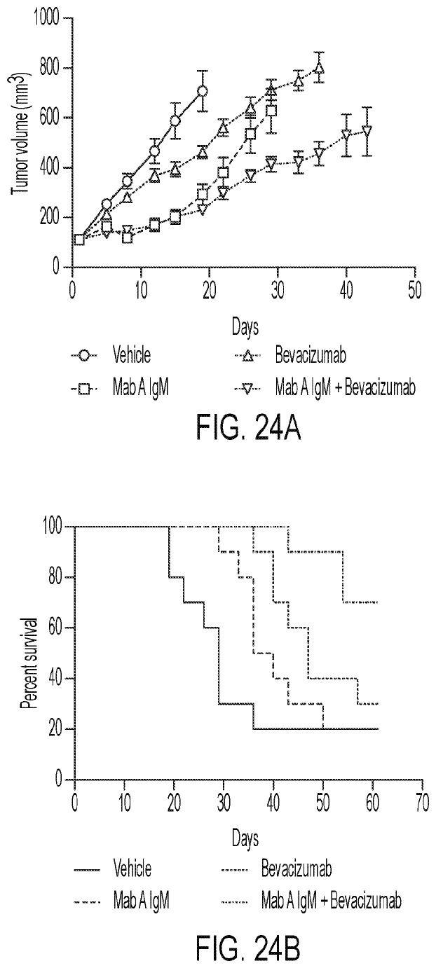

2×10⁶ Colo205 tumor cells were implanted subcutaneously in the flanks of female NCr nude mice. When mean tumor volume reached 100-150 mm³, mice were dosed with either vehicle i.v. every other day for a total of 7 doses, 5 mg/kg of anti-DR5 IgM Mab A i.v. every other day for a total of 7 doses, 5 mg/kg of bevacizumab i.p. biweekly for 5 weeks, or a combination of the anti-DR5 IgM Mab A and bevacizumab treatment regimens. Tumor volume (n=10 animals/group) is shown in FIG. 24A and overall survival is shown in FIG. 24B. On day 19 (the last day that all control animals were on study), the combination therapy with anti-DR5 IgM Mab A and bevacizumab significantly reduced tumor volume compared to bevacizumab alone. The combined treatment also significantly extended overall survival compared to either single agent alone.

TABLE 16
Other Sequences in the Disclosure

| SEQ | | |
|---|---|---|
| ID | Nickname (source) | Sequence |
| 91 | Human IgM Constant | GSASAPTLFPLVSCENSPSDTSSVAVGCLAQDFLPDSITFSWKYKNN |
| | region IMGT allele | SDISSTRGFPSVLRGGKYAATSQVLLPSKDVMQGTDEHVVCKVQHPN |
| | IGHM*03 (GenBank: | GNKEKNVPLPVIAELPPKVSVFVPPRDGFFGNPRKSKLICQATGFSP |
| | pir\|S37768\|) | RQIQVSWLREGKQVGSGVTTDQVQAEAKESGPTTYKVTSTLTIKESD |
| | | WLSQSMFTCRVDHRGLTFQQNASSMCVPDQDTAIRVFAIPPSFASIF |
| | | LTKSTKLTCLVTDLTTYDSVTISWTRQNGEAVKTHTNISESHPNATF |
| | | SAVGEASICEDDWNSGERFTCTVTHTDLPSPLKQTISRPKGVALHRP |
| | | DVYLLPPAREQLNLRESATITCLVTGFSPADVFVQWMQRGQPLSPEK |
| | | YVTSAPMPEPQAPGRYFAHSILTVSEEEWNTGETYTCVVAHEALPNR |
| | | VTERTVDKSTGKPTLYNVSLVMSDTAGTCY |
| 92 | Human IgM Constant | GSASAPTLFPLVSCENSPSDTSSVAVGCLAQDFLPDSITFSWKYKNN |
| | region IMGT allele | SDISSTRGFPSVLRGGKYAATSQVLLPSKDVMQGTDEHVVCKVQHPN |
| | IGHM*04 (GenBank: | GNKEKNVPLPVIAELPPKVSVFVPPRDGFFGNPRKSKLICQATGFSP |
| | sp\|P01871.4\|) | RQIQVSWLREGKQVGSGVTTDQVQAEAKESGPTTYKVTSTLTIKESD |
| | | WLGQSMFTCRVDHRGLTFQQNASSMCVPDQDTAIRVFAIPPSFASIF |
| | | LTKSTKLTCLVTDLTTYDSVTISWTRQNGEAVKTHTNISESHPNATF |
| | | SAVGEASICEDDWNSGERFTCTVTHTDLPSPLKQTISRPKGVALHRP |
| | | DVYLLPPAREQLNLRESATITCLVTGFSPADVFVQWMQRGQPLSPEK |
| | | YVTSAPMPEPQAPGRYFAHSILTVSEEEWNTGETYTCVVAHEALPNR |
| | | VTERTVDKSTGKPTLYNVSLVMSDTAGTCY |
| 93 | Human IgA1 heavy | ASPTSPKVFPLSLCSTQPDGNVVIACLVQGFFPQEPLSVTWSESGQG |
| | chain constant | VTARNFPPSQDASGDLYTTSSQLTLPATQCLAGKSVTCHVKHYTNPS |
| | region, e.g., amino | QDVTVPCPVPSTPPTPSPSTPPTPSPSCCHPRLSLHRPALEDLLLGS |
| | acids 144 to 496 of | EANLTCTLTGLRDASGVTFTWTPSSGKSAVQGPPERDLCGCYSVSSV |
| | GenBank AIC59035.1 | LPGCAEPWNHGKTFTCTAAYPESKTPLTATLSKSGNTFRPEVHLLPP |
| | | PSEELALNELVTLTCLARGFSPKDVLVRWLQGSQELPREKYLTWASR |
| | | QEPSQGTTTFAVTSILRVAAEDWKKGDTFSCMVGHEALPLAFTQKTI |
| | | DRLAGKPTHVNVSVVMAEVDGTCY |
| 94 | Human IgA2 heavy | ASPTSPKVFPLSLDSTPQDGNVVVACLVQGFFPQEPLSVTWSESGQN |
| | chain constant | VTARNFPPSQDASGDLYTTSSQLTLPATQCPDGKSVTCHVKHYTNSS |
| | region, e.g., amino | QDVTVPCRVPPPPPCCHPRLSLHRPALEDLLLGSEANLTCTLTGLRD |
| | acids 1 to 340 of | ASGATFTWTPSSGKSAVQGPPERDLCGCYSVSSVLPGCAQPWNHGET |
| | GenBank P01877.4 | FTCTAAHPELKTPLTANITKSGNTFRPEVHLLPPPSEELALNELVTL |
| | | TCLARGFSPKDVLVRWLQGSQELPREKYLTWASRQEPSQGTTTYAVT |
| | | SILRVAAEDWKKGETFSCMVGHEALPLAFTQKTIDRMAGKPTHINVS |
| | | VVMAEADGTCY |
| 95 | Precursor Human | MLLFVLTCLLAVFPAISTKSPIFGPEEVNSVEGNSVSITCYYPPTSV |
| | Secretory Component | NRHTRKYWCRQGARGGCITLISSEGYVSSKYAGRANLTNFPENGTFV |
| | | VNIAQLSQDDSGRYKCGLGINSRGLSFDVSLEVSQGPGLLNDTKVYT |
| | | VDLGRTVTINCPFKTENAQKRKSLYKQIGLYPVLVIDSSGYVNPNYT |
| | | GRIRLDIQGTGQLLFSVVINQLRLSDAGQYLCQAGDDSNSNKKNADL |
| | | QVLKPEPELVYEDLRGSVTFHCALGPEVANVAKFLCRQSSGENCDVV |
| | | VNTLGKRAPAFEGRILLNPQDKDGSFSVVITGLRKEDAGRYLCGAHS |
| | | DGQLQEGSPIQAWQLFVNEESTIPRSPTVVKGVAGGSVAVLCPYNRK |
| | | ESKSIKYWCLWEGAQNGRCPLLVDSEGWVKAQYEGRLSLLEEPGNGT |
| | | FTVILNQLTSRDAGFYWCLTNGDTLWRTTVEIKIIEGEPNLKVPGNV |
| | | TAVLGETLKVPCHFPCKFSSYEKYWCKWNNTGCQALPSQDEGPSKAF |
| | | VNCDENSRLVSLTLNLVTRADEGWYWCGVKQGHFYGETAAVYVAVEE |
| | | RKAAGSRDVSLAKADAAPDEKVLDSGFREIENKAIQDPRLFAEEKAV |
| | | ADTRDQADGSRASVDSGSSEEQGGSSRALVSTLVPLGIVLAVGAVAV |
| | | GVARARHRKNVDRVSIRSYRTDISMSDFENSREFGANDNMGASSITQ |
| | | ETSLGGKEEFVATTESTTETKEPKKAKRSSKEEAEMAYKDFLLQSST |
| | | VAAEAQDGPQEA |
| 96 | Precursor Human J | MKNHLLFWGVLAVFIKAVHVKAQEDERIVLVDNKCKCARITSRIIRS |
| | Chain | SEDPNEDIVERNIRIIVPLNNRENI3DPTSPLRTRFVYHLSDLCKKC |
| | | DPTEVELDNQIVTATQSNICDEDSATETCYTYDRNKCYTAVVPLVYG |
| | | GETKMVETALTPDACYPD |
| 97 | Mature Human J Chain | QEDERIVLVDNKCKCARITSRIIRSSEDPNEDIVERNIRIIVPLNNR |
| | | ENISDPTSPLRTRFVYHLSDLCKKCKPTEVELDNQIVTATQSNICDE |
| | | DSATETCYTYDRNKCYTAVVPLVYGGETKMVETALTPDACYPD |
| 98 | J Chain Y102A | QEDERIVLVDNKCKCARITSRIIRSSEDPNEDIVERNIRIIVPLNNR |
| | mutation | ENISQRTSPLRTRFVYHLSDLCKKCDPTEVELDNQIVTATQSNICDE |
| | | DSATETCATYDRNKCYTAVVPLVYGGETKMVETALTPDACYPD |
| 99 | “5” Peptide linker | GGGGS |
| 100 | “10” Peptide linker | GGGGSGGGGS |
| 101 | “15” Peptide linker | GGGGSGGGGSGGGGS |
| 102 | “20” Peptide linker: | GGGGSGGGGSGGGGSGGGGS |
| 103 | “25” Peptide Linker | GGGGSGGGGSGGGGSGGGGSGGGGS |

What is claimed is:

1. A method for inhibiting, delaying, or reducing malignant cell growth in a subject with cancer in need of treatment, comprising administering to the subject a combination therapy comprising:
(a) an effective amount of a pentameric or hexameric IgM or IgM-like antibody or a dimeric IgA or IgA-like antibody, or a multimerized antigen-binding fragment, variant, or derivative thereof that specifically and agonistically binds to DR5, wherein three to twelve of the antigen binding domains of the IgM or IgM-like antibody or multimerized antigen-binding fragment, variant, or derivative thereof or three or four of the antigen binding domains of the IgA or IgA-like antibody or multimerized antigen-binding fragment, variant, or derivative thereof are DR5-specific and agonistic; and
(b) an effective amount of a cancer therapy, wherein the cancer therapy comprises a second mitochondria-derived activator of caspases (SMAC) mimetic, radiation, a folic acid analog, a platinum-based agent, a taxane, a topoisomerase II inhibitor, a vinca alkaloid, a Bruton's tyrosine kinase (BTK) inhibitor, a phosphoinositide 3-kinase delta (PI3Kδ) inhibitor, a myeloid cell leukemia-1 (Mcl-1) inhibitor, an anti-VEGF antibody, or any combination thereof.

2. The method of claim 1, wherein the cancer therapy comprises a SMAC mimetic, and wherein the SMAC mimetic comprises a bivalent SMAC mimetic.

3. The method of claim 2, wherein the SMAC mimetic comprises birinapant.

4. The method of claim 1, wherein the cancer therapy comprises leucovorin, oxaliplatin, carboplatin, paclitaxel, an anthracycline, etoposide, vincristine, ibrutinib, idelalisib, MIK665, bevacizumab, birinapant, GDC-0152, HGS-1029/AEG40826, Debio1143, APG-1387, ASTX660, or any combination thereof.

5. The method of claim 1, further comprising administering an effective amount of an additional cancer therapy.

6. The method of claim 5, wherein the additional cancer therapy comprises a topoisomerase I inhibitor, a nucleoside analog, a platinum-based agent, or any combination thereof.

7. The method of claim 6, wherein the additional cancer therapy comprises irinotecan, topotecan, fluorouracil (5-FU), gemcitabine, or any combination thereof.

8. The method of claim 1, wherein the cancer is a hematologic cancer.

9. The method of claim 8, wherein the hematologic cancer is leukemia, lymphoma, myeloma, any metastases thereof, or any combination thereof.

10. The method of claim 8, wherein the hematologic cancer is acute myeloid leukemia (AML), chronic myeloid leukemia (CML), acute lymphocytic leukemia (ALL), small lymphocytic lymphoma (SLL), chronic lymphocytic leukemia, hairy cell leukemia, Hodgkin lymphoma, non-Hodgkin lymphoma, multiple myeloma, any metastases thereof, or any combination thereof.

11. The method of claim 1, wherein the cancer is a solid tumor.

12. The method of claim 11, wherein the cancer is bladder cancer, colorectal cancer, sarcoma, gastric cancer, lung cancer, pancreatic cancer, head and neck cancer, melanoma, ovarian cancer, or breast cancer.

13. The method of claim 12, wherein the cancer is fibrosarcoma, chondrosarcoma, osteosarcoma, non-small cell lung cancer (NSCLC), head and neck sarcoma, or triple negative breast cancer (TNBC).

14. The method of claim 1, wherein the three or four antigen-binding domains or the three to twelve antigen-binding domains of the antibody or multimerized antigen-binding fragment, variant, or derivative thereof comprise a heavy chain variable region (VH) and a light chain variable region (VL), wherein the VH and VL comprise:
(a) six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3 comprise the CDRs of an antibody comprising the VH and VL amino acid sequences SEQ ID NO: 1 and SEQ ID NO: 2; SEQ ID NO: 3 and SEQ ID NO: 4: SEQ ID NO: 5 or SEQ ID NO: 90 and SEQ ID NO: 6; SEQ ID NO: 7 and SEQ ID NO: 8; SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; SEQ ID NO: 23 and SEQ ID NO: 24; SEQ ID NO: 25 and SEQ ID NO: 26; SEQ ID NO: 27 and SEQ ID NO: 28; SEQ ID NO: 29 and SEQ ID NO: 30; SEQ ID NO: 31 and SEQ ID NO: 32; SEQ ID NO: 33 and SEQ ID NO: 34, SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46; SEQ ID NO: 47 and SEQ ID NO: 48; SEQ ID NO: 49 and SEQ ID NO: 50; SEQ ID NO: 51 and SEQ ID NO: 52; SEQ ID NO: 53 and SEQ ID NO: 54; SEQ ID NO: 55 and SEQ ID NO: 56; SEQ ID NO: 82 and SEQ ID NO: 83; SEQ ID NO: 84 and SEQ ID NO: 85; SEQ ID NO: 86 and SEQ ID NO: 87; or SEQ ID NO: 88 and SEQ ID NO: 89; respectively, or the ScFv sequence SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, or SEQ ID NO: 73, or the six CDRs with one or two amino acid substitutions in one or more of the CDRs; and/or
(b) amino acid sequences at least 90% identical to SEQ ID NO: 1 and SEQ ID NO: 2; SEQ ID NO: 3 and SEQ ID NO: 4; SEQ ID NO: 5 or SEQ ID NO: 90 and SEQ ID NO: 6; SEQ ID NO: 7 and SEQ ID NO: 8; SEQ ID NO: 9 and SEQ ID NO: 10; SEQ ID NO: 11 and SEQ ID NO: 12; SEQ ID NO: 13 and SEQ ID NO: 14; SEQ ID NO: 15 and SEQ ID NO: 16; SEQ ID NO: 17 and SEQ ID NO: 18; SEQ ID NO: 19 and SEQ ID NO: 20; SEQ ID NO: 21 and SEQ ID NO: 22; SEQ ID NO: 23 and SEQ ID NO: 24; SEQ ID NO: 25 and SEQ ID NO: 26; SEQ ID NO: 27 and SEQ ID NO: 28; SEQ ID NO: 29 and SEQ ID NO: 30; SEQ ID NO: 31 and SEQ ID NO: 32; SEQ ID NO: 33 and SEQ ID NO: 34; SEQ ID NO: 35 and SEQ ID NO: 36; SEQ ID NO: 37 and SEQ ID NO: 38; SEQ ID NO: 39 and SEQ ID NO: 40; SEQ ID NO: 41 and SEQ ID NO: 42; SEQ ID NO: 43 and SEQ ID NO: 44; SEQ ID NO: 45 and SEQ ID NO: 46; SEQ ID NO: 47 and SEQ ID NO: 48; SEQ ID NO: 49 and SEQ ID NO: 50; SEQ ID NO: 51 and SEQ ID NO: 52; SEQ ID NO: 53 and SEQ ID NO: 54; SEQ ID NO: 55 and SEQ ID NO: 56; SEQ ID NO: 82 and SEQ ID NO: 83; SEQ ID NO: 84 and SEQ ID NO: 85; SEQ ID NO: 86 and SEQ ID NO: 87; or SEQ ID NO: 88 and SEQ ID NO: 89; respectively, or wherein the VH and VL are contained in an ScFv with an amino acid sequence at least 90% identical to SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, or SEQ ID NO: 73, respectively.

15. The method of claim 14, wherein the three or four antigen-binding domains or the three to twelve antigen-binding domains of the antibody or multimerized antigen-binding fragment, variant, or derivative thereof comprise a heavy chain variable region (VH) and a light chain variable region (VL), wherein the VH and VL comprise:
(a) six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3 comprise the CDRs of an antibody comprising the VH and VL amino acid sequences SEQ ID NO: 5 or SEQ ID NO: 90 and SEQ ID NO: 6; or SEQ ID NO: 7 and SEQ ID NO: 8, respectively; and/or
(b) amino acid sequences at least 90% identical to SEQ ID NO: 5 or SEQ ID NO: 90 and SEQ ID NO: 6; or SEQ ID NO: 7 and SEQ ID NO: 8, respectively.

16. The method of claim 15, wherein the three or four antigen-binding domains or the three to twelve antigen-binding domains of the antibody or multimerized antigen-binding fragment, variant, or derivative thereof comprise a heavy chain variable region (VH) and a light chain variable region (VL), wherein the VH and VL comprise:
(a) six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3 comprise the CDRs of an antibody comprising the VH and VL amino acid sequences SEQ ID NO: 7 and SEQ ID NO: 8, respectively; and/or
(b) amino acid sequences at least 90% identical to SEQ ID NO: 7 and SEQ ID NO: 8, respectively.

17. The method of any one of claims 1 to 16, wherein the antibody or multimerized antigen-binding fragment, variant, or derivative thereof is a dimeric IgA or IgA-like antibody comprising two bivalent IgA binding units or multimerizing fragments thereof and a J-chain or fragment or variant thereof, wherein each binding unit comprises two IgA heavy chain constant regions or multimerizing fragments thereof each associated with an antigen-binding domain, and wherein the IgA heavy chain constant regions or multimerizing fragments thereof each comprise a Cα3-tp domain.

18. The method of claim 17, wherein the IgA heavy chain constant regions or multimerizing fragments thereof each comprise a Cα1 domain and/or a Cα2 domain.

19. The method of claim 17, wherein the IgA heavy chain constant region is a human IgA constant region.

20. The method of claim 17, wherein each binding unit comprises two IgA heavy chains each comprising a VH situated amino terminal to the IgA constant region or multimerizing fragment thereof, and two immunoglobulin light chains each comprising a VL situated amino terminal to an immunoglobulin light chain constant region.

21. The method of any one of claims 1 to 16, wherein the antibody or multimerized antigen-binding fragment, variant, or derivative thereof is a pentameric or a hexameric IgM antibody comprising five or six bivalent IgM binding units, respectively, wherein each binding unit comprises two IgM heavy chain constant regions or multimerizing fragments thereof each associated with an antigen-binding domain, and wherein the IgM heavy chain constant regions or multimerizing fragments thereof each comprise a Cμ4-tp domain.

22. The method of claim 21, wherein the IgM heavy chain constant regions or multimerizing fragments thereof each comprise a Cμ 1 domain, a Cμ2 domain, and/or a Cμ3 domain.

23. The method of claim 21, wherein the antibody or multimerized antigen-binding fragment, variant, or derivative thereof is pentameric, and further comprises a J-chain, or functional fragment thereof, or variant thereof.

24. The method of claim 21, wherein the IgM heavy chain constant region is a human IgM constant region.

25. The method of claim 21, wherein each binding unit comprises two IgM heavy chains each comprising a VH situated amino terminal to the IgM constant region or multimerizing fragment thereof, and two immunoglobulin light chains each comprising a VL situated amino terminal to an immunoglobulin light chain constant region.

26. The method of claim 23, wherein the J-chain or functional fragment or variant thereof is a variant J-chain comprising one or more single amino acid substitutions, deletions, or insertions relative to a wild-type J-chain that can affect serum half-life of the multimeric binding molecule; and wherein the multimeric binding molecule exhibits an increased serum half-life upon administration to an animal relative to a reference multimeric binding molecule that is identical except for the one or more single amino acid substitutions, deletions, or insertions, and is administered in the same way to the same animal species.

27. The method of claim 26, wherein the J-chain or functional fragment thereof comprises:
(a) an amino acid substitution at the amino acid position corresponding to amino acid Y102 of the wild-type human J-chain (SEQ ID NO: 97),
(b) an alanine (a) substitution at the amino acid position corresponding to amino acid Y102 of the wild-type human J-chain (SEQ ID NO: 97), or
(c) the amino acid sequence SEQ ID NO: 98.

28. The method of claim 26, wherein the J-chain or functional fragment or variant thereof further comprises a heterologous polypeptide, wherein the heterologous polypeptide is directly or indirectly fused to the J-chain or functional fragment or variant thereof.

29. The method of any one of claims 1 to 16, wherein administration of the combination therapy results in enhanced therapeutic efficacy relative to administration of the antibody or multimerized antigen-binding fragment, variant, or derivative thereof or the cancer therapy alone.

30. The method of claim 29, wherein the enhanced therapeutic efficacy comprises a reduced tumor growth rate, tumor regression, or increased survival.

31. The method of any one of claims 1 to 16, wherein the subject is human.


SEQUENCE LISTING (103 sequences)

SEQ ID NO: 1 - protein, 121 aa, Artificial Sequence
  Description of Artificial Sequence: Synthetic polypeptide
EVQLVQSGGGVERPGGSLRLSCAASGFTFDDYGMSWVRQAPGKGLEWVSGINWNGGSTGYADSVKGRVTISRDNAKNSLYLQMNSLRAEDTAVYYCAKILGAGRGWYFDLWGKGTTVTVSS

SEQ ID NO: 2 - protein, 108 aa, Artificial Sequence
  Description of Artificial Sequence: Synthetic polypeptide
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 3 - protein, 121 aa, Artificial Sequence [sequence omitted]

SEQ ID NO: 4 - protein, 107 aa, Artificial Sequence [sequence omitted]

SEQ ID NO: 5 - protein, 122 aa, Artificial Sequence
  Description of Artificial Sequence: Synthetic polypeptide
QVQLQESGPGLVKPSQTLSLTCTVSGGSISSGDYFWSWIRQLPGKGLECIGHIHNSGTTYYNPSLKSRVTISVDTSKKQFSLRLSSVTAADTAVYYCARDRGGDYYYGMDVWGQGTTVTVSS

SEQ ID NO: 6 - protein, 108 aa, Artificial Sequence
  Description of Artificial Sequence: Synthetic polypeptide
EIVLTQSPGTLSLSPGERATLSCRASQGISRSYLAWYQQKPGQAPSLLIYGASSRATGIPDRFSGSGSGTDFTLTISRLEPEDFAVYYCQQFGSSPWTFGQGTKVEIK

SEQ ID NO: 7 - protein, 119 aa, Artificial Sequence [sequence omitted]

SEQ ID NO: 8 - protein, 106 aa, Artificial Sequence [sequence omitted]

SEQ ID NO: 9 - protein, 112 aa, Artificial Sequence
  Description of Artificial Sequence: Synthetic polypeptide
QIQLVQSGPELKKPGETVKISCKASGYTFTDFSMNWVKQAPGKGLKWMGWINTETGEPTYADDFKGRFALSMETSASTAYLQINNLKNEDTATYFCVRIDYWGQGTTLTVSS

SEQ ID NO: 10 - protein, 113 aa, Artificial Sequence
  Description of Artificial Sequence: Synthetic polypeptide
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCFQSTHVPHTFGGGTKLEIKR

SEQ ID NO: 11 - protein, 145 aa, Artificial Sequence
  Description of Artificial Sequence: Synthetic polypeptide
MDWTWRILFLVAAATSAHSQVQLVQSGAEMKKPGASVKVSCKTSGYTFTNYKINWVRQAPGQGLEWMGWMNPDTDSTGYPQKFQGRVTMTRNTSISTAYMELSSLRSEDTAVYYCARSYGSGSYYRDYYYGMDVWGQGTTVTVSS

SEQ ID NO: 12 - protein, 128 aa, Artificial Sequence
  Description of Artificial Sequence: Synthetic polypeptide
MEAPAQLLFLLLLWLPDTTGEIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIKR

SEQ ID NO: 13 - protein, 122 aa, Artificial Sequence
  Description of Artificial Sequence: Synthetic polypeptide
EVQLQQSGPELVKPGASVKISCKASGYSFIGYFMNWMKQSHGKSLEWIGRFNPYNGDTFYNQKFKGKATLTVDKSSTTAHMELLSLTSEDSAVYFCGRSAYYFDSGGYFDYWGQGTTLTVSS

SEQ ID NO: 14 - protein, 112 aa, Artificial Sequence
  Description of Artificial Sequence: Synthetic polypeptide
DVVMTQTPLSLPVSLGDQASISCRSSQSLVHSNGNTYLHWYLQKPGQSPKLLIYKVSNRFSGVPDRFSGSGSGTDFTLKISRVEAEDLGIYFCSQSTHVPWTFGGGTKLEIK

SEQ ID NO: 15 - protein, 112 aa, Artificial Sequence
  Description of Artificial Sequence: Synthetic polypeptide
QVQLVQSGSELKKPGASVKVSCKASGYTFTDFSMNWVRQAPGQGLEWMGWINTETGEPTYADDFKGRFVFSLDTSVSTAYLQISSLKAEDTAVYYCARIDYWGQGTTVTVSS

SEQ ID NO: 16 - protein, 113 aa, Artificial Sequence
  Description of Artificial Sequence: Synthetic polypeptide
DIVMTQSPLSLPVTPGEPASISCRSSQSLVHSNGNTYLHWYLQKPGQSPQLLIYKVSNRFSGVPDRFSGSGSGTDFTLKISRVEAEDVGVYYCFQSTHVPHTFGQGTKLEIKR

SEQ ID NO: 17 - protein, 124 aa, Artificial Sequence
  Description of Artificial Sequence: Synthetic polypeptide
GVQCEVHLVESGGGLVRPGGSLKLSCAASGFAFSSYDMSWVRQTPEKRLEWVAYISDGGGITYYPDTMKGRFTISRDNAKNTLSLQMSSLKSEDTAMYYCARHITMVVGPFAYWGQGTLVTVSA

SEQ ID NO: 18 - protein, 108 aa, Artificial Sequence
  Description of Artificial Sequence: Synthetic polypeptide
DIQMTQSSSSFSVSLGDRVTITCKASEDIYNRLAWYQQKPGNAPRLLISGATSLETGVPSRFSGSGSGKDYTLSITSLQTEDVATYYCQQYWSTPLTFGAGTKLELKR

SEQ ID NO: 19 - protein, 117 aa, Artificial Sequence
  Description of Artificial Sequence: Synthetic polypeptide
EVQLQQSGPELVKPGASVRMSCKASGYTFTSYFIHWVKQRPGQGLEWIGWIYPGNVNTKYSEKFKGKATLTADKSSSTAYMQFSSLTSEDSAVYFCARGEAGYFDYWGQGTTLTVSS

SEQ ID NO: 20 - protein, 107 aa, Artificial Sequence
  Description of Artificial Sequence: Synthetic polypeptide
DIVMTQSHKFMSTSVGDRVSITCKASQDVSTAVAWYQQKPGQSPRLLIYWASTRHTGVPDRFTGSGSGTDYTLTISSVQAEDQALYYCQQHYRTPWTFGGGTKLEIK

SEQ ID NO: 21 - protein, 121 aa, Artificial Sequence
  Description of Artificial Sequence: Synthetic polypeptide
QVQLVQSGAEVKKPGASVKVSCKASGYTFTSYDINWVRQATGQGLEWMGWMNPNSDNTGYAQKFQGRVTMTRNTSISTAYMELSSLRSEDTAVYYCARWNHYGSGSHFDYWGQGTLVTVSS

SEQ ID NO: 22 - protein, 107 aa, Artificial Sequence
  Description of Artificial Sequence: Synthetic polypeptide
DIQMTQSPSSLSASVGDRVTITCRASQSISIYLNWYQQKPGKAPKLLIYAASSLQSGVPLRFSGSGSGTDFTLTISSLQPEDIATYYCQQSYKTPLTFGGGTKVEIK

SEQ ID NO: 23 - protein, 120 aa, Artificial Sequence
  Description of Artificial Sequence: Synthetic polypeptide
QVQLQESGPGLVKPSQTLSLTCTVSGGSISSGGHYWSWIRQHPGKGLEWIGYIYYSGSTYYNPSLKSRVTISVDTSKNQFSLKLSSVTAADTAVYYCARDDSSGWGFDYWGQGILVTVSS

SEQ ID NO: 24 - protein, 107 aa, Artificial Sequence
  Description of Artificial Sequence: Synthetic polypeptide
DIQMTQSPSSLSASVGDRVTITCRASQGLRNDLGWFQQKPGKVTKRLIYAASSLQRGVPSRFSGSGSGTEFTLTISSLQPEDFATYYCLQHYSFPWTFGQGTKVEIK

SEQ ID NO: 25 - protein, 120 aa, Artificial Sequence
  Description of Artificial Sequence: Synthetic polypeptide
QVQLQESGPGLVKPSQTLSLTCTVSGGSISSGGHYWSWIRQHPGKGLEWIGYIYYSGSAYYNPSLKSRVTISVDTSKNQFSLKLSSVTAADTAVYYCARDDSSGWGFDYWGQGILVTVSS

SEQ ID NO: 26 - protein, 107 aa, Artificial Sequence
  Description of Artificial Sequence: Synthetic polypeptide
DIQMTQSPSSLSASVGDRVTITCRASQGLRNDLGWFQQKPGKAPKRLIYAASSLQRGVPSRFSGSGSGTEFTLTISSLQPEDFTTYFCLQHNSFPWTFGQGTKVEIK

SEQ ID NO: 27 - protein, 120 aa, Artificial Sequence; identical to SEQ ID NO: 25
  Description of Artificial Sequence: Synthetic polypeptide

SEQ ID NO: 28 - protein, 107 aa, Artificial Sequence; identical to SEQ ID NO: 26
  Description of Artificial Sequence: Synthetic polypeptide

SEQ ID NO: 29 - protein, 121 aa, Artificial Sequence
  Description of Artificial Sequence: Synthetic polypeptide
QVQLVESGGGLVKPGGSLRLSCAASGFTFSDYYMNWIRQAPGKGLEWVSHISSSGSILDYADSVKGRFTISRDNAKNSLYLQMNSLRVEDTAVYYCARDGAAAGTDAFDLWGQGTMVTVSS

SEQ ID NO: 30 - protein, 107 aa, Artificial Sequence
  Description of Artificial Sequence: Synthetic polypeptide
DIQMTQSPSSLSASVGDRVTITCRSSQSISNYINWYQQRPGKAPNLLIHDVSSFQSAVPSRFSRSGSGTVFTLTISSLQPEDFATYFCQQTYITPFTFGPGTKVDIK

SEQ ID NO: 31 - protein, 122 aa, Artificial Sequence
  Description of Artificial Sequence: Synthetic polypeptide
QVQLVESGGGVVQPGRSLRLSCAASGFTFSYYGIHWVRQAPGKGLEWVAVIWYDGSNKYYADSVKGRFTISRDNSKNTLYLQMNSLRAEDTAVYYCARGRYSSSSWWYFDLWGRGTLVTVSS

SEQ ID NO: 32 - protein, 107 aa, Artificial Sequence
  Description of Artificial Sequence: Synthetic polypeptide
DIQMTQSPSSLSASVGDRVTITCRASQGISNYLAWYQQKPGKVPKLLIYAASTLQSGVPSRFSGSGSGTDFTLTISSLQPEDVATYYCQKYNSAPLTFGGGTKVEIK

SEQ ID NO: 33 - protein, 122 aa, Artificial Sequence
  Description of Artificial Sequence: Synthetic polypeptide
QVQAEQSGPGLVKPSETLSLTCTVSGGSISNYYWSWIRQPPGKGLEWIGYIYYSGSTKYNPSLKSRVTISVDTSKNQFSLKLTSVTTADTAVYYCARDSPRGFSGYEAFDSWGQGTLVTVSS

SEQ ID NO: 34 - protein, 113 aa, Artificial Sequence
  Description of Artificial Sequence: Synthetic polypeptide
DIVMTQSPDSLAVSLGERATINCKSSQSVLYRSNNKIYLAWYQQKPGQPPKLLIYWASTRESGVPDRFSGSGSGTDFTLTISSLLAEDVAVYYCQQYYSTPFTFGPGTKVDIK

SEQ ID NO: 35 - protein, 120 aa, Artificial Sequence
  Description of Artificial Sequence: Synthetic polypeptide
QVQLQESGPGLVKPSQTLSLTCTVSGGSISSDNYYWSWIRQHPGKGLEWIGYIYYSGSTYYNPSLKSRVTISVDTSKNQFSLKLSSVTAADTAVYYCARGVNWNFLFDIWGQGTMVTVSS

SEQ ID NO: 36 - protein, 112 aa, Artificial Sequence
  Description of Artificial Sequence: Synthetic polypeptide
DIVMTQSPLSLPVTPGEPASISCRSSQSLLRRNGYNYLDWYLQKPGQSPQLLIYLGSNRASGVPDRFSGSGSGTDFTLKISRVEAEDVGVYYCMQALQTPLTFGGGTEVEIK

SEQ ID NO: 37 - protein, 116 aa, Artificial Sequence
  Description of Artificial Sequence: Synthetic polypeptide
QVQLVESGGGLVKPGGSLRLSCAASGFTFSDYYMSWIRQAPGKGLEWVSYISRSGSTIYYADSVKGRFTISRDNAKNSLYLQMNSLRAEDTAVYYCARSLGGMDVWGQGTTVTVSS

SEQ ID NO: 38 - protein, 113 aa, Artificial Sequence
  Description of Artificial Sequence: Synthetic polypeptide
DIVMTQFPDSLAVSLGERATINCKSSQSVLHSSNNKNYLTWYQLKPGQPPKLLIYWASTRESGVPDRFSGSGSGTDFTLTISSLQAEDVAVYYCHQYYSTPSSFGQGTKLEIK

SEQ ID NO: 39 - protein, 129 aa, Artificial Sequence
  Description of Artificial Sequence: Synthetic polypeptide
QVQLVESGGGVVQPGRSLRLSCAASGFTFNNYGMHWVRQAPGKGLEWVAVIWYDGSNKYYADSVKGRFTISRDNSKNTLYLQMNSLRAEDTAVYYCARDRTVYSNSSPFYYYYYGMDVWGQGTTVTVSS

SEQ ID NO: 40 - protein, 107 aa, Artificial Sequence
  Description of Artificial Sequence: Synthetic polypeptide
DIQMTQSPSSLSASVGDRVTITCRTSQSISTYLNWYQQKPGKAPKLLISATSSLQSGVPSRFSGSGSGTDFTLTISSLQPEDFATYYCQQSYSTPLTFGGGTKVEIK

SEQ ID NO: 41 - protein, 129 aa, Artificial Sequence
  Description of Artificial Sequence: Synthetic polypeptide
QVQLVESGGGVVQPGRSLRLSCAASGFTFSTYGMHWVRQAPGKGLEWVAVIWYDGSNKYYADSVKGRFTISRDNSKNTLYLQMNSLRAEDTAVYYCARDRTVYSSSSPFYYYYYGMDVWGQGTTVTVSS

SEQ ID NO: 42 - protein, 107 aa, Artificial Sequence
  Description of Artificial Sequence: Synthetic polypeptide
DIQMTQSPSSLSASVGDRVTITCRASQSISSYLNWYQQKPGKAPKLLISATSSFQSGVPSRFSGSGSGTDFTLTISSLQPEDFAAYYCQQSYSTPLTFGGGTKVEIK

SEQ ID NO: 43 - protein, 119 aa, Artificial Sequence
  Description of Artificial Sequence: Synthetic polypeptide
QVQLQQWGARLLKPSETLSLTCAVYGGSFSGYYWSWIRQPPGKGLEWIGEINHSGSTNYNPSLKSRVTISVDTSKNQFSLKLRSVTAADTAVYYCARGGSSGYWYFDLWGRGTLVTVSS

SEQ ID NO: 44 - protein, 113 aa, Artificial Sequence
  Description of Artificial Sequence: Synthetic polypeptide
DIVMTQSPDSLAVSLGERATINCKSSQSVLHSSNNKNYLVWYQQKPGQPPKLLIYWASTRESGVPDRFSGSGSGTDFTLTISSLQAEDVAVYYCQQYYSTPLTFGGGTKVEIK

SEQ ID NO: 45 - protein, 121 aa, Artificial Sequence
  Description of Artificial Sequence: Synthetic polypeptide
EVQVVESGGGLVKPGGSLRLSCAASGFTFSSYSMNWVRQAPGKGLEWVSSISSSSSYIYYADSVKGRFTISRDNAKNSLYLQMNSLRAEDTAVYYCARGGSSWYGDWFDPWGQGTLVTVSS

SEQ ID NO: 46 - protein, 107 aa, Artificial Sequence
  Description of Artificial Sequence: Synthetic polypeptide
DIQMTQSPSSVSASVGDRVTITCRASQGISSWLVWYQQKPGKAPKLLIYAASSLQSGVPSRFSGSGSGTDFTLTISSLQPEDFATYYCQQANSFPFTFGGGTKVEIK

SEQ ID NO: 47 - protein, 126 aa, Artificial Sequence
  Description of Artificial Sequence: Synthetic polypeptide
QLVESGGGVVQPGRSLRLSCAASGFTFSSYGMHWVRQAPGKGLEWVAVIWYDGRNKYYADSVKGRFTISRDNSKNTLYLQMNSLRAEDTAVYYCAREVGYCTNGVCSYYYYGMDVWGQGTTVTVSS

SEQ ID NO: 48 - protein, 107 aa, Artificial Sequence
  Description of Artificial Sequence: Synthetic polypeptide
DIQMTQSPSSLSASVGDRVTITCRASQGISNYLAWFQQKPGKAPKSLIYAASSLQSGVPSKFSGSGSGTDFTLTISSLQPEDFATYYCQQYNSYPLTFGGGTKVEIK

SEQ ID NO: 49 - protein, 122 aa, Artificial Sequence
  Description of Artificial Sequence: Synthetic polypeptide
QVQLQESGPGLVKPSQTLSLTCSVSGGSISSGGYYWSWIRQHPGKGLEWIGYIYYSGSTYCNPSLKSRVTISVDTSKNQFSLKLSSVTAADTAVYYCARDNGSGSYDWFDPWGQGILVTVSS

SEQ ID NO: 50 - protein, 107 aa, Artificial Sequence
  Description of Artificial Sequence: Synthetic polypeptide
DIQMTQSPSSVSASVGDRVTITCRASQGISSWLAWYQQKPGKAPKFLIFVASSFQSGVPSRFSGSGSGTDFTLTISSLQPEDFATYYCQQANSFPRTFGQGTKVEIK

SEQ ID NO: 51 - protein, 122 aa, Artificial Sequence
  Description of Artificial Sequence: Synthetic polypeptide
QVQMQESGPGLVKPSQTLSLTCTVSGGSISSGDYYWSWIRQHPGKNLEWIGYIYYSGSTYYNPSLKSRVTISVDTSKNQFSLKLSSVTAADTAVYYCARDNGSGSYDWFDPWGQGTLVTVSS

SEQ ID NO: 52 - protein, 107 aa, Artificial Sequence
  Description of Artificial Sequence: Synthetic polypeptide
DIQMTQSPSSVSASVGDRVTITCRASQGISSWLAWYQQKPGKAPKFLIFVASSLQSGVPSRFSGSGSGTDFTLTISSLQPEDFATYYCQQANSFPRTFGQGTKVEIK

SEQ ID NO: 53 - protein, 118 aa, Artificial Sequence
  Description of Artificial Sequence: Synthetic polypeptide
KVQLQQSGAELVKPGASVKLSCKASGYTFTDYTIHWVKQRSGQGLEWIGWFYPGGGYIKYNEKFKDRATLTADKSSNTVYMELSRLTSEGSAVYFCARHEEGIYFDYWGQGTTLTVSS

SEQ ID NO: 54 - protein, 109 aa, Artificial Sequence
  Description of Artificial Sequence: Synthetic polypeptide
DIAMTQSHKFMSTLVGDRVSITCKASQDVNTAIAWYQQKPGQSPKLLIYWASTRHTGVPDRFTGSGSGTDYTLTISSMEAEDAATYYCQQWSSNPLTFGAGTKLELKRA

SEQ ID NO: 55 - protein, 118 aa, Artificial Sequence
  Description of Artificial Sequence: Synthetic polypeptide
KVQLQQSGAELVKPGASVKLSCKASGYTFTDYTIHWVKQRSGQGLEWIGWFYPGGGYIKYNEKFKDRATLTADKSSNTVYMELSRLTSEDSAVYFCARHEEGIYFDYWGQGTTLTVSS

SEQ ID NO: 56 - protein, 104 aa, Artificial Sequence
  Description of Artificial Sequence: Synthetic polypeptide
DIVMTQSHKFMSTSVGDRVSITCKASQDVNTAIAWYQQKPGQSPKLLIYWASTRHTGVPDRFTGSGSGTDYTLTISSVQAEDLALYYCQQHYTTPFTFGSGTKL

SEQ ID NO: 57 - protein, 244 aa, Artificial Sequence
  Description of Artificial Sequence: Synthetic polypeptide
EVQLVQSGGGVERPGGSLRLSCAASGFTFDDYGMSWVRQAPGKGLEWVSGINWNGGSTGYADSVKGRVTISRDNAKNSLYLQMNSLRAEDTAVYYCAKILGAGRGWYFDLWGKGTTVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLG

SEQ ID NO: 58 - protein, 245 aa, Artificial Sequence
  Description of Artificial Sequence: Synthetic polypeptide
EVQLVETGGGLVQPGGSLRLSCAASGFTFSSYAMSWVRQAPGKGLEWVSAISGSGGSTYYADSVKGRFTISRDNSKNTLYLQMNSLRAEDTAVYHCARGGYSSSRSAAYDIWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLG

SEQ ID NO: 59 - protein, 246 aa, Artificial Sequence
  Description of Artificial Sequence: Synthetic polypeptide
QVQLVQSGAEVKKPGASVKISCEGSGYTFNSYTLHWLRQAPGQRLEWMGRINAGNGNTKYSQNFQGRLSITRDTSATTAYMELSSLRSEDTGVYYCARVFTYSFGMDVWGRGTLVTVSSGGGGSGGGGSGGGGSAQSVLTQPPSASGTPGQRVTISCSGGGSNIGRNSVSWYQQLPGTAPKLILYSNNQRPSGVPDRFSGSKSGTSASLAISGLRSEDEALYYCAAWDDSLSGGVFGGGTKLTVLG

SEQ ID NO: 60 - protein, 244 aa, Artificial Sequence
  Description of Artificial Sequence: Synthetic polypeptide
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKVHRPGRSGYFDYWGRGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLG

SEQ ID NO: 61 - protein, 235 aa, Artificial Sequence
  Description of Artificial Sequence: Synthetic polypeptide
QVQLQQSGAEVKKPGASVRVSCQASGYSLSEYYIHWVRQAPGQGLEWMGWLNPNSGVTDYAQKFQGRVSMTRDTSISTAYMELSSLTFNDTAVYFCARGNGDYWGKGTLVTVSPGGGGSGGGGSGGGGSSELTQDPAVSVALGQTVRITCQGDSLRSYYTNWFQQKPGQAPLLVVYAKNKRPSGIPDRFSGSSSGNTASLTITGAQAEDEADYYCHSRDSSGWVFGGGTKLTVLG

SEQ ID NO: 62 - protein, 245 aa, Artificial Sequence
  Description of Artificial Sequence: Synthetic polypeptide
QVQLVQSGGGVVQPGRSLRLSCAASGFTFSPDAMHWVRQAPGKGLEWMGVISFDGSQTFYADSVKGRFTISRDNSQNTLYLQMNSLRSDDTAVYYCARAPARFFPLHFDIWGRGTMVTVSSGGGGSGGGGSGGGGSALSSELTQDPAVSVALGQTVRITCQGDSLRTHYASWYHQRPGRAPVLVNYPKDSRPSGIPDRFSGSSSGNTASLTIIGAQAADEGDYYCQSRDSSGVLFGGGTKVTVLG

SEQ ID NO: 63 - protein, 247 aa, Artificial Sequence
  Description of Artificial Sequence: Synthetic polypeptide
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARDFSGYGDYLDYWGKGTLVTVSSGGGGSGGGGSGGGGSAQSALTQPPSASGSPGQSVTISCTGTSSDIGNYNYVSWYQQHPGKAPKLMIYEVNERPSGVPDRFSGSKSGNTASLTVSGLRPEDEADYYCSSYAGNNAVIFGGGTQLTVLG

SEQ ID NO: 64 - protein, 255 aa, Artificial Sequence
  Description of Artificial Sequence: Synthetic polypeptide
QVQLVQSGAEVKKPGASVKVSCKASGYTFTTHAMHWVRQAPGQSLEWMGWINTGNGNTKYSQSFQGRVSITRDTSANTAYMELSSLKSEDTAMYYCARASRDSSGYYYVPPGDFFDIWGQGTLVTVSSGGGGSGGGGSGGGGSAQSALTQPASVSGSPGQSITISCTGSRSDIGGYNFVSWYQQHPGKAPKLLIYDVYNRPSGISDHFSGSKSDNTASLTISGLQSEDDADYYCSSYAGYHTWIFGGGTKVTVLG

SEQ ID NO: 65 - protein, 248 aa, Artificial Sequence
  Description of Artificial Sequence: Synthetic polypeptide
EVQLVQSGAEVKKPGASVKLSCKASGYTLVNYFMHWVRQAPGQGPEWMGMINPSGGTTKNRQKFQDRVTMTRDTSTRTVYMELSGLTSEDTAVYYCATDFKGTDILFRDWGRGTLVTVSSGGGGSGGGGSGGGGSAQSVLTQPPSASGTPGQRVSISCSGSSSNIGSNTVIWYQQLPGTAPKLLMYSNDRRPSGVPDRFSGSKSGTSASLAISGLQSEDEADYYCATWDDSLNGHYVFGTGTKLTVLG

SEQ ID NO: 66 - protein, 243 aa, Artificial Sequence
  Description of Artificial Sequence: Synthetic polypeptide
QMQLVQSGGGLVKPGGSLRLSCAASGFTFSDYYMSWIRQAPGKGLEWVSAISGSGGSTYYADSVKGRFTISRDNSKNTLYLQMNSLRAEDTAVYYCARGGSTFDIWGRGTMVTVSSGGGGSGGGGSGGGGSAQPVLTQPPSASGTPGQRVTISCSGSNSNIGSRPVNWYQQLPGTAPKLLIQGNNQRPSGVPDRFSGSKSGTSASLAISGLQSEDEADYYCAAWDDSLTGYVFGPGTKLTVLG

SEQ ID NO: 67 - protein, 240 aa, Artificial Sequence
  Description of Artificial Sequence: Synthetic polypeptide
QMQLVQSGGAVVQPGRSLRLSCAASGFTFSSYGMHWVRQAPGKGLEWVAVISYDGSIKYYADSVKGRFTISRDNSKNTLYLQMNSLRAEDTAVYYCARERLRGLDPWGQGTMVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLG

SEQ ID NO: 68 - protein, 243 aa, Artificial Sequence
  Description of Artificial Sequence: Synthetic polypeptide
EVQLVETGGGLVQPGGSLRLSCAASGFTFSPYYMSWVRQAPGKGLEWVSAISGSGGSIYYADSVKGRFTISRDNSKNTLYLQMNSLRAEDTALYYCARGASGPDYWGRGTMVTVSSGGGGSGGGGSGGGGSAQSVLTQPPSVSAAPGQKVTISCSGSTSNIGNNYVSWYQQVPGTAPKLLIYDNNKRPSGIPDRFSGSKSGTSATLGITGLQTGDEADYYCGTWDSSLSALVFGGGTKVTVLG

SEQ ID NO: 69 - protein, 253 aa, Artificial Sequence
  Description of Artificial Sequence: Synthetic polypeptide
QVQLQQSGAEVKTPGSSVKVSCKASGGTFRNNAISWVRQAPGQGLEWMGGFIPKFGTTNHAQKFQGRVTMTADDSTNTVYMELSSLRSEDTAVYYCARGGAYCGGGRCYLYGMDVWGQGTLVTVSSGGGGSGGGGSGGGGSAQAVVIQEPSLTVSPGGTVTLTCGSSTGAVTSGHYPYWFQQKPGQAPRTLIYDTSNKRSWTPARFSGSLLGGKAALTLSGAQPEDEAEYYCLVSYSGSLVVFGGGTKLTVLG

SEQ ID NO: 70 - protein, 243 aa, Artificial Sequence
  Description of Artificial Sequence: Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCVKGAWLDYWGRGTMVTVSSGGGGSGGGGSGGGGSALNFMLTQPHSVSESPGKTVTISCTGSSGSVARNYVQWYQQRPGSAPTIVIYEDNRRPSGVPGRFSGSIDRSSNSASLTISGLQTEDEADYYCQSYNYNTWVFGGGTKLTVLG

SEQ ID NO: 71 - protein, 247 aa, Artificial Sequence
  Description of Artificial Sequence: Synthetic polypeptide
EVQLVQSGAEVKKPGASVKVSCRASGYTFTSYGITWVRQAPGQGLEWMGWISAYNGKTNYVQELQGRVTMTTDTSTSTVYMELTSLRSDDTAVYYCARRGNNYRFGYFDFWGQGTLVTVSSGGGGSGGGGSGGGGSALETTLTQSPGTLSLSPGERATLSCRASQSISSSNLAWYQQKPGRAPRLLIYGASSRAIGIPDRFSGSGSGTDFTLTISRLEAEDFAVYYCQQYGSSPITFGQGTRLEIKR

SEQ ID NO: 72 - protein, 248 aa, Artificial Sequence
  Description of Artificial Sequence: Synthetic polypeptide
QVQLQQSGPGLVKPSQTLSLTCAISGDSVSSTTVAWDWIRQSPSRGLEWLGRTYYRSKWYNEYAVSVKSRITINVDTSKNQISLQLNSVTPEDTAVYYCAREPDAGRGAFDIWGQGTTVTSPLRWGRFGWRGLGRGWLRSPVTQSPGTLSLSPGERATLSCRASQSVSSSHLAWYQQKPGQAPRLLIYGASSRATGIPDRFSGSGSGTDFTLTISSLEPEDFAVYYCQQRSNWPPRAVFGQGTRLEIK

SEQ ID NO: 73 - protein, 248 aa, Artificial Sequence
  Description of Artificial Sequence: Synthetic polypeptide
QVQLQQSGPGRVQPSQTLSLTCAISGDSVSNNNAAWYWIRQSPSRGLEWLGRTYYRSKWYNDYAVSVKSRITISPDTSKNQFSLQLNSVTPEDTAVYYCARRGDGNSYFDYWGQGTLVTVSSGILRWGRFGWRGLGRGWLEIVLTQSPGTLSLSPGERATLSCRASQSVSSGYVSWYRQKPGQAPRLLIYGASTRATGIPDRFSGSGSGTDFTLTISRLEPEDFAVYYCHQYGSSPNTYGQGTKVGIK

SEQ ID NO: 74-81 - skipped (8 entries)

SEQ ID NO: 82 - protein, 146 aa, Artificial Sequence
  Description of Artificial Sequence: Synthetic polypeptide
MDLMCKKMKHLWFFLLLVAAPRWVLSQLQLQESGPGLVKPSETLSLTCTVSGGSIISKSSYWGWIRQPPGKGLEWIGSIYYSGSTFYNPSLKSRVTISVDTSKNQFSLKLSSVTAADTAVYYCARLTVAEFDYWGQGTLVTVSSAS

SEQ ID NO: 83 - protein, 129 aa, Artificial Sequence
  Description of Artificial Sequence: Synthetic polypeptide
MEAPAQLLFLLLLWLPDTTGEIVLTQSPATLSLSPGERATLSCRASQSVSSFLAWYQQKPGQAPRLLIYDASNRATGIPARFSGSGSGTDFTLTISSLEPEDFAVYYCQQRSNWPLTFGPGTKVDIKRT

SEQ ID NO: 84 - protein, 146 aa, Artificial Sequence
  Description of Artificial Sequence: Synthetic polypeptide
MDLMCKKMKHLWFFLLLVAAPRWVLSQLQLQESGPGLVKPSETLSLTCTVSGGSISSRSNYWGWIRQPPGKGLEWIGNVYYRGSTYYNSSLKSRVTISVDTSKNQFSLKLSSVTVADTAVYYCARLSVAEFDYWGQGILVTVSSAS

SEQ ID NO: 85 - protein, 129 aa, Artificial Sequence
  Description of Artificial Sequence: Synthetic polypeptide
MEAPAQLLFLLLLWLPDTTGEIVLTQSPATLSLSPGERATLSCRASQSVSSFLAWYQQKPGQAPRLLIYDASNRATGSPARFSGSGSGTDFTLTISSLEPEDFAVYYCQQRSDWPLTFGPGTKVDIKRT

SEQ ID NO: 86 - protein, 154 aa, Artificial Sequence
  Description of Artificial Sequence: Synthetic polypeptide
MDLMCKKMKHLWFFLLLVAAPRWVLSQLQLQESGPGLVKPSETLSLTCTVSGGSISSSSYYWGWVRQPPGKGLEWIGSIHYSGSTFYNPSLKSRVTISVDTSKNQFSLKLSSVTAADTTVYYCARQGSTVVRGVYYYGMDVWGQGTTVTVSSAS

SEQ ID NO: 87 - protein, 131 aa, Artificial Sequence
  Description of Artificial Sequence: Synthetic polypeptide
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLYTFGQGTKLEIKRT

SEQ ID NO: 88 - protein, 120 aa, Artificial Sequence
  Description of Artificial Sequence: Synthetic polypeptide
EVQLLESGGGLVQPGRSLRLSCAASGFTFSSYAMSWVRQAPGKGLEWVSAISGSGGSRYYADSVKGRFTISRDNSKNTLYLQMNSLRAEDTAVYYCAKESSGWFGAFDYWGQGTLVTVSS

SEQ ID NO: 89 - protein, 108 aa, Artificial Sequence
  Description of Artificial Sequence: Synthetic polypeptide
EIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPITFGQGTRLEIKR

SEQ ID NO: 90 - protein, 121 aa, Artificial Sequence
  Description of Artificial Sequence: Synthetic polypeptide
QVQLQESGPGLVKPSQTLSLTCTVSGGSISSGDYFWSWIRQLPGKGLEWGHIHNSGTTYYNPSLKSRVTISVDTSKKQFSLRLSSVTAADTAVYYCARDRGGDYYYGMDVWGQGTTVTVSS

SEQ ID NO: 91 - protein, 453 aa, Homo sapiens [sequence omitted]

SEQ ID NO: 92 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 93 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 94 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 95 - protein, 764 aa, Homo sapiens
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 96 - protein, 159 aa, Homo sapiens
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 97 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 98 - protein, 137 aa, Artificial Sequence
  Description of Artificial Sequence: Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 99 - protein, 5 aa, Artificial Sequence
  Description of Artificial Sequence: Synthetic peptide
GGGGS

SEQ ID NO: 100 - protein, 10 aa, Artificial Sequence
  Description of Artificial Sequence: Synthetic peptide
GGGGSGGGGS

SEQ ID NO: 101 - protein, 15 aa, Artificial Sequence
  Description of Artificial Sequence: Synthetic peptide
GGGGSGGGGSGGGGS

SEQ ID NO: 102 - protein, 20 aa, Artificial Sequence
  Description of Artificial Sequence: Synthetic peptide
GGGGSGGGGSGGGGSGGGGS

SEQ ID NO: 103 - protein, 25 aa, Artificial Sequence
  Description of Artificial Sequence: Synthetic peptide
GGGGSGGGGSGGGGSGGGGSGGGGS